(19) United States
(12) Patent Application Publication
(10) Pub. No.: US 2026/0159480 A1
(43) Pub. Date: Jun. 11, 2026

(54) IONIZABLE LIPID AND USE THEREOF

(71) Applicant: AXTER THERAPEUTICS (BEIJING) CO., LTD., Beijing (CN)

(72) Inventors: Yuanyuan ZHANG, Beijing (CN); Ke LI, Beijing (CN); Hanqing GU, Beijing (CN); Huicong ZHANG, Beijing (CN); Jin SONG, Beijing (CN); Huiya HUANG, Beijing (CN); Zengjun XU, Beijing (CN)

(73) Assignee: AXTER THERAPEUTICS (BEIJING) CO., LTD., Beijing (CN)

(21) Appl. No.: 19/434,032

(22) Filed: Dec. 29, 2025

Related U.S. Application Data
(63) Continuation-in-part of application No. PCT/CN2024/099640, filed on Jun. 17, 2024.

(30) Foreign Application Priority Data
Jun. 29, 2023 (CN) 202310778315.8

(51) Int. Cl. C07D 207/14; A61K 9/51; A61K 31/7088; A61K 47/68; A61K 47/69

(52) U.S. Cl. CPC C07D 207/14; A61K 9/5123; A61K 31/7088; A61K 47/6835; A61K 47/6929

(57) ABSTRACT

An ionizable lipid and a drug delivery system including the ionizable lipid are provided. Specifically, an ionizable lipid having the structure of formula (I), or a pharmaceutically acceptable salt, tautomer or stereoisomer thereof are provided. Lipid nanoparticles constructed by using the ionizable lipid can realize safe and efficient delivery of nucleic acid drugs, small molecule drugs, peptide drugs and protein drugs.

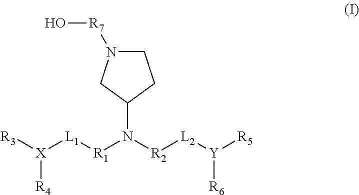

### CROSS REFERENCE TO THE RELATED APPLICATIONS

This application is a continuation-in-part application of International Application No. PCT/CN2024/099640, filed on Jun. 17, 2024, which is based upon and claims priority to Chinese Patent Application No. 202310778315.8, filed on Jun. 29, 2023, the entire contents of which are incorporated herein by reference.

### TECHNICAL FIELD

The present disclosure relates to the field of biomedicine, and specifically relates to an ionizable lipid and use thereof in drug delivery.

### BACKGROUND

In recent years, messenger RNA drugs have become a key therapeutic means for preventing and treating infectious diseases and tumors. The messenger RNA drug technology has been recognized by the industry. Benefiting from its short research and development cycle, low risk of insertional mutagenesis and diversity in encoding proteins. mRNA is highly suitable for the development of vaccines or therapeutic drugs. However, mRNA itself is highly unstable and susceptible to degradation by ubiquitous RNases. Additionally, due to the inherent negative charge and large molecular weight (typically greater than 10⁶ Da) of mRNA, mRNA molecules are restricted from entering cells. Therefore, the development of appropriate delivery carriers to protect fragile mRNA molecules and deliver them into the cytoplasm holds great significance.

Currently, a variety of mRNA delivery carriers have been developed, including lipid nanoparticles (LNPs), inorganic nanoparticles, polymeric nanoparticles, viral vectors and exosomes, etc. At present, LNPs are widely used as drug delivery carriers, and their main components include ionizable lipids, phospholipids, cholesterol and polyethylene glycol-containing lipids. The most important component in LNPs is the ionizable lipid. Early permanently positively charged cationic lipids exhibit short in vivo circulation time, high toxicity and severe allergic reactions, which is because their inherent positive charge causes them to adsorb proteins during in vivo circulation, making them liable to be captured and cleared by the reticuloendothelial system. The inherent positive charge interacts with negatively charged cell membranes, causing membrane destabilization and subsequent severe toxicity; permanently positively charged cationic lipids can activate the complement system, leading to allergic reactions. Ionizable lipids are electrically neutral under physiological pH conditions. Therefore, LNPs prepared from ionizable lipids exhibit relatively high safety. The ionizable lipid endows the LNP with lysosomal escape capability, and through the proton sponge effect and membrane fusion mechanism, enables the LNP to escape and release mRNA into the cytoplasm, where the mRNA binds to the protein-encoding ribosomes for translation of the encoded protein.

In short, the development of a suitable ionizable lipid is one of the keys for developing LNPs with high safety and high lysosomal escape efficiency.

Therefore, it is of great significance to develop an ionizable lipid with low toxicity and high delivery efficiency.

### SUMMARY

The present disclosure provides an ionizable lipid with low toxicity and high delivery efficiency.

In a first aspect of the present disclosure, provides an ionizable lipid, or a pharmaceutically acceptable salt, a tautomer or a stereoisomer thereof, wherein, the ionizable lipid has the structure of Formula I below:

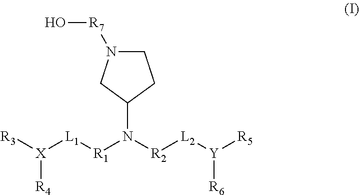

wherein,
R₁ and R₂ are each independently selected from —(CH₂)ₙ—, wherein n is a positive integer ranging from 1 to 14;
X and Y are each independently —CH— or N;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L₂b is —(CH₂)ₙ—, wherein n is selected from 0, 1, 2, 3 or 4;
R₃, R₄, R₅ and R₆ are each independently H, CH₃, C₂-C₃₀ hydrocarbyl group (e.g., C₂-C₃₀ alkyl, C₂-C₃₀ alkenyl, C₂-C₃₀ alkynyl), or —(CH₂)ₛ—R^{a}—(CH₂)_{g}—R^{b}—(CH₂)ₘ—CH₃, wherein, s, g are each independently selected from a positive integer ranging from 1 to 20, m is selected from a integer ranging from 0 to 20, preferably s+g+m is 2-35;
R^{a}, R^{b} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—;
and, R₃ and R₄ are not both H; and R₅ and R₆ are not both H;
R₇ is selected from C₁-C₅ hydrocarbyl group, or —(CH₂)ₘO(CH₂)ₙ—, wherein m, n are each independently selected from 1, 2, 3.

In another preferred embodiment, the R₃, R₄, R₅ and R₆ are each independently C₄-C₃₀ hydrocarbyl group (e.g., C₄-C₃₀ alkyl, C₄-C₃₀ alkenyl, C₄-C₃₀ alkynyl), preferably is C₄-C₂₀ hydrocarbyl group (e.g., C₄-C₂₀ alkyl, C₄-C₂₀ alkenyl, C₄-C₂₀ alkynyl).

In another preferred embodiment, at least 2, 3, or 4 of the R₃, R₄, R₅ and R₆ are C₂-C₃₀ hydrocarbyl group (e.g., C₂-C₃₀ alkyl, C₂-C₃₀ alkenyl, C₂-C₃₀ alkynyl), or —(CH₂)ₛ—R^{a}—(CH₂)_{g}—R^{b}—(CH₂)ₘ—CH₃, wherein s, g, m, R^{a} and R^{b} are defined as above.

In another preferred embodiment, the s+g+m is 3-20, more preferably is 4-15.

In another preferred embodiment, the R₃ has the structure of —R₃ₐ—R_{3b}—R_{3c}—R_{3d}—R₃ₑ, wherein, R₃ₐ and R_{3c} are each independently —(CH₂)ₙ—, wherein n is selected from a positive integer ranging from 1 to 14;
R_{3b} and R_{3d} are each independently absent or selected from the following functional groups: —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₃ₑ is C₂-C₂₀ hydrocarbyl group.

In another preferred embodiment, the R₄ has the structure of —R₄ₐ—R_{4b}—R_{4c}—R_{4d}—R₄ₑ;
wherein, R₄ₐ and R_{4c} are each independently —(CH₂)ₙ—, wherein n is selected from a positive integer ranging from 1 to 14;
R_{4b} and R_{4d} are each independently absent or selected from the following functional groups: —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C═C)—, preferably —(C═O)O—, —O(C═O)—, —(S—S)—, —(C═C)—(CH₂)—(C═C)—, —CH(OH)—;
R₄ₑ is C₂-C₂₀ hydrocarbyl group.

In another preferred embodiment, the R₅ has the structure of —R₅ₐ—R_{5b}—R_{5c}—R_{5d}—R₅ₑ,
wherein, R₅ₐ and R_{5c} are each independently —(CH₂)ₙ—, wherein n is selected from a positive integer ranging from 1 to 14;
R_{5b} and R_{5d} are each independently absent or selected from the following functional groups: O, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₅ₑ is C₂-C₂₀ hydrocarbyl group.

In another preferred embodiment, the R₆ has the structure of —R₆ₐ—R_{6b}—R_{6c}—R_{6d}—R₆ₑ;
wherein, R₆ₐ and R₆ₑ are each independently —(CH₂)ₙ—, wherein n is selected from a positive integer ranging from 1 to 14;
R_{6b} and R_{6d} are each independently absent or selected from the following functional groups: O, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₆ₑ is C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

In another preferred embodiment, the X and Y are —CH—.

In another preferred embodiment, the X and Y are N.

In another preferred embodiment, the ionizable lipid has the structure represented by the Formula below:

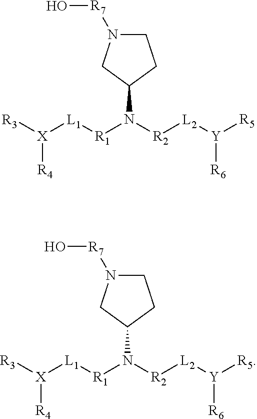

In another preferred embodiment, the ionizable lipid has the substructure represented by Formula (I-1) below:

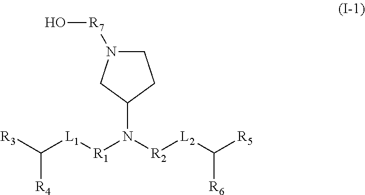

wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; preferably selected from —O—, —(C═O)O—, —O(C═O)—, —CH(OH)—; L₂b is —(CH₂)ₙ—, wherein n is selected from 0, 1, 2, 3 or 4;
R₃, R₄, R₅ and R₆ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3. In another preferred embodiment, the X is N, Y is —CH—.

In another preferred embodiment, the R₆ is H.

In another preferred embodiment, L₁ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—;
L₂ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—;
R₃, R₄, R₅ and R₆ are each independently selected from: H, C₂-C₂₀ hydrocarbyl group;
R₇ is selected form C₁-C₅ hydrocarbyl group.

In another preferred embodiment, n is selected from 1, 2, 3, 4, 5, 6, 7, 8, 9, 10, 11, 12, 13, 14.

In another preferred embodiment, n is selected from an integer ranging from 5 to 8, such as 5, 6, 7, 8.

In another preferred embodiment, L₁ is selected from: —(C═O)O—, —O(C═O)—, —(C═O)NH—, —NH(C═O)—.

In another preferred embodiment, L₂ is selected from: —(C═O)O—, —O(C═O)—, —(C═O)NH—, —NH(C═O)—.

In another preferred embodiment, R₇ is C₂-C₄ hydrocarbyl group.

In another preferred embodiment, the ionizable lipid has the structure represented by Formula (I-2) below:

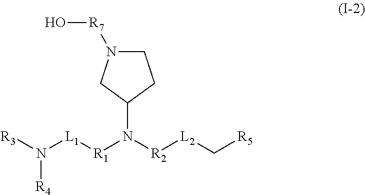

wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L₂b is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ, R₅ has the structure of —R₅ₐ—R_{5b}—R_{5c}—R_{5d}—R₅ₑ;
wherein, R₃ₐ, R_{3c}, R₄ₐ, R_{4c}, R₅ₐ, R_{5c} are each independently —(CH₂)ₙ—, wherein n is selected from a positive integer ranging from 1 to 14;
R_{3b}, R_{3d}, R_{4b}, R_{4d}, R_{5b}, R_{5d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₃ₑ, R₄ₑ, R₅ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

In another preferred embodiment, the ionizable lipid has the structure represented by Formula (I-3) below:

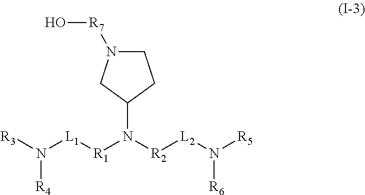

wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L₂b is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14;
R_{3b}, R_{3d}, R_{4b}, R_{4d}, R_{5b}, R_{5d}, R_{6b} and R_{6d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₃ₑ, R₄ₑ, R₅ₑ, R₆ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

In another preferred embodiment, the ionizable lipid has the structure represented by Formula (I-1), and in the Formula (I-1):
R₁ and R₂ are each independently selected from —(CH₂)ₙ—, wherein n is a positive integer ranging from 2 to 8;
L₁ is selected from the following functional groups: —(C═O)O—, —O(C═O)—;
L₂ is selected from the following functional groups: —(C═O)O—, —O(C═O)—;
R₃, R₄, R₅ and R₆ are each independently C₅-C₂₀ hydrocarbyl group;
R₇ is selected from C₁-C₅ hydrocarbyl group, or —(CH₂)ₘO(CH₂)ₙ—, wherein m, n are each independently selected from 1 or 2.

In another preferred embodiment, the ionizable lipid has the structure represented by Formula (I-2), and in the Formula (I-2):
R₁ and R₂ are each independently selected from —(CH₂)ₙ—, wherein n is a positive integer ranging from 2 to 8;
L₁ is absent or —O—(CH₂)ₙ—; wherein n is selected from 0, 1, 2, 3 or 4;
L₂ is selected from the following functional groups: —(C═O)O—, —O(C═O)—;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ;
wherein, R₃ₐ, R_{3c}, R₄ₐ, R_{4c}, R₅ₐ, R_{5c} are each independently —(CH₂)ₙ—, wherein n is selected from a positive integer ranging from 1 to 3;
R_{3b} and R_{4b} are each independently selected from the following group: —(C═O)O—, —O(C═O)—;
R_{3d}, R_{4d}, R_{5d} are each independently selected from the following group: —CH₂—, —(S—S)—;
R_{5b} is absent;
R₃ₑ, R₄ₑ, R₅ₑ are each independently C₂-C₁₅ hydrocarbyl group;
R₇ is selected from C₁-C₅ hydrocarbyl group, or —(CH₂)ₘO(CH₂)ₙ—, wherein m, n are each independently selected from 1 or 2.

In another preferred embodiment, the ionizable lipid has the structure represented by Formula (I-1), and in the Formula (I-1):
R₁ and R₂ are each independently selected from —(CH₂)ₙ—, wherein n a positive integer ranging from 2 to 8;
L₁ is selected from the following functional groups: —NH(C═O)—, —(C═O)NH—;
L₂ is selected from the following functional groups: —NH(C═O)—, —(C═O)NH—;
R₃, R₄, R₅ and R₆ are each independently C₅-C₂₀ hydrocarbyl group;
R₇ is selected from C₁-C₅ hydrocarbyl group, or —(CH₂)ₘO(CH₂)ₙ—, wherein m, n are each independently selected from 1 or 2.

In another preferred embodiment, the ionizable lipid has the structure represented by Formula (I-2), and in the Formula (I-2):
R₁ and R₂ are each independently selected from —(CH₂)ₙ—, wherein n is a positive integer ranging from 2 to 8;
L₁ is selected from the following functional groups: —(C═O)S—, —S(C═O)—;
L₂ is selected from the following functional groups: —(C═O)S—, —S(C═O)—;
R₃, R₄, R₅ and R₆ are each independently C₅-C₂₀ hydrocarbyl group;
R₇ is selected from C₁-C₅ hydrocarbyl group, or —(CH₂)ₘO(CH₂)ₙ—, wherein m, n are each independently selected from 1 or 2.

In another preferred embodiment, the ionizable lipid has the structure represented by Formula (I-3), and in the Formula (I-3):
R₁ and R₂ are each independently selected from —(CH₂)ₙ—, wherein n is a positive integer ranging from 2 to 8;
L₁ is absent or —O—(CH₂)ₙ—; wherein n is selected from 0, 1, 2, 3 or 4;
L₂ is absent or —O—(CH₂)ₙ—; wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ; R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ, R_{6c} are each independently —(CH₂)ₙ—, wherein n is selected from a positive integer ranging from 1 to 3;
R_{3b}, R_{4b}, R_{5b} and R_{6b} are each independently selected from the following group: —(C═O)O—, —O(C═O)—, —CH(OH)—;
R_{3d}, R_{4d}, R_{5d} and R_{6d} are each independently selected from the following group: —CH₂—, —(S—S)—;
R₃ₑ, R₄ₑ, R₅ₑ and R₆ₑ are each independently C₂-C₁₅ hydrocarbyl group;
R₇ is selected from C₁-C₅ hydrocarbyl group, or —(CH₂)ₘO(CH₂)ₙ—, wherein m, n are each independently selected from 1 or 2.

In another preferred embodiment, R₃, R₄, R₅ and R₆ are each independently selected from the following group: C₅-C₁₅ alkyl, C₅-C₁₅ alkenyl, C₅-C₁₅ alkynyl;
R₇ is selected from C₁-C₅ alkyl, —(CH₂)O(CH₂)—, or —(CH₂)₂O(CH₂)₂—, wherein m, n are each independently selected from 1, 2, 3.

In another preferred embodiment, the ionizable lipid has the structure selected from Table 1 below:

TABLE 1

| Serial number | Structural formula |
|---|---|
| AXT-1 | 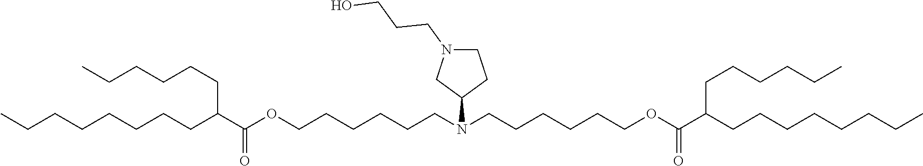 [SMILES: CCCCCCCCC(CCCCCC)C(=O)OCCCCCCN(CCCCCCOC(=O)C(CCCCCC)CCCCCCCC)[C@@H]1CCN(CCCO)C1] |
| AXT-2 | 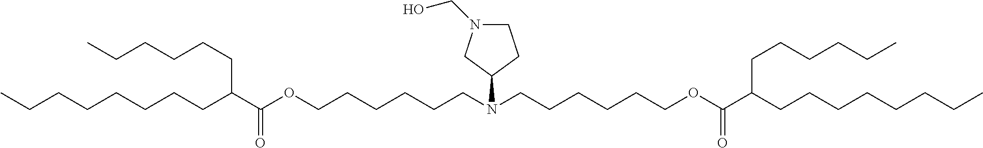 [SMILES: CCCCCCCCC(CCCCCC)C(=O)OCCCCCCN(CCCCCCOC(=O)C(CCCCCC)CCCCCCCC)[C@@H]1CCN(CO)C1] |
| AXT-3 | 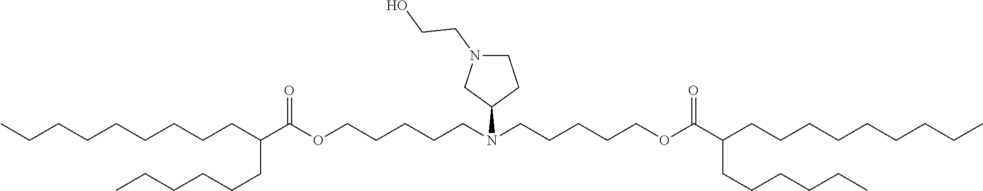 [SMILES: CCCCCCCCCC(CCCCCC)C(=O)OCCCCCN(CCCCCOC(=O)C(CCCCCC)CCCCCCCCC)[C@@H]1CCN(CCO)C1] |
| AXT-4 | 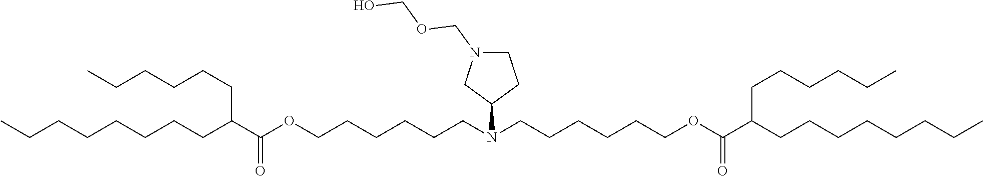 [SMILES: CCCCCCCCC(CCCCCC)C(=O)OCCCCCCN(CCCCCCOC(=O)C(CCCCCC)CCCCCCCC)[C@@H]1CCN(COCO)C1] |
| AXT-5 | 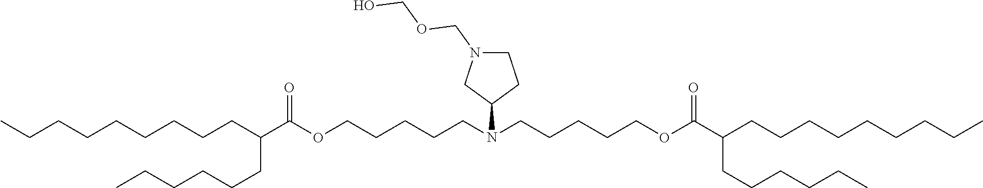 [SMILES: CCCCCCCCCC(CCCCCC)C(=O)OCCCCCN(CCCCCOC(=O)C(CCCCCC)CCCCCCCCC)[C@@H]1CCN(COCO)C1] |
| AXT-6 | 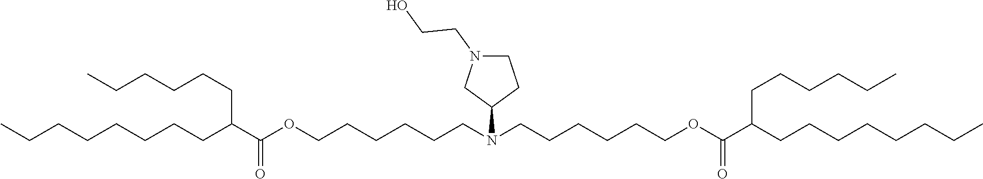 [SMILES: CCCCCCCCC(CCCCCC)C(=O)OCCCCCCN(CCCCCCOC(=O)C(CCCCCC)CCCCCCCC)[C@@H]1CCN(CCO)C1] |
| AXT-7 | 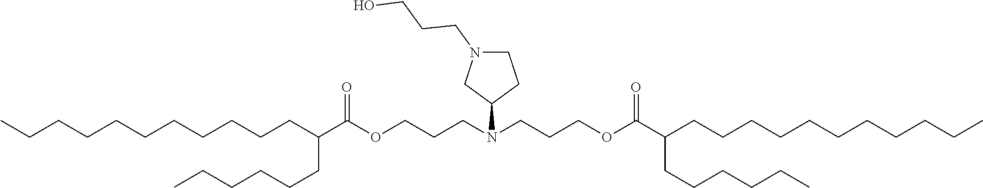 [SMILES: CCCCCCCCCCCC(CCCCCC)C(=O)OCCCN(CCCOC(=O)C(CCCCCC)CCCCCCCCCCC)[C@@H]1CCN(CCCO)C1] |
| AXT-8 | 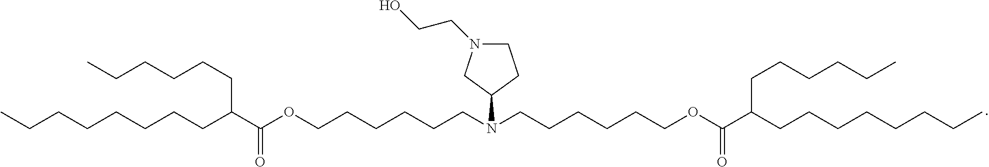 [SMILES: CCCCCCCCC(CCCCCC)C(=O)OCCCCCCN(CCCCCCOC(=O)C(CCCCCC)CCCCCCCC)[C@@H]1CCN(CCO)C1] |
| AXT-9 | 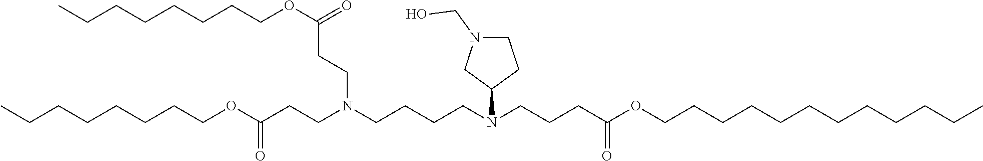 [SMILES: CCCCCCCCCCCCOC(=O)CCCN(CCCCN(CCC(=O)OCCCCCCCC)CCC(=O)OCCCCCCCC)[C@@H]1CCN(CO)C1] |
| AXT-10 | 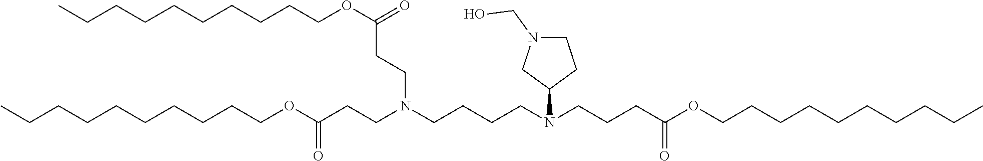 [SMILES: CCCCCCCCCCOC(=O)CCCN(CCCCN(CCC(=O)OCCCCCCCCCC)CCC(=O)OCCCCCCCCCC)[C@@H]1CCN(CO)C1] |
| AXT-11 | 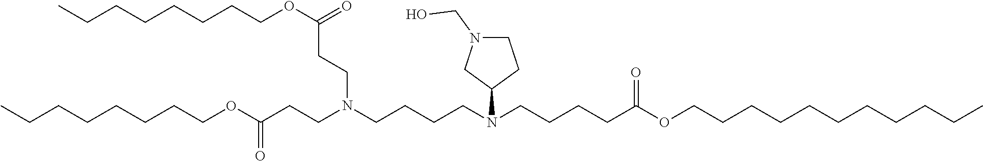 [SMILES: CCCCCCCCCCCOC(=O)CCCCN(CCCCN(CCC(=O)OCCCCCCCC)CCC(=O)OCCCCCCCC)[C@@H]1CCN(CO)C1] |
| AXT-12 | 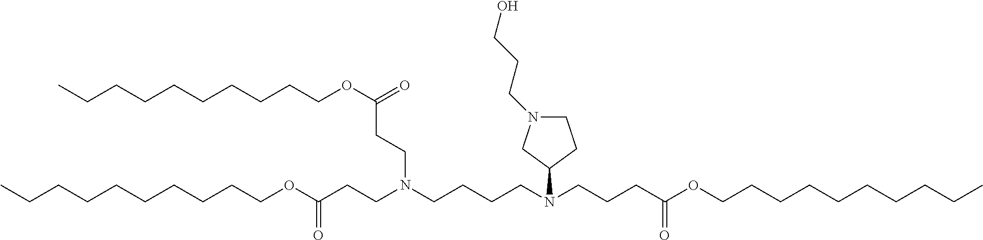 [SMILES: CCCCCCCCCCOC(=O)CCCN(CCCCN(CCC(=O)OCCCCCCCCCC)CCC(=O)OCCCCCCCCCC)[C@@H]1CCN(CCCO)C1] |
| AXT-13 | 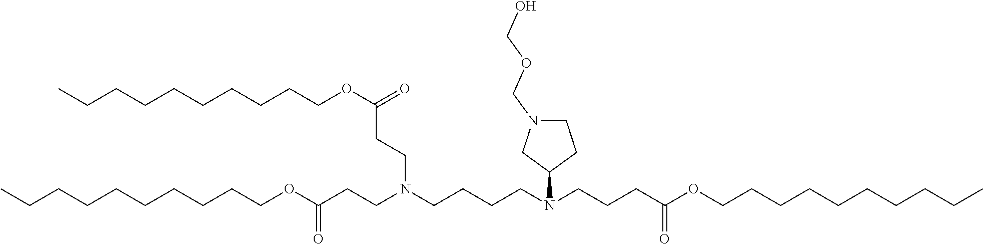 [SMILES: CCCCCCCCCCOC(=O)CCCN(CCCCN(CCC(=O)OCCCCCCCCCC)CCC(=O)OCCCCCCCCCC)[C@@H]1CCN(COCO)C1] |
| AXT-14 | 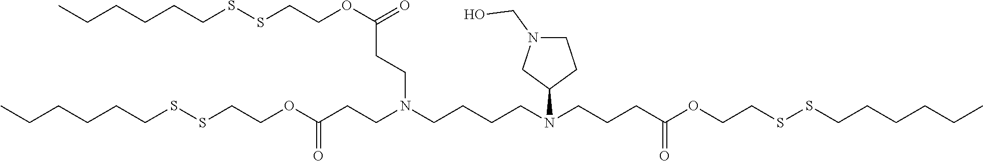 [SMILES: CCCCCCSSCCOC(=O)CCCN(CCCCN(CCC(=O)OCCSSCCCCCC)CCC(=O)OCCSSCCCCCC)[C@@H]1CCN(CO)C1] |
| AXT-15 | 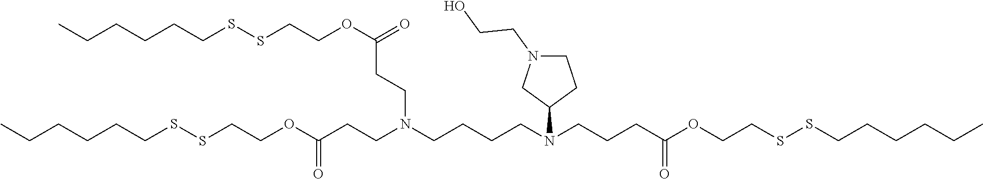 [SMILES: CCCCCCSSCCOC(=O)CCCN(CCCCN(CCC(=O)OCCSSCCCCCC)CCC(=O)OCCSSCCCCCC)[C@@H]1CCN(CCO)C1] |
| AXT-16 | 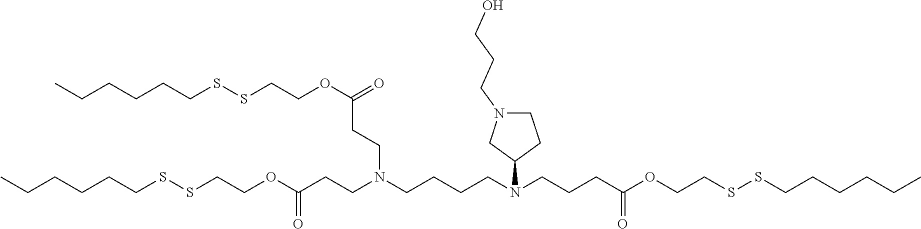 [SMILES: CCCCCCSSCCOC(=O)CCCN(CCCCN(CCC(=O)OCCSSCCCCCC)CCC(=O)OCCSSCCCCCC)[C@@H]1CCN(CCCO)C1] |
| AXT-17 | 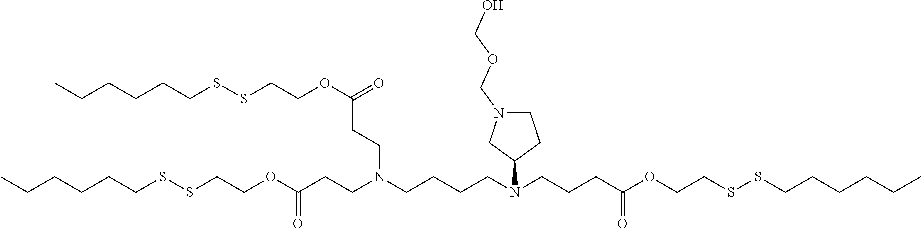 [SMILES: CCCCCCSSCCOC(=O)CCCN(CCCCN(CCC(=O)OCCSSCCCCCC)CCC(=O)OCCSSCCCCCC)[C@@H]1CCN(COCO)C1] |
| AXT-18 | 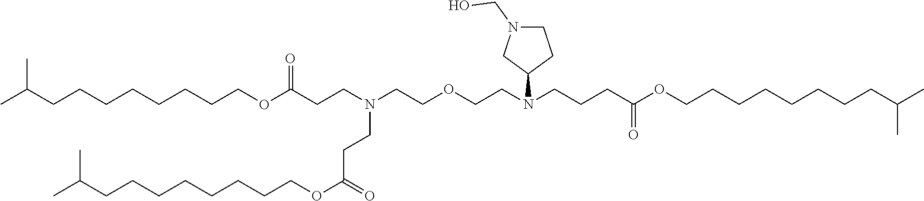 [SMILES: CC(C)CCCCCCCCOC(=O)CCCN(CCOCCN(CCC(=O)OCCCCCCCCC(C)C)CCC(=O)OCCCCCCCCC(C)C)[C@@H]1CCN(CO)C1] |
| AXT-19 | 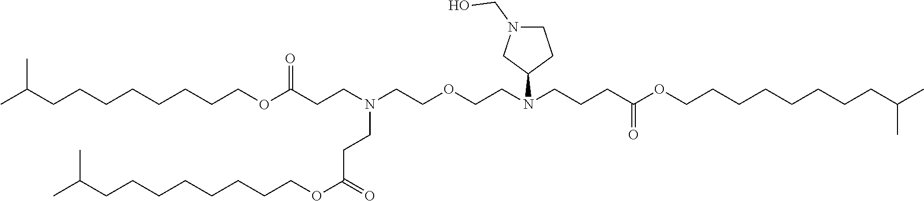 [SMILES: CC(C)CCCCCCCCOC(=O)CCCN(CCOCCN(CCC(=O)OCCCCCCCCC(C)C)CCC(=O)OCCCCCCCCC(C)C)[C@@H]1CCN(CO)C1] |
| AXT-20 | 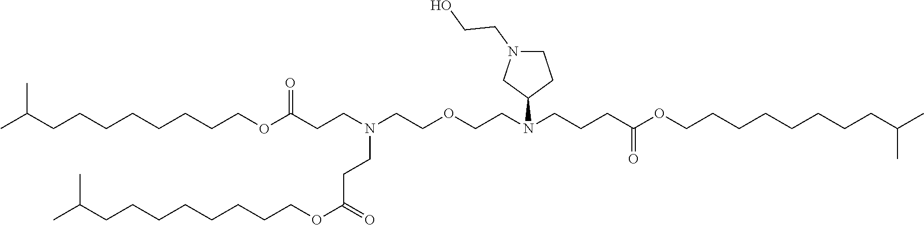 [SMILES: CC(C)CCCCCCCCOC(=O)CCCN(CCOCCN(CCC(=O)OCCCCCCCCC(C)C)CCC(=O)OCCCCCCCCC(C)C)[C@@H]1CCN(CCO)C1] |
| AXT-21 | 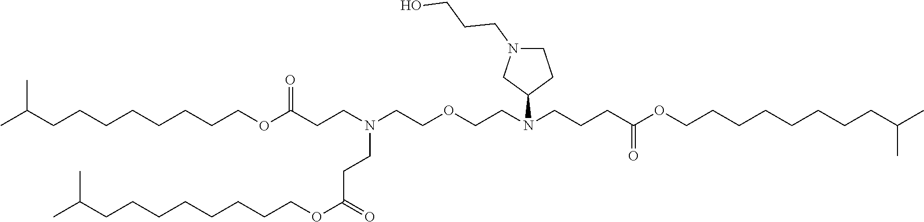 [SMILES: CC(C)CCCCCCCCOC(=O)CCCN(CCOCCN(CCC(=O)OCCCCCCCCC(C)C)CCC(=O)OCCCCCCCCC(C)C)[C@@H]1CCN(CCCO)C1] |
| AXT-22 | 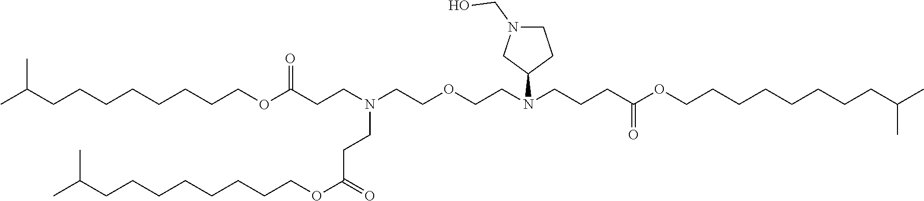 [SMILES: CC(C)CCCCCCCCOC(=O)CCCN(CCOCCN(CCC(=O)OCCCCCCCCC(C)C)CCC(=O)OCCCCCCCCC(C)C)[C@@H]1CCN(CO)C1] |
| AXT-23 | 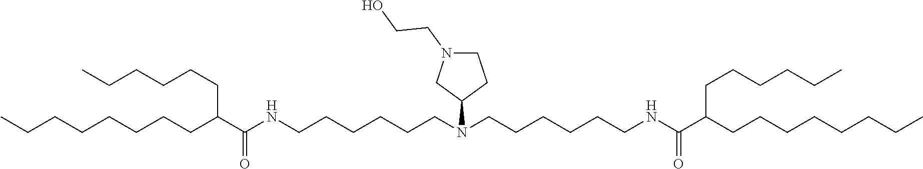 [SMILES: CCCCCCCCC(CCCCCC)C(=O)NCCCCCCN(CCCCCCNC(=O)C(CCCCCC)CCCCCCCC)[C@@H]1CCN(CCO)C1] |
| AXT-24 | 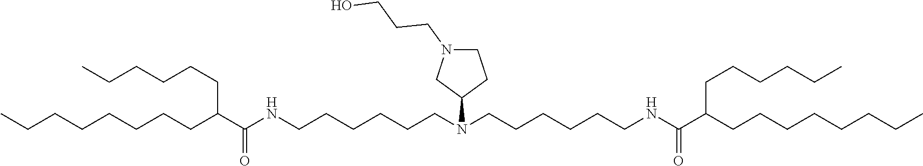 [SMILES: CCCCCCCCC(CCCCCC)C(=O)NCCCCCCN(CCCCCCNC(=O)C(CCCCCC)CCCCCCCC)[C@@H]1CCN(CCCO)C1] |
| AXT-25 | 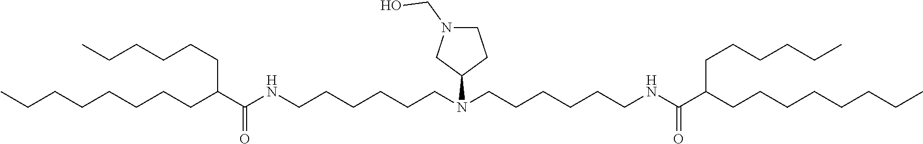 [SMILES: CCCCCCCCC(CCCCCC)C(=O)NCCCCCCN(CCCCCCNC(=O)C(CCCCCC)CCCCCCCC)[C@@H]1CCN(CO)C1] |
| AXT-26 | 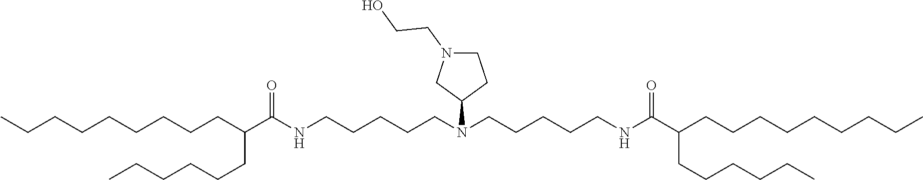 [SMILES: CCCCCCCCCC(CCCCCC)C(=O)NCCCCCN(CCCCCNC(=O)C(CCCCCC)CCCCCCCCC)[C@@H]1CCN(CCO)C1] |
| AXT-27 | 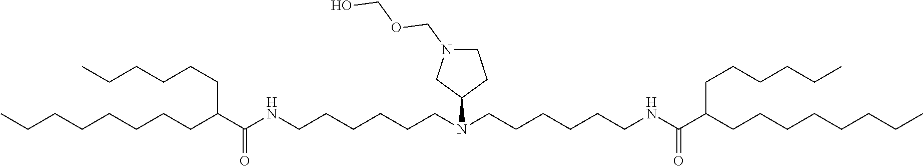 [SMILES: CCCCCCCCC(CCCCCC)C(=O)NCCCCCCN(CCCCCCNC(=O)C(CCCCCC)CCCCCCCC)[C@@H]1CCN(COCO)C1] |
| AXT-28 | 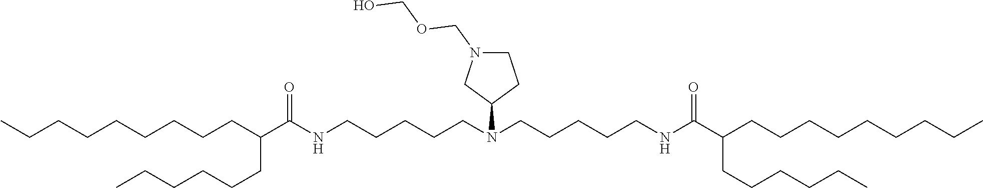 [SMILES: CCCCCCCCCC(CCCCCC)C(=O)NCCCCCN(CCCCCNC(=O)C(CCCCCC)CCCCCCCCC)[C@@H]1CCN(COCO)C1] |
| AXT-29 | 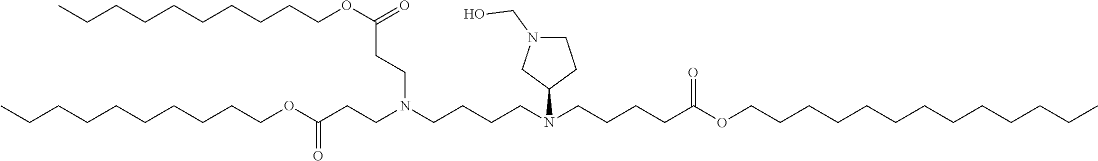 [SMILES: CCCCCCCCCCCCCOC(=O)CCCCN(CCCCN(CCC(=O)OCCCCCCCCCC)CCC(=O)OCCCCCCCCCC)[C@@H]1CCN(CO)C1] |
| AXT-30 | 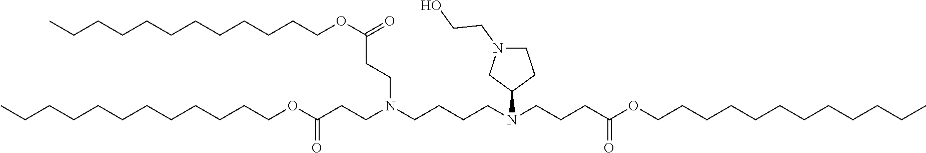 [SMILES: CCCCCCCCCCCCOC(=O)CCCN(CCCCN(CCC(=O)OCCCCCCCCCCCC)CCC(=O)OCCCCCCCCCCCC)[C@@H]1CCN(CCO)C1] |
| AXT-31 | 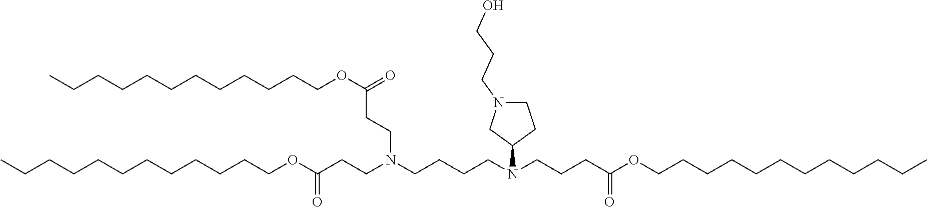 [SMILES: CCCCCCCCCCCCOC(=O)CCCN(CCCCN(CCC(=O)OCCCCCCCCCCCC)CCC(=O)OCCCCCCCCCCCC)[C@@H]1CCN(CCCO)C1] |
| AXT-32 | 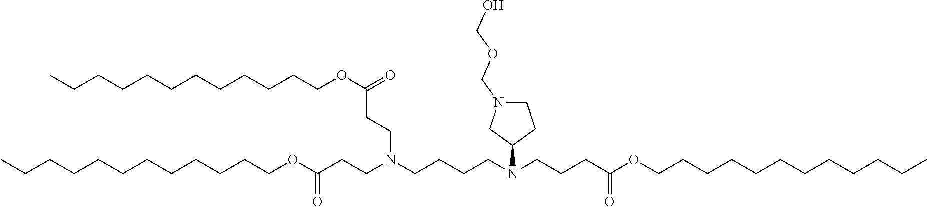 [SMILES: CCCCCCCCCCCCOC(=O)CCCN(CCCCN(CCC(=O)OCCCCCCCCCCCC)CCC(=O)OCCCCCCCCCCCC)[C@@H]1CCN(COCO)C1] |
| AXT-33 | 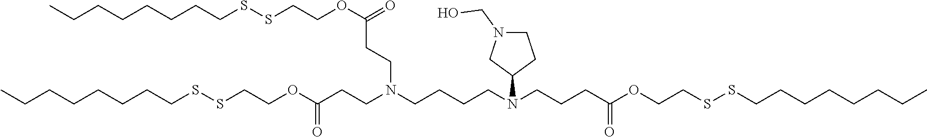 [SMILES: CCCCCCCCSSCCOC(=O)CCCN(CCCCN(CCC(=O)OCCSSCCCCCCCC)CCC(=O)OCCSSCCCCCCCC)[C@@H]1CCN(CO)C1] |
| AXT-34 | 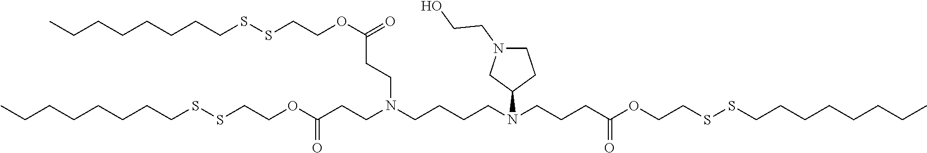 [SMILES: CCCCCCCCSSCCOC(=O)CCCN(CCCCN(CCC(=O)OCCSSCCCCCCCC)CCC(=O)OCCSSCCCCCCCC)[C@@H]1CCN(CCO)C1] |
| AXT-35 | 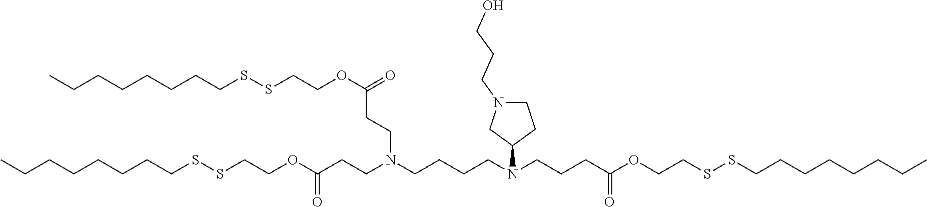 [SMILES: CCCCCCCCSSCCOC(=O)CCCN(CCCCN(CCC(=O)OCCSSCCCCCCCC)CCC(=O)OCCSSCCCCCCCC)[C@@H]1CCN(CCCO)C1] |
| AXT-36 | 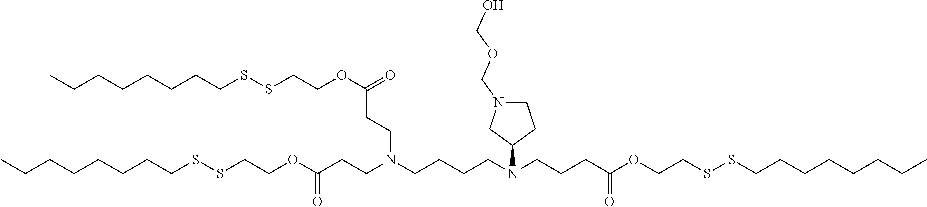 [SMILES: CCCCCCCCSSCCOC(=O)CCCN(CCCCN(CCC(=O)OCCSSCCCCCCCC)CCC(=O)OCCSSCCCCCCCC)[C@@H]1CCN(COCO)C1] |
| AXT-37 | 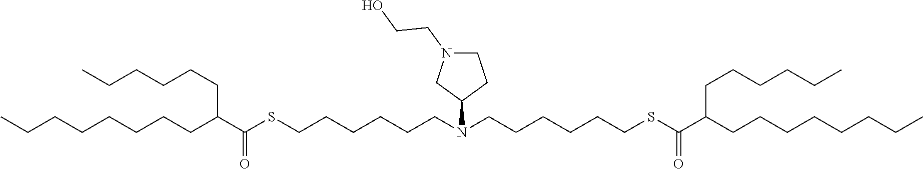 [SMILES: CCCCCCCCC(CCCCCC)C(=O)SCCCCCCN(CCCCCCSC(=O)C(CCCCCC)CCCCCCCC)[C@@H]1CCN(CCO)C1] |
| AXT-38 | 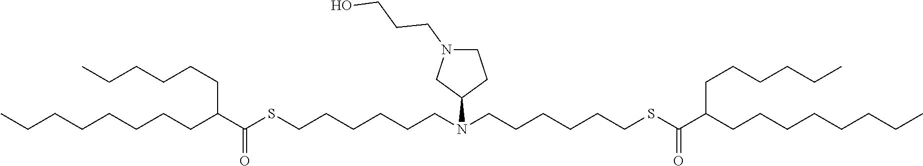 [SMILES: CCCCCCCCC(CCCCCC)C(=O)SCCCCCCN(CCCCCCSC(=O)C(CCCCCC)CCCCCCCC)[C@@H]1CCN(CCCO)C1] |
| AXT-39 | 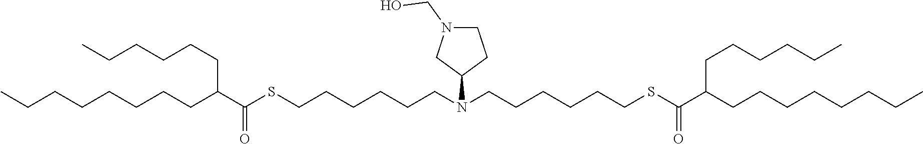 [SMILES: CCCCCCCCC(CCCCCC)C(=O)SCCCCCCN(CCCCCCSC(=O)C(CCCCCC)CCCCCCCC)[C@@H]1CCN(CO)C1] |
| AXT-40 | 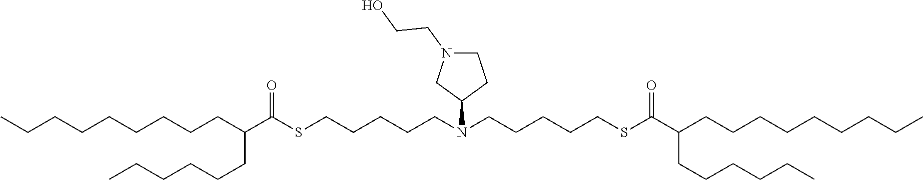 [SMILES: CCCCCCCCCC(CCCCCC)C(=O)SCCCCCN(CCCCCSC(=O)C(CCCCCC)CCCCCCCCC)[C@@H]1CCN(CCO)C1] |
| AXT-41 | 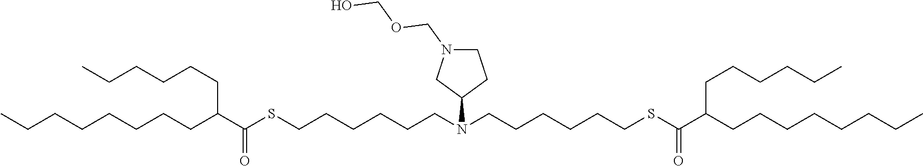 [SMILES: CCCCCCCCC(CCCCCC)C(=O)SCCCCCCN(CCCCCCSC(=O)C(CCCCCC)CCCCCCCC)[C@@H]1CCN(COCO)C1] |
| AXT-42 | 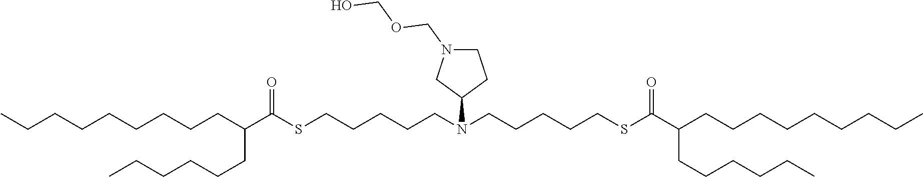 [SMILES: CCCCCCCCCC(CCCCCC)C(=O)SCCCCCN(CCCCCSC(=O)C(CCCCCC)CCCCCCCCC)[C@@H]1CCN(COCO)C1] |
| AXT-43 | 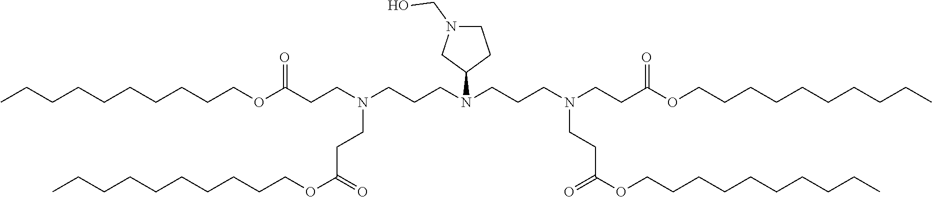 [SMILES: CCCCCCCCCCOC(=O)CCN(CCCN(CCCN(CCC(=O)OCCCCCCCCCC)CCC(=O)OCCCCCCCCCC)[C@@H]1CCN(CO)C1)CCC(=O)OCCCCCCCCCC] |
| AXT-44 | 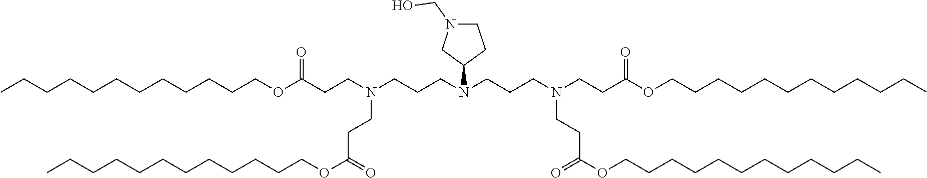 [SMILES: CCCCCCCCCCCCOC(=O)CCN(CCCN(CCCN(CCC(=O)OCCCCCCCCCCCC)CCC(=O)OCCCCCCCCCCCC)[C@@H]1CCN(CO)C1)CCC(=O)OCCCCCCCCCCCC] |
| AXT-45 | 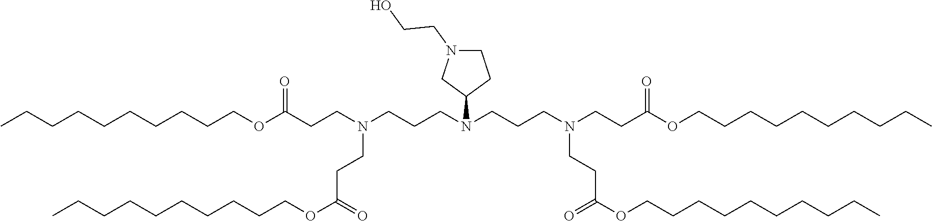 [SMILES: CCCCCCCCCCOC(=O)CCN(CCCN(CCCN(CCC(=O)OCCCCCCCCCC)CCC(=O)OCCCCCCCCCC)[C@@H]1CCN(CCO)C1)CCC(=O)OCCCCCCCCCC] |
| AXT-46 | 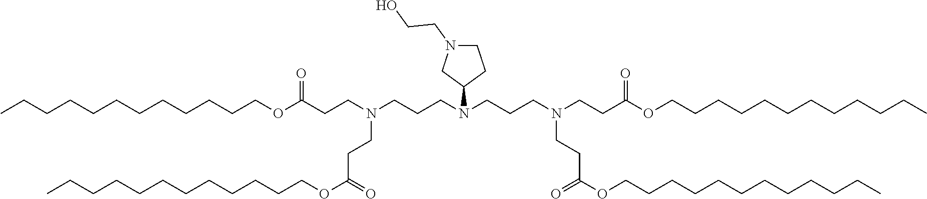 [SMILES: CCCCCCCCCCCCOC(=O)CCN(CCCN(CCCN(CCC(=O)OCCCCCCCCCCCC)CCC(=O)OCCCCCCCCCCCC)[C@@H]1CCN(CCO)C1)CCC(=O)OCCCCCCCCCCCC] |
| AXT-47 | 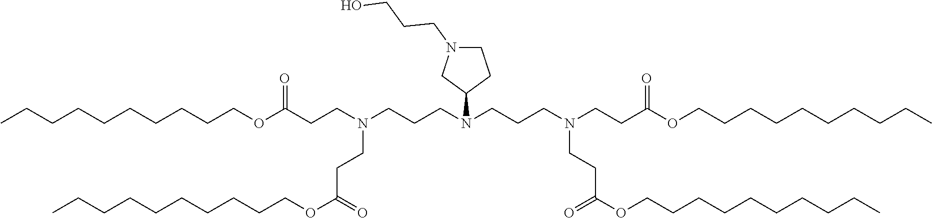 [SMILES: CCCCCCCCCCOC(=O)CCN(CCCN(CCCN(CCC(=O)OCCCCCCCCCC)CCC(=O)OCCCCCCCCCC)[C@@H]1CCN(CCCO)C1)CCC(=O)OCCCCCCCCCC] |
| AXT-48 | 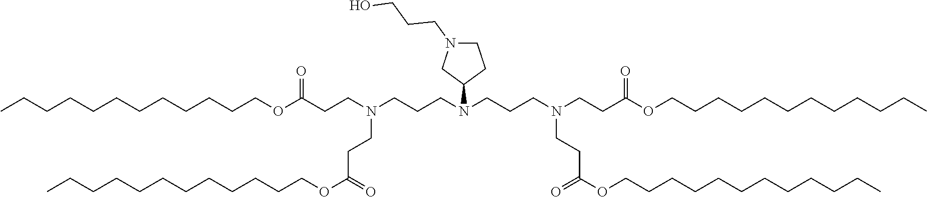 [SMILES: CCCCCCCCCCCCOC(=O)CCN(CCCN(CCCN(CCC(=O)OCCCCCCCCCCCC)CCC(=O)OCCCCCCCCCCCC)[C@@H]1CCN(CCCO)C1)CCC(=O)OCCCCCCCCCCCC] |
| AXT-49 | 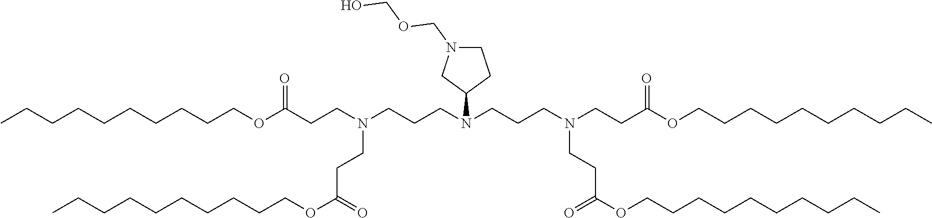 [SMILES: CCCCCCCCCCOC(=O)CCN(CCCN(CCCN(CCC(=O)OCCCCCCCCCC)CCC(=O)OCCCCCCCCCC)[C@@H]1CCN(COCO)C1)CCC(=O)OCCCCCCCCCC] |
| AXT-50 | 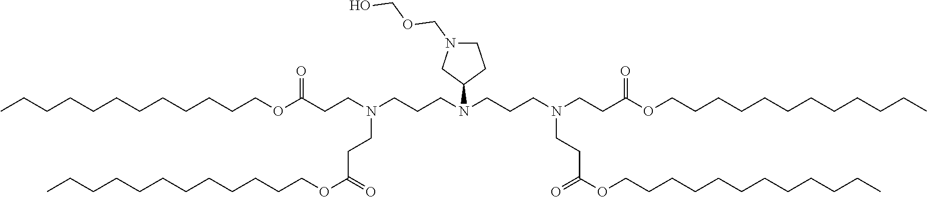 [SMILES: CCCCCCCCCCCCOC(=O)CCN(CCCN(CCCN(CCC(=O)OCCCCCCCCCCCC)CCC(=O)OCCCCCCCCCCCC)[C@@H]1CCN(COCO)C1)CCC(=O)OCCCCCCCCCCCC] |
| AXT-51 | 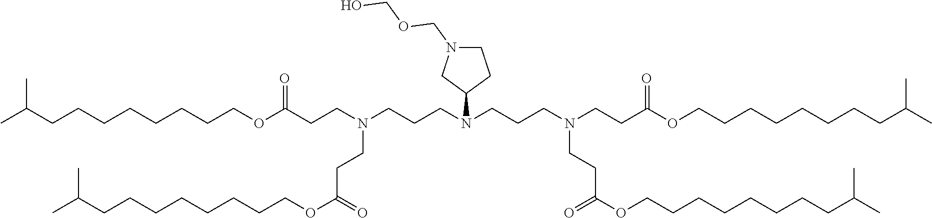 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(COCO)C1)CCC(=O)OCCCCCCCCC(C)C] |
| AXT-52 | 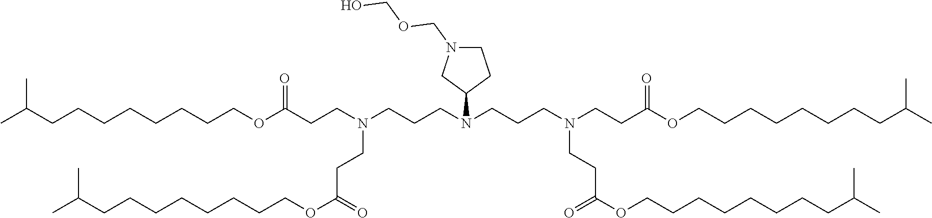 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(COCO)C1)CCC(=O)OCCCCCCCCC(C)C] |
| AXT-53 | 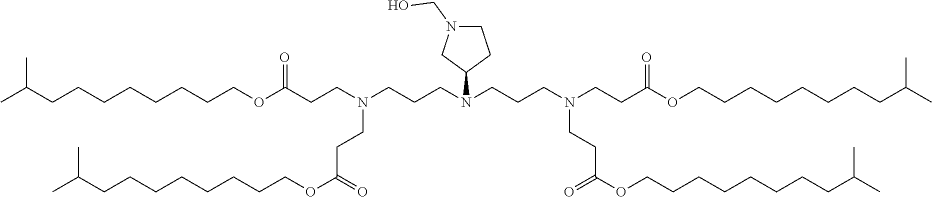 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CO)C1)CCC(=O)OCCCCCCCCC(C)C] |
| AXT-54 | 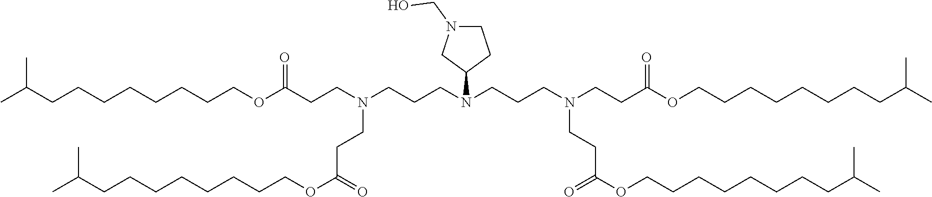 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CO)C1)CCC(=O)OCCCCCCCCC(C)C] |
| AXT-55 | 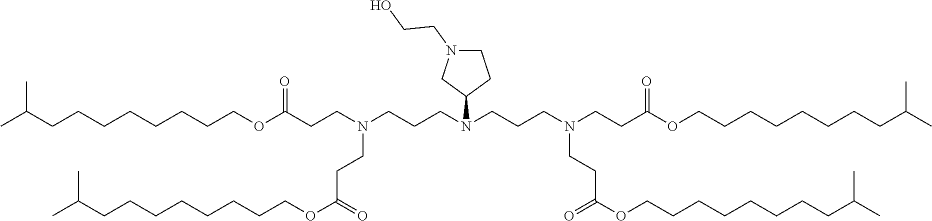 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CCO)C1)CCC(=O)OCCCCCCCCC(C)C] |
| AXT-56 | 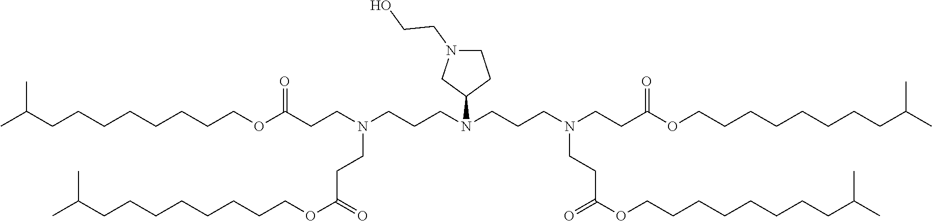 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CCO)C1)CCC(=O)OCCCCCCCCC(C)C] |
| AXT-57 | 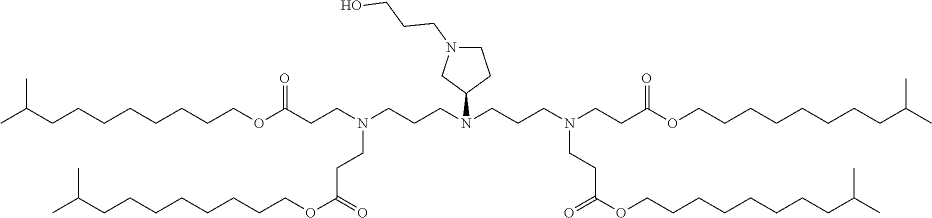 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CCCO)C1)CCC(=O)OCCCCCCCCC(C)C] |
| AXT-58 | 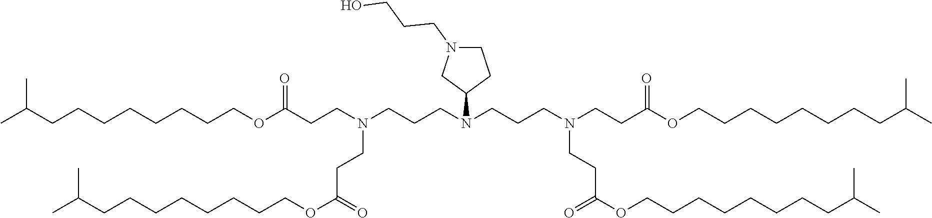 [SMILES: CC(C)CCCCCCCCOC(=O)CCN(CCCN(CCCN(CCC(=O)OCCCCCCCCC(C)C)CCC(=O)OCCCCCCCCC(C)C)[C@@H]1CCN(CCCO)C1)CCC(=O)OCCCCCCCCC(C)C] |
| AXT-59 | 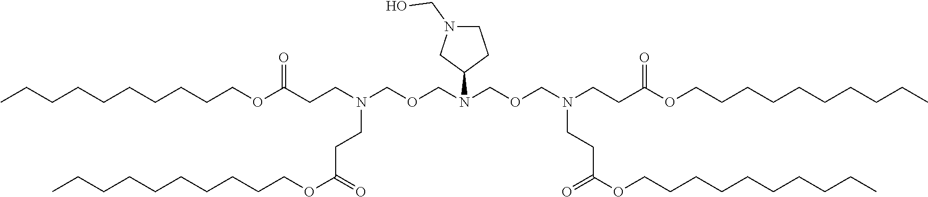 [SMILES: CCCCCCCCCCOC(=O)CCN(CCC(=O)OCCCCCCCCCC)COCN(COCN(CCC(=O)OCCCCCCCCCC)CCC(=O)OCCCCCCCCCC)[C@@H]1CCN(CO)C1] |
| AXT-60 | 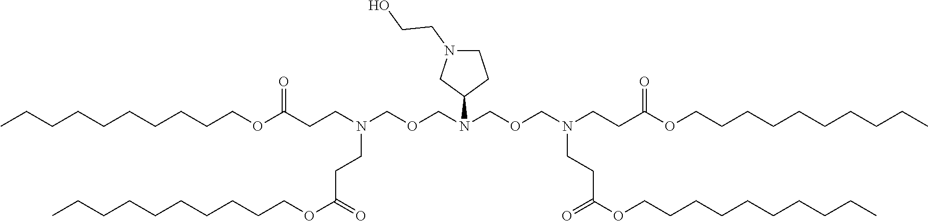 [SMILES: CCCCCCCCCCOC(=O)CCN(CCC(=O)OCCCCCCCCCC)COCN(COCN(CCC(=O)OCCCCCCCCCC)CCC(=O)OCCCCCCCCCC)[C@@H]1CCN(CCO)C1] |
| AXT-61 | 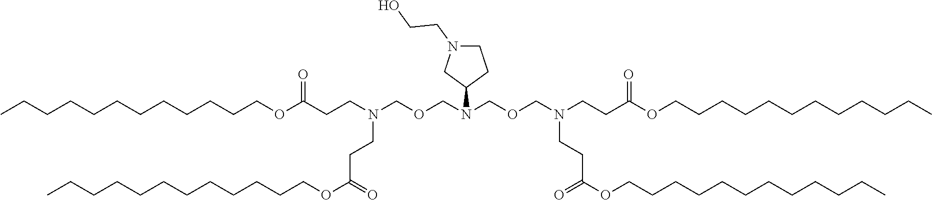 [SMILES: CCCCCCCCCCCCOC(=O)CCN(CCC(=O)OCCCCCCCCCCCC)COCN(COCN(CCC(=O)OCCCCCCCCCCCC)CCC(=O)OCCCCCCCCCCCC)[C@@H]1CCN(CCO)C1] |
| AXT-62 | 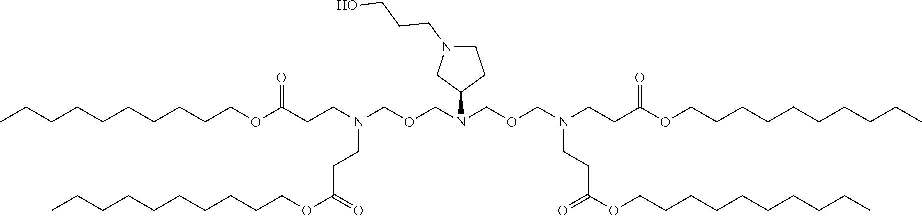 [SMILES: CCCCCCCCCCOC(=O)CCN(CCC(=O)OCCCCCCCCCC)COCN(COCN(CCC(=O)OCCCCCCCCCC)CCC(=O)OCCCCCCCCCC)[C@@H]1CCN(CCCO)C1] |
| AXT-63 | 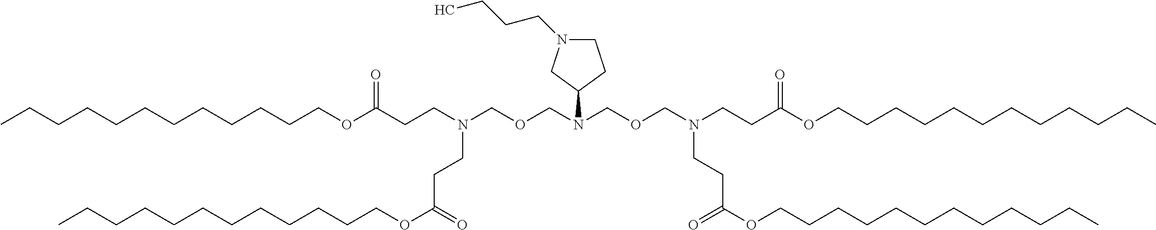 [SMILES: CCCCCCCCCCCCOC(=O)CCN(CCC(=O)OCCCCCCCCCCCC)COCN(COCN(CCC(=O)OCCCCCCCCCCCC)CCC(=O)OCCCCCCCCCCCC)[C@@H]1CCN(CCCC)C1] |
| AXT-64 | 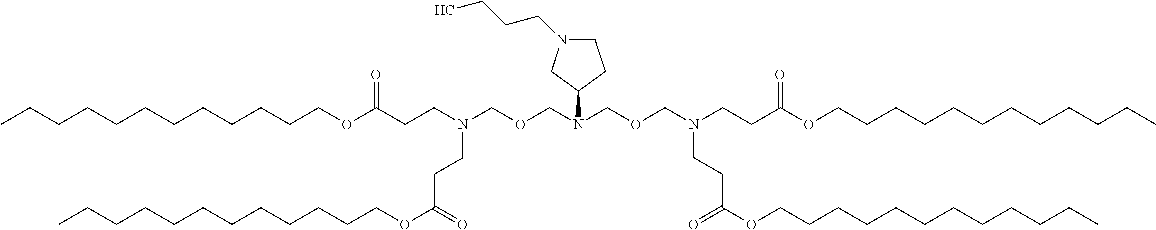 [SMILES: CCCCCCCCCCCCOC(=O)CCN(CCC(=O)OCCCCCCCCCCCC)COCN(COCN(CCC(=O)OCCCCCCCCCCCC)CCC(=O)OCCCCCCCCCCCC)[C@@H]1CCN(CCCC)C1] |
| AXT-65 | 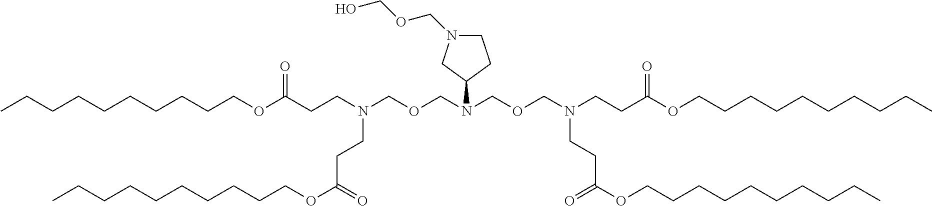 [SMILES: CCCCCCCCCCOC(=O)CCN(CCC(=O)OCCCCCCCCCC)COCN(COCN(CCC(=O)OCCCCCCCCCC)CCC(=O)OCCCCCCCCCC)[C@@H]1CCN(COCO)C1] |
| AXT-66 | 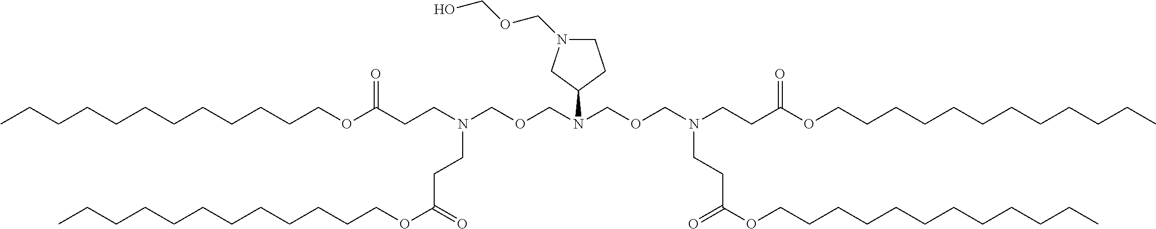 [SMILES: CCCCCCCCCCCCOC(=O)CCN(CCC(=O)OCCCCCCCCCCCC)COCN(COCN(CCC(=O)OCCCCCCCCCCCC)CCC(=O)OCCCCCCCCCCCC)[C@@H]1CCN(COCO)C1] |
| AXT-67 | 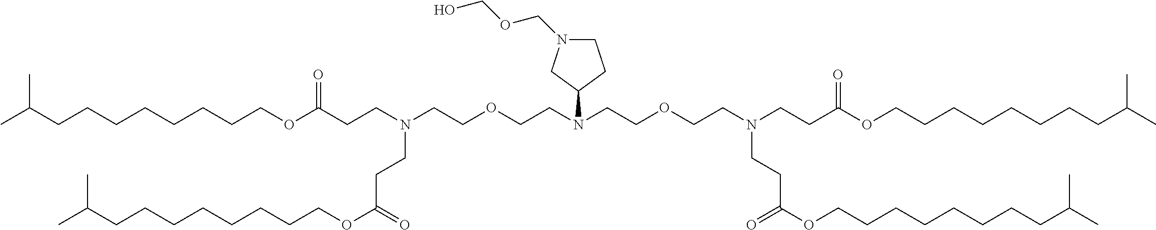 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@@H]1CCN(COCO)C1)CCC(=O)OCCCCCCCCC(C)C] |
| AXT-68 | 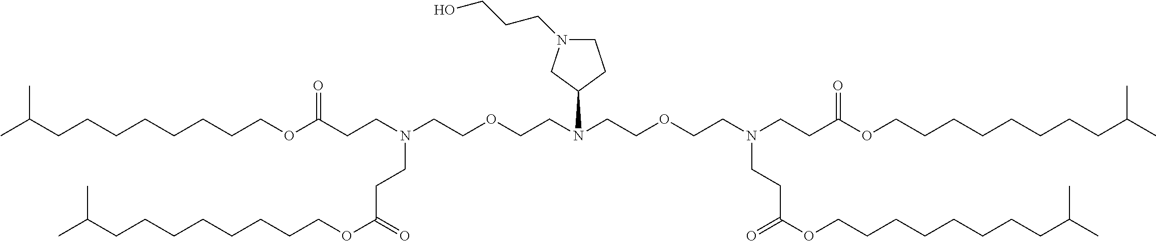 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@@H]1CCN(CCCO)C1)CCC(=O)OCCCCCCCCC(C)C] |
| AXT-69 | 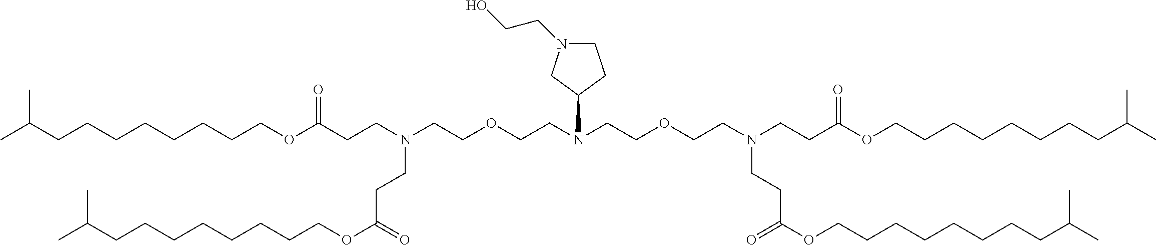 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@@H]1CCN(CCO)C1)CCC(=O)OCCCCCCCCC(C)C] |
| AXT-70 | 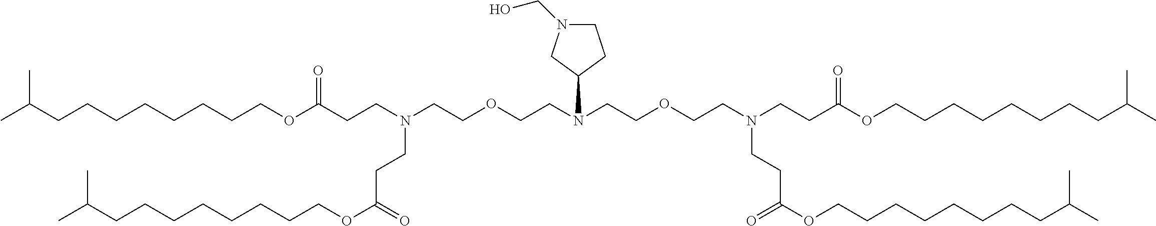 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@@H]1CCN(CO)C1)CCC(=O)OCCCCCCCCC(C)C] |
| AXT-71 | 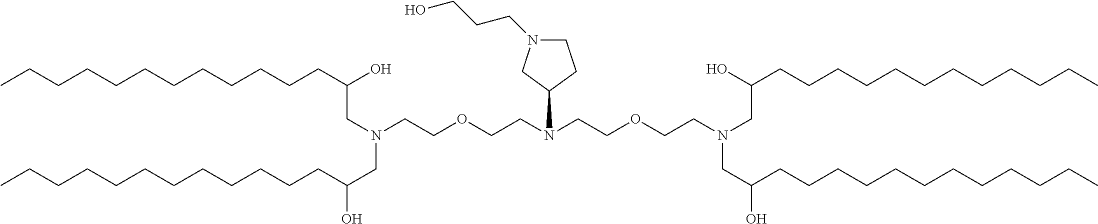 [SMILES: CCCCCCCCCCCCC(O)CN(CCOCCN(CCOCCN(CC(O)CCCCCCCCCCCC)CC(O)CCCCCCCCCCCC)[C@@H]1CCN(CCCO)C1)CC(O)CCCCCCCCCCCC] |
| AXT-72 | 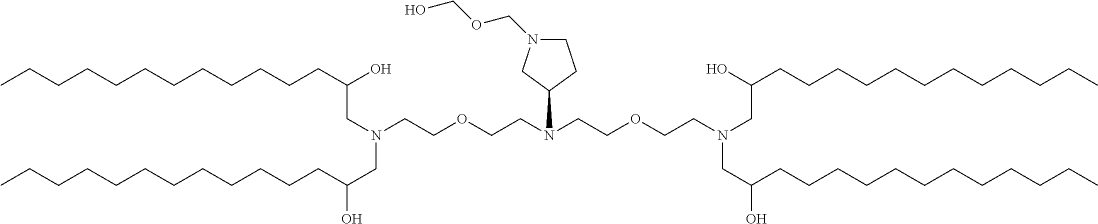 [SMILES: CCCCCCCCCCCCC(O)CN(CCOCCN(CCOCCN(CC(O)CCCCCCCCCCCC)CC(O)CCCCCCCCCCCC)[C@@H]1CCN(COCO)C1)CC(O)CCCCCCCCCCCC] |
| AXT-73 | 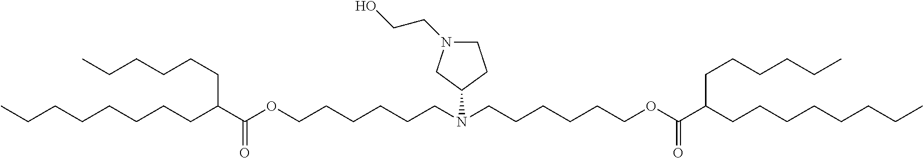 [SMILES: CCCCCCCCC(CCCCCC)C(=O)OCCCCCCN(CCCCCCOC(=O)C(CCCCCC)CCCCCCCC)[C@H]1CCN(CCO)C1] |
| AXT-74 | 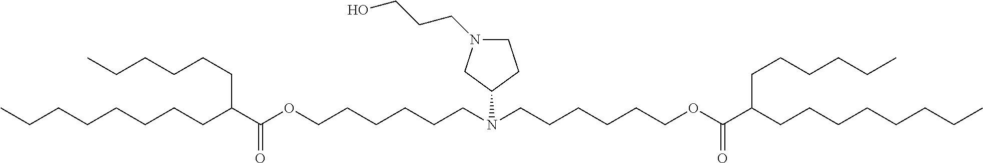 [SMILES: CCCCCCCCC(CCCCCC)C(=O)OCCCCCCN(CCCCCCOC(=O)C(CCCCCC)CCCCCCCC)[C@H]1CCN(CCCO)C1] |
| AXT-75 | 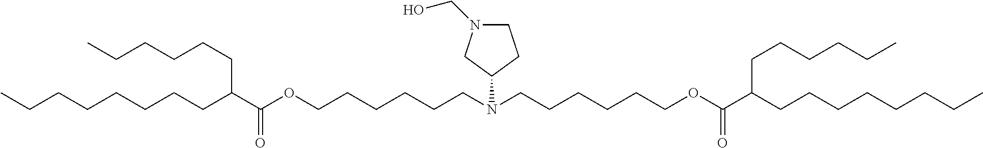 [SMILES: CCCCCCCCC(CCCCCC)C(=O)OCCCCCCN(CCCCCCOC(=O)C(CCCCCC)CCCCCCCC)[C@H]1CCN(CO)C1] |
| AXT-76 | 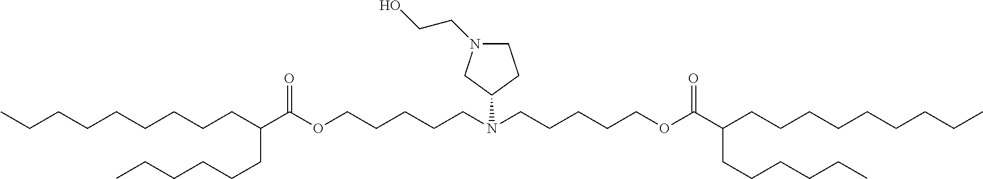 [SMILES: CCCCCCCCCC(CCCCCC)C(=O)OCCCCCN(CCCCCOC(=O)C(CCCCCC)CCCCCCCCC)[C@H]1CCN(CCO)C1] |
| AXT-77 | 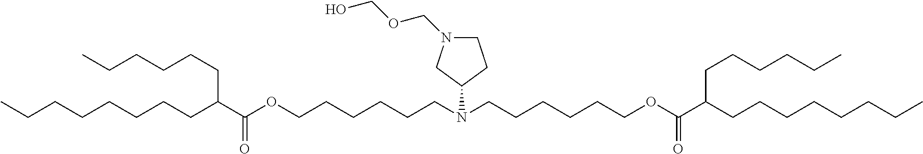 [SMILES: CCCCCCCCC(CCCCCC)C(=O)OCCCCCCN(CCCCCCOC(=O)C(CCCCCC)CCCCCCCC)[C@H]1CCN(COCO)C1] |
| AXT-78 | 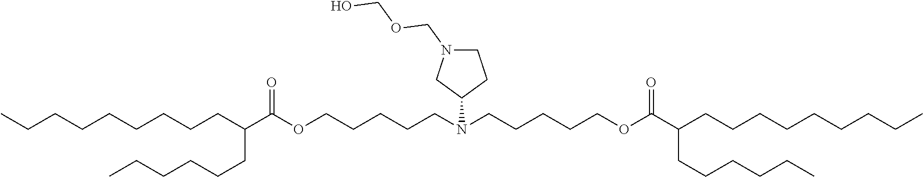 [SMILES: CCCCCCCCCC(CCCCCC)C(=O)OCCCCCN(CCCCCOC(=O)C(CCCCCC)CCCCCCCCC)[C@H]1CCN(COCO)C1] |
| AXT-79 | 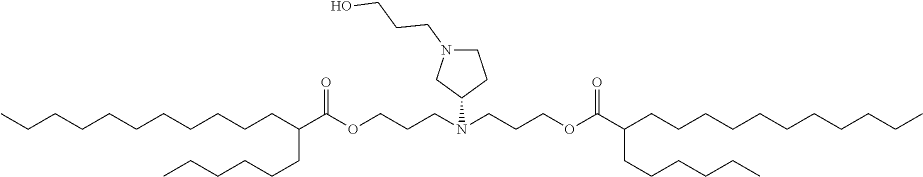 [SMILES: CCCCCCCCCCCC(CCCCCC)C(=O)OCCCN(CCCOC(=O)C(CCCCCC)CCCCCCCCCCC)[C@H]1CCN(CCCO)C1] |
| AXT-80 | 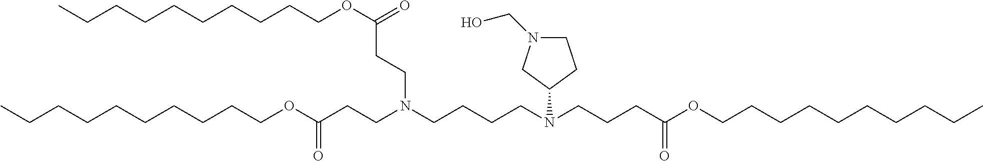 [SMILES: CCCCCCCCCCOC(=O)CCCN(CCCCN(CCC(=O)OCCCCCCCCCC)CCC(=O)OCCCCCCCCCC)[C@H]1CCN(CO)C1] |
| AXT-81 | 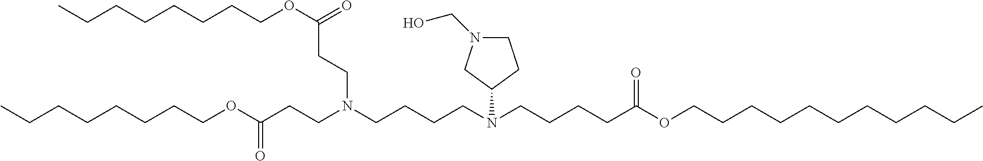 [SMILES: CCCCCCCCCCCOC(=O)CCCCN(CCCCN(CCC(=O)OCCCCCCCC)CCC(=O)OCCCCCCCC)[C@H]1CCN(CO)C1] |
| AXT-82 | 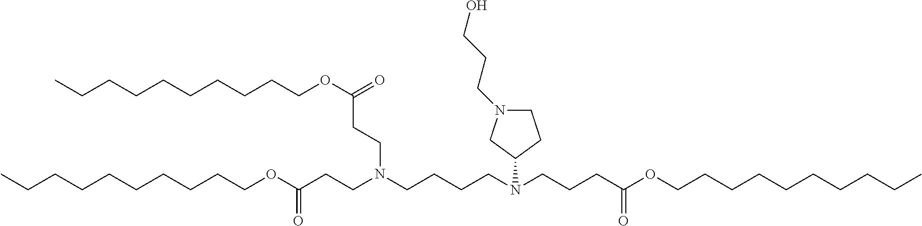 [SMILES: CCCCCCCCCCOC(=O)CCCN(CCCCN(CCC(=O)OCCCCCCCCCC)CCC(=O)OCCCCCCCCCC)[C@H]1CCN(CCCO)C1] |
| AXT-83 | 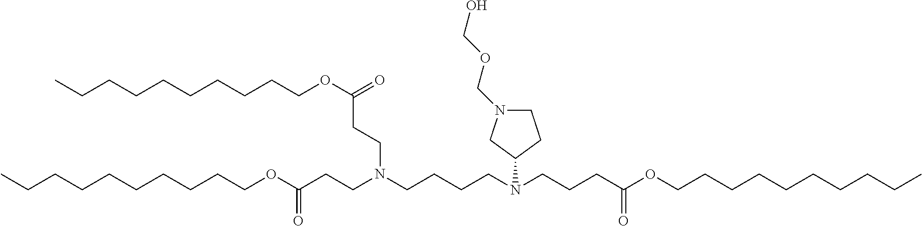 [SMILES: CCCCCCCCCCOC(=O)CCCN(CCCCN(CCC(=O)OCCCCCCCCCC)CCC(=O)OCCCCCCCCCC)[C@H]1CCN(COCO)C1] |
| AXT-84 | 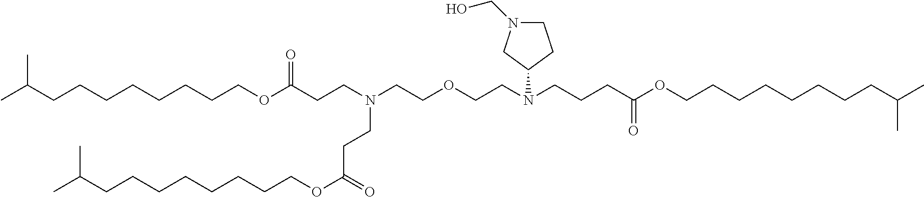 [SMILES: CC(C)CCCCCCCCOC(=O)CCCN(CCOCCN(CCC(=O)OCCCCCCCCC(C)C)CCC(=O)OCCCCCCCCC(C)C)[C@H]1CCN(CO)C1] |
| AXT-85 | 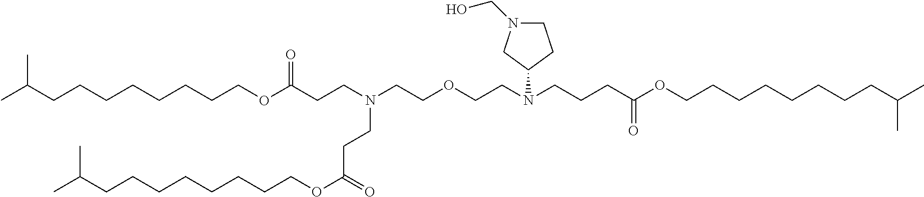 [SMILES: CC(C)CCCCCCCCOC(=O)CCCN(CCOCCN(CCC(=O)OCCCCCCCCC(C)C)CCC(=O)OCCCCCCCCC(C)C)[C@H]1CCN(CO)C1] |
| AXT-86 | 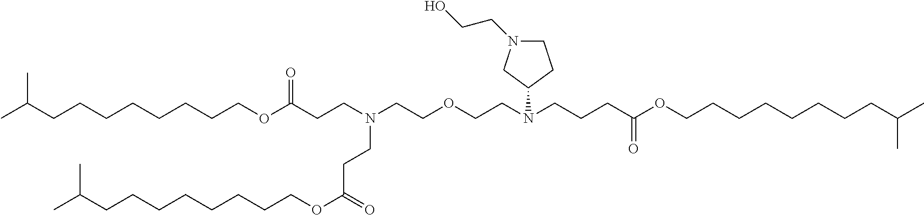 [SMILES: CC(C)CCCCCCCCOC(=O)CCCN(CCOCCN(CCC(=O)OCCCCCCCCC(C)C)CCC(=O)OCCCCCCCCC(C)C)[C@H]1CCN(CCO)C1] |
| AXT-87 | 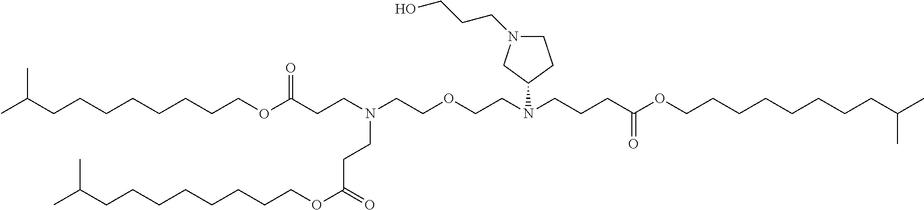 [SMILES: CC(C)CCCCCCCCOC(=O)CCCN(CCOCCN(CCC(=O)OCCCCCCCCC(C)C)CCC(=O)OCCCCCCCCC(C)C)[C@H]1CCN(CCCO)C1] |
| AXT-88 | 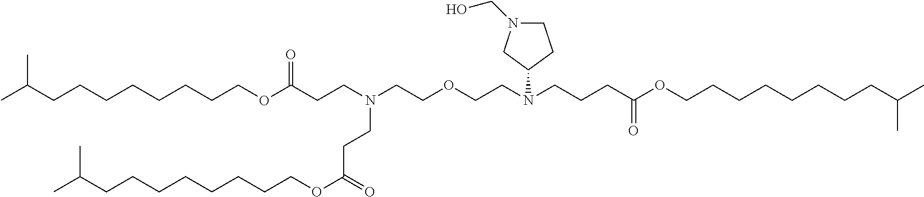 [SMILES: CC(C)CCCCCCCCOC(=O)CCCN(CCOCCN(CCC(=O)OCCCCCCCCC(C)C)CCC(=O)OCCCCCCCCC(C)C)[C@H]1CCN(CO)C1] |
| AXT-89 | 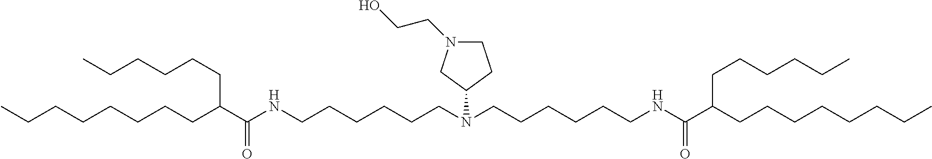 [SMILES: CCCCCCCCC(CCCCCC)C(=O)NCCCCCCN(CCCCCCNC(=O)C(CCCCCC)CCCCCCCC)[C@H]1CCN(CCO)C1] |
| AXT-90 | 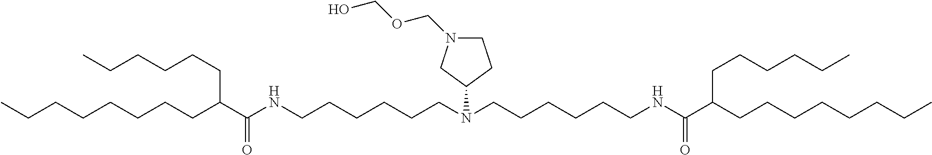 [SMILES: CCCCCCCCC(CCCCCC)C(=O)NCCCCCCN(CCCCCCNC(=O)C(CCCCCC)CCCCCCCC)[C@H]1CCN(COCO)C1] |
| AXT-91 | 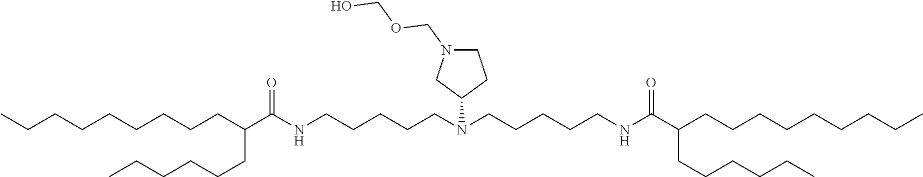 [SMILES: CCCCCCCCCC(CCCCCC)C(=O)NCCCCCN(CCCCCNC(=O)C(CCCCCC)CCCCCCCCC)[C@H]1CCN(COCO)C1] |
| AXT-92 | 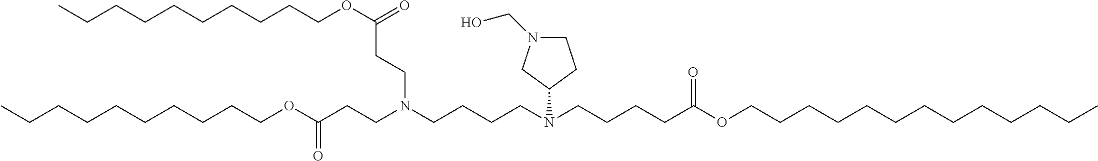 [SMILES: CCCCCCCCCCCCCOC(=O)CCCCN(CCCCN(CCC(=O)OCCCCCCCCCC)CCC(=O)OCCCCCCCCCC)[C@H]1CCN(CO)C1] |
| AXT-93 | 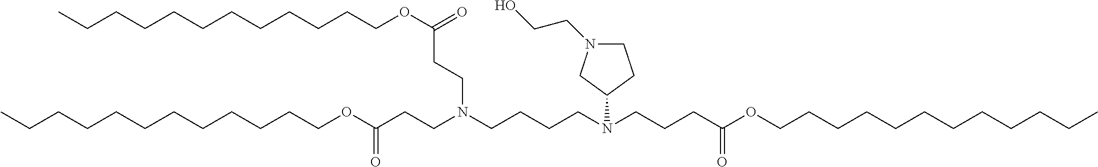 [SMILES: CCCCCCCCCCCCOC(=O)CCCN(CCCCN(CCC(=O)OCCCCCCCCCCCC)CCC(=O)OCCCCCCCCCCCC)[C@H]1CCN(CCO)C1] |
| AXT-94 | 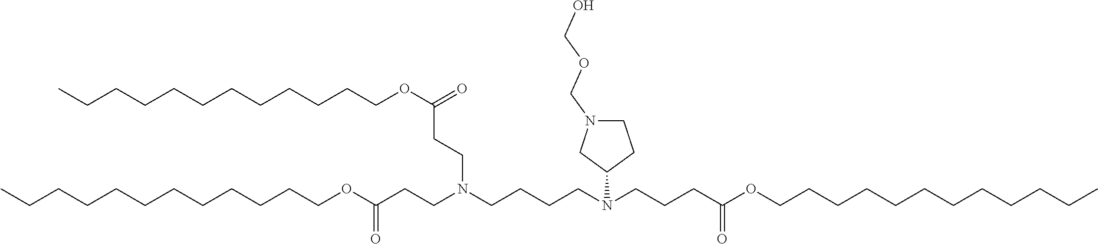 [SMILES: CCCCCCCCCCCCOC(=O)CCCN(CCCCN(CCC(=O)OCCCCCCCCCCCC)CCC(=O)OCCCCCCCCCCCC)[C@H]1CCN(COCO)C1] |
| AXT-95 | 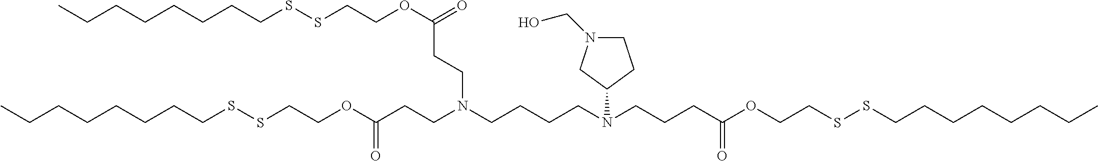 [SMILES: CCCCCCCCSSCCOC(=O)CCCN(CCCCN(CCC(=O)OCCSSCCCCCCCC)CCC(=O)OCCSSCCCCCCCC)[C@H]1CCN(CO)C1] |
| AXT-96 | 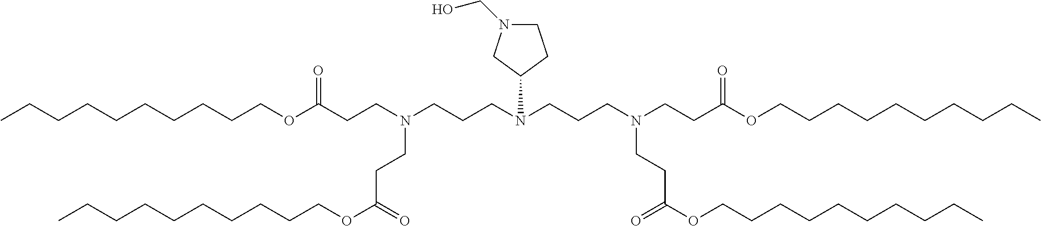 [SMILES: CCCCCCCCCCOC(=O)CCN(CCCN(CCCN(CCC(=O)OCCCCCCCCCC)CCC(=O)OCCCCCCCCCC)[C@H]1CCN(CO)C1)CCC(=O)OCCCCCCCCCC] |
| AXT-97 | 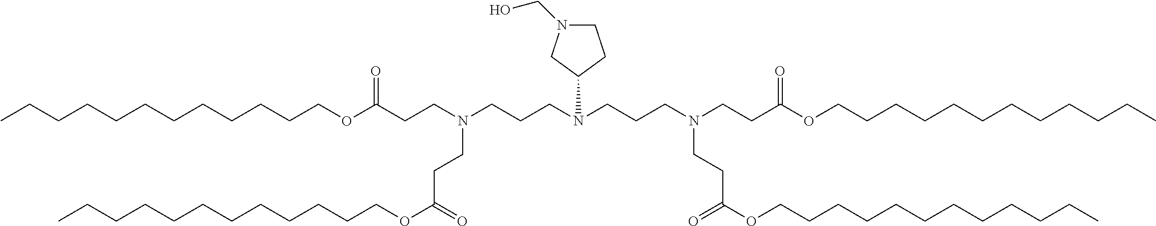 [SMILES: CCCCCCCCCCCCOC(=O)CCN(CCCN(CCCN(CCC(=O)OCCCCCCCCCCCC)CCC(=O)OCCCCCCCCCCCC)[C@H]1CCN(CO)C1)CCC(=O)OCCCCCCCCCCCC] |
| AXT-98 | 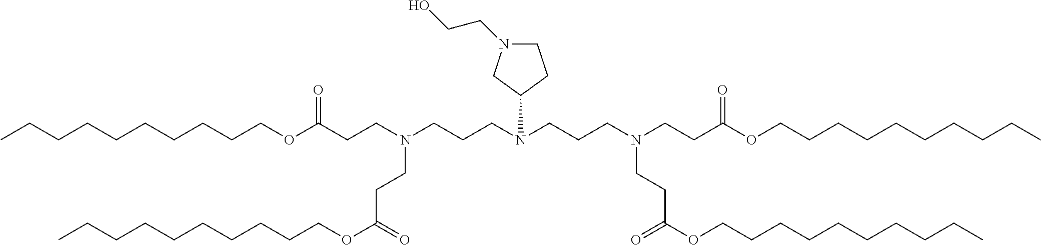 [SMILES: CCCCCCCCCCOC(=O)CCN(CCCN(CCCN(CCC(=O)OCCCCCCCCCC)CCC(=O)OCCCCCCCCCC)[C@H]1CCN(CCO)C1)CCC(=O)OCCCCCCCCCC] |
| AXT-99 | 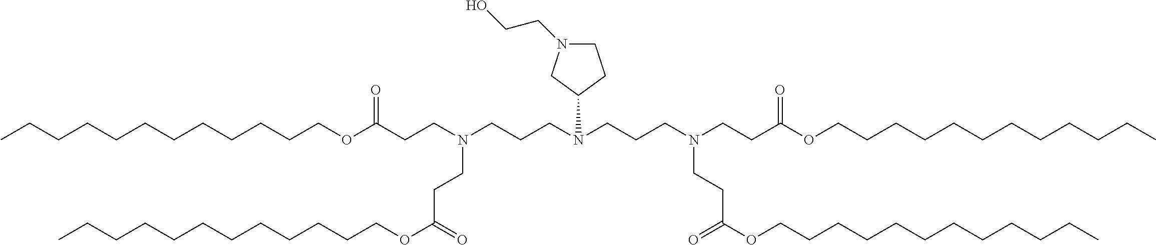 [SMILES: CCCCCCCCCCCCOC(=O)CCN(CCCN(CCCN(CCC(=O)OCCCCCCCCCCCC)CCC(=O)OCCCCCCCCCCCC)[C@H]1CCN(CCO)C1)CCC(=O)OCCCCCCCCCCCC] |
| AXT-100 | 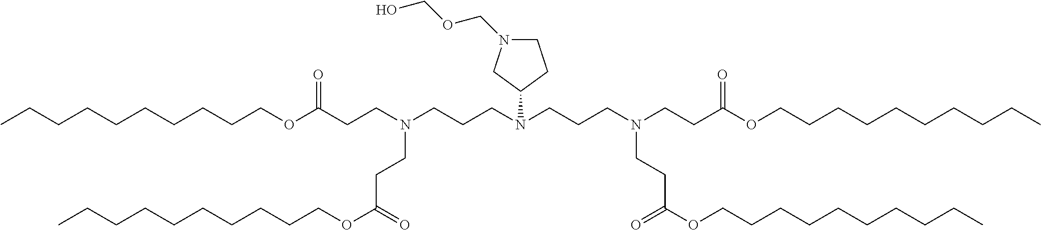 [SMILES: CCCCCCCCCCOC(=O)CCN(CCCN(CCCN(CCC(=O)OCCCCCCCCCC)CCC(=O)OCCCCCCCCCC)[C@H]1CCN(COCO)C1)CCC(=O)OCCCCCCCCCC] |
| AXT-101 | 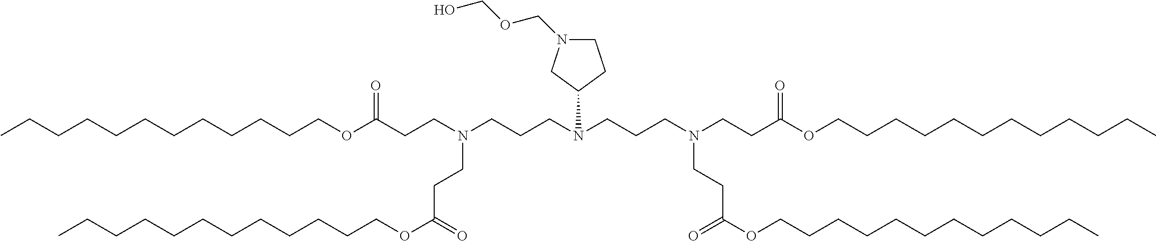 [SMILES: CCCCCCCCCCCCOC(=O)CCN(CCCN(CCCN(CCC(=O)OCCCCCCCCCCCC)CCC(=O)OCCCCCCCCCCCC)[C@H]1CCN(COCO)C1)CCC(=O)OCCCCCCCCCCCC] |
| AXT-102 | 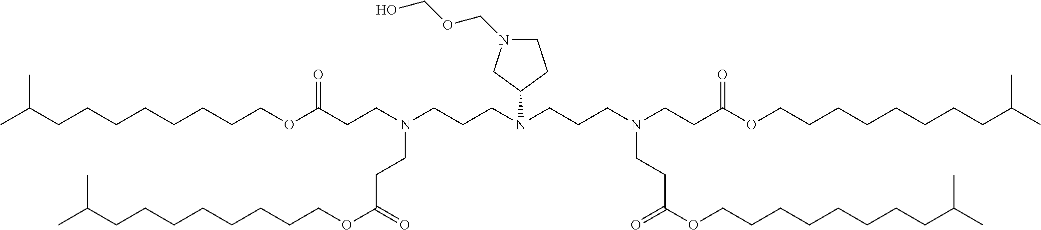 [SMILES: CC(C)CCCCCCCCOC(=O)CCN(CCCN(CCCN(CCC(=O)OCCCCCCCCC(C)C)CCC(=O)OCCCCCCCCC(C)C)[C@H]1CCN(COCO)C1)CCC(=O)OCCCCCCCCC(C)C] |
| AXT-103 | 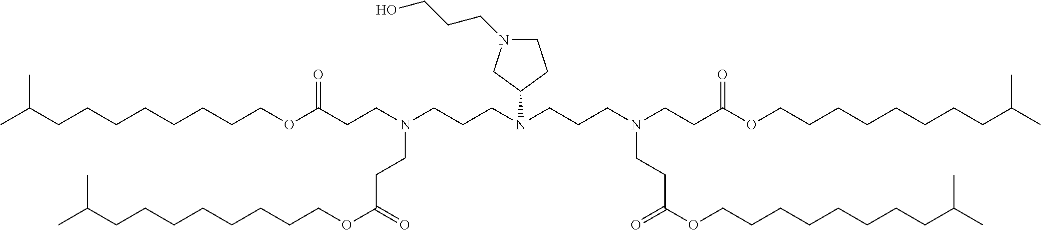 [SMILES: CC(C)CCCCCCCCOC(=O)CCN(CCCN(CCCN(CCC(=O)OCCCCCCCCC(C)C)CCC(=O)OCCCCCCCCC(C)C)[C@H]1CCN(CCCO)C1)CCC(=O)OCCCCCCCCC(C)C] |
| AXT-104 | 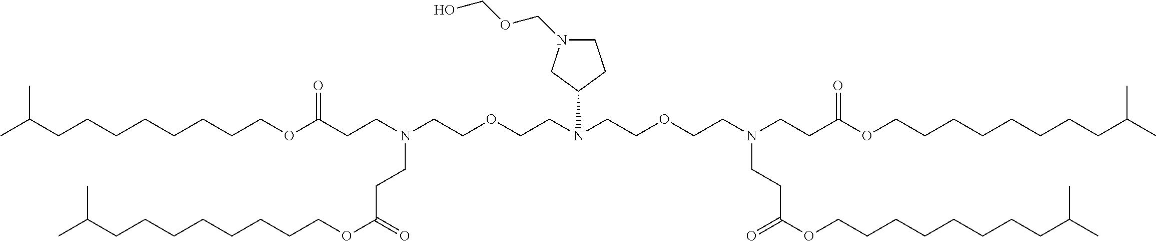 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@H]1CCN(COCO)C1)CCC(=O)OCCCCCCCCC(C)C] |
| AXT-105 | 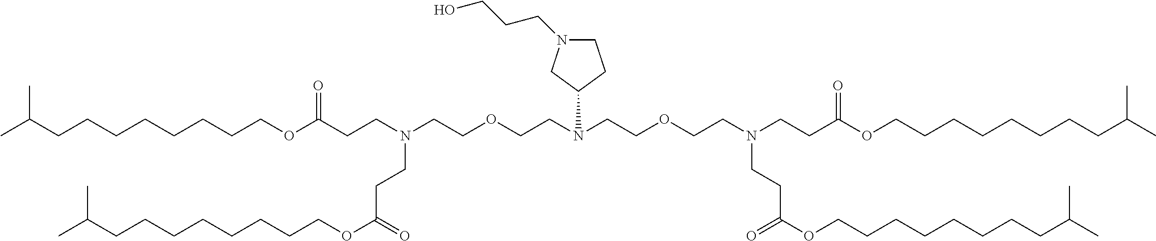 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@H]1CCN(CCCO)C1)CCC(=O)OCCCCCCCCC(C)C] |
| AXT-106 | 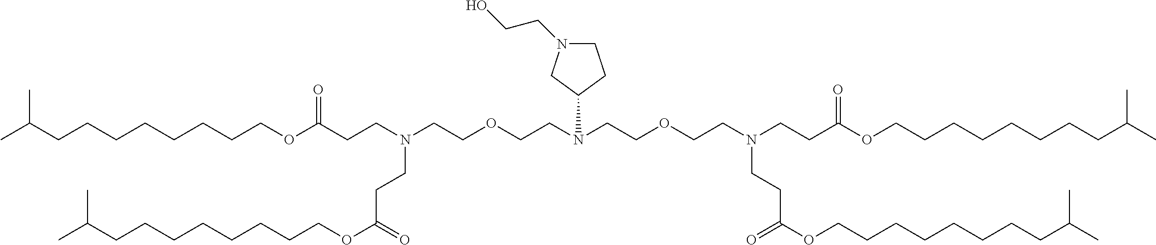 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@H]1CCN(CCO)C1)CCC(=O)OCCCCCCCCC(C)C] |
| AXT-107 | 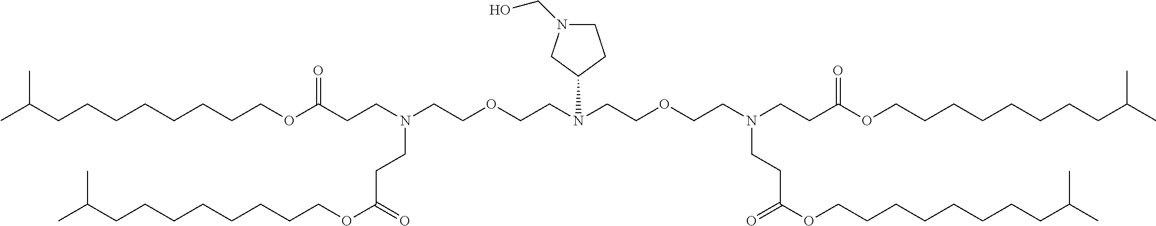 [SMILES: CC(C)CCCCCCCCOC(=O)CCN(CCOCCN(CCOCCN(CCC(=O)OCCCCCCCCC(C)C)CCC(=O)OCCCCCCCCC(C)C)[C@H]1CCN(CO)C1)CCC(=O)OCCCCCCCCC(C)C] |
| AXT-108 | 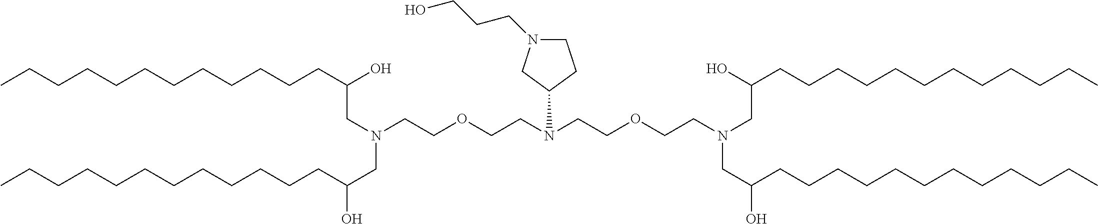 [SMILES: CCCCCCCCCCCCC(O)CN(CCOCCN(CCOCCN(CC(O)CCCCCCCCCCCC)CC(O)CCCCCCCCCCCC)[C@H]1CCN(CCCO)C1)CC(O)CCCCCCCCCCCC] |
| AXT-109 | 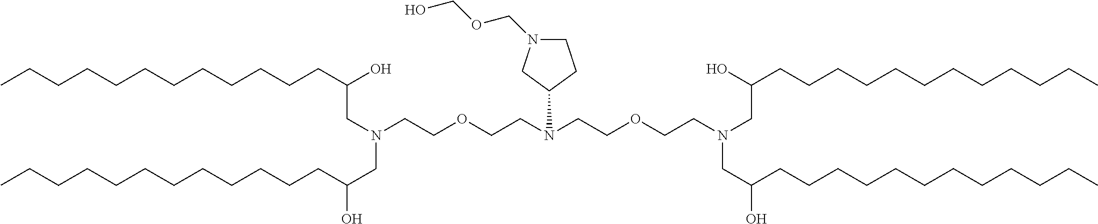 [SMILES: CCCCCCCCCCCCC(O)CN(CCOCCN(CCOCCN(CC(O)CCCCCCCCCCCC)CC(O)CCCCCCCCCCCC)[C@H]1CCN(COCO)C1)CC(O)CCCCCCCCCCCC] |
| AXT-110 | 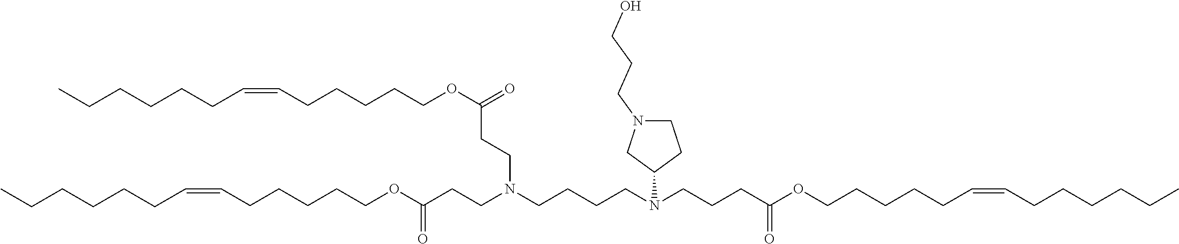 [SMILES: CCCCCC/C=C\CCCCCOC(=O)CCCN(CCCCN(CCC(=O)OCCCCC/C=C\CCCCCC)CCC(=O)OCCCCC/C=C\CCCCCC)[C@H]1CCN(CCCO)C1] |
| AXT-111 | 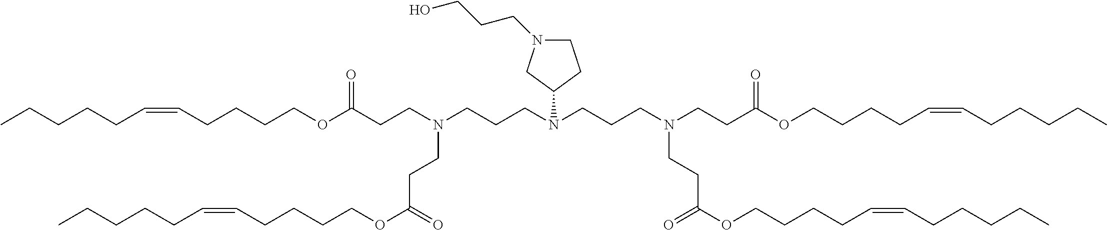 [SMILES: CCCCC/C=C\CCCCOC(=O)CCN(CCCN(CCCN(CCC(=O)OCCCC/C=C\CCCCC)CCC(=O)OCCCC/C=C\CCCCC)[C@H]1CCN(CCCO)C1)CCC(=O)OCCCC/C=C\CCCCC] |
| AXT-112 | 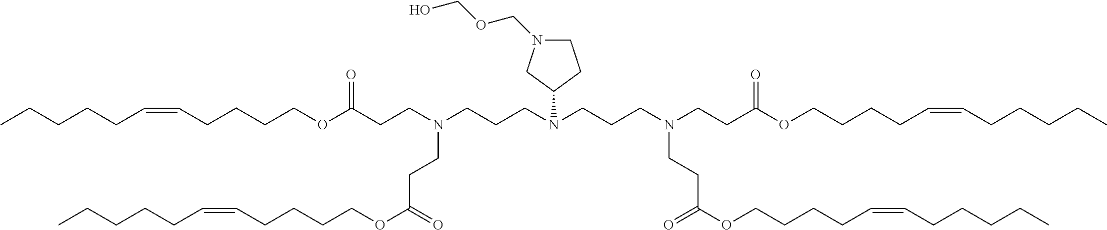 [SMILES: CCCCC/C=C\CCCCOC(=O)CCN(CCCN(CCCN(CCC(=O)OCCCC/C=C\CCCCC)CCC(=O)OCCCC/C=C\CCCCC)[C@H]1CCN(COCO)C1)CCC(=O)OCCCC/C=C\CCCCC] |
| AXT-113 | 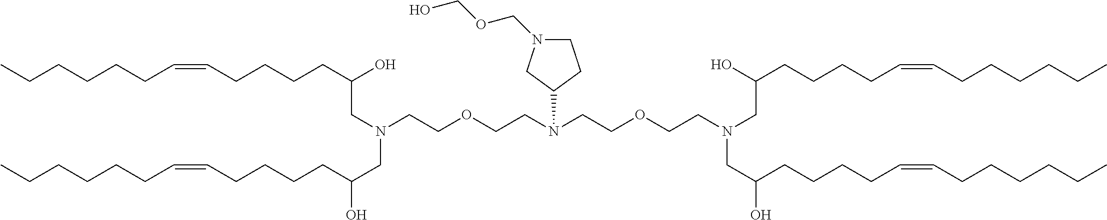 |
| AXT-114 | 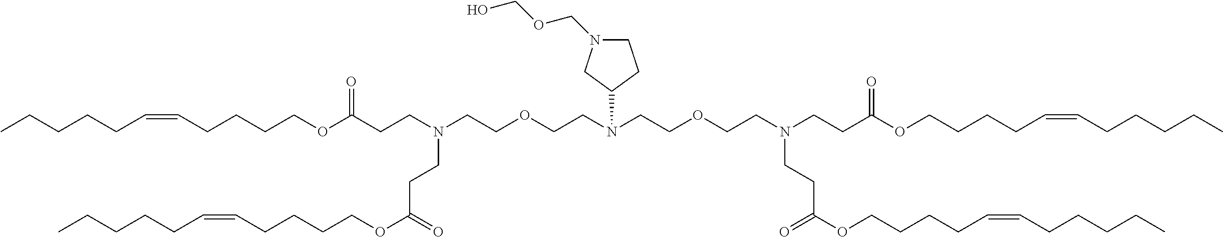 |

In another preferred embodiment, the ionizable lipid has the structure selected from Table 2a below:

TABLE 2a

| Serial number | Structural formula |
|---|---|
| AL08-001-R | 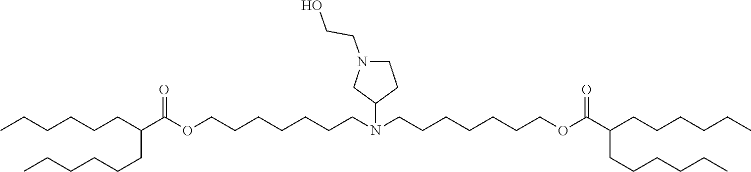 |
| AL08-002-R | 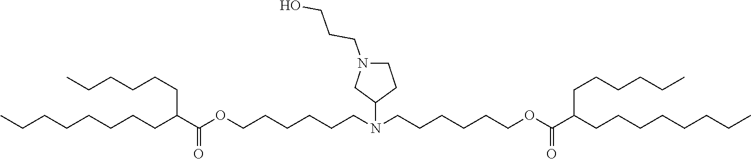 [SMILES: CCCCCCCCC(CCCCCC)C(=O)OCCCCCCN(CCCCCCOC(=O)C(CCCCCC)CCCCCCCC)C1CCN(CCCO)C1] |
| AL08-003-R | 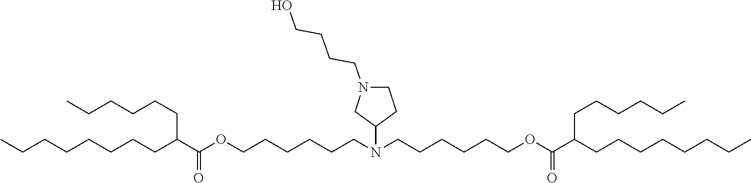 [SMILES: CCCCCCCCC(CCCCCC)C(=O)OCCCCCCN(CCCCCCOC(=O)C(CCCCCC)CCCCCCCC)C1CCN(CCCCO)C1] |
| AL08-004-R | 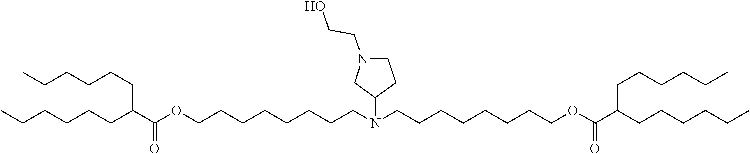 [SMILES: CCCCCCC(CCCCCC)C(=O)OCCCCCCCCN(CCCCCCCCOC(=O)C(CCCCCC)CCCCCC)C1CCN(CCO)C1] |
| AL08-006-R | 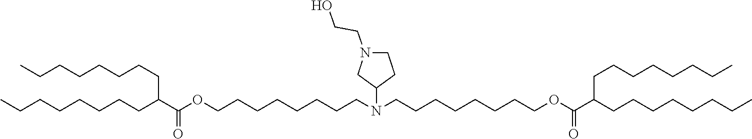 [SMILES: CCCCCCCCC(CCCCCCCC)C(=O)OCCCCCCCCN(CCCCCCCCOC(=O)C(CCCCCCCC)CCCCCCCC)C1CCN(CCO)C1] |
| AL08-007-R | 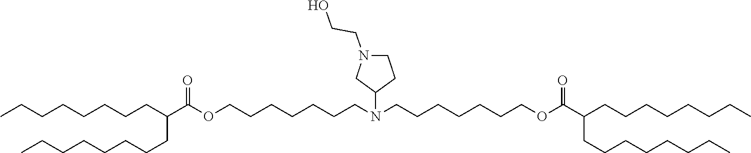 [SMILES: CCCCCCCCC(CCCCCCCC)C(=O)OCCCCCCCN(CCCCCCCOC(=O)C(CCCCCCCC)CCCCCCCC)C1CCN(CCO)C1] |
| AL08-008-R | 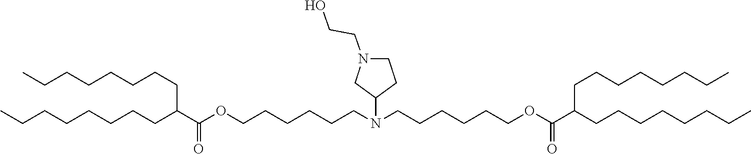 [SMILES: CCCCCCCCC(CCCCCCCC)C(=O)OCCCCCCN(CCCCCCOC(=O)C(CCCCCCCC)CCCCCCCC)C1CCN(CCO)C1] |
| AL08-009-R | 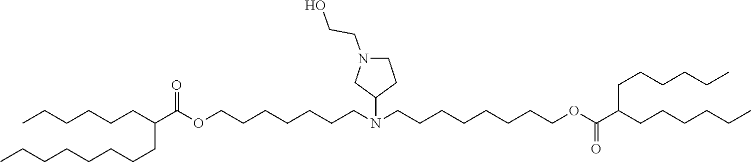 [SMILES: CCCCCCCCC(CCCCCC)C(=O)OCCCCCCCN(CCCCCCCCOC(=O)C(CCCCCC)CCCCCC)C1CCN(CCO)C1] |
| AL08-010-R | 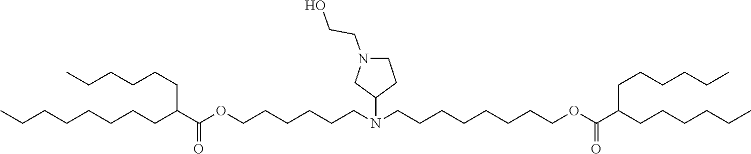 [SMILES: CCCCCCCCC(CCCCCC)C(=O)OCCCCCCN(CCCCCCCCOC(=O)C(CCCCCC)CCCCCC)C1CCN(CCO)C1] |
| AL08-011-R | 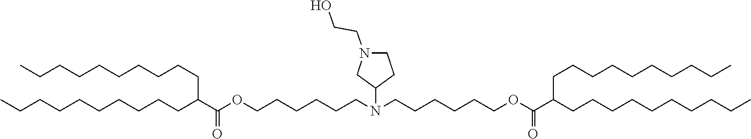 [SMILES: CCCCCCCCCCC(CCCCCCCCCC)C(=O)OCCCCCCN(CCCCCCOC(=O)C(CCCCCCCCCC)CCCCCCCCCC)C1CCN(CCO)C1] |
| AL08-013-R | 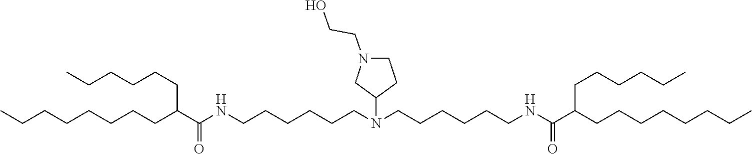 [SMILES: CCCCCCCCC(CCCCCC)C(=O)NCCCCCCN(CCCCCCNC(=O)C(CCCCCC)CCCCCCCC)C1CCN(CCO)C1] |
| AL08-014-R | 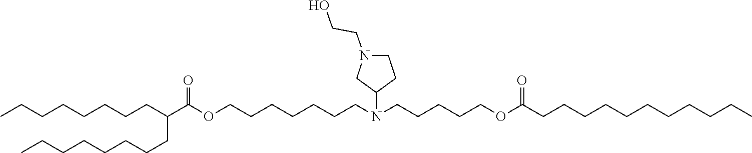 [SMILES: CCCCCCCCCCCC(=O)OCCCCCN(CCCCCCCOC(=O)C(CCCCCCCC)CCCCCCCC)C1CCN(CCO)C1] |
| AL08-015-R | 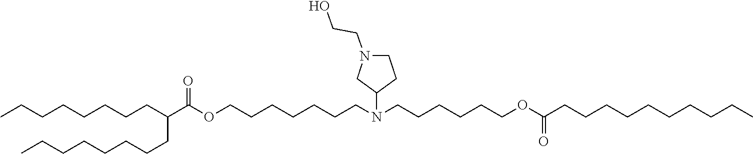 [SMILES: CCCCCCCCCCC(=O)OCCCCCCN(CCCCCCCOC(=O)C(CCCCCCCC)CCCCCCCC)C1CCN(CCO)C1] |
| AL08-016-R | 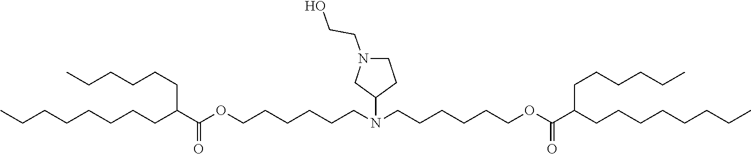 [SMILES: CCCCCCCCC(CCCCCC)C(=O)OCCCCCCN(CCCCCCOC(=O)C(CCCCCC)CCCCCCCC)C1CCN(CCO)C1] |
| AL08-017-R | 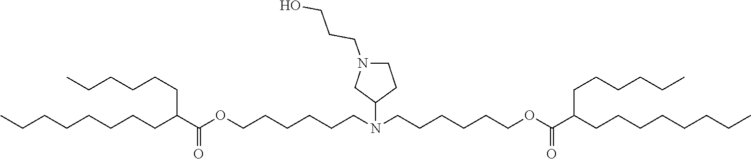 [SMILES: CCCCCCCCC(CCCCCC)C(=O)OCCCCCCN(CCCCCCOC(=O)C(CCCCCC)CCCCCCCC)C1CCN(CCCO)C1] |
| AL08-018-R | 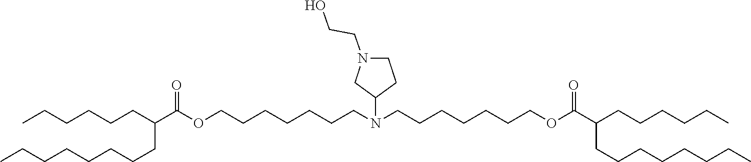 [SMILES: CCCCCCCCC(CCCCCC)C(=O)OCCCCCCCN(CCCCCCCOC(=O)C(CCCCCC)CCCCCCCC)C1CCN(CCO)C1] |
| AL08-019-R | 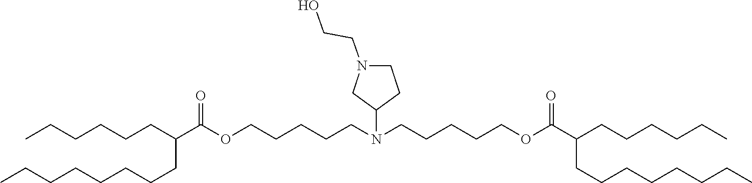 [SMILES: CCCCCCCCC(CCCCCC)C(=O)OCCCCCN(CCCCCOC(=O)C(CCCCCC)CCCCCCCC)C1CCN(CCO)C1] |
| AL08-021-R | 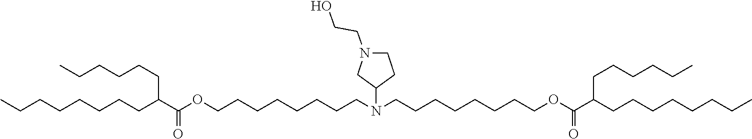 [SMILES: CCCCCCCCC(CCCCCC)C(=O)OCCCCCCCCN(CCCCCCCCOC(=O)C(CCCCCC)CCCCCCCC)C1CCN(CCO)C1] |
| AL08-022-R | 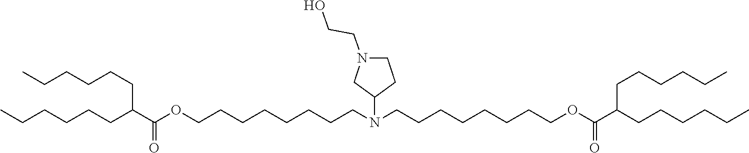 [SMILES: CCCCCCC(CCCCCC)C(=O)OCCCCCCCCN(CCCCCCCCOC(=O)C(CCCCCC)CCCCCC)C1CCN(CCO)C1] |
| AL08-024-R | 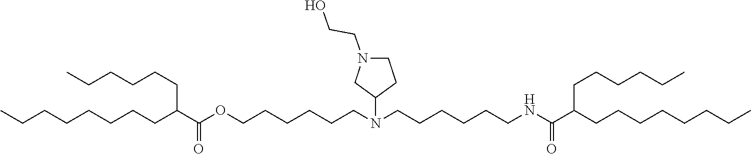 [SMILES: CCCCCCCCC(CCCCCC)C(=O)NCCCCCCN(CCCCCCOC(=O)C(CCCCCC)CCCCCCCC)C1CCN(CCO)C1] |
| AL08-026-R | 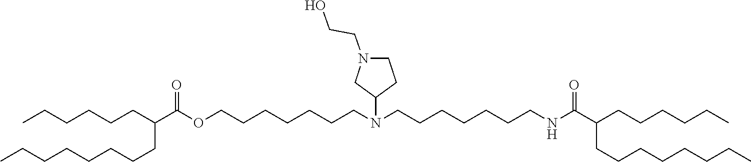 [SMILES: CCCCCCCCC(CCCCCC)C(=O)NCCCCCCCN(CCCCCCCOC(=O)C(CCCCCC)CCCCCCCC)C1CCN(CCO)C1] |

In another preferred embodiment, the ionizable lipid has the structure selected from Table 2b below:

TABLE 2b

| | |
|---|---|
| AL08-001 | 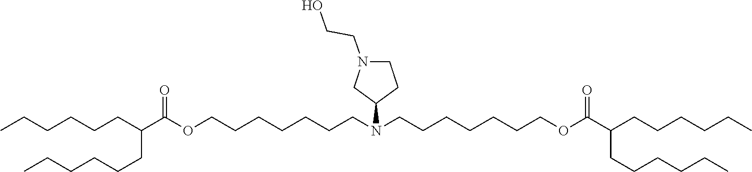 [SMILES: CCCCCCC(CCCCCC)C(=O)OCCCCCCCN(CCCCCCCOC(=O)C(CCCCCC)CCCCCC)[C@@H]1CCN(CCO)C1] |
| AL08-002 | 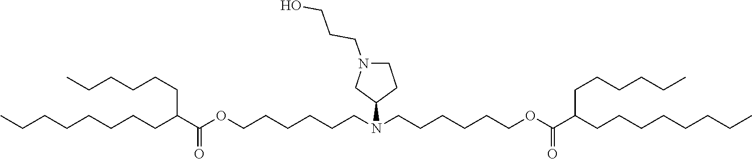 [SMILES: CCCCCCCCC(CCCCCC)C(=O)OCCCCCCN(CCCCCCOC(=O)C(CCCCCC)CCCCCCCC)[C@@H]1CCN(CCCO)C1] |
| AL08-003 | 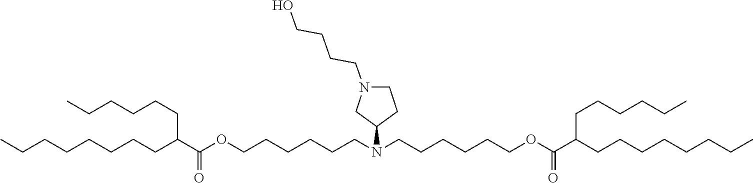 [SMILES: CCCCCCCCC(CCCCCC)C(=O)OCCCCCCN(CCCCCCOC(=O)C(CCCCCC)CCCCCCCC)[C@@H]1CCN(CCCCO)C1] |
| AL08-004 | 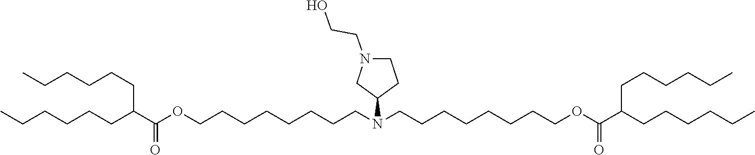 [SMILES: CCCCCCC(CCCCCC)C(=O)OCCCCCCCCN(CCCCCCCCOC(=O)C(CCCCCC)CCCCCC)[C@@H]1CCN(CCO)C1] |
| AL08-005 | 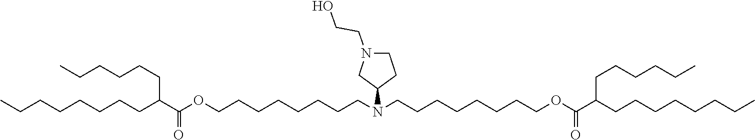 [SMILES: CCCCCCCCC(CCCCCC)C(=O)OCCCCCCCCN(CCCCCCCCOC(=O)C(CCCCCC)CCCCCCCC)[C@@H]1CCN(CCO)C1] |
| AL08-006 | 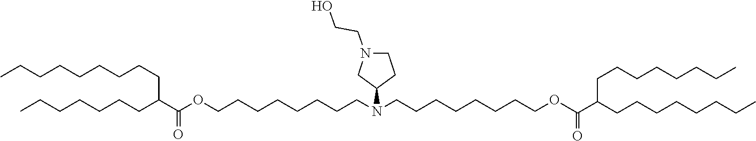 [SMILES: CCCCCCCCCC(CCCCCCC)C(=O)OCCCCCCCCN(CCCCCCCCOC(=O)C(CCCCCCCC)CCCCCCCC)[C@@H]1CCN(CCO)C1] |
| AL08-007 | 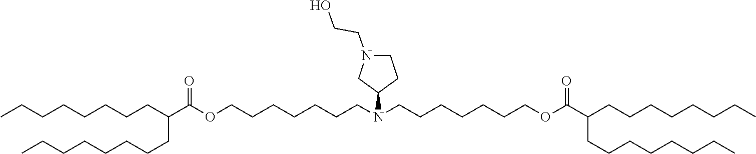 [SMILES: CCCCCCCCC(CCCCCCCC)C(=O)OCCCCCCCN(CCCCCCCOC(=O)C(CCCCCCCC)CCCCCCCC)[C@@H]1CCN(CCO)C1] |
| AL08-008 | 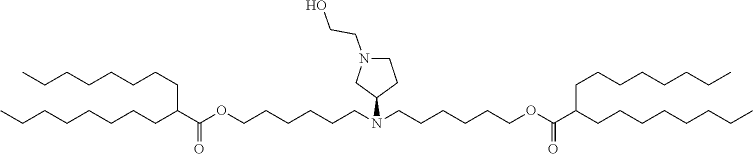 [SMILES: CCCCCCCCC(CCCCCCCC)C(=O)OCCCCCCN(CCCCCCOC(=O)C(CCCCCCCC)CCCCCCCC)[C@@H]1CCN(CCO)C1] |
| AL08-009 | 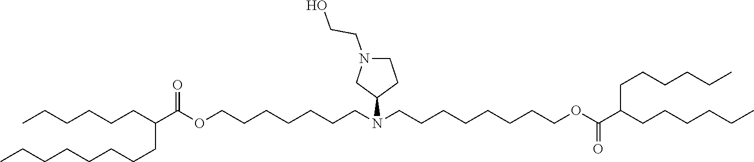 [SMILES: CCCCCCCCC(CCCCCC)C(=O)OCCCCCCCN(CCCCCCCCOC(=O)C(CCCCCC)CCCCCC)[C@@H]1CCN(CCO)C1] |
| AL08-010 | 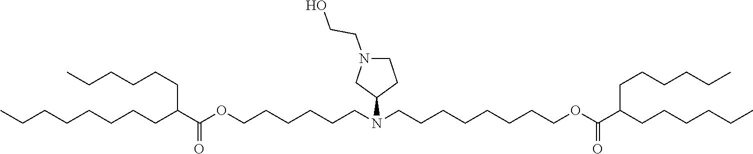 [SMILES: CCCCCCCCC(CCCCCC)C(=O)OCCCCCCN(CCCCCCCCOC(=O)C(CCCCCC)CCCCCC)[C@@H]1CCN(CCO)C1] |
| AL08-011 | 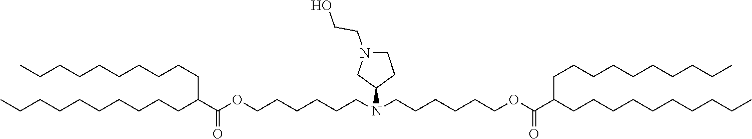 [SMILES: CCCCCCCCCCC(CCCCCCCCCC)C(=O)OCCCCCCN(CCCCCCOC(=O)C(CCCCCCCCCC)CCCCCCCCCC)[C@@H]1CCN(CCO)C1] |
| AL08-012 | 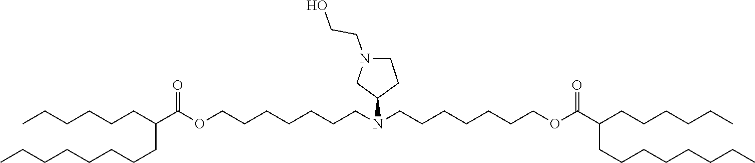 [SMILES: CCCCCCCCC(CCCCCC)C(=O)OCCCCCCCN(CCCCCCCOC(=O)C(CCCCCC)CCCCCCCC)[C@@H]1CCN(CCO)C1] |
| AL08-013 | 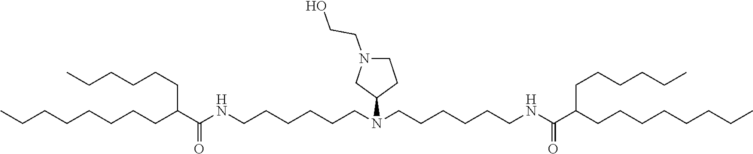 [SMILES: CCCCCCCCC(CCCCCC)C(=O)NCCCCCCN(CCCCCCNC(=O)C(CCCCCC)CCCCCCCC)[C@@H]1CCN(CCO)C1] |
| AL08-014 | 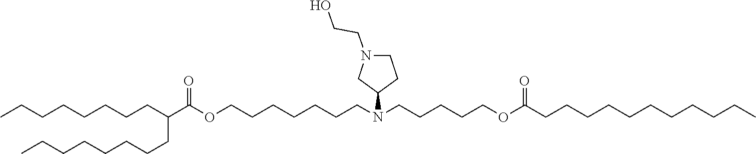 [SMILES: CCCCCCCCCCCC(=O)OCCCCCN(CCCCCCCOC(=O)C(CCCCCCCC)CCCCCCCC)[C@@H]1CCN(CCO)C1] |
| AL08-015 | 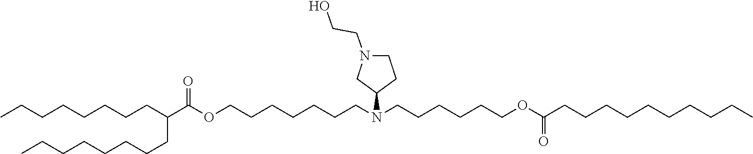 [SMILES: CCCCCCCCCCC(=O)OCCCCCCN(CCCCCCCOC(=O)C(CCCCCCCC)CCCCCCCC)[C@@H]1CCN(CCO)C1] |
| AL08-016 | 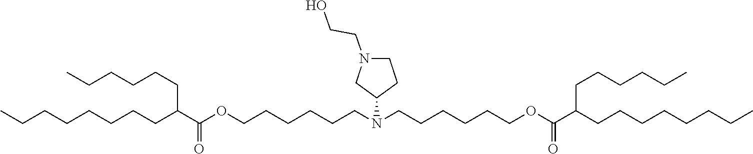 [SMILES: CCCCCCCCC(CCCCCC)C(=O)OCCCCCCN(CCCCCCOC(=O)C(CCCCCC)CCCCCCCC)[C@H]1CCN(CCO)C1] |
| AL08-017 | 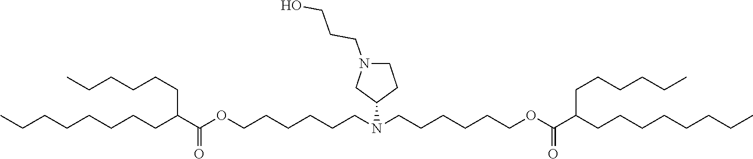 [SMILES: CCCCCCCCC(CCCCCC)C(=O)OCCCCCCN(CCCCCCOC(=O)C(CCCCCC)CCCCCCCC)[C@H]1CCN(CCCO)C1] |
| AL08-018 | 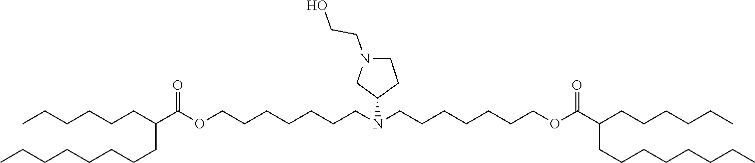 [SMILES: CCCCCCCCC(CCCCCC)C(=O)OCCCCCCCN(CCCCCCCOC(=O)C(CCCCCC)CCCCCCCC)[C@H]1CCN(CCO)C1] |
| AL08-019 | 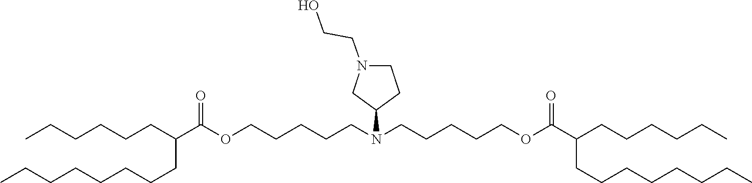 [SMILES: CCCCCCCCC(CCCCCC)C(=O)OCCCCCN(CCCCCOC(=O)C(CCCCCC)CCCCCCCC)[C@@H]1CCN(CCO)C1] |
| AL08-020 | 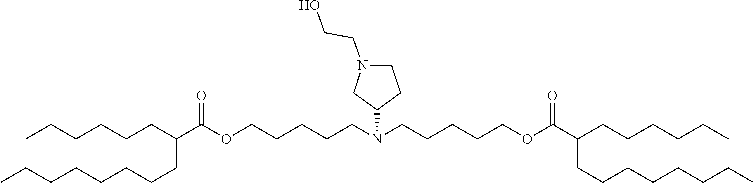 |
| AL08-021 | 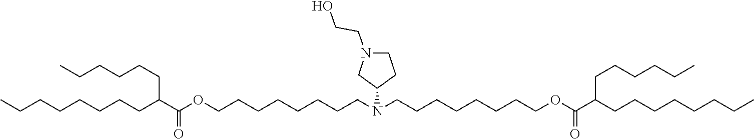 [SMILES: CCCCCCCCC(CCCCCC)C(=O)OCCCCCCCCN(CCCCCCCCOC(=O)C(CCCCCC)CCCCCCCC)[C@H]1CCN(CCO)C1] |
| AL08-022 | 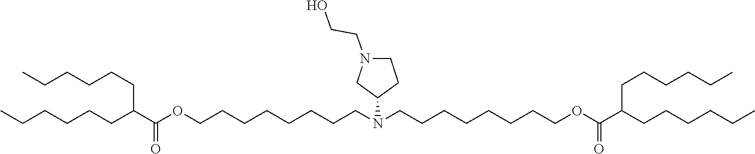 [SMILES: CCCCCCC(CCCCCC)C(=O)OCCCCCCCCN(CCCCCCCCOC(=O)C(CCCCCC)CCCCCC)[C@H]1CCN(CCO)C1] |
| AL08-023 | 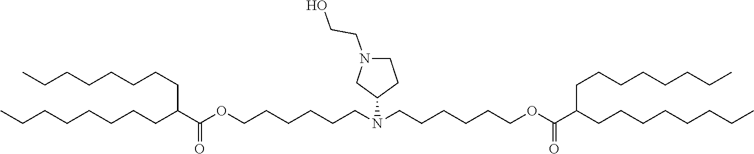 [SMILES: CCCCCCCCC(CCCCCCCC)C(=O)OCCCCCCN(CCCCCCOC(=O)C(CCCCCCCC)CCCCCCCC)[C@H]1CCN(CCO)C1] |
| AL08-024 | 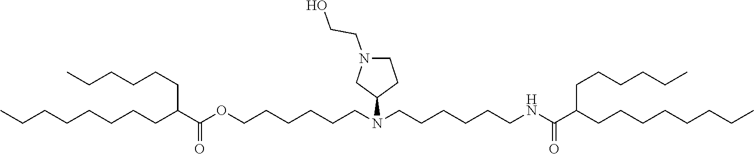 [SMILES: CCCCCCCCC(CCCCCC)C(=O)NCCCCCCN(CCCCCCOC(=O)C(CCCCCC)CCCCCCCC)[C@@H]1CCN(CCO)C1] |
| AL08-025 | 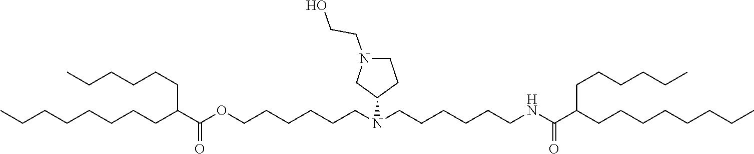 [SMILES: CCCCCCCCC(CCCCCC)C(=O)NCCCCCCN(CCCCCCOC(=O)C(CCCCCC)CCCCCCCC)[C@H]1CCN(CCO)C1] |
| AL08-026 | 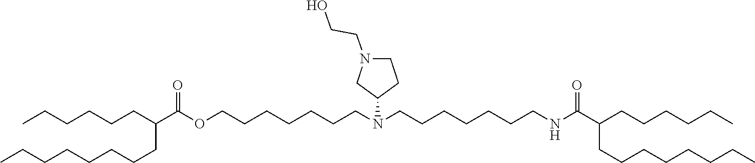 [SMILES: CCCCCCCCC(CCCCCC)C(=O)NCCCCCCCN(CCCCCCCOC(=O)C(CCCCCC)CCCCCCCC)[C@H]1CCN(CCO)C1] |
| AL08-027 | 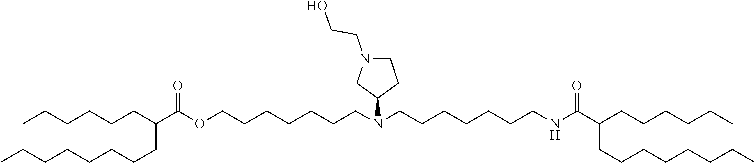 [SMILES: CCCCCCCCC(CCCCCC)C(=O)NCCCCCCCN(CCCCCCCOC(=O)C(CCCCCC)CCCCCCCC)[C@@H]1CCN(CCO)C1] |

In another preferred embodiment, R₃, R₄, R₅ and R₆ are each independently C₅-C₁₅ alkyl.

In another preferred embodiment, the ionizable lipid has the structure represented by Formula (I-1) below:

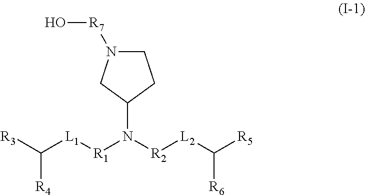

wherein,
R₁ and R₂ are each independently selected from —(CH₂)ₙ—, wherein n is a positive integer ranging from 4 to 8;
L₁ is selected from the following functional groups: —O—, —(C═O)O—, —O(C═O)—, —CH(OH)—;
L₂ is selected from the following functional groups: —O—, —(C═O)O—, —O(C═O)—, —CH(OH)—;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14; and R₃, R₄, R₅ and R₆ each independently have 4-20 CH₂ structural moieties;
R_{3b}, R_{3d}, R_{4b}, R_{4d}, R_{5b}, R_{5d}, R_{6b} and R_{6d} are each independently absent or selected from the following functional groups: —(C═O)O—, —O(C═O)—, —(S—S)—, —(C═C)—(CH₂)—(C═C)—, —CH(OH)—;
R₃ₑ, R₄ₑ, R₅ₑ, R₆ₑ are each independently C₂-C₂₀ hydrocarbyl group;
R₇ is C₁-C₃ hydrocarbyl group, or —(CH₂)₂—O—(CH₂)₂—.

In another preferred embodiment, the ionizable lipid has the structure represented by the Formula below:

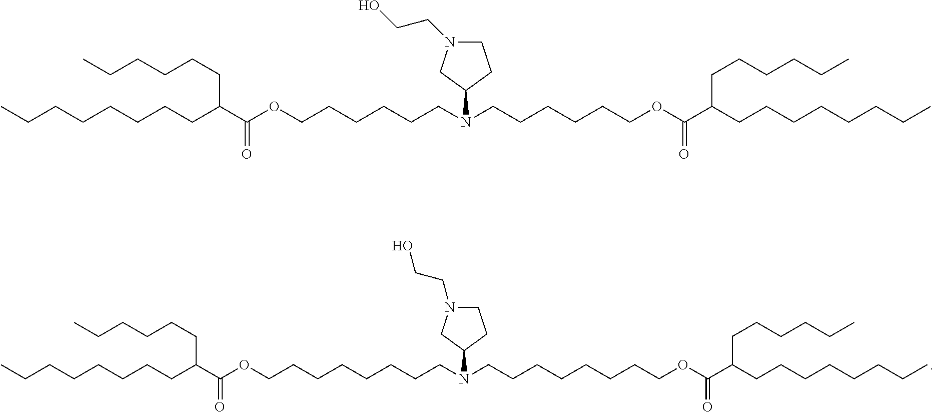

In a second aspect of the present disclosure, provides a method for preparing the ionizable lipid, or pharmaceutically acceptable salt, tautomer or stereoisomer thereof according to the first aspect of the present disclosure, the method comprises: method I, method II and method III.

Wherein, method I comprises the following steps:
(S1) under the protection of an inert gas, compounds A1 and A2 are reacted to obtain compound A3;
(S2) under the protection of an inert gas, the tert-butoxycarbonyl (Boc) group of A3 is removed to obtain A4;
(S3) under the protection of an inert gas, A4 is reacted with A5 or A6 to obtain A7 (i.e., the compound represented by (I-1));

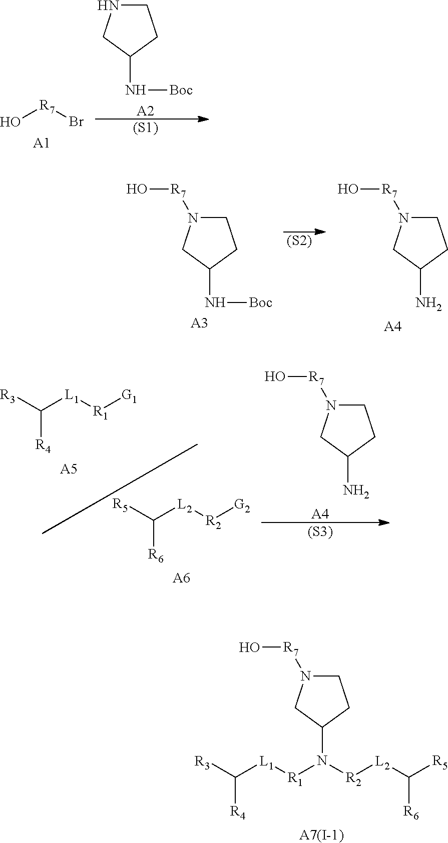

wherein, R₁ and R₂ are each independently selected from —(CH₂)ₙ—, wherein n is a positive integer ranging from 1 to 14;
G₁ and G₂ are each independently selected from an active functional group, preferably selected from a carbonyl group, halogen (fluorine, chlorine, bromine, iodine, etc.), an ethylene oxide group, etc.
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: O, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from O, —(C═O)O—, —O(C═O)—, —CH(OH)—; L₂b is —(CH₂)ₙ—, wherein n is selected from 0, 1, 2, 3 or 4;
R₃, R₄, R₅ and R₆ are each independently C₂-C₂₀ hydrocarbyl group.
R₇ is selected from C₁-C₅ hydrocarbyl group, or —(CH₂)ₘO(CH₂)ₙ—, wherein m, n are each independently selected from 1, 2, 3.

In another preferred embodiment, R₁ and R₂ are each independently —(CH₂)ₙ—, wherein n is an integer ranging from 1 to 14, prefer an integer ranging from 5-8;
G₁ and G₂ are each independently selected from aldehyde group, halogen;
L₁ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —(C═O)O—, —O(C═O)—, —(C═O)NH—, —NH(C═O)—;
L₂ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; preferably selected from —(C═O)O—, —O(C═O)—, —(C═O)NH—, —NH(C═O)—;
R₃, R₄, R₅ and R₆ are each independently selected from: H, C₂-C₂₀ hydrocarbyl group;
R7 is selected form C₁-C₅ hydrocarbyl group, preferably selected from C₂-C₄ hydrocarbyl group.

Method II comprises the following steps:
(D1) under the protection of an inert gas, compound B1 is reacted with B2 to obtain compound B3;
(D2) under the protection of an inert gas, compound B3 is reacted with B4 to obtain compound B5;
(D3) under the protection of an inert gas, compound B5 is reacted with B6 to obtain B7, i.e., the compound represented by (I-2);

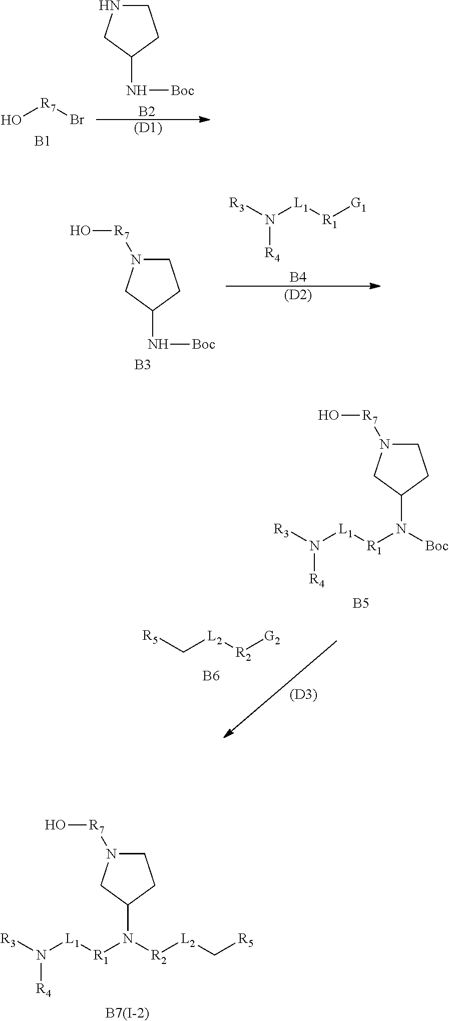

wherein, R₁ and R₂ are each independently selected from —(CH₂)ₙ—, wherein n is a positive integer ranging from 1 to 14;
G₁ and G₂ are each independently selected from an active functional group, preferably selected from a carbonyl group, halogen (fluorine, chlorine, bromine, iodine, etc.), an ethylene oxide group, etc.
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L₂b is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ;
wherein, R₃ₐ, R_{3c}, R₄ₐ, R_{4c}, R₅ₐ, R_{5c} are each independently —(CH₂)ₙ—, wherein n is selected from a positive integer ranging from 1 to 14;
R_{3b}, R_{3d}, R_{4b}, R_{4d}, R_{5b}, R_{5d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₃ₑ, R₄ₑ, R₅ₑ are each independently C₂-C₂₀ hydrocarbyl group;
R₆ is H;
R₇ is selected from C₁-C₅ hydrocarbyl group, or —(CH₂)ₘO(CH₂)ₙ—, wherein m, n are each independently selected from 1, 2, 3.

Method III comprises the following steps:
(M1) under the protection of an inert gas, compounds C1 and C2 are reacted to obtain C3;
(M2) under the protection of an inert gas, compound C3 undergoes de-Boc deprotection to obtain compound C4;
(M3) under the protection of an inert gas, compound C4 is reacted with C5 or C6 to obtain compound C7, i.e., the compound represented by Formula (I-3);

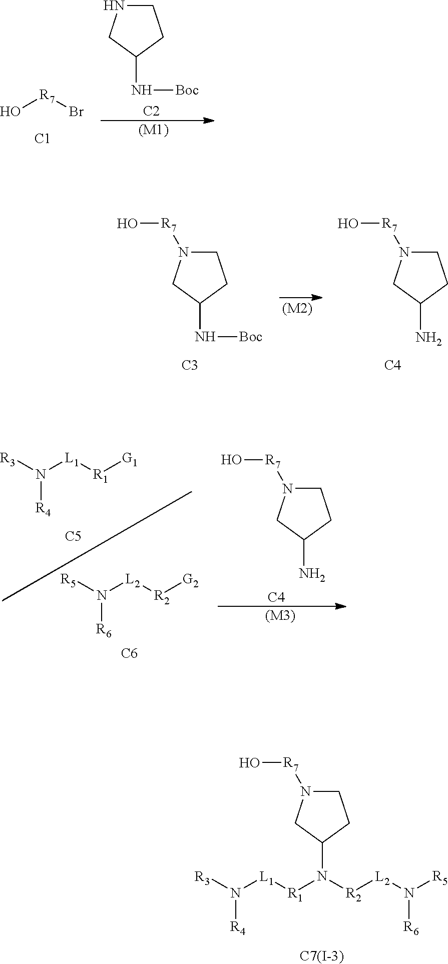

wherein, R₁ and R₂ are each independently selected from —(CH₂)ₙ—, wherein n is a positive integer ranging from 1 to 14;
G₁ and G₂ are each independently selected from an active functional group, preferably selected from a carbonyl group, halogen (fluorine, chlorine, bromine, iodine, etc.), an ethylene oxide group, etc.
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —O—, —(C═O)O—, —O(C═O)—, —CH(OH)—; L₂b is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14;
R_{3b}, R_{3d}, R_{4b}, R_{4d}, R_{5b}, R_{5d}, R_{6b} and R_{6d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—, preferably —(C═O)O—, —O(C═O)—, —(S—S)—, —(C═C)—(CH₂)—(C═C)—, —CH(OH)—;
R₃ₑ, R₄ₑ, R₅ₑ, R₆ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

In a third aspect of the present disclosure, provides a lipid nanoparticle (LNP), the lipid nanoparticle comprises the ionizable lipid, or pharmaceutically acceptable salt, tautomer or stereoisomer thereof according to the first aspect of the present disclosure.

In another preferred embodiment, the lipid nanoparticle further comprises an auxiliary lipid.

In another preferred embodiment, in the lipid nanoparticle, the content of the ionizable lipid accounts for 30-65 mol % of the total lipid content.

In another preferred embodiment, the auxiliary lipid includes helper phospholipids, sterols, polymer-conjugated lipids, or a combination thereof.

In another preferred embodiment, the auxiliary lipid is a combination of helper phospholipids, sterols and polymer-conjugated lipids.

In another preferred embodiment, the helper phospholipid is preferably selected from: 1,2-distearoyl-sn-glycero-3-phosphocholine (DSPC), 1,2-dioleoyl-sn-glycero-3-phosphoethanolamine (DOPE), dioleoylphosphatidylcholine (DOPC), 1,2-dioleoyl-sn-glycero-3-phosphocholine, 1,2-dipalmitoyl-sn-glycero-3-phosphocholine, 1,2-dipalmitoyl-sn-glycero-3-phosphoethanolamine, 1,2-dimyristoyl-sn-glycero-3-phosphoethanolamine, sodium 1,2-dioleoyl-sn-glycero-3-phospho-rac-(1-glycerol), 1,2-dipalmitoylphosphatidylglycerol, 1-palmitoyl-2-oleoylphosphatidylcholine, 1-palmitoyl-2-oleoylphosphoethanolamine, distearoylphosphoethanolamine, 1-stearoyl-2-oleoylphosphatidylcholine, 1-stearoyl-2-oleoylphosphoethanolamine, or a combination thereof.

In another preferred embodiment, the sterol includes cholesterol or cholesterol derivatives.

In another preferred embodiment, the polymer-conjugated lipid is pegylated (PEG) lipids.

In another preferred embodiment, the pegylated lipid is preferably selected from the following group: DMG-PEG2000, DSPE-PEG2000, DSG-PEG2000, DSPE-PEG-Mannose, DMG-PEG2000-(polypeptides, proteins, amino acids, vitamins and the other active substances), or a combination thereof.

In another preferred embodiment, the lipid nanoparticle comprises an ionizable lipid, DSPC, cholesterol and DMG-PEG2000, wherein the molar ratio of the ionizable lipid:DSPC:cholesterol:DMG-PEG2000 is (30-65):(5-30):(30-55):(1-5), preferably (30-65):(10-30):(20-50):(1-5), (40-50):(10-15):(38-45):(1.5-2), (40-60):(10-20):(25-45):(1-2) or (40-60):(10-20):(28.5-38.5):1.5.

In another preferred embodiment, the lipid nanoparticle is a lipid nanoparticle modified with a targeting moiety.

In another preferred embodiment, the lipid nanoparticle modified with a targeting moiety comprises an ionizable lipid, DSPC, cholesterol, DSPE-PEG2000 or DMG-PEG2000, and DSPE-PEG5000-MAL conjugates (or targeting moieties), wherein the molar ratio is (30-65):(10-30):(20-50):(0.5-5):(0.1-2), preferably (40-60):(10-20):(25-45):(0.5-2):(0.1-2).

In another preferred embodiment, the lipid nanoparticle modified with a targeting moiety comprises an ionizable lipid, DSPC, cholesterol, DSPE-PEG2000 or DMG-PEG2000, and DSPE-PEG5000-MAL conjugates (or targeting moieties), wherein the molar ratio is (40-60):(10-20):(28.5-38.5):(0.5-1.5):(0.1-1).

In another preferred embodiment, the targeting moiety is selected from at least one from: ligands, receptors, antibodies, antigen-binding fragments of antibodies, aptamers, and polypeptides.

In another preferred embodiment, the targeting moiety comprises antibodies and antigen-binding fragments thereof that target the target protein.

In another preferred embodiment, the antibodies comprise monoclonal antibodies and polyclonal antibodies.

In another preferred embodiment, the antibodies and antigen-binding fragments thereof are selected from: an intact antibody, a nanobody (VHH), a Fab fragment, a Fab′ fragment, an F(ab′)2 fragment, an F(ab′)3 fragment, Fv, a single-chain variable fragment (“scFv”), a di-scFv, and a (scFv)2.

In another preferred embodiment, the modification comprises covalent conjugation, non-covalent mixing, and/or other chemical bonding interactions.

In another preferred embodiment, the target protein is an immune cell surface protein.

In another preferred embodiment, the immune cells include T cells, B cells, NK cells, or a combination thereof.

In another preferred embodiment, the T cells are selected from: primary human T cells, JM cells, Jurkat cells, or a combination thereof.

In another preferred embodiment, the primary human T cells comprise helper T cells, cytotoxic T cells, regulatory T cells, memory T cells.

In another preferred embodiment, the target protein is a cell surface protein selected from: CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR1, CCR2, CCR4, CCR6, CCR7, or a combination thereof.

In another preferred embodiment, the target protein is a cell surface protein selected from: CD3, CD4, CD5, CD8, CD7, CD28, CD45, CD2, or a combination thereof.

In another preferred embodiment, the lipid nanoparticle further comprises a bioactive substance encapsulated in the lipid nanoparticle.

In another preferred embodiment, the bioactive substance is selected from the following group: nucleic acids, proteins, polypeptides, small molecules, or a combination thereof.

In another preferred embodiment, the nucleic acid includes DNA, plasmids, messenger RNA (mRNA), small interfering RNA (siRNA), antisense oligonucleotides, small RNAs, ribosomal RNAs, microRNAs and transfer RNAs, preferably is mRNA.

In a fourth aspect of the present disclosure, provides a lipid nanoparticle pharmaceutical formulation, the lipid nanoparticle pharmaceutical formulation comprises:
i) the lipid nanoparticle according to the third aspect of the present disclosure;
ii) a biologically active substance encapsulated in the lipid nanoparticle; and
iii) a pharmaceutically acceptable carrier.

In another preferred embodiment, the bioactive substance is selected from the following group: nucleic acids, proteins, polypeptides, small molecules, or a combination thereof.

In another preferred embodiment, the nucleic acid includes DNA, plasmids, messenger RNA (mRNA), small interfering RNA (siRNA), antisense oligonucleotides, small RNAs, ribosomal RNAs, microRNAs and transfer RNAs, preferably is mRNA.

In another preferred embodiment, the bioactive substance is a nucleic acid, and in the lipid nanoparticle pharmaceutical formulation, the molar ratio of ionizable N atoms in the ionizable lipid molecules to phosphate groups in the nucleic acid molecules is (2-10):1, further preferably (4-8):1, more preferably 3:1, 4:1, 5:1 or 6:1.

In another preferred embodiment, the hydrated particle size of the lipid nanoparticle pharmaceutical formulation is 50-200 nm, more preferably 70-150 nm, and most preferably 75-110 nm.

In another preferred embodiment, the particle size of the lipid nanoparticle is 80-170 nm, 85-150 nm, 90-135 nm, 95-125 nm, 88.68-132.78 nm, or 88.08-99.84 nm.

In another preferred embodiment, the PDI (Polydispersity Index) of the lipid nanoparticle is ≤0.25, or the PDI is ≤0.22.

In another preferred embodiment, the PDI of the lipid nanoparticle is 0.04-0.22, 0.05-0.20, 0.07-0.16, or 0.073-0.136.

In another preferred embodiment, the encapsulation efficiency of the lipid nanoparticle is ≥60%, ≥80%, or ≥85%.

In another preferred embodiment, the encapsulation efficiency of the lipid nanoparticle is 70-98%, 85-98%, 90-98%, 66.53-96.28%, 90.33%-97.81%, or 93.0-95.77%.

In another preferred embodiment, the cell viability rate of the lipid nanoparticle when transfecting cells is ≥50%, ≥70%, ≥85%, or ≥90%.

In another preferred embodiment, the cell viability rate of the lipid nanoparticle when transfecting cells is 88-99%, 93-99%, 92.26%-98.31%, or 97.86%-99.38%.

In another preferred embodiment, the transfection efficiency of the lipid nanoparticle is >74.54%, or the transfection efficiency of the lipid nanoparticles is 75-99% or 75-89%.

In another preferred embodiment, the expression level of the lipid nanoparticle is >6338.1 RFU, or the expression level of the lipid nanoparticles is 6500-12400 RFU or 6600-9100 RFU.

In another preferred embodiment, the lipid nanoparticle pharmaceutical formulation can be used for treating and/or preventing a tumor, an infectious disease and a rare disease.

In another preferred embodiment, the dosage form of the lipid nanoparticle pharmaceutical formulation is selected from the following group: injections, lyophilized preparations, aerosol inhalants, and topical formulations.

In another preferred embodiment, the lipid nanoparticle pharmaceutical formulation is administered via injection, i.e., intravenous, intramuscular, intradermal, subcutaneous, intrathecal, intraduodenal or intraperitoneal injection.

In another preferred embodiment, the lipid nanoparticle pharmaceutical formulation is administered via inhalation, e.g., intranasal administration.

In another preferred embodiment, the lipid nanoparticle pharmaceutical formulation is administered transdermally, e.g., via transdermal application or iontophoresis.

In a fifth aspect of the present disclosure, provides a method for preparing the lipid nanoparticle pharmaceutical formulation according to the fourth aspect of the present disclosure, the method comprises:
(a) mixing the ionizable lipid, or pharmaceutically acceptable salt, tautomer or stereoisomer thereof according to the first aspect of the present disclosure, and optional auxiliary lipid with an organic solvent to obtain a lipid organic phase.
(b) mixing a biologically active substance with an aqueous solvent to obtain an aqueous phase containing the biologically active substance;
(c) mixing the lipid organic phase in step (a) with the aqueous phase in step (b) to obtain the lipid nanoparticle pharmaceutical formulation.

In another preferred embodiment, the organic solvents include ethanol, methanol, isopropanol, acetonitrile, dimethylformamide, dimethyl sulfoxide, dioxane, tetrahydrofuran or a combination thereof, preferably ethanol.

In another preferred embodiment, the aqueous solvent is a buffer solution.

In another preferred embodiment, the aqueous solvent is a buffer solution with a pH range of 3-7.

In another preferred embodiment, the acidic buffer solution is a citrate buffer solution with a pH of 4.0.

In another preferred embodiment, the volume ratio of the lipid organic phase to the aqueous phase containing the bioactive substance is 1:(2-5), more preferably 1:(3-4).

In another preferred embodiment, in step (c), the lipid organic phase and the aqueous phase are mixed via a microfluidic chip.

In another preferred embodiment, the method further comprises step (d): purifying, concentrating and sterilizing by filtration the lipid nanoparticle pharmaceutical formulation obtained in step (c).

In a sixth aspect of the present disclosure, provides a use of the ionizable lipid, or pharmaceutically acceptable salt, tautomer or stereoisomer thereof according to the first aspect of the present disclosure in the manufacture of a drug delivery system.

In another aspect of the present invention, provides the use of the ionizable lipid, or a pharmaceutically acceptable salt, tautomer or stereoisomer thereof, or the ionizable lipid prepared by the method, or a pharmaceutically acceptable salt, tautomer or stereoisomer thereof, in the manufacture of a drug delivery system.

In another preferred embodiment, the delivery system includes lipid nanoparticles (LNPs), liposomes, polymer nanoparticles, etc, and is preferably used for preparing lipid nanoparticles.

In another preferred embodiment, the drug delivery system is used for delivering drugs for treating and/or preventing a tumor, an infectious disease and a rare disease.

In another aspect of the present invention, the drug delivery system is used for delivering prophylactic vaccines.

In a seventh aspect of the present disclosure, provides a use of the lipid nanoparticle according to the third aspect of the present disclosure in the manufacture of a medicament for treating and/or preventing a tumor, an infectious disease and a rare disease.

In another aspect of the present invention, provides the use of the lipid nanoparticle according to the third aspect of the present invention in the manufacture of a prophylactic vaccine.

In an eighth aspect of the present disclosure, provides a use of the ionizable lipid, or pharmaceutically acceptable salt thereof according to the first aspect of the present disclosure, the use in the manufacture of the lipid nanoparticle pharmaceutical formulation. The lipid nanoparticle pharmaceutical formulation is used for delivering a bioactive substance to the cells of a subject in need thereof.

In another aspect of the present invention, the bioactive substance is selected from: nucleic acids, proteins, polypeptides, small molecules, or a combination thereof.

In another preferred embodiment, the bioactive substance is selected from the following group: DNA, RNA (mRNA, tRNA, rRNA, miRNA), and natural and synthetic oligonucleotides (including antisense oligonucleotides (ASO), interfering RNA (RNAi) and small interfering RNA (siRNA)).

In another preferred embodiment, the bioactive substance is mRNA.

In another preferred embodiment, the mRNA is transfected into cells and expressed in the cells.

In another preferred embodiment, the cells are immune cells.

In another preferred embodiment, the cells include T cells, B cells, NK cells, or a combination thereof.

In another preferred embodiment, the T cells are selected from the following group: human primary T cells, JM cells, Jurkat cells, or a combination thereof.

In another preferred embodiment, the human primary T cells include helper T cells, regulatory T cells, and memory T cells.

In another preferred embodiment, the drug delivery system or the lipid nanoparticle pharmaceutical formulation is further modified with a targeting moiety.

In another preferred embodiment, the targeting moiety is selected from at least one from: ligands, receptors, antibodies, antigen-binding fragments of antibodies, aptamers, and polypeptides.

In another preferred embodiment, the targeting moiety comprises antibodies and antigen-binding fragments thereof that target the target protein.

In another preferred embodiment, the antibodies comprise monoclonal antibodies and polyclonal antibodies.

In another preferred embodiment, the antibodies and antigen-binding fragments thereof are selected from: an intact antibody, a nanobody (VHH), a Fab fragment, a Fab′ fragment, an F(ab′)2 fragment, an F(ab′)3 fragment, Fv, a single-chain variable fragment (“scFv”), a di-scFv, and a (scFv)2.

In another preferred embodiment, the targeting moiety modifies the lipid nanoparticle via the following approaches: covalent conjugation, non-covalent mixing, and/or other chemical bonding interactions.

In another preferred embodiment, the target protein is a cell surface protein selected from: CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR1, CCR2, CCR4, CCR6, CCR7, or a combination thereof.

In another preferred embodiment, the target protein is a cell surface protein selected from: CD3, CD4, CD5, CD8, CD7, CD28, CD45, CD2, or a combination thereof.

In another preferred embodiment, the ionizable lipid has the structure represented by the Formula below:

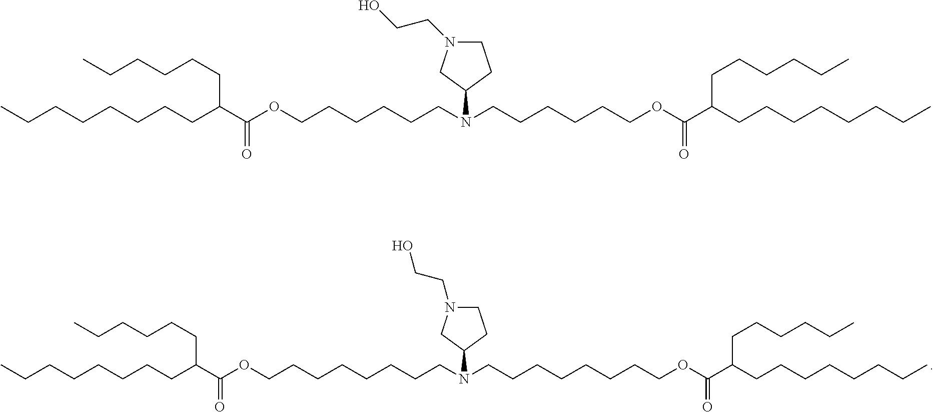

It should be understood that within the scope of the present disclosure, the aforementioned technical features of the present disclosure and the various technical features specifically described hereinafter (e.g., the examples) can be combined with each other to form new or preferred technical solutions. Due to space limitations, no further enumeration will be provided herein.

### BRIEF DESCRIPTION OF THE DRAWINGS

FIG. 1 shows the particle size characterization results of LNP-mRNA formulations with different compositions composed of AXT-8, with the particle size ranging from 80 nm to 130 nm, which demonstrates that the LNP prepared as described above have similar physicochemical properties. (Note: In the legends above, LNP(AXT-8)-Luc-1 refers to the LNP composed of molecule AXT-8 encapsulating Luc mRNA, where “1” denotes Formulation 1 among the series of LNPs encapsulating Luc mRNA, and the rest can be deduced by analogy; LNP(AXT-8)-hEPO-1 refers to the LNP composed of molecule AXT-8 encapsulating hEPO mRNA, where “1” denotes Formulation 1 among the series of LNPs encapsulating hEPO mRNA, and the rest can be deduced by analogy; LNP(AXT-8)-eGFP-1 refers to the LNP composed of molecule AXT-8 encapsulating eGFP mRNA, where “1” denotes Formulation 1 among the series of LNPs encapsulating eGFP mRNA; LNP(AXT-8)-mCherry-1 refers to the LNP composed of molecule AXT-8 encapsulating mCherry mRNA, where “1” denotes Formulation 1 among the series of LNPs encapsulating mCherry mRNA.)

FIG. 2 shows the particle size distribution characterization results of LNP-mRNA formulations with different compositions composed of AXT-8, with PDI<0.2, which demonstrates that the nanoparticles composed of AXT-8 have a narrow particle size distribution.

FIG. 3 shows the encapsulation efficiency characterization results of LNP-mRNA formulations with different compositions composed of AXT-8, with encapsulation efficiencies >90%, which demonstrates that the encapsulation effect is favorable.

FIG. 4 shows the transfection results of LNP-mRNA with different ratios composed of AXT-8 in 293T cells. As illustrated in the figure, with the increase in mRNA concentration, the cellular expression results of multiple formulations of LNP(AXT-8)-mRNA are all superior to those of Lipofectamine.

FIG. 5 shows the cytotoxicity results of LNP(AXT-8)-mRNA composed of AXT-8. As illustrated in the figure, when the mRNA concentration of the LNP(AXT-8) product series is below 1 g/mL, the cell inhibition rate thereof is nearly 0.

FIG. 6 shows the in vivo hEPO expression results of LNP-mRNA formulations with different compositions composed of AXT-8. As illustrated in the figure, for the different formulations, the trend of in vivo mRNA expression increases first and then decreases, and the peak expression is achieved at approximately 6 h for all formulations.

FIG. 7 shows the in vitro transfection expression results of LNP-mRNA formulations composed of AXT-8 in JM cells and Jurkat cells. As illustrated in the figure, LNP(AXT-8)-mRNA can be successfully expressed in both JM cells and Jurkat cells, with the expression levels being superior to those of Lipofectamine Max.

FIG. 8 shows the in vitro transfection expression results of LNP-mRNA formulations composed of AXT-8 in human primary T cells. As illustrated in the figure, LNP(AXT-8)-mRNA can be successfully expressed in human primary T cells, with the expression levels being superior to those of Lipofectamine Max.

FIG. 9 shows the ¹H NMR spectrum of AL08-001.

FIG. 10 shows ¹H NMR spectrum of AL08-003.

FIG. 11 shows ¹H NMR spectrum of AL08-004.

FIG. 12 shows ¹H NMR spectrum of AL08-005.

FIG. 13 shows ¹H NMR spectrum of AL08-006.

FIG. 14 shows ¹H NMR spectrum of AL08-007.

FIG. 15 shows ¹H NMR spectrum of AL08-008.

FIG. 16 shows ¹H NMR spectrum of AL08-009.

FIG. 17 shows ¹H NMR spectrum of AL08-010.

FIG. 18 shows ¹H NMR spectrum of AL08-011.

FIG. 19 shows ¹H NMR spectrum of AL08-012.

FIG. 20 shows ¹H NMR spectrum of AL08-014.

FIG. 21 shows ¹H NMR spectrum of AL08-015.

FIG. 22 shows ¹H NMR spectrum of AL08-018.

FIG. 23 shows ¹H NMR spectrum of AL08-019.

FIG. 24 shows ¹H NMR spectrum of AL08-020.

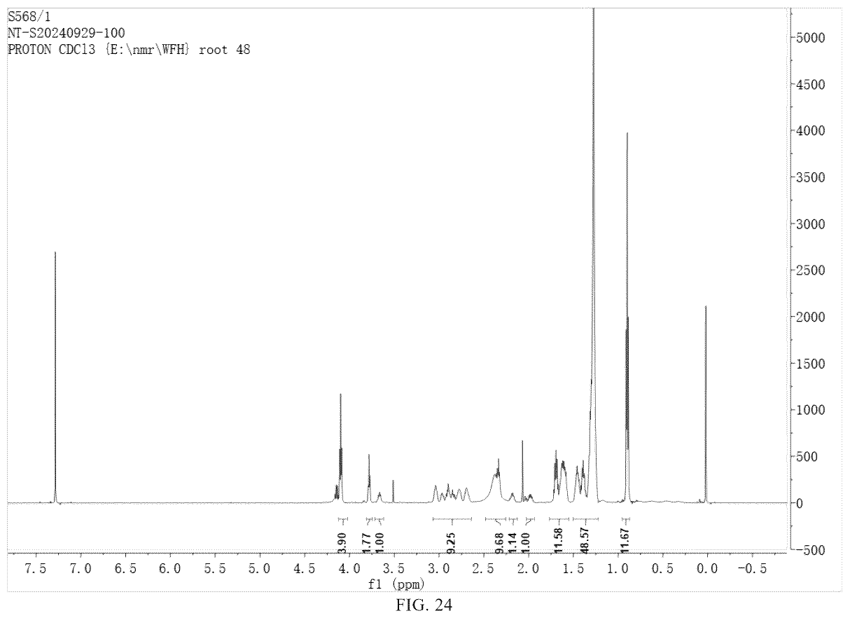

FIG. 25 shows ¹H NMR spectrum of AL08-021.

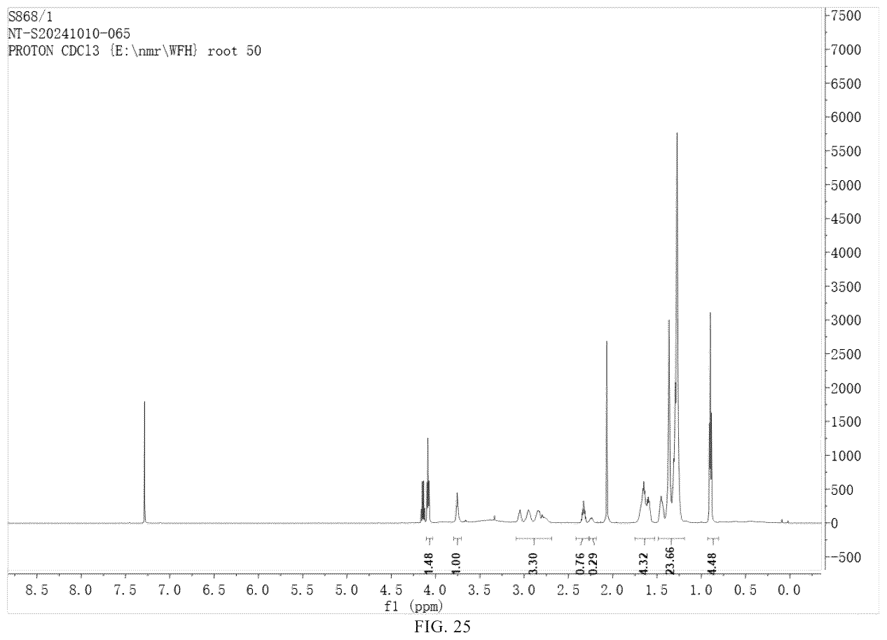

FIG. 26 shows ¹H NMR spectrum of AL08-022.

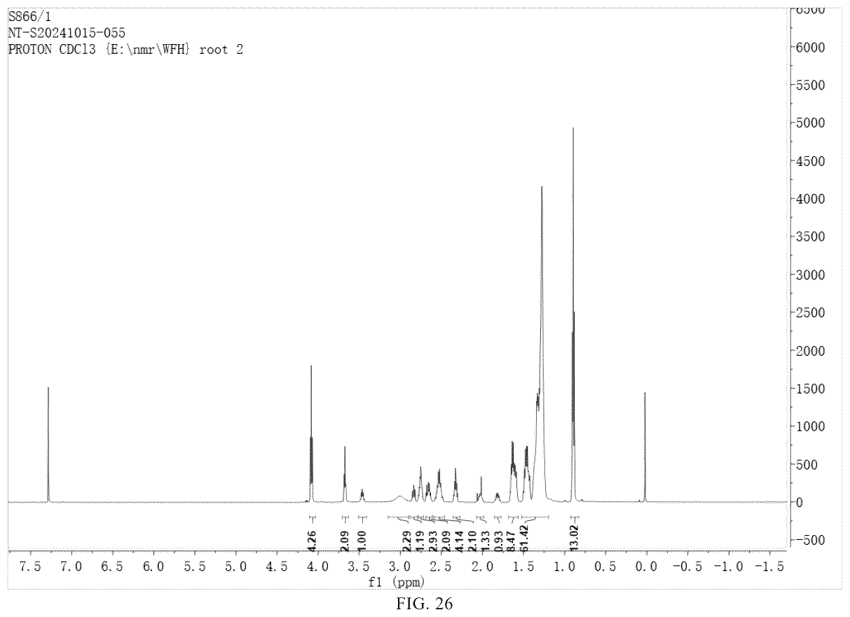

FIG. 27 shows ¹H NMR spectrum of AL08-023.

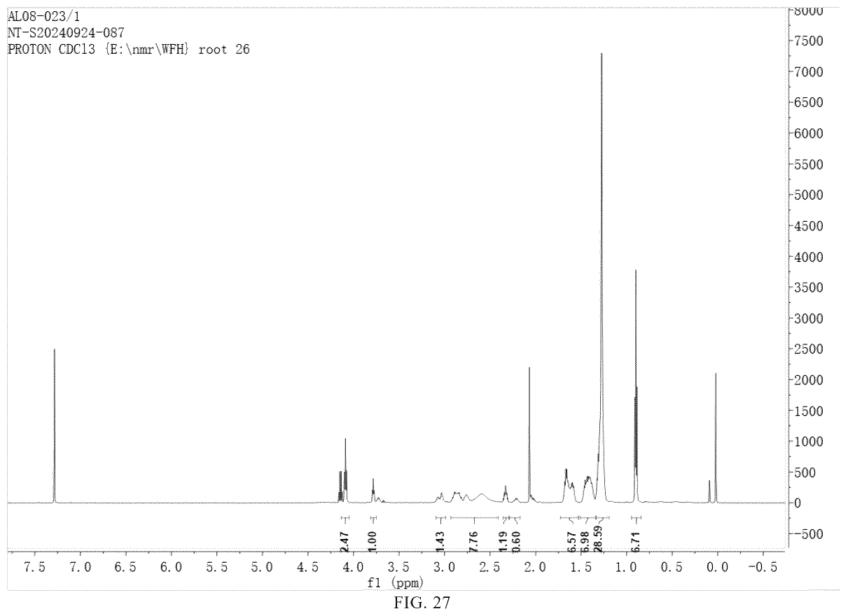

FIG. 28 shows ¹H NMR spectrum of AL08-024.

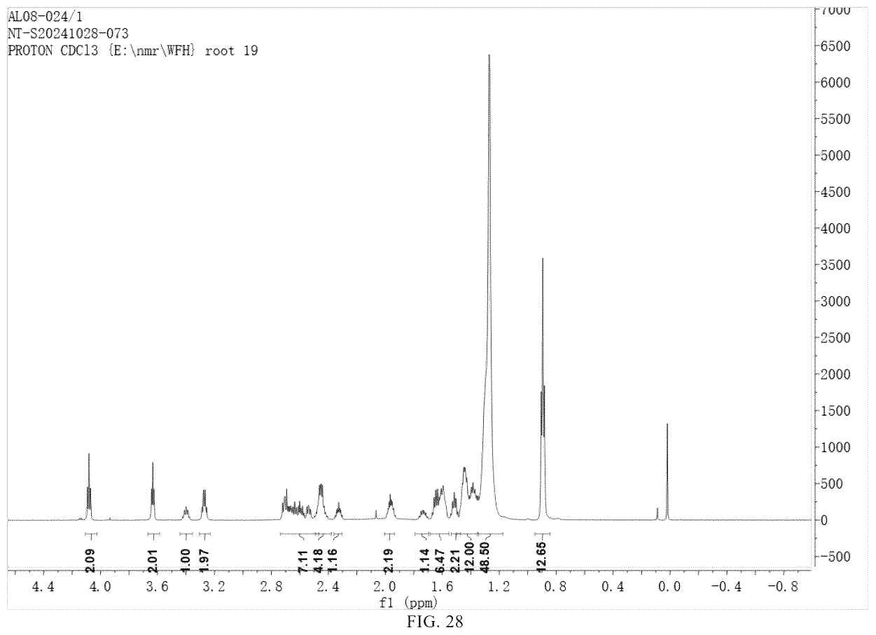

FIG. 29 shows ¹H NMR spectrum of AL08-025.

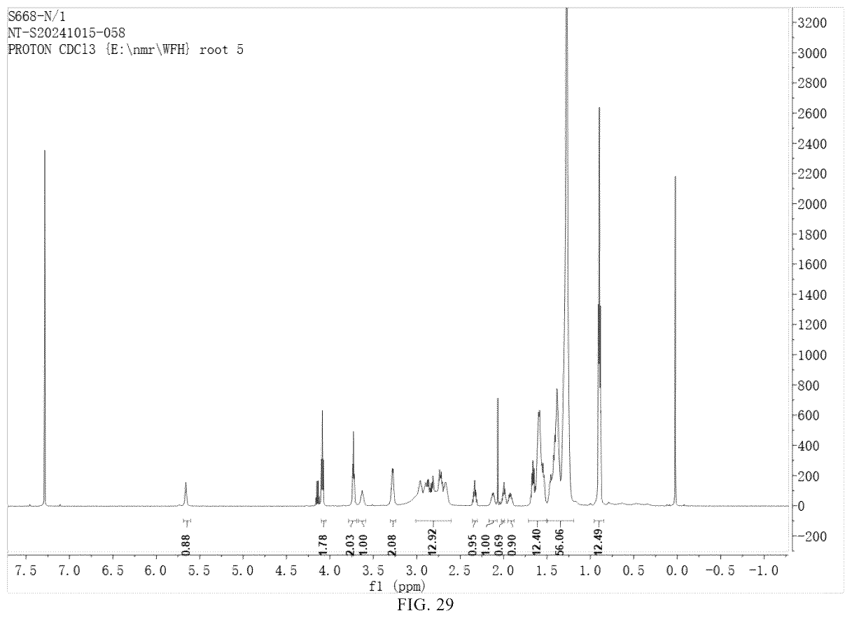

FIG. 30 shows ¹H NMR spectrum of AL08-026.

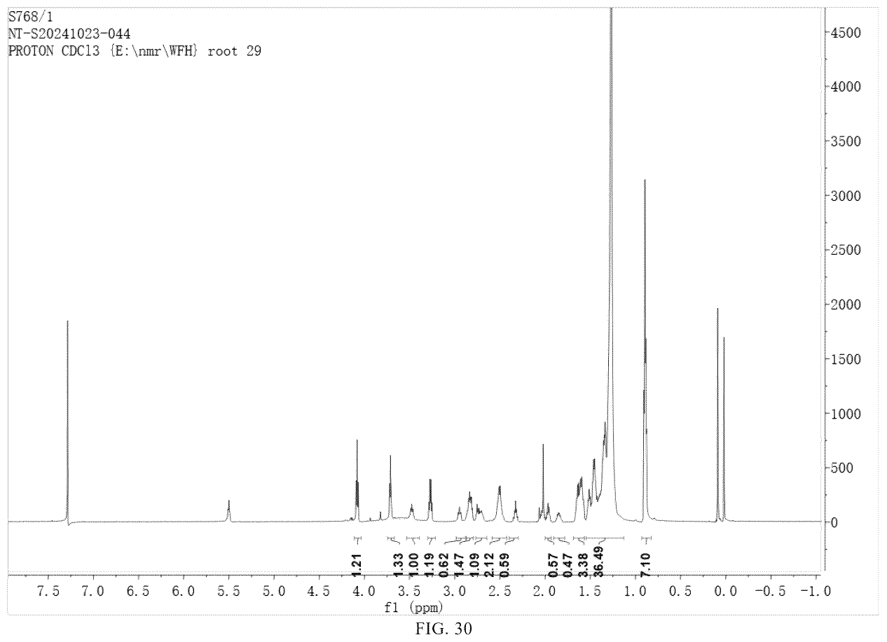

FIG. 31 shows ¹H NMR spectrum of AL08-027.

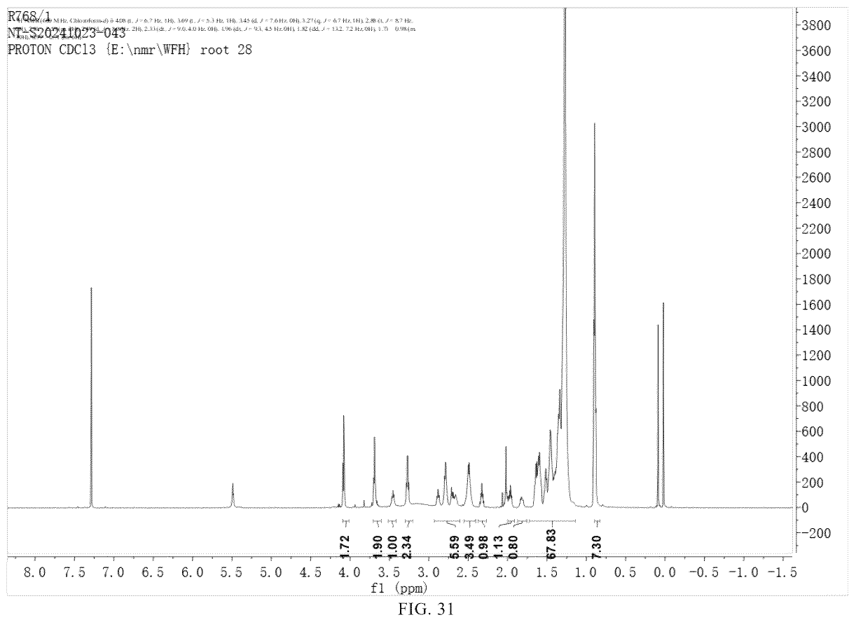

FIG. 32 shows the Flow cytometry results of the transfection of human primary T cells with LNP-mRNA (formulations AXT-8 LNP, ALP08-005-1, ALP08-012-1, ALP08-019-1, ALP08-022-1, ALP08-025-2, ALP08-025-2, ALP08-016-1 and ALP08-017-2 encapsulating mCherry).

FIG. 33 shows the results of Ab-LNP transfection of hPBMCs; other cell types include: B cells, NK cells, monocytes, etc.

### DETAILED DESCRIPTION OF THE EMBODIMENTS

After extensive and in-depth research was conducted by the Applicant, an ionizable lipid was unexpectedly discovered for the first time. The ionizable lipid was characterized by stable physicochemical properties and low toxicity. The drug delivery system obtained by encapsulating a drug payload (e.g., mRNA) with the ionizable lipid of the present disclosure exhibited high delivery efficiency and low toxicity. While enabling efficient delivery of the drug payload and increasing the expression level of the drug payload, the safety of the drug delivery system was improved, thereby rendering the preventive and therapeutic effects of the drug delivery system more prominent. On the basis of the aforementioned findings, the present disclosure was completed.

### Definition

To facilitate a better understanding of the present disclosure, certain terms are first defined. As used in the present disclosure, unless explicitly defined otherwise herein, each of the following terms shall have the meaning as set forth below.

The term “alkyl” refers to a saturated carbon chain having 1 to 20 carbon atoms, which can be linear, branched, or a combination thereof, unless the carbon chain is otherwise defined. Examples of alkyl includes methyl, ethyl, propyl, isopropyl, butyl, sec-butyl, tert-butyl, pentyl, hexyl, heptyl, octyl, etc.

Unless otherwise specifically stated in the description, the alkyl is optionally substituted. The term “unsaturated hydrocarbyl group” refers to a group containing at least one C═C double bond (alkenyl) or at least one C≡C triple bond (alkynyl). The “alkyl”, “alkenyl” and “alkynyl” can be collectively referred to as “the hydrocarbyl group”.

In the claims of the present disclosure, when “C1-C30 hydrocarbyl group” is described, it refers to the alkyl, alkenyl, or alkynyl having 1-30 carbon atoms (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms). “C₁-C₅ hydrocarbyl group”, “C₄-C₃₀ hydrocarbyl group”, “C₂-C₃₀ hydrocarbyl group” have analogous meanings.

When “alkyl” is described, it refers to a saturated hydrocarbyl having a specified number of carbon atoms (e.g., 2, 3, 4, 5, 6, 7, 8, 9, 10, 11, 12, 13, 14, 15, 16, 17, 18, 19, or 20 carbon atoms), which can be linear or branched, and is generally a chain group without a cyclic structure. The alkyl satisfying the aforementioned carbon atom number fall within the scope of this term.

When “alkenyl” is described, it refers to an alkenyl group having the specified number of carbon atoms (e.g., 2, 3, 4, 5, 6, 7, 8, 9, 10, 11, 12, 13, 14, 15, 16, 17, 18, 19, or 20 carbon atoms). The group can be linear or have a branched structure, and is generally a chain group without cyclic structures. All alkenyl groups satisfying the aforementioned carbon atom number fall within the scope of this term. In different embodiments of the present disclosure, the alkenyl can be derived from a monoalkene or a polyalkene (e.g., a diene).

When “C₂-C₃₀ linear or branched alkenyl” is described, it refers to the alkenyl having the specified number of carbon atoms (e.g., 2, 3, 4, 5, 6, 7, 8, 9, 10, 11, 12, 13, 14, 15, 16, 17, 18, 19, or 20 carbon atoms). The group can be linear or have a branched structure, and is generally a chain group without cyclic structures. All alkenyl satisfying the aforementioned carbon atom number fall within the scope of this term. In different embodiments of the present disclosure, the alkenyl can be monoalkenyl or polyalkenyl (e.g., diene).

When “C₁-C₁₀ alkylene” is described, it refers to the alkylene having 1 to 10 carbon atoms (e.g., 1, 2, 3, 4, 5, 6, 7, 8, 9, or 10 carbon atoms). The alkylene can be linear or branched, and is generally a chain group without a cyclic structure.

In the present application, when the definition of a divalent group includes “absent”, it refers to the adjacent structural moieties linked by the divalent group are directly connected via a chemical bond.

### Ionizable Lipid

As used herein, the terms “ionizable lipid of the present disclosure” and “ionizable cationic lipid of the present disclosure” are used interchangeably, and both refer to the lipid compounds having the structure of Formula I, or pharmaceutically acceptable salt, tautomer, or stereoisomer thereof.

The ionizable lipid becomes protonated and converted into a cationic lipid at a relatively low pH value, whereas it is converted back into a helper phospholipid under normal physiological pH conditions. The helper phospholipid has less interaction with the anionic cell membranes of blood cells, thereby enhancing the biocompatibility of the lipid nanoparticles. After the lipid nanoparticles are endocytosed by cells, the pH value in the endosomes is relatively low, causing the lipids to become protonated and positively charged. This impairs or even disrupts the stability of the membrane structure, thereby facilitating the endosomal escape of the lipid nanoparticles. In summary, the pH-sensitive property of the lipids is conducive to the in vivo delivery of lipid nanoparticles loaded with bioactive ingredients (e.g., mRNA molecules).

### Auxiliary Lipid

As used herein, the term “auxiliary lipid” refers to a type of lipid other than the ionizable lipid in a lipid nanoparticle, including helper phospholipids, sterols, polymer-conjugated lipids, or a combination thereof. The auxiliary lipid is mainly used to improve the performance of lipid nanoparticles, such as stability, delivery efficiency, tolerability and biodistribution, etc.

In some embodiments, the helper phospholipid includes (but is not limited to) 1,2-distearoyl-sn-glycero-3-phosphocholine (DSPC), 1,2-dioleoyl-sn-glycero-3-phosphoethanolamine (DOPE), dioleoylphosphatidylcholine (DOPC), 1,2-dioleoyl-sn-glycero-3-phosphocholine, 1,2-dipalmitoyl-sn-glycero-3-phosphocholine, 1,2-dipalmitoyl-sn-glycero-3-phosphoethanolamine, 1,2-dimyristoyl-sn-glycero-3-phosphoethanolamine, sodium 1,2-dioleoyl-sn-glycero-3-phospho-rac-(1-glycerol), 1,2-dipalmitoylphosphatidylglycerol, 1-palmitoyl-2-oleoylphosphatidylcholine, 1-palmitoyl-2-oleoylphosphoethanolamine, distearoyl phosphoethanolamine, 1-stearoyl-2-oleoylphosphatidylcholine, 1-stearoyl-2-oleoylphosphoethanolamine, or a combination thereof.

In a preferred embodiment of the present disclosure, the helper phospholipid is DSPC (1,2-distearoyl-sn-glycero-3-phosphocholine, also named distearoyl phosphatidylcholine). DSPC is a commonly used phosphatidylcholine; the tail group of DSPC is a saturated alkane chain, with a melting point of −54° C. and a cylindrical morphology. It forms a lamellar structure in lipid nanoparticles, thereby rendering the structure of the lipid nanoparticles more stable.

In a preferred embodiment of the present disclosure, the helper phospholipid is DOPE (1,2-dioleoyl-sn-glycero-3-phosphoethanolamine, also named dioleoyl phosphatidylethanolamine). DOPE is a commonly used phosphatidylethanolamine; the tail group of DOPE is two unsaturated alkane chains, with a melting point of −30° C. and a conical morphology. It readily forms an inverted hexagonal phase in lipid nanoparticles, which destabilizes the endosomal membrane and facilitates the endosomal escape of the lipid nanoparticles.

In some embodiments, the sterol includes (but is not limited to), cholesterol or cholesterol derivatives. Cholesterol can modulate the integrity and rigidity of the lipid membrane, thereby enhancing the stability of lipid nanoparticles. While the structural characteristics of cholesterol derivatives can affect the delivery efficiency and biodistribution of lipid nanoparticles, for example, the chain length of the hydrophobic tail group, the flexibility of the sterol ring, and the polarity of the hydroxyl group in cholesterol analogs. Cholesterol also affects the morphology of lipid nanoparticles, whereas cholesterol derivatives enable the formed lipid nanoparticles to adopt a lipid-separated multilamellar polyhedral structure rather than a spherical morphology. At same time, cholesterol also affects the targeting selectivity of lipid nanoparticles: lipid nanoparticles containing cholesteryl oleate exhibit higher selectivity for liver endothelial cells than for hepatocytes; whereas those containing cholesterol with oxidatively modified tail groups show higher accumulation in liver endothelial cells and Kupffer cells than in hepatocytes.

In some embodiments, the polymer-conjugated lipid is a polyethylene glycol (PEG)-conjugated lipid, also referred to as a pegylated lipid or PEGylated lipid. PEGylated lipids exert multiple effects on the properties of lipid nanoparticles: the dosage of PEGylated lipids affects the particle size and zeta potential of lipid nanoparticles, reduces particle aggregation to enhance the stability of lipid nanoparticles, decreases the rate of particle clearance mediated by the kidneys and the Mononuclear Phagocyte System (MPS), thereby prolonging the circulation time of the particles; the functional groups on the surface can be modified with ligands to improve targeted delivery capability. The molar mass and lipid chain length affect the properties of PEGylated lipids. Both DMG-PEG2000 and DSG-PEG2000 are neutral phospholipids with saturated alkyl chain lengths of C14, C16, or C18, respectively. However, DMG-PEG2000 can dissociate from lipid nanoparticles more rapidly, which facilitates cellular uptake of the nanoparticles and endosomal escape. Thus, DMG-PEG2000 exhibits superior delivery efficiency compared with DSG-PEG2000.

In a preferred embodiment of the present disclosure, the auxiliary lipid is a combination of DSPC, cholesterol, and DMG-PEG2000.

### Lipid Nanoparticle (LNP)

As used herein, the terms “lipid nanoparticle”, “lipid nanoparticulate” or “LNP” refer to particles with a diameter of about 5 to 500 nm. In some embodiments, the lipid nanoparticle contains one or more active agents (bioactive substances). In some embodiments, the lipid nanoparticle comprises nucleic acids. In some embodiments, the nucleic acids are condensed inside the nanoparticle with cationic lipids, polymers or multivalent small molecules, as well as an outer lipid coating that interacts with the biological environment. Due to the repulsion between phosphate groups, nucleic acids are inherently rigid polymers and tend to adopt an elongated conformation. In cells, to cope with volume constraints, DNA can package itself under appropriate solution conditions with the assistance of ions and other molecules. Generally, DNA condensation is defined as the collapse of extended DNA strands into compact, ordered particles containing only one or a few molecules. By binding to phosphate groups, cationic lipids can condense DNA by neutralizing phosphate charges and enabling its tight packing.

In some embodiments, the bioactive substance is encapsulated in the LNP. In some embodiments, the bioactive substance can be an anionic compound, including but not limited to DNA, RNA (messenger RNA, transfer RNA, ribosomal RNA, microRNA, etc.), natural and synthetic oligonucleotides (including antisense oligonucleotides, interfering RNA, and small interfering RNA), nucleoproteins, peptides, nucleic acids, ribozymes, DNA-containing nucleoproteins, e.g., fully or partially deproteinized viral particles (virions), and oligomeric and polymeric anionic compounds (e.g., acidic polysaccharides and glycoproteins) other than DNA. In some embodiments, the bioactive substance can be mixed with an adjuvant.

In LNP vaccine products, the bioactive substance is typically contained within the interior of the LNP. In some embodiments, the bioactive substance comprises nucleic acids. Generally, water-soluble nucleic acids are condensed with cationic lipids or polycationic polymers inside the particles, while the particle surface is enriched with helper phospholipids or PEG-lipid derivatives. Additional ionizable cationic lipids can also be located on the surface. Upon entering the lysosome of the cell, the ionizable cationic lipids become positively charged via ionization in the acidic environment of the lysosome, interact with the lysosomal membrane, and thereby facilitate endosomal escape.

With respect to LNPs, ionizable lipids can have different properties or functions. Owing to the pKa of amino groups, the ionizable lipid molecules will become protonated and positively charged when the external pH is lower than the pKa of the lipids. Under these conditions, the lipid molecules can electrostatically bind to the phosphate groups of nucleic acids, which enables LNP formation and nucleic acid encapsulation, while the LNPs exhibit a substantially neutral surface charge in biological fluids (e.g., blood) at physiological pH. A high surface charge of LNPs is associated with toxicity, rapid clearance by fixed and free macrophages in the circulation, hemolytic toxicity, and immune activation (Filion et al., Biochim Biophys Acta. 23 Oct. 1997; 1329 (2): 345-56).

In some embodiments, the pKa can be sufficiently high to enable the ionizable cationic lipid to adopt a positively charged form at the acidic endosomal pH. In this way, the cationic lipid can bind to endogenous endosomal anionic lipids, facilitating the formation of membrane-lytic non-bilayer structures, e.g., the hexagonal HII phase, thereby achieving more efficient intracellular delivery. In some embodiments, the pKa ranges from 6.2-6.5. For example, the pKa can be about 6.2, about 6.3, about 6.4, or about 6.5. Unsaturated tails also contribute to the ability of the lipid to form non-bilayer structures (Jayaraman et al., Angew Chem Int Ed Engl, 20 Aug. 2012; 51 (34): 8529-33).

The release of nucleic acids in the LNP formulation, as well as other characteristics such as liposome clearance rate and circulation half-life, can be modulated by the presence of polyethylene glycol and/or sterols (e.g., cholesterol) or other potential additives in the LNP, along with the overall chemical structure (including the pKa of any ionizable cationic lipids that are part of the formulation).

In one aspect of the present disclosure, provides a lipid nanoparticle (LNP), the lipid nanoparticle comprises the ionizable lipid, or pharmaceutically acceptable salt, tautomer or stereoisomer thereof according to the first aspect of the present disclosure. Further, the lipid nanoparticle also comprises one or more auxiliary lipids, the auxiliary lipids include helper phospholipids, steroids, and polymer-conjugated lipids. Further, the lipid nanoparticle comprises a targeting moiety modification.

In some embodiments, the targeting moiety is ligands, receptors, antibodies, antigen-binding fragments of antibodies, aptamers, and polypeptides. In some embodiments, the targeting moiety comprises antibodies and antigen-binding fragments thereof that target the target protein. In some embodiments, the antibodies comprise monoclonal antibodies and polyclonal antibodies. In some embodiments, the antibodies and antigen-binding fragments thereof are selected from: an intact antibody, a nanobody (VHH), a Fab fragment, a Fab′ fragment, an F(ab′)2 fragment, an F(ab′)3 fragment, Fv, a single-chain variable fragment (“scFv”), a di-scFv, and a (scFv)2. In some embodiments, the targeting moiety modifies the lipid nanoparticle via the following approaches: covalent conjugation, non-covalent mixing, and/or other chemical bonding interactions. In some embodiments, the target protein is a cell surface protein selected from: CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR1, CCR2, CCR4, CCR6, CCR7, or a combination thereof.

### Lipid Nanoparticle Pharmaceutical Formulation

In another aspect of the present disclosure, provides a lipid nanoparticle pharmaceutical formulation (or a lipid nanoparticle pharmaceutical composition, or an LNP composition). The lipid nanoparticle pharmaceutical formulation comprises the lipid nanoparticle according to the third aspect of the present disclosure, a bioactive substance encapsulated in the lipid nanoparticle, a pharmaceutically acceptable carrier, and an optionally targeting moiety modifying the lipid nanoparticle. The lipid nanoparticle pharmaceutical formulation is used for delivering a bioactive substance to the cells of a subject in need thereof.

In some embodiments, the targeting moiety is ligands, receptors, antibodies, antigen-binding fragments of antibodies, aptamers, and polypeptides. In some embodiments, the targeting moiety comprises antibodies and antigen-binding fragments thereof that target the target protein. In some embodiments, the antibodies comprise monoclonal antibodies and polyclonal antibodies. In some embodiments, the antibodies and antigen-binding fragments thereof are selected from: an intact antibody, a nanobody (VHH), a Fab fragment, a Fab′ fragment, an F(ab′)2 fragment, an F(ab′)3 fragment, Fv, a single-chain variable fragment (“scFv”), a di-scFv, and a (scFv)2. In some embodiments, the targeting moiety modifies the lipid nanoparticle via the following approaches: covalent conjugation, non-covalent mixing, and/or other chemical bonding interactions. In some embodiments, the target protein is a cell surface protein selected from: CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR1, CCR2, CCR4, CCR6, CCR7, or a combination thereof.

In some embodiments, the bioactive substance is encapsulated in the LNP. In some embodiments, the bioactive substance can be an anionic compound, including but not limited to DNA, RNA (messenger RNA, transfer RNA, ribosomal RNA, microRNA, etc.), natural and synthetic oligonucleotides (including antisense oligonucleotides, interfering RNA and small interfering RNA), nucleoproteins, peptides, nucleic acids, ribozymes, DNA-containing nucleoproteins, e.g., complete or partially deproteinized viral particles (virions), as well as oligomeric and polymeric anionic compounds other than DNA (e.g., acidic polysaccharides and glycoproteins). In some embodiments, the bioactive substance can mix with an adjuvant.

In some embodiments, the LNP composition comprises: a nucleic acid; the ionizable cationic lipid having the structure represented by Formula (I); a helper phospholipid (e.g., DSPC, DOPE, DOPC, or a combination thereof); a sterol (e.g., cholesterol, a cholesterol derivative, or a phytosterol such as β-sitosterol); and a polymer-conjugated lipid (e.g., DMG-PEG2000). In some embodiments, the LNP composition comprises: a nucleic acid; the ionizable cationic lipid having the structure represented by Formula (I), in an amount of 30-65% of the total lipids in the composition (molar ratio, the same below); a helper phospholipid (e.g., DSPC, DOPE, DOPC, or a combination thereof), in an amount of 5-30% of the total lipids in the composition; a sterol (e.g., cholesterol, a cholesterol derivative, or a phytosterol such as β-sitosterol), in an amount of 30-55% of the total lipids in the composition; and a polymer-conjugated lipid (e.g., DMG-PEG2000), in an amount of 1-5% of the total lipids in the composition. And, in the LNP composition, the molar ratio of the ionizable N atoms in the ionizable lipid molecules to the phosphate groups in the nucleic acid molecules is (2-10):1, further preferably (4-8):1.

In a preferred embodiment of the present disclosure, the LNP composition comprises: a nucleic acid; the ionizable cationic lipid having the structure represented by Formula (I); a helper phospholipid (e.g., DSPC, DOPE, DOPC, etc., or a combination thereof); a sterol (e.g., cholesterol, a cholesterol derivative, or a phytosterol such as β-sitosterol); and a polymer-conjugated lipid (e.g., DMG-PEG2000, etc.). In a more preferred embodiment of the present disclosure, the LNP composition comprises: a nucleic acid; the ionizable cationic lipid having the structure represented by Formula (I); a helper phospholipid (e.g., DSPC, DOPE, DOPC, etc., or a combination thereof); a sterol (e.g., cholesterol, a cholesterol derivative, or a phytosterol such as β-sitosterol); and a polymer-conjugated lipid (e.g., DMG-PEG2000, etc).

As used herein, the terms “encapsulation” and “encapsulated” refer to the incorporation of mRNA, DNA, siRNA, or other nucleic acid drugs within or associated with lipid nanoparticles. As used herein, the term “encapsulation” refers to complete encapsulation or partial encapsulation. For example, mRNA can be selected to treat and/or prevent the associated diseases when a lipid nanoparticle composition comprising mRNA is administered to a subject in need thereof.

As used herein, the term “pharmaceutically acceptable carrier” includes, but is not limited to, any adjuvant, carrier, excipient, scintillant, sweetener, diluent, preservative, dye/colorant, flavor enhancer, surfactant, wetting agent, dispersant, suspending agent, stabilizer, isotonic agent, solvent, or emulsifier approved by the Food and Drug Administration for use in humans or livestock.

### Method for Preparing a Lipid Nanoparticle Pharmaceutical Formulation

In another aspect of the present disclosure, provides a method for preparing a lipid nanoparticle pharmaceutical formulation. The method comprises: (a) mixing the ionizable lipid according to the first aspect of the present disclosure and an optional auxiliary lipid with an organic solvent to obtain a lipid organic phase; (b) mixing a bioactive substance with an aqueous solvent to obtain an aqueous phase containing the bioactive substance (c) mixing the lipid organic phase in step (a) with the aqueous phase in step (b) to obtain the lipid nanoparticle pharmaceutical formulation. Further, the method also comprises step (d): purifying, concentrating and sterilizing by filtration the lipid nanoparticle pharmaceutical formulation obtained in step (c).

In some embodiments, the organic solvent includes (but is not limited to) ethanol, methanol, isopropanol, acetonitrile, dimethylformamide, dimethyl sulfoxide, dioxane, or tetrahydrofuran, or a combination thereof. In some embodiments, the lipid organic phase comprises a small percentage of water or a pH buffer solution. The lipid organic phase can contain up to 60% by volume of water, e.g., up to about 0.05%, 0.1%, 0.5%, 1%, 2%, 3%, 4%, 5%, 10%, 15%, 20%, 25%, 30%, 35%, 40%, 45%, 50%, 55%, or 60% by volume of water. In one embodiment, the lipid organic phase comprises water in an amount ranging from about 0.05% to 60% by volume, e.g., ranging from about 0.05% to 50%, from about 0.05% to 40%, or from about 5% to 20% by volume of water.

In some embodiments, the lipid organic phase comprises a single type of lipid, e.g., an ionizable cationic lipid, a helper phospholipid, a sterol, or a polymer-conjugated lipid. In some embodiments, the lipid organic phase comprises multiple types of lipids. In one embodiment of the present disclosure, the lipid organic phase comprises the ionizable cationic lipid having the structure represented by Formula (I), a helper phospholipid (e.g., DSPC, DOPE, DOPC, or a combination thereof), a sterol (e.g., cholesterol, a cholesterol derivative, or a phytosterol such as β-sitosterol), and a polymer-conjugated lipid (e.g., DMG-PEG2000). In a preferred embodiment of the present disclosure, the lipid organic phase comprises the ionizable cationic lipid having the structure represented by Formula (I), a helper phospholipid (e.g., DSPC, DOPE, DOPC, or a combination thereof), a sterol (e.g., cholesterol, a cholesterol derivative, or a phytosterol such as β-sitosterol), and a polymer-conjugated lipid (e.g., DMG-PEG2000). In a more preferred embodiment of the present disclosure, the lipid organic phase comprises the ionizable cationic lipid having the structure represented by Formula (I), a helper phospholipid (e.g., DSPC, DOPE, DOPC, or a combination thereof), a sterol (e.g., cholesterol, a cholesterol derivative, or a phytosterol such as β-sitosterol), and a polymer-conjugated lipid (e.g., DMG-PEG2000). In a specific embodiment of the present disclosure, the lipid organic phase comprises the ionizable cationic lipid having the structure represented by Formula (I), DSPC, cholesterol, and DMG-PEG2000.

In some embodiments, the aqueous solvent is water. In some embodiments, the aqueous solvent is an aqueous buffer solution with a pH ranging from 3 to 8 (e.g., a pH of about 3, about 4, about 5, or about 6, etc.). A bioactive substance, e.g., a nucleic acid (e.g., mRNA), is dissolved in the aqueous solvent to obtain an aqueous phase containing the bioactive substance. The aqueous phase can contain a small percentage of a water-miscible organic solvent. The aqueous phase can contain up to 60% by volume of at least one water-miscible organic solvent, e.g., up to about 0.05%, 0.1%, 0.5%, 1%, 2%, 3%, 4%, 5%, 6%, 7%, 8%, 9%, 10, 1%, 20%, 25%, 30%, 35%, 40%, 45%, 50%, 55%, 60%, or any percentage by volume in between of an organic solvent (e.g., a water-miscible organic solvent). In one embodiment, the aqueous phase contains an organic solvent in an amount ranging from about 0.05% to 60% by volume, e.g., the organic solvent (e.g., a water-miscible organic solvent) in an amount ranging from about 0.05% to 50%, from about 0.05% to 40%, or from about 5% to 20% by volume. The aqueous buffer solution can be a citrate buffer, a Tris-HCl buffer solution, a sodium acetate buffer solution, a PBS buffer solution, or a combination thereof. In some embodiments, the aqueous buffer solution is a citrate buffer with a pH ranging from 4 to 6 (e.g., a pH of about 4, about 5, or about 6). In one embodiment, the aqueous buffer solution is a citrate buffer with a pH of about 4.

In some embodiments, the solution comprising the mixture of the lipid organic phase and the aqueous phase containing the bioactive substance, which contains the LNP suspension, can be diluted. In some embodiments, the pH of the solution comprising the mixture of the lipid organic phase and the aqueous phase containing the bioactive substance, which contains the LNP suspension, can be adjusted. The LNP suspension can be diluted or its pH can be adjusted by adding water, acid, base, or an aqueous buffer. In some embodiments, no dilution or pH adjustment of the LNP suspension is performed. In some embodiments, both dilution and pH adjustment are performed on the LNP suspension.

In some embodiments, excess reagents, solvents, and unencapsulated nucleic acids can be removed from the LNP suspension by tangential flow filtration (TFF) (e.g., diafiltration). Organic solvents (e.g., ethanol) and buffers can also be removed from the LNP suspension by TFF. In some embodiments, the LNP suspension is subjected to dialysis. In some embodiments, the LNP suspension is subjected to TFF. In some embodiments, the LNP suspension is subjected to both dialysis and TFF.

### The Main Advantages of the Present Disclosure Include:

(a) The ionizable lipids in the present disclosure can form stable nanoparticles with other components, e.g., helper phospholipids (DSPC, DOPE, DOPC, DOPS, etc.), sterols (e.g., cholesterol or cholesterol derivatives), and PEG derivatives (e.g., DMG-PEG lipids, or DMG-PEG substances modified with other groups, or other PEG derivatives).
(b) After the ionizable lipids in the present disclosure form nanoparticles with other components, the nanoparticles can encapsulate mRNA with uniform particle size, high encapsulation efficiency and good stability. They can improve the transfection efficiency of mRNA in targeted tissues or cells with low toxicity, thus making the preventive and therapeutic effects of mRNA vaccines/medicines more prominent.
(c) After the ionizable lipid of the present invention forms nanoparticles with other components, high T cell transfection efficiency can be achieved without activating T cells; after coupling with antibodies, the nanoparticles can accurately deliver drugs to target cells or tissues, improve drug delivery efficiency and bioavailability, and reduce damage and toxic reactions to normal cells.

The present disclosure was further illustrated below with reference to specific examples. It should be understood that these examples are merely intended to illustrate the present disclosure and not to limit the scope thereof. For the experimental methods without specified conditions in the following examples, they were generally carried out in accordance with conventional conditions or the conditions recommended by the manufacturers. Unless otherwise specified, all percentages and parts were by weight.

### Example 1

### The Preparation of Ionizable Lipid

1.1: Synthesis and characterization of compound AXT-8:

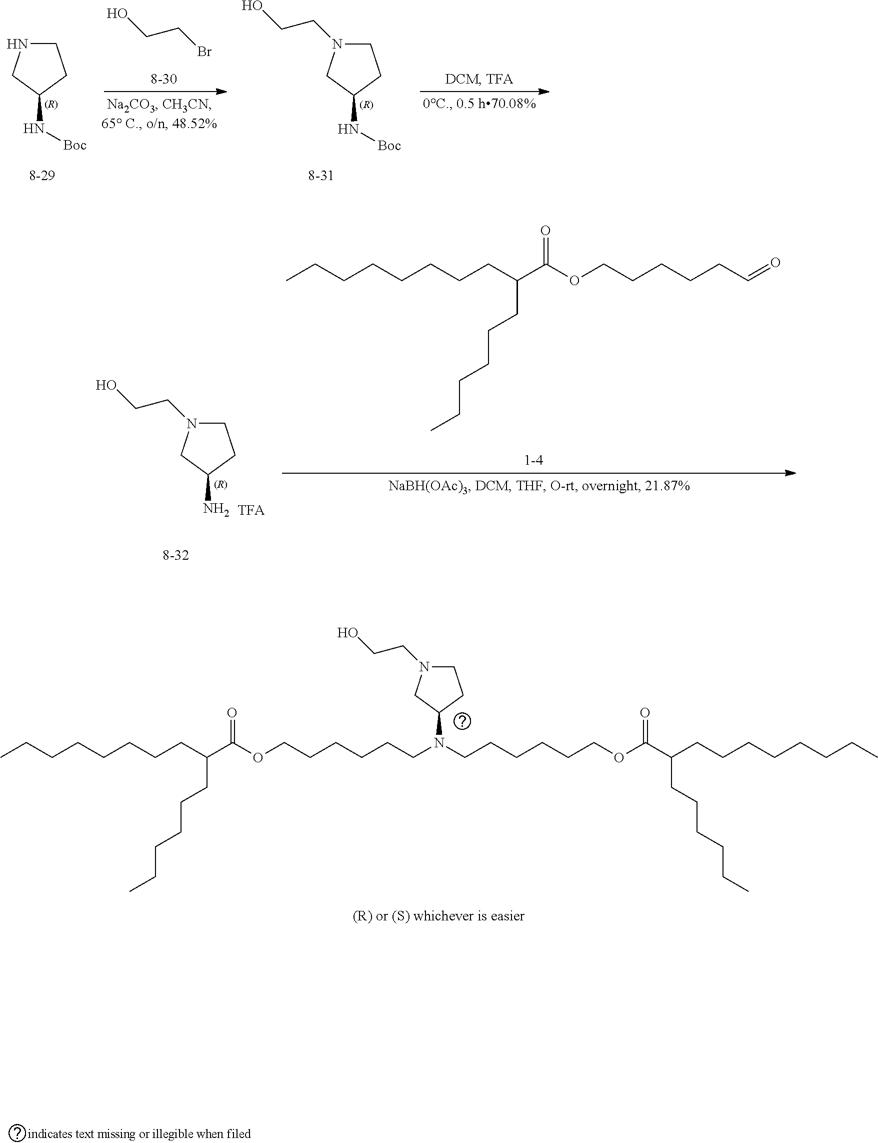

Synthesis flow chart of compound AXT-8

### 1.1.1: Synthesis and Characterization of Product 8-31

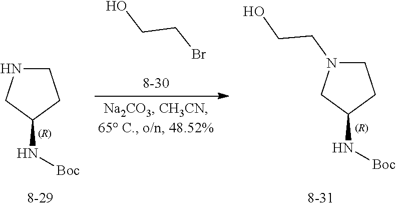

Tert-butyl N-[(3R)-pyrrolidin-3-yl]carbamate (1.2 g, 6.443 mmol, 1 equiv), 2-bromoethanol (1.21 g, 9.665 mmol, 1.5 equiv), Na₂CO₃ (1.37 g, 12.886 mmol, 2 equiv) and CH₃CN (24 mL) were placed in a 40 mL round-bottom flask. Under the protection of inert nitrogen atmosphere, the resulting solution was stirred overnight at 65° C.

Then, the reaction mixture was cooled to ambient temperature and filtered, and the filtrate was concentrated under reduced pressure. The residue was solubilized with 50 mL of water, and the resulting solution was extracted with 3×50 mL of ethyl acetate. The organic layers were combined, dried over anhydrous sodium sulfate, and concentrated.

As a result, tert-butyl N-[(3R)-1-(2-hydroxyethyl)pyrrolidin-3-yl]carbamate (0.72 g, yield 48.52%) was obtained as a colorless oil. The mass spectrometry and nuclear magnetic resonance data were shown as follows:

LC-MS-8-31: (ES, m/z): 231 [M+H]+;

H-NMR-8-31: 1H NMR (300 MHz, Chloroform-d, ppm) δ 4.998 (brs, 1H), 4.249 (brs, 1H), 3.702 (t, J=5.4 Hz, 2H), 3.025 (brs, 1H), 2.776-2.725 (m, 4H), 2.495-2.381 (m, 2H), 2.353-2.294 (m, 1H), 1.688 (t, J=8.4 Hz, 1H), 1.464 (s, 9H).

### 1.1.2: Synthesis and Characterization of Product 8-32

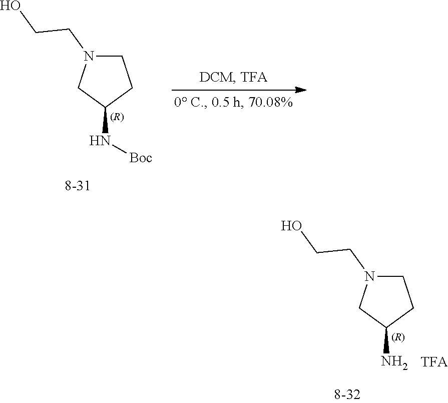

Tert-butyl N-[(3R)-1-(2-hydroxyethyl)pyrrolidin-3-yl]carbamate (0.72 g, 3.126 mmol, 1 equiv) and DCM (7.2 mL) were placed in a 100 mL three-necked round-bottom flask, which was protected by an inert nitrogen atmosphere.

Then, at 0° C., trifluoroacetaldehyde (1.45 mL) was added dropwise. The resulting solution was stirred at 0° C. for 30 minutes and monitored by LCMS. The resulting mixture was concentrated.

As a result, 2-[(3R)-3-aminopyrrolidin-1-yl]ethanol; trifluoroacetaldehyde adduct (0.5 g, yield: 70.08%) was obtained as a pale yellow oil. The results of mass spectrometry and nuclear magnetic resonance characterization were shown as follows:

LC-MS-8-32: (ES, m/z): 131 [M+H]+;

H-NMR-8-32: 1H NMR (300 MHz, Chloroform-d, ppm) δ 4.128 (brs, 1H), 3.830 (t, J=4.8 Hz, 2H), 3.653-3.392 (m, 4H), 3.353-3.279 (m, 2H), 2.612-2.488 (m, 1H), 2.235-2.142 (m, 1H).

1.1.3: Synthesis and characterization of final product

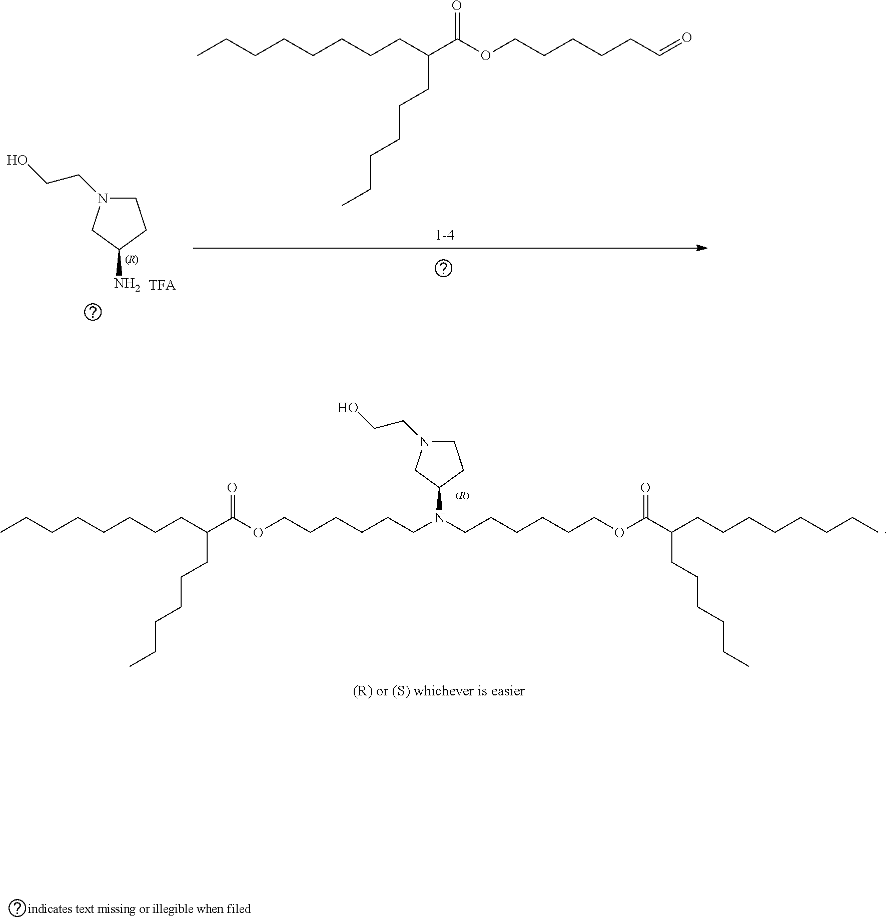

### Synthesis Procedures:

1) 2-[(3R)-3-aminopyrrolidin-1-yl]ethanol was placed in a 500 mL three-necked round-bottom flask. Under the protection of an inert nitrogen atmosphere, trifluoroacetaldehyde (300 mg, 1.315 mmol, 1 equiv), 6-oxohexyl-2-hexyldecanoate (1025.43 mg, 2.893 mmol, 2.2 equiv), DCM (15 mL) and THF (15 mL) were successively added. The resulting solution was stirred at room temperature for 0.5 hours;
2) At 0° C., diacetyl peroxide was added dropwise; sodium acetate borate (835.82 mg, 3.945 mmol, 3 equiv) was added with the dropping duration controlled within 5 minutes;
3) Stirring was continued at 0° C. for 0.5 hours, followed by stirring at room temperature overnight;
4) 50 mL of saturated NH₄Cl solution was added to quench the reaction;
5) The resulting mixture was extracted with 3×50 mL of DCM, and the organic layers were combined.
6) The combined organic phase was dried over anhydrous sodium sulfate and then concentrated under reduced pressure.
7) The residue was further purified by flash preparative HPLC under the following conditions (IntelFlash-1): column, C18 silica gel; mobile phase: A: H₂O (0.05% TFA) B: CH₃CN=55, linearly adjusted to H₂O (0.05% TFA) B: CH₃CN=95 within 15 minutes and maintained for 10 min; detector, UV detector at 220 nm; and
8) The qualified fractions were combined and concentrated. The residue was dissolved in 50 mL of DCM, and the organic phase was washed with 2×50 mL of NaHCO₃ (aq, 2M), 1×50 mL of water and 50 mL of brine, then dried over anhydrous Na₂SO₄.

The filtrate obtained after filtration was concentrated under reduced pressure. Thus, 6-({6-[(2-hexyldecyl)oxy]hexyl}[(3R)-1-(2-hydroxyethyl)pyrrolidin-3-yl]amino)hexyl 2-hexyldecanoate (0.2321 g, yield: 21.87%) was obtained as a yellow oil. The characterization results of mass spectrometry, nuclear magnetic resonance and HPLC were shown as follows.

LC-MS: (ES, m/z): 807.8 [M+H]⁺;

H-NMR: 1H NMR (300 MHz, Chloroform-d, ppm) δ 4.062 (t, J=6.6 Hz, 4H), 3.639 (t, J=5.1 Hz, 2H), 3.419 (t, J=6.9 Hz, 1H), 2.745-2.569 (m, 6H), 2.473 (t, J=7.8 Hz, 4H), 2.337-2.279 (m, 2H), 2.062-1.926 (m, 1H), 1.822-1.708 (m, 1H), 1.692-1.504 (m, 8H), 1.492-1.188 (m, 56H), 0.876 (t, J=5.7 Hz, 12H);

The results of purity analysis of AXT-8 by HPLC-CAD showed that the retention time of the product was 18.039 min, with a purity of over 94%.

### 1.2: Synthesis of Common Intermediates

The synthesis of common intermediates is as follows:

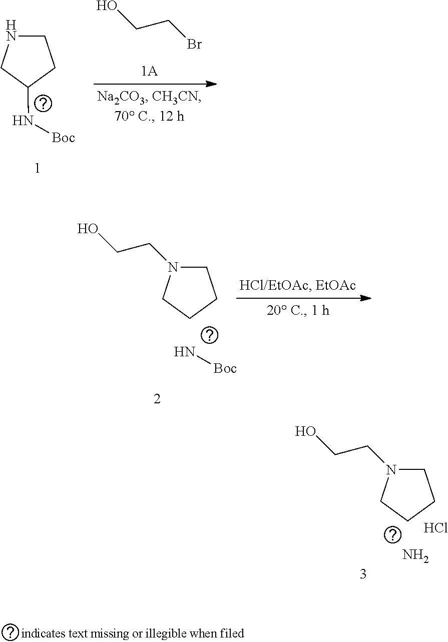

Specifically, it is divided into the following steps:

### Step 1:

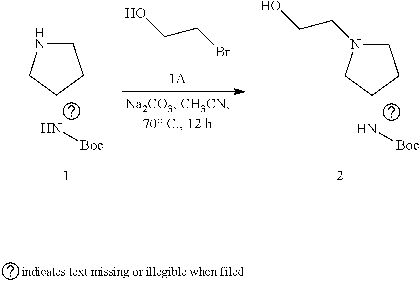

In a 250 mL reaction flask, under N₂ protection, sequentially add compound 1 (5 g, 0.0269 mol), compound 1A (3.72 g, 0.0296 mol, 1.5 equiv), Na₂CO₃ (5.70 g, 0.0538 mol, 2 eq) and CH₃CN (24 mL). The reaction is stirred overnight at 70° C., then cooled to room temperature and filtered under reduced pressure. The filtrate is concentrated under reduced pressure. 50 mL of water is added, extracted with ethyl acetate (3×50 mL). The organic phase is dried over anhydrous sodium sulfate, filtered and concentrated. Compound 2 (4.8 g, 0.0209 mol) was obtained as a colorless oil.

### Step 2:

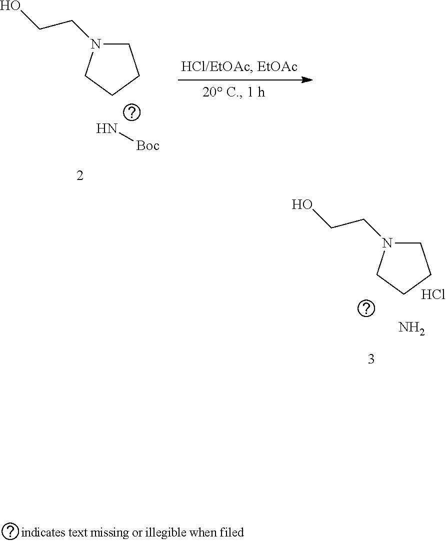

In a 100 mL single-neck flask, under N₂ protection, dissolve compound 2 (4.8 g, 0.0209 mol) in 10 mL ethyl acetate. Under stirring, dropwise add HCl in ethyl acetate (30 mL, 4 M). React at 20° C. for 30 minutes. LCMS shows the reaction is complete. The reaction solution is concentrated. Compound 3 (2.8 g, 0.02154 mol) was obtained as a light yellow oil.

The above is the synthesis of compound 3 with S-configuration amino head. The synthesis of R-configuration amino head is similar to the above steps, only requiring replacement of compound 1 with the corresponding R-configuration.

### 1.3: Synthesis of AL-08-001-027 Molecules

### 1.3.1: Synthesis of AL-08-005 Molecule

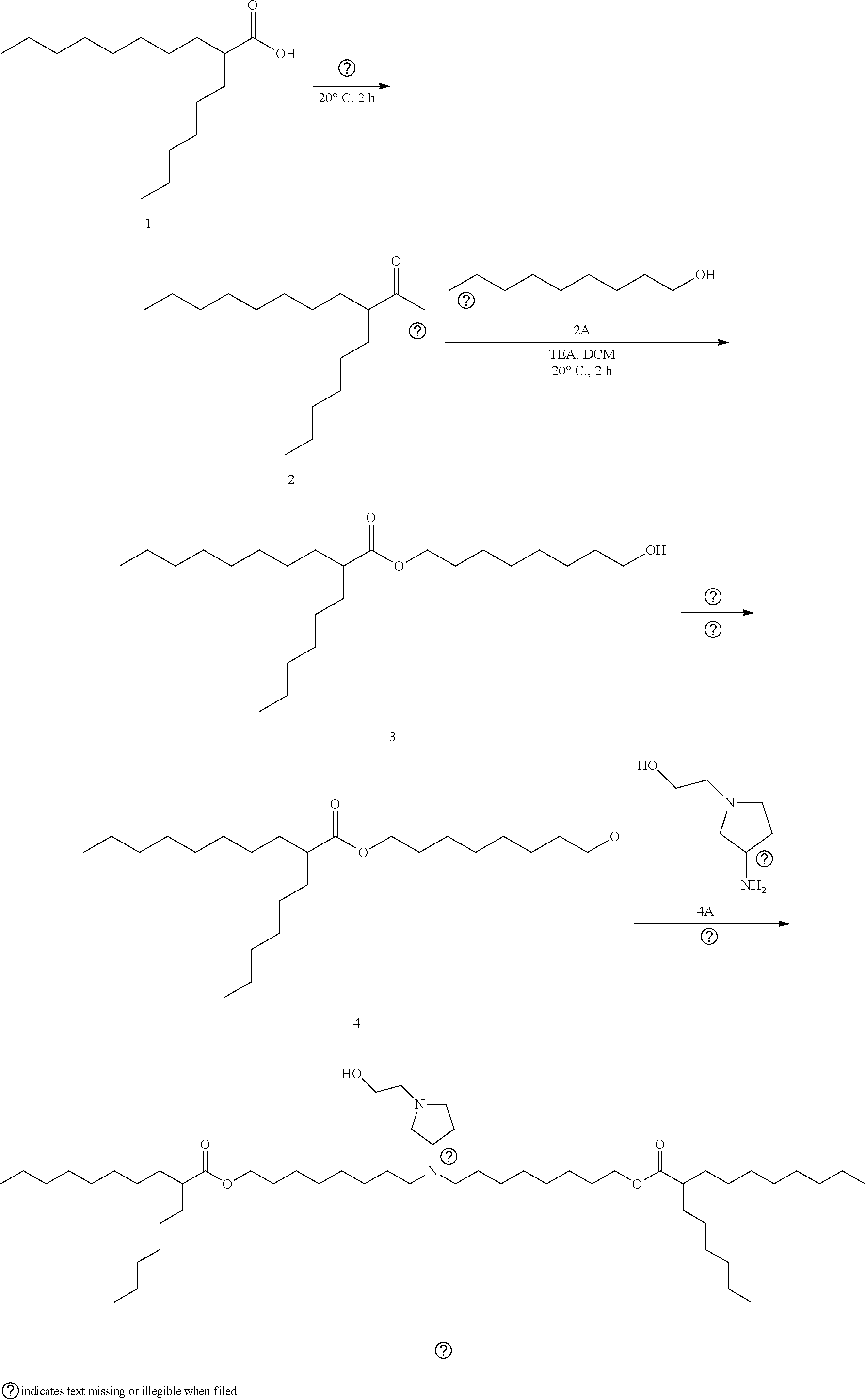

### Step 1:

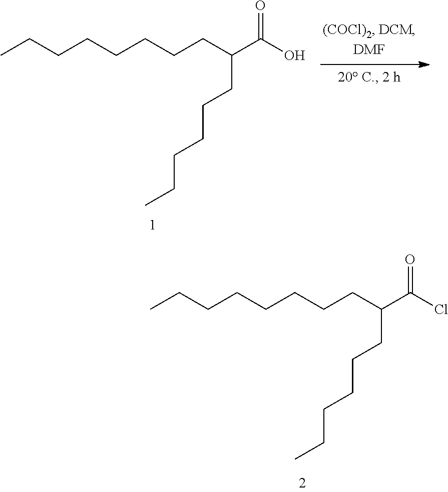 [SMILES: CCCCCCCCC(CCCCCC)C(=O)O ; CCCCCCCCC(CCCCCC)C(=O)Cl]

Dissolve compound 1 (8 g, 0.03112 mol) in DCM (150 mL) in a 250 mL three-neck flask. Replace with nitrogen three times. Cool the reaction to 0° C. with an ice-water bath. Under stirring, dropwise add COCl₂ (11.88 g, 0.09360 mol) and 0.5 mL DMF. After addition, warm to room temperature and stir for 2 hours. After reaction, take 2 drops of reaction solution and add to 0.5 mL methanol. Sample and run TLC (PE:EtOAc=10:1, Rf=0.6), which shows complete consumption of starting material and formation of a new spot. Pour the reaction solution into a 250 mL single-neck flask and concentrate under reduced pressure. Wash and concentrate with DCM (100 mL) twice. Compound 2 (8.2 g, 0.0299 mol) was obtained as a yellow liquid.

### Step 2:

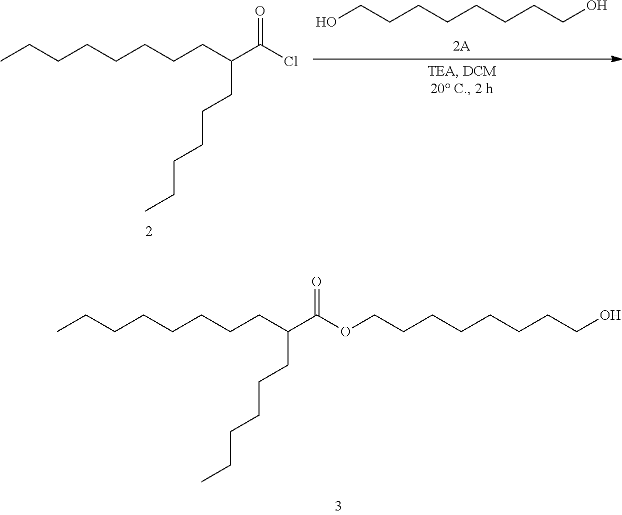 [SMILES: CCCCCCCCC(CCCCCC)C(=O)Cl ; OCCCCCCCCO ; CCCCCCCCC(CCCCCC)C(=O)OCCCCCCCCO]

Dissolve compound 2A (2.56 g, 0.0175 mol) in DCM (100 mL) in a 250 mL three-neck flask. Replace with nitrogen three times. Cool the reaction to 0° C. with an ice-water bath. Under stirring, dropwise add TEA (4.43 g, 0.0438 mol), then add 2 (4 g, 0.0146 mol). After addition, warm to room temperature and stir for 2 hours. After reaction, TLC (PE:EtOAc=3:1, Rf=0.4) shows complete consumption of starting material and formation of a new spot. Add 100 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography, and the product was eluted when the mobile phase was EtOAc PE (20%). Compound 3 (4 g, 0.0104 mol) was obtained as a colorless oil.

### Step 3:

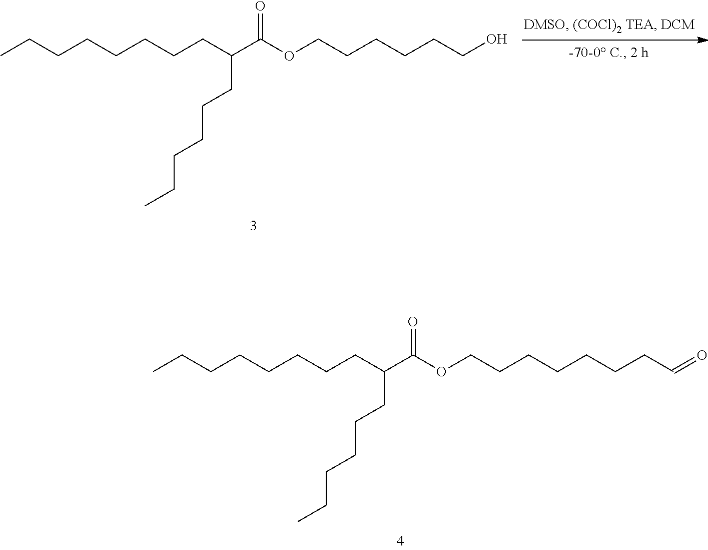 [SMILES: CCCCCCCCC(CCCCCC)C(=O)OCCCCCCO ; CCCCCCCCC(CCCCCC)C(=O)OCCCCCCCC=O]

Dissolve DMSO (2.12 g, 0.0312 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (3.99 g, 0.0312 mol). Stir at −60° C. for 30 min. Dissolve compound 3 (4 g, 0.0104 mol) in 10 mL DCM and slowly add dropwise to the above reaction solution. After addition, maintain temperature below −60° C. and stir for 1 h. Dropwise add TEA (5.26 g, 0.0520 mol) to the reaction solution below −60° C. Stir for 30 min after addition. Take 2 drops of reaction solution, add dichloromethane and water, extract and spot the organic phase. TLC (PE:EtOAc=3:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate under reduced pressure, dissolve, load sample. Column chromatography with mobile phase at EtOAc (8%) yields the product. Compound 4 (2.5 g, 0.0065 mol) was obtained as a light yellow oil at room temperature.

### Step 4:

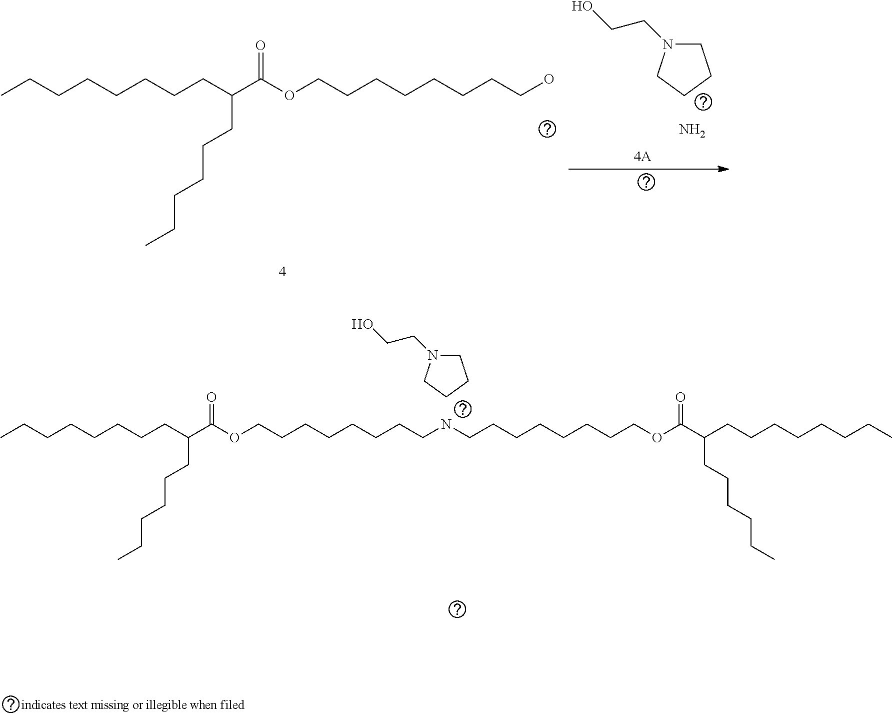

Add compound 4A (200 mg, 0.0015 mol) to a 100 mL single-neck flask. Add MeOH (15 mL) to dissolve, then add TEA (0.1 mL). Under stirring, add compound 4 (1.30 g, 0.0034 mol). Add 50 mg glacial acetic acid. Add sodium cyanoborohydride (483.38 mg, 0.0077 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate under reduced pressure. Dissolve crude product for sample loading. Column chromatography with mobile phase of 12% MeOH:88% EtOAc. Concentrate to obtain compound AL08-005 (400 mg, 0.00046 mol) as a light yellow oil.

### 1.3.2: Synthesis of AL-08-012 Molecule

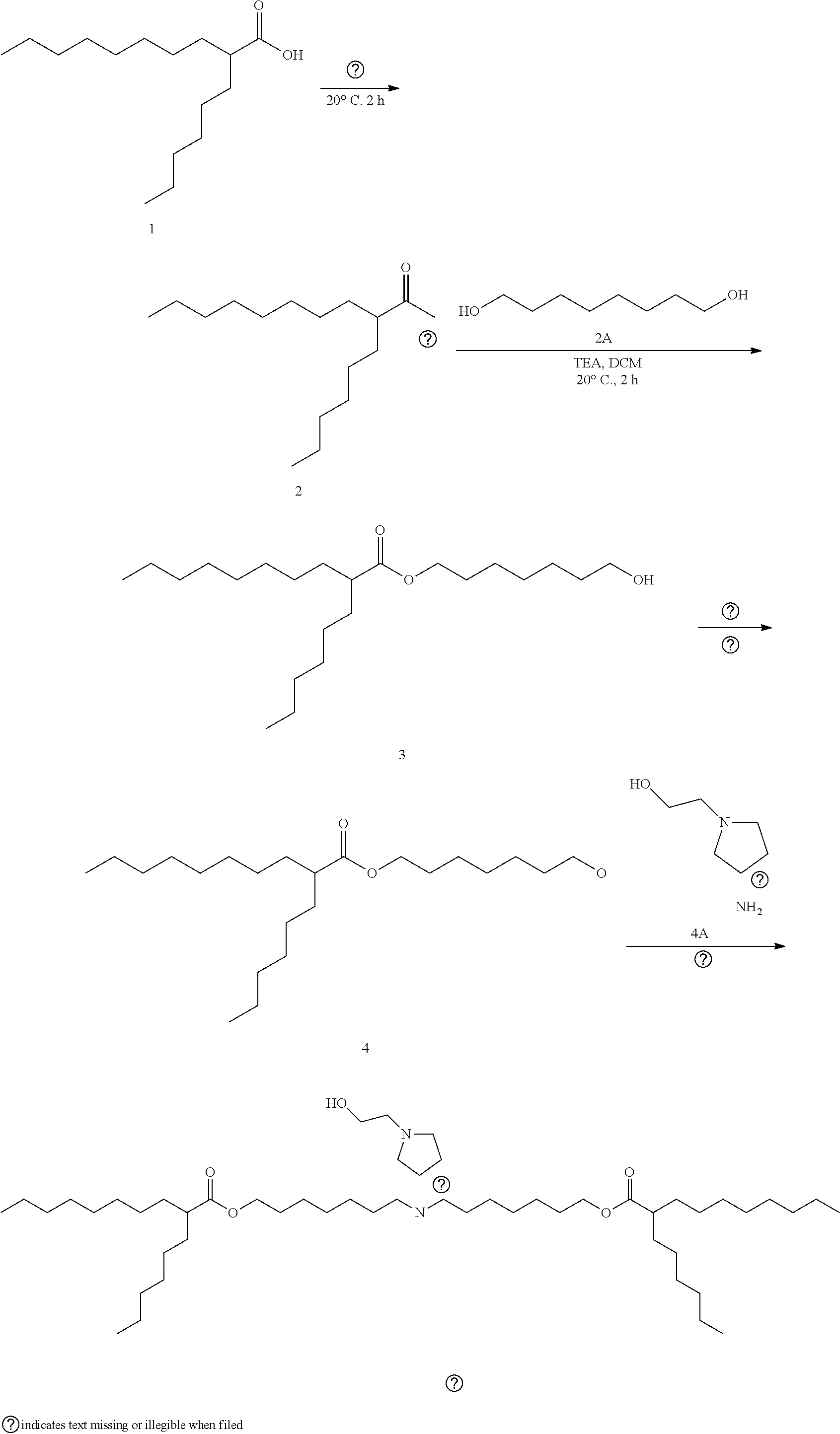

### Step 1:

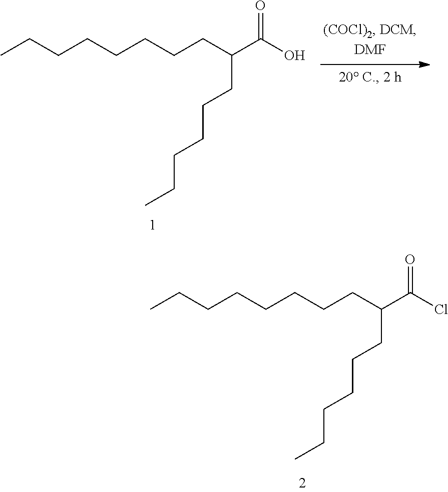 [SMILES: CCCCCCCCC(CCCCCC)C(=O)O ; CCCCCCCCC(CCCCCC)C(=O)Cl]

Dissolve compound 1 (8 g, 0.03112 mol) in DCM (150 mL) in a 250 mL three-neck flask. Replace with nitrogen three times. Cool the reaction to 0° C. with an ice-water bath. Under stirring, dropwise add COCl₂ (11.88 g, 0.09360 mol) and 0.5 mL DMF. After addition, warm to room temperature and stir for 2 hours. After reaction, take two drops of reaction solution and add to 0.5 mL methanol. Sample and run TLC (PE:EtOAc=5:1, Rf=0.6), which shows complete consumption of starting material and formation of a new spot. Pour the reaction solution into a 250 mL single-neck flask and concentrate under reduced pressure. Wash and concentrate with DCM (100 mL) twice. Compound 2 (8.2 g, 0.0299 mol) was obtained as a yellow liquid.

### Step 2:

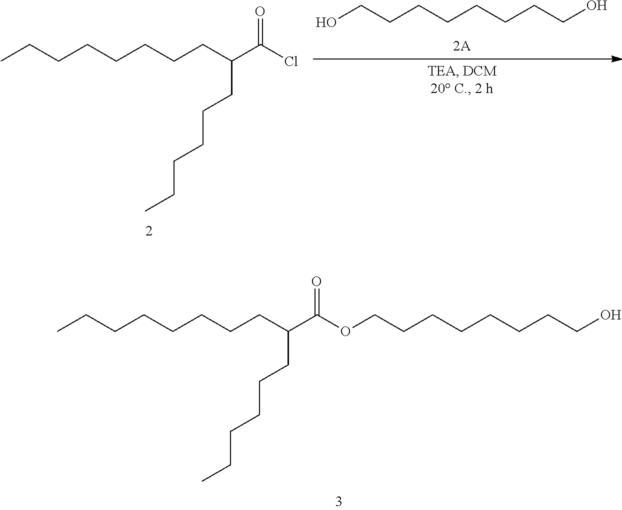 [SMILES: CCCCCCCCC(CCCCCC)C(=O)Cl ; OCCCCCCCCO ; CCCCCCCCC(CCCCCC)C(=O)OCCCCCCCCO]

Dissolve compound 2A (2.31 g, 0.0175 mol) in DCM (100 mL) in a 250 mL three-neck flask. Replace with nitrogen three times. Cool the reaction to 0° C. with an ice-water bath. Under stirring, dropwise add TEA (4.43 g, 0.0438 mol), then add compound 2 (4 g, 0.0146 mol). After addition, warm to room temperature and stir for 2 hours. After reaction, TLC (PE:EtOAc=3:1, Rf=0.6) shows complete consumption of starting material and formation of a new spot. Add 100 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (20%) yields the product. Compound 3 (3.5 g, 0.0095 mol) was obtained as a colorless oil.

### Step 3:

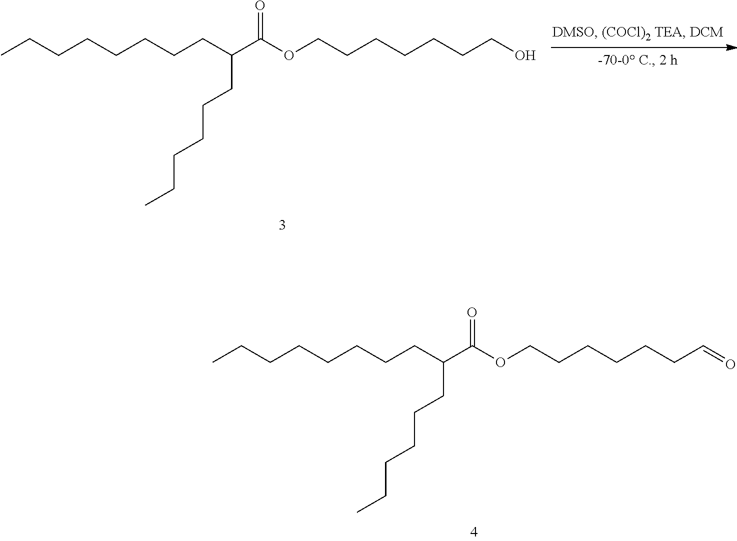 [SMILES: CCCCCCCCC(CCCCCC)C(=O)OCCCCCCCO ; CCCCCCCCC(CCCCCC)C(=O)OCCCCCCC=O]

Dissolve DMSO (1.94 g, 0.0285 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (3.65 g, 0.0285 mol). Stir at −60° C. for 30 min. Dissolve compound 3 (3.5 g, 0.0095 mol) in 10 mL DCM and slowly add dropwise to the above reaction solution. After addition, maintain temperature below −60° C. and stir for 1 h. Dropwise add TEA (4.81 g, 0.0475 mol) to the reaction solution below −60° C. Stir at 0° C. for 30 min after addition. Take 2 drops of reaction solution, add dichloromethane and water, extract and spot the organic phase. TLC (PE:EtOAc=3:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 4 (2 g, 0.0054 mol) was obtained as a light yellow oil at room temperature.

### Step 4:

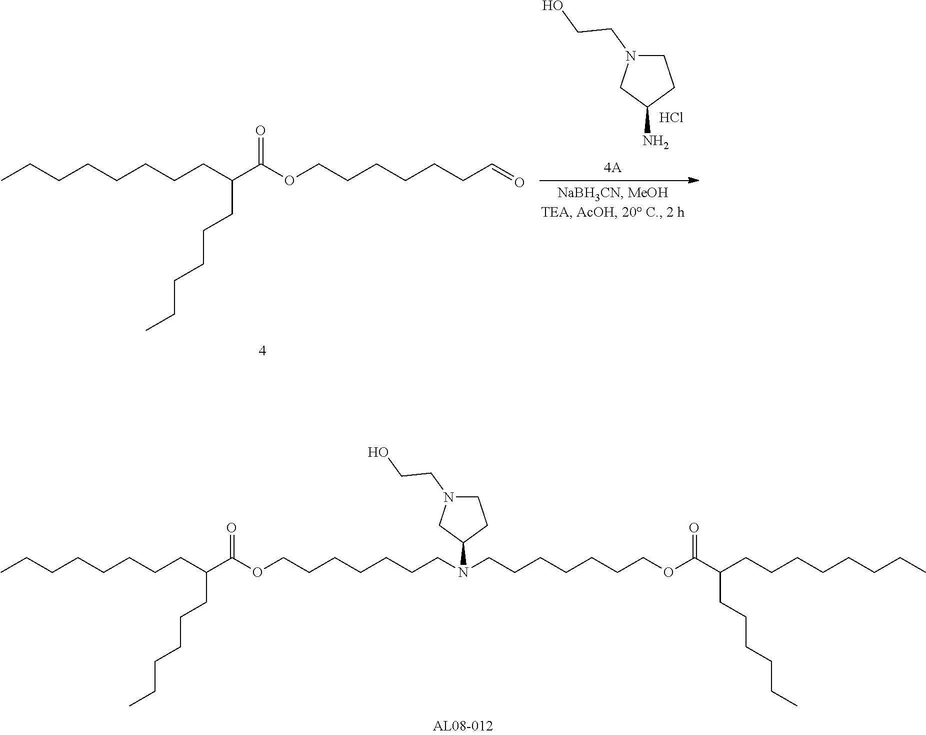 [SMILES: CCCCCCCCC(CCCCCC)C(=O)OCCCCCCC=O ; N[C@@H]1CCN(CCO)C1 ; Cl ; CCCCCCCCC(CCCCCC)C(=O)OCCCCCCCN(CCCCCCCOC(=O)C(CCCCCC)CCCCCCCC)[C@@H]1CCN(CCO)C1]

Add compound 4A (200 mg, 0.0015 mol) to a 100 mL single-neck flask. Add MeOH (15 mL) to dissolve, then add TEA (0.1 mL). Under stirring, add compound 4 (1.25 g, 0.0034 mol). Add 50 mg glacial acetic acid. Add sodium cyanoborohydride (483.38 mg, 0.0077 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate under reduced pressure. Dissolve crude product for sample loading. Column chromatography with mobile phase of 12% MeOH:88% EtOAc. Concentrate to obtain compound AL08-012 (371 mg, 0.00044 mol) as a light yellow oil.

### 1.3.3: Synthesis of AL08-001/003/004/006/007/008/011/018/019/020/021/022/023 Molecules

The synthesis of AL08-001/003/004/006/007/008/011/018/019/020/021/022/023 molecules is similar to the steps shown in 1.2.1˜1.2.2 above, thus not repeated here.

### 1.3.4: Synthesis of AL08-024 Molecule

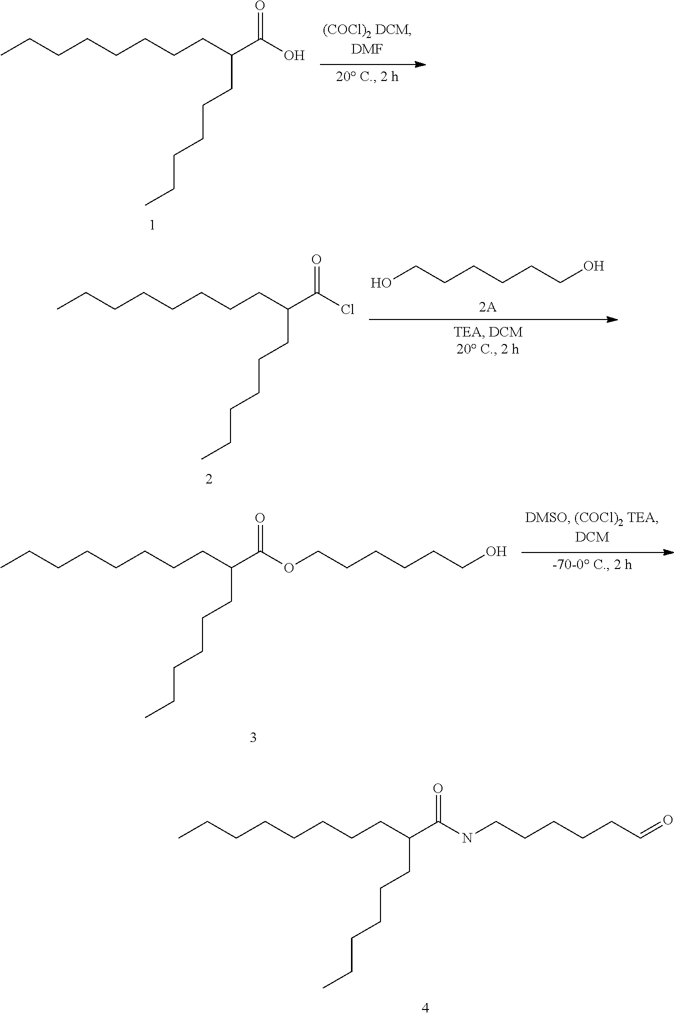 [SMILES: CCCCCCCCC(CCCCCC)C(=O)O ; CCCCCCCCC(CCCCCC)C(=O)Cl ; OCCCCCCO ; CCCCCCCCC(CCCCCC)C(=O)OCCCCCCO ; CCCCCCCCC(CCCCCC)C(=O)NCCCCCC=O]

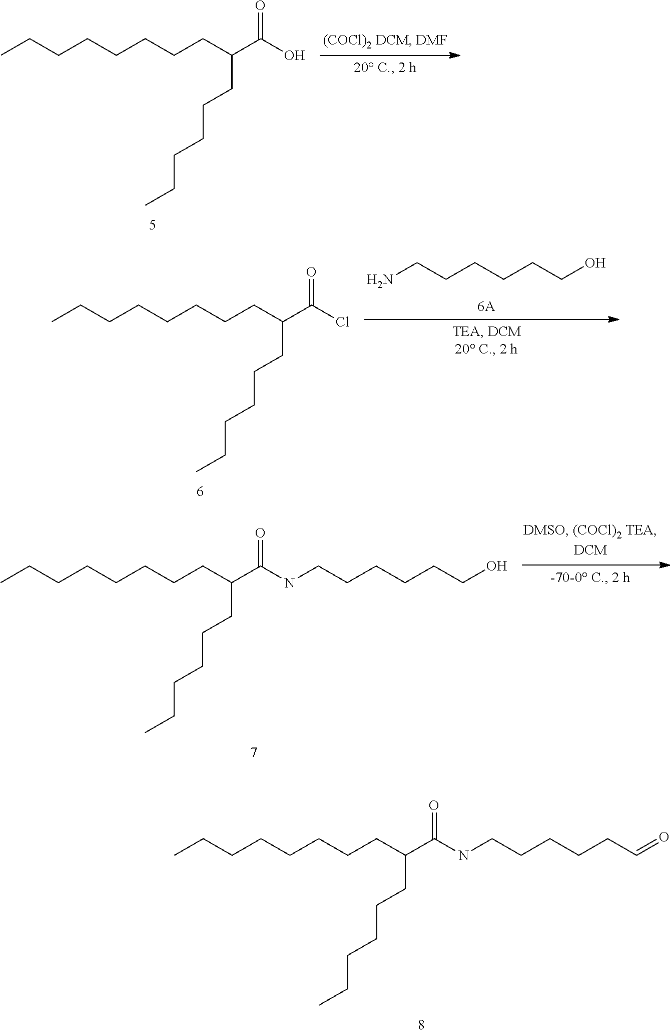 [SMILES: CCCCCCCCC(CCCCCC)C(=O)O ; CCCCCCCCC(CCCCCC)C(=O)Cl ; NCCCCCCO ; CCCCCCCCC(CCCCCC)C(=O)NCCCCCCO ; CCCCCCCCC(CCCCCC)C(=O)NCCCCCC=O]

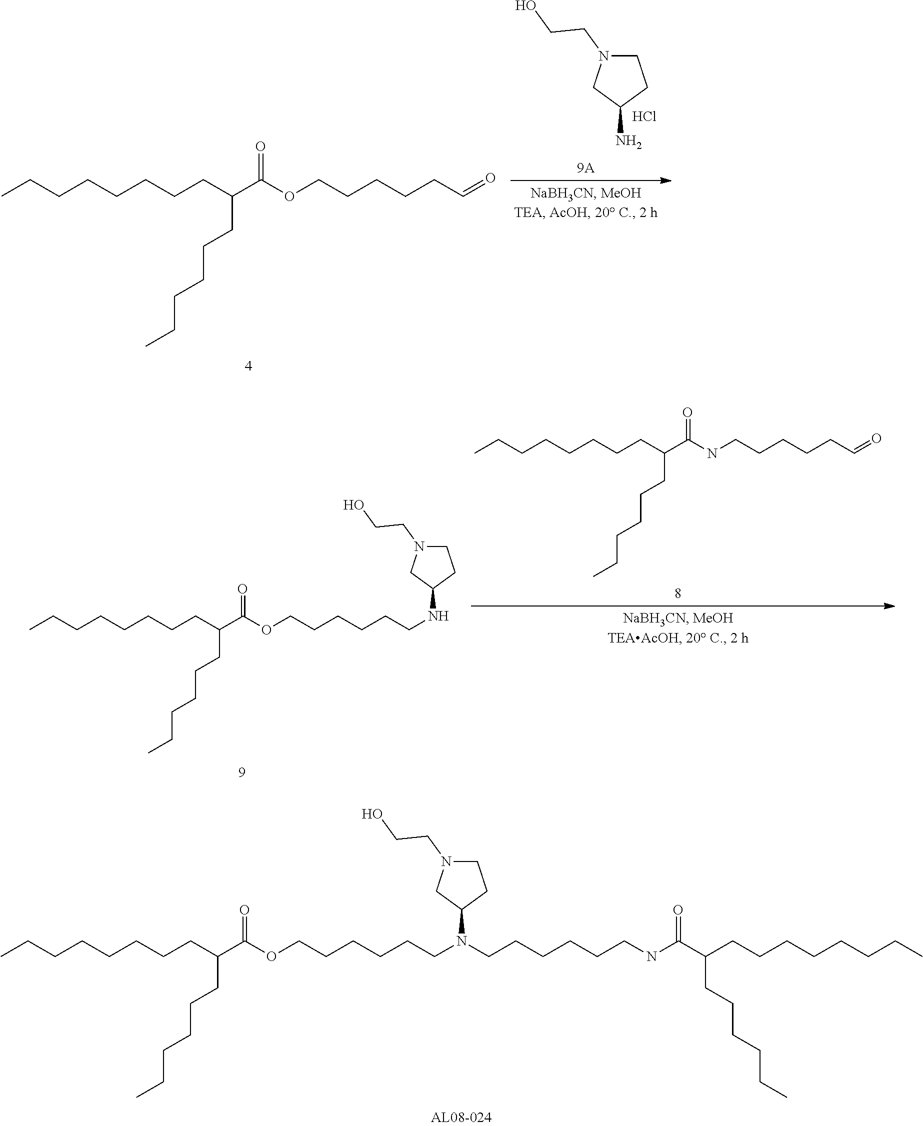 [SMILES: N[C@@H]1CCN(CCO)C1 ; Cl ; CCCCCCCCC(CCCCCC)C(=O)OCCCCCC=O ; CCCCCCCCC(CCCCCC)C(=O)OCCCCCCN[C@@H]1CCN(CCO)C1 ; CCCCCCCCC(CCCCCC)C(=O)NCCCCCC=O ; CCCCCCCCC(CCCCCC)C(=O)NCCCCCCN(CCCCCCOC(=O)C(CCCCCC)CCCCCCCC)[C@@H]1CCN(CCO)C1]

### Step 1:

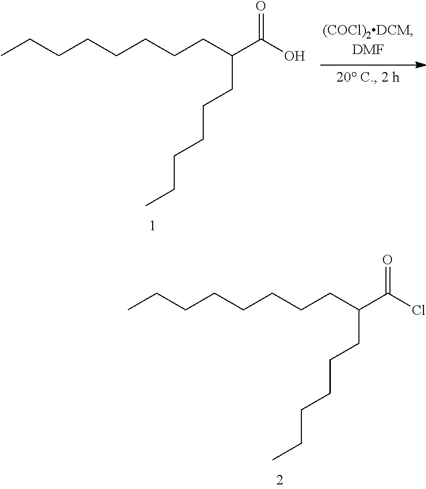 [SMILES: CCCCCCCCC(CCCCCC)C(=O)O ; CCCCCCCCC(CCCCCC)C(=O)Cl]

Dissolve compound 1 (4 g, 0.01556 mol) in DCM (150 mL) in a 250 mL three-neck flask. Replace with nitrogen three times. Cool the reaction to 0° C. with an ice-water bath. Under stirring, dropwise add COCl₂ (5.94 g, 0.0468) and 0.5 mL DMF. After addition, warm to room temperature and stir for 2 hours. After reaction, take 2 drops of reaction solution and add to 0.5 mL methanol. Sample and run TLC (PE:EtOAc=10:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 2 (4.1 g, 0.01495 mol) was obtained as a yellow liquid.

### Step 2:

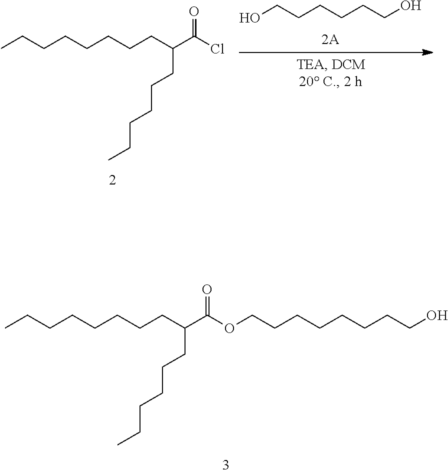 [SMILES: CCCCCCCCC(CCCCCC)C(=O)Cl ; OCCCCCCO ; CCCCCCCCC(CCCCCC)C(=O)OCCCCCCCCO]

Dissolve compound 2A (2.21 g, 0.0187 mol) in DCM (80 mL) in a 250 mL three-neck flask. Replace with nitrogen three times. Cool the reaction to 0° C. with an ice-water bath. Under stirring, dropwise add TEA (3.16 g, 0.0312 mol), then add compound 2 (4 g, 0.0156 mol). After addition, warm to room temperature and stir for 2 hours. After reaction, TLC (PE:EtOAc=3:1, Rf=0.6) shows complete consumption of starting material and formation of a new spot. Add 200 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (20%) yields the product. Compound 3 (3 g, 0.0084 mol) was obtained as a colorless oil.

### Step 3:

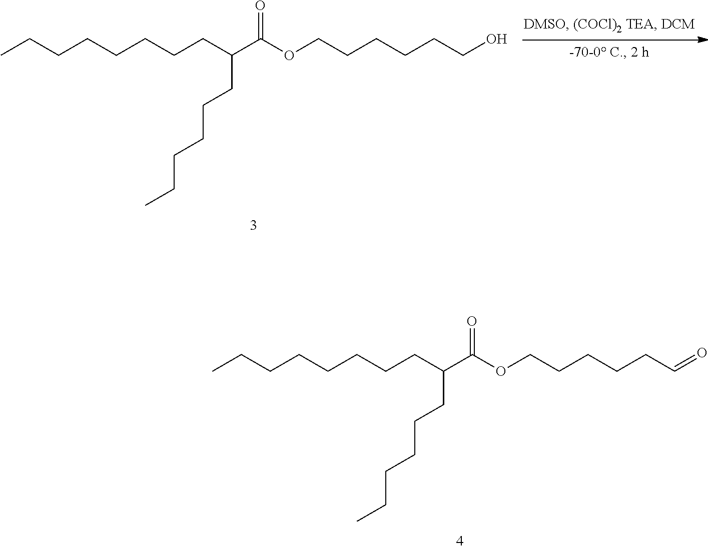 [SMILES: CCCCCCCCC(CCCCCC)C(=O)OCCCCCCO ; CCCCCCCCC(CCCCCC)C(=O)OCCCCCC=O]

Dissolve DMSO (1.719 g, 0.0253 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (3.160 g, 0.0253 mol). Stir at −60° C. for 30 min. Dissolve compound 3 (3 g, 0.0084 mol) in 10 mL DCM and slowly add dropwise to the above reaction solution. After addition, maintain temperature below −60° C. and stir for 1.5 h. Dropwise add TEA (4.264 g, 0.0421 mol) to the reaction solution below −60° C. Stir at 0° C. for 30 min after addition. Take 2 drops of reaction solution, add dichloromethane and water, extract and spot the organic phase. TLC (PE:EtOAc=3:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 4 (2 g, 0.0056 mol) was obtained as a light yellow oil at room temperature.

### Step 4:

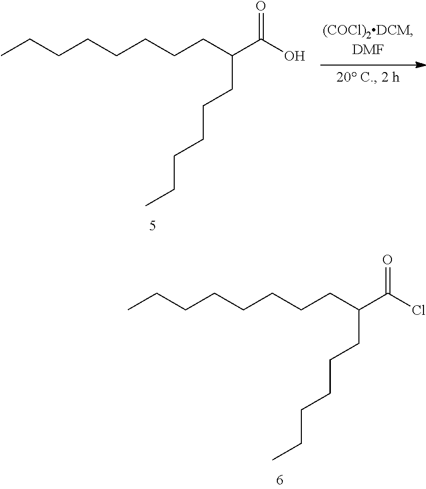 [SMILES: CCCCCCCCC(CCCCCC)C(=O)O ; CCCCCCCCC(CCCCCC)C(=O)Cl]

Dissolve compound 5 (4 g, 0.01561 mol) in DCM (150 mL) in a 250 mL three-neck flask. Replace with nitrogen three times. Cool the reaction to 0° C. with an ice-water bath. Under stirring, dropwise add COCl₂ (11.88 g, 0.02341 mol) and 0.5 mL DMF. After addition, warm to room temperature and stir for 2 hours. After reaction, take 2 drops of reaction solution and add to 0.5 mL methanol. Sample and run TLC (PE:EtOAc=10:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 6 (3.8 g, 0.0139 mol) was obtained as a yellow liquid.

### Step 5:

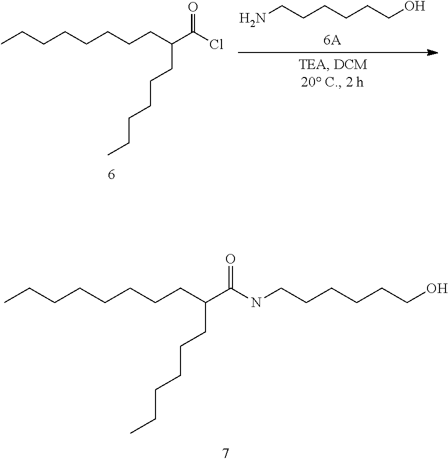 [SMILES: CCCCCCCCC(CCCCCC)C(=O)Cl ; NCCCCCCO ; CCCCCCCCC(CCCCCC)C(=O)NCCCCCCO]

Dissolve compound 6A (1.95 g, 0.0167 mol) in DCM (80 mL) in a 250 mL three-neck flask. Replace with nitrogen three times. Cool the reaction to 0° C. with an ice-water bath. Under stirring, dropwise add TEA (4.88 g, 0.0417 mol), then add compound 6 (3.8 g, 0.0139 mol). After addition, warm to room temperature and stir for 2 hours. After reaction, TLC (PE:EtOAc=1:1, Rf=0.4) shows complete consumption of starting material and formation of a new spot. Add 200 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (25%) yields the product. Compound 7 (2.8 g, 0.0079 mol) was obtained as a colorless oil.

### Step 6:

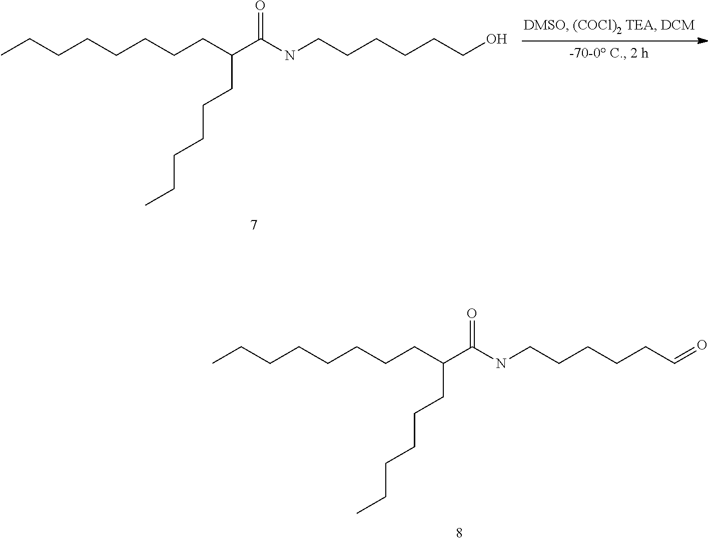 [SMILES: CCCCCCCCC(CCCCCC)C(=O)NCCCCCCO ; CCCCCCCCC(CCCCCC)C(=O)NCCCCCC=O]

Dissolve DMSO (1.612 g, 0.0237 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (3.034 g, 0.0237 mol). Stir at −60° C. for 30 min. Dissolve compound 7 (2.8 g, 0.0079 mol) in 10 mL DCM and slowly add dropwise to the above reaction solution. After addition, maintain temperature below −60° C. and stir for 1.5 h. Dropwise add TEA (3.997 g, 0.0395 mol) to the reaction solution below −60° C. Stir for 30 min after addition. Take 2 drops of reaction solution, add dichloromethane and water, extract and spot the organic phase. TLC (PE:EtOAc=1:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 8 (1.7 g, 0.0048 mol) was obtained as a light yellow oil at room temperature.

### Step 7:

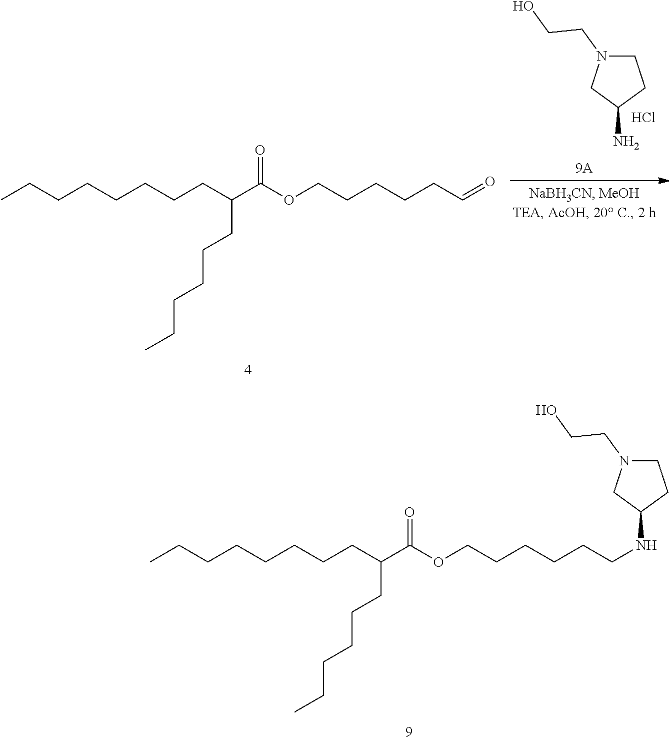 [SMILES: N[C@@H]1CCN(CCO)C1 ; Cl ; CCCCCCCCC(CCCCCC)C(=O)OCCCCCC=O ; CCCCCCCCC(CCCCCC)C(=O)OCCCCCCN[C@@H]1CCN(CCO)C1]

Add compound 9A (200 mg, 0.0015 mol) to a 100 mL single-neck flask. Add MeOH (15 mL) to dissolve, then add TEA (0.1 mL). Under stirring, add compound 4 (478.32 mg, 0.0014 mol). Add 50 mg glacial acetic acid. Add sodium cyanoborohydride (283.5 mg, 0.0045 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate to obtain compound 9 (600 mg, 0.0013 mol, crude product) as a colorless oil.

### Step 8:

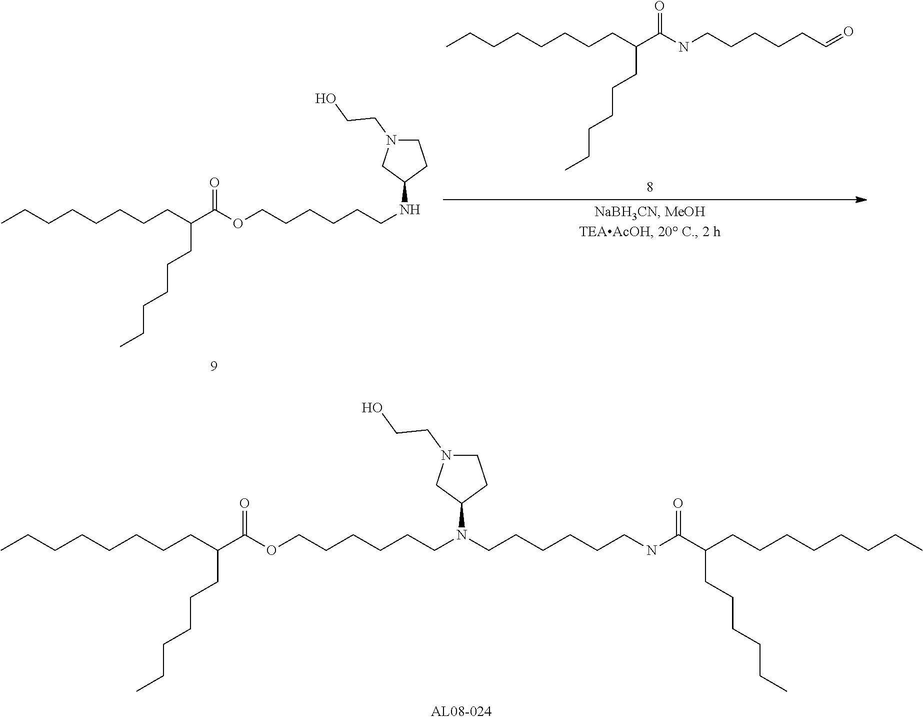 [SMILES: CCCCCCCCC(CCCCCC)C(=O)OCCCCCCN[C@@H]1CCN(CCO)C1 ; CCCCCCCCC(CCCCCC)C(=O)NCCCCCC=O ; CCCCCCCCC(CCCCCC)C(=O)NCCCCCCN(CCCCCCOC(=O)C(CCCCCC)CCCCCCCC)[C@@H]1CCN(CCO)C1]

Add 9 (200 mg, 0.00043 mol) to a 100 mL single-neck flask. Add MeOH (15 mL) to dissolve, then add TEA (0.1 mL). Under stirring, add 8 (151.50 mg, 0.00043 mol). Add 50 mg glacial acetic acid. Add sodium cyanoborohydride (283.5 mg, 0.0045 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate, dissolve, load sample. Column chromatography with mobile phase at MeOH (228%) yields the product. Compound AL08-024 (76.9 mg, 0.000096 mol) was obtained as a light yellow oil at room temperature.

### 1.3.5: Synthesis of AL08-027 Molecule

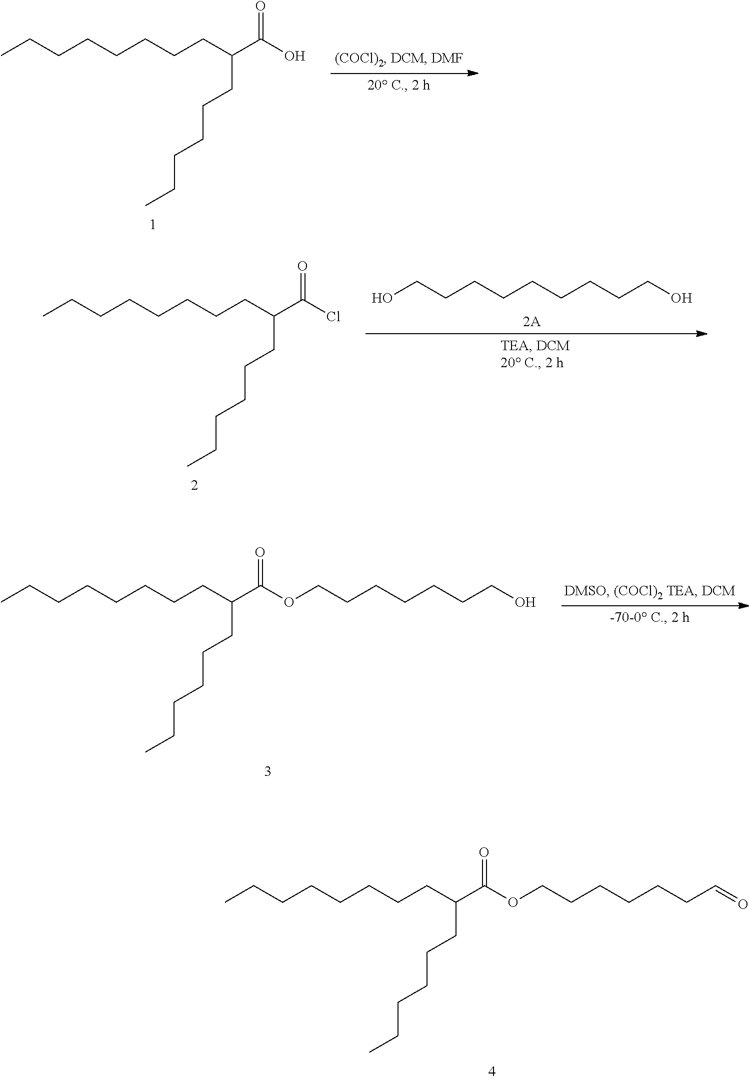 [SMILES: CCCCCCCCC(CCCCCC)C(=O)O ; CCCCCCCCC(CCCCCC)C(=O)Cl ; OCCCCCCCCCO ; CCCCCCCCC(CCCCCC)C(=O)OCCCCCCCO ; CCCCCCCCC(CCCCCC)C(=O)OCCCCCCC=O]

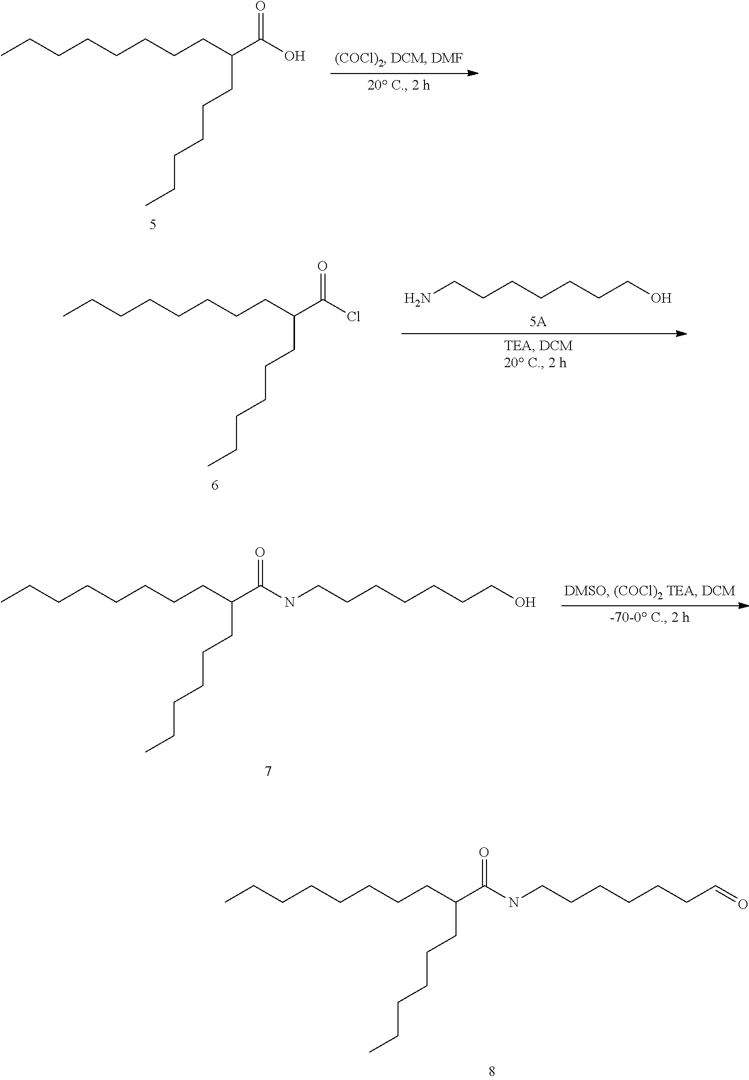 [SMILES: CCCCCCCCC(CCCCCC)C(=O)O ; CCCCCCCCC(CCCCCC)C(=O)Cl ; NCCCCCCCO ; CCCCCCCCC(CCCCCC)C(=O)NCCCCCCCO ; CCCCCCCCC(CCCCCC)C(=O)NCCCCCCC=O]

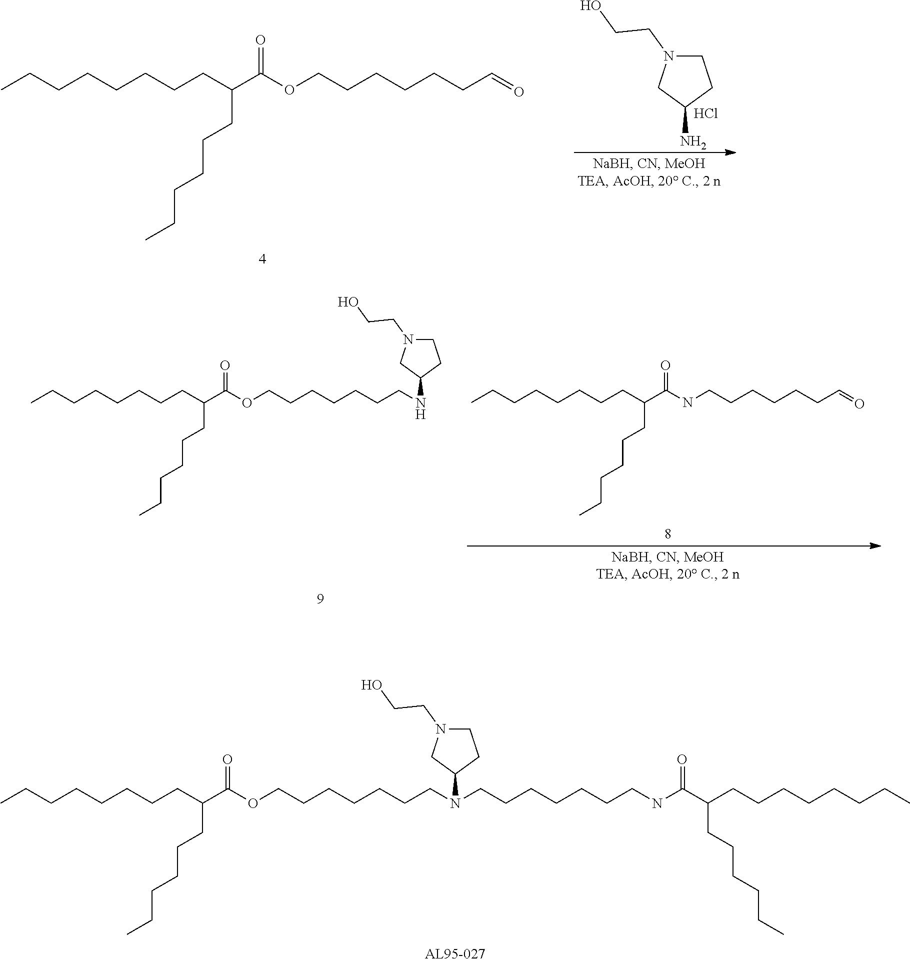 [SMILES: CCCCCCCCC(CCCCCC)C(=O)OCCCCCCC=O ; N[C@@H]1CCN(CCO)C1 ; Cl ; CCCCCCCCC(CCCCCC)C(=O)OCCCCCCCN[C@@H]1CCN(CCO)C1 ; CCCCCCCCC(CCCCCC)C(=O)NCCCCCCC=O ; CCCCCCCCC(CCCCCC)C(=O)NCCCCCCCN(CCCCCCCOC(=O)C(CCCCCC)CCCCCCCC)[C@@H]1CCN(CCO)C1]

### Step 1:

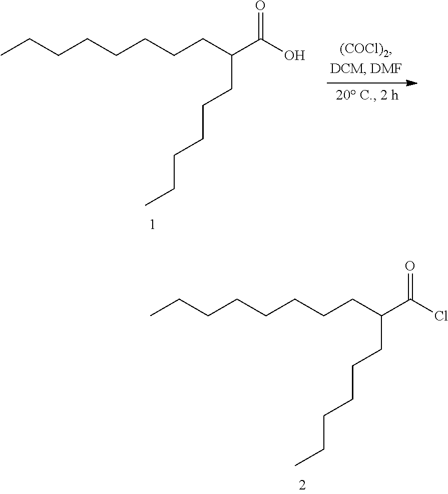 [SMILES: CCCCCCCCC(CCCCCC)C(=O)O ; CCCCCCCCC(CCCCCC)C(=O)Cl]

Dissolve compound 1 (5 g, 0.01951 mol) in DCM (150 mL) in a 250 mL three-neck flask. Replace with nitrogen three times. Cool the reaction to 0° C. with an ice-water bath. Under stirring, dropwise add COCl₂ (5.94 g, 0.03903 mol) and 0.5 mL DMF. After addition, warm to room temperature and stir for 2 hours. After reaction, take 2 drops of reaction solution and add to 0.5 mL methanol. Sample and run TLC (PE:EtOAc=10:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 2 (5.1 g, 0.01860 mol) was obtained as a yellow liquid.

### Step 2:

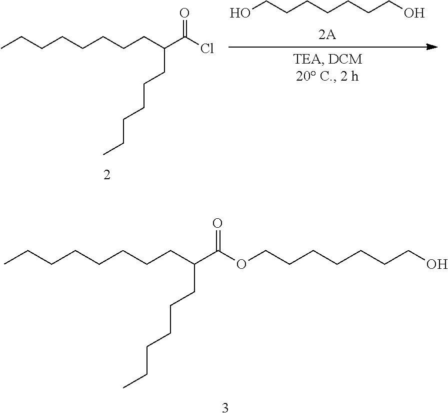 [SMILES: OCCCCCCCO ; CCCCCCCCC(CCCCCC)C(=O)Cl ; CCCCCCCCC(CCCCCC)C(=O)OCCCCCCCO]

Dissolve 2A (2.95 g, 0.02232 mol) in DCM (80 mL) in a 250 mL three-neck flask. Replace with nitrogen three times. Cool the reaction to 0° C. with an ice-water bath. Under stirring, dropwise add TEA (5.65, 0.0558 mol), then add 2 (5.1 g, 0.01860 mol). After addition, warm to room temperature and stir for 2 hours. After reaction, TLC (PE:EtOAc=3:1, Rf=0.6) shows complete consumption of starting material and formation of a new spot. Add 200 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (20%) yields the product. Compound 3 (5.3 g, 0.01431 mol) was obtained as a colorless oil.

### Step 3:

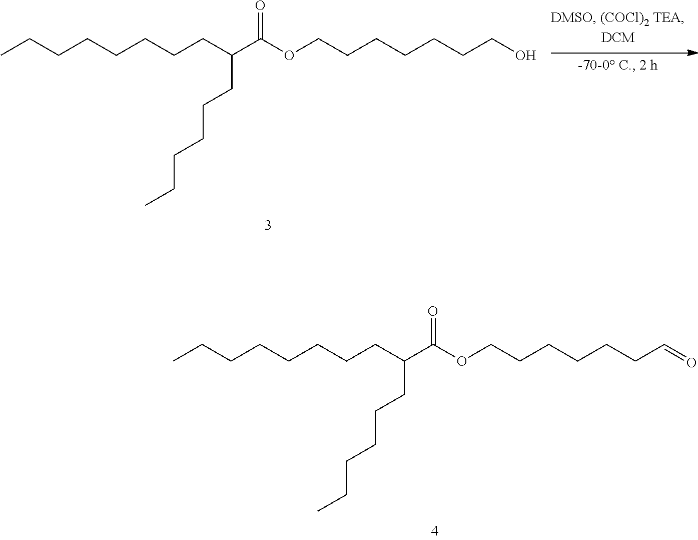 [SMILES: CCCCCCCCC(CCCCCC)C(=O)OCCCCCCCO ; CCCCCCCCC(CCCCCC)C(=O)OCCCCCCC=O]

Dissolve DMSO (2.92 g, 0.0429 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (5.49 g, 0.0429 mol). Stir at −60° C. for 30 min. Dissolve 3 (5.3 g, 0.0143 mol) in 10 mL DCM and slowly add dropwise to the above reaction solution. After addition, maintain temperature below −60° C. and stir for 1.5 h. Dropwise add TEA (7.23 g, 0.0715 mol) to the reaction solution below −60° C. Stir at 0° C. for 30 min after addition. Take 2 drops of reaction solution, add dichloromethane and water, extract and spot the organic phase. TLC (PE:EtOAc=3:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 4 (3 g, 0.00814 mol) was obtained as a light yellow oil at room temperature.

### Step 4:

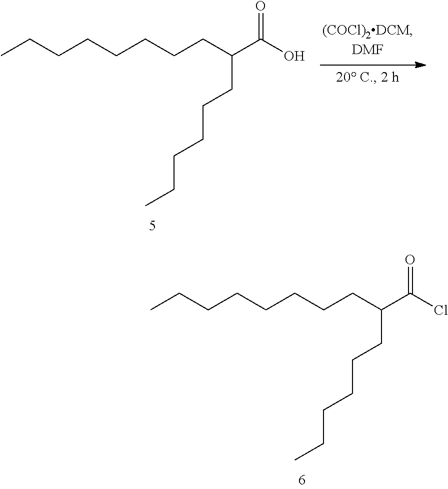 [SMILES: CCCCCCCCC(CCCCCC)C(=O)O ; CCCCCCCCC(CCCCCC)C(=O)Cl]

Dissolve compound 5 (4 g, 0.01561 mol) in DCM (150 mL) in a 250 mL three-neck flask. Replace with nitrogen three times. Cool the reaction to 0° C. with an ice-water bath. Under stirring, dropwise add COCl₂ (11.88 g, 0.02341 mol) and 0.5 mL DMF. After addition, warm to room temperature and stir for 2 hours. After reaction, take 2 drops of reaction solution and add to 0.5 mL methanol. Sample and run TLC (PE:EtOAc=10:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 6 (3.8 g, 0.0139 mol) was obtained as a yellow liquid.

### Step 5:

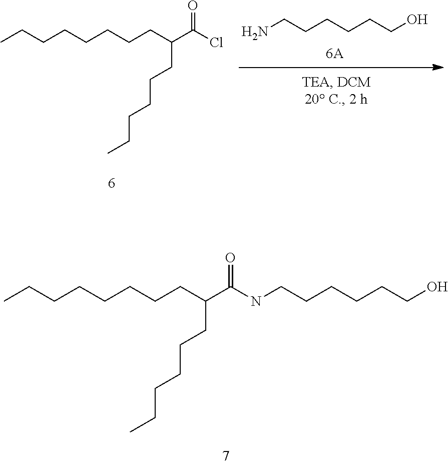 [SMILES: CCCCCCCCC(CCCCCC)C(=O)Cl ; NCCCCCCO ; CCCCCCCCC(CCCCCC)C(=O)NCCCCCCO]

Dissolve compound 6A (1.95 g, 0.0149 mol) in DCM (80 mL) in a 250 mL three-neck flask. Replace with nitrogen three times. Cool the reaction to 0° C. with an ice-water bath. Under stirring, dropwise add TEA (4.88 g, 0.0417 mol), then add compound 6 (3.8 g, 0.0139 mol). After addition, warm to room temperature and stir for 2 hours. After reaction, TLC (PE:EtOAc=1:1, Rf=0.4) shows complete consumption of starting material and formation of a new spot. Add 200 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (25%) yields the product. Compound 7 (2.8 g, 0.0079 mol) was obtained as a colorless oil.

### Step 6:

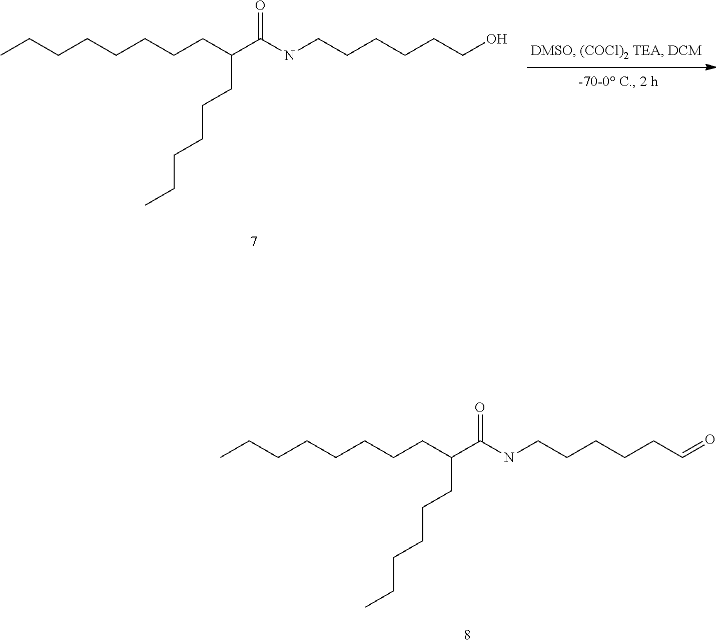 [SMILES: CCCCCCCCC(CCCCCC)C(=O)NCCCCCCO ; CCCCCCCCC(CCCCCC)C(=O)NCCCCCC=O]

Dissolve DMSO (1.612 g, 0.0237 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (3.034 g, 0.0237 mol). Stir at −60° C. for 30 min. Dissolve compound 7 (2.8 g, 0.0079 mol) in 10 mL DCM and slowly add dropwise to the above reaction solution. After addition, maintain temperature below −60° C. and stir for 1 h. Dropwise add TEA (3.997 g, 0.0395 mol) to the reaction solution below −60° C. Stir at 0° C. for 30 min after addition. Take 2 drops of reaction solution, add dichloromethane and water, extract and spot the organic phase. TLC (PE:EtOAc=1:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 8 (1.7 g, 0.0048 mol) was obtained as a light yellow oil at room temperature.

### Step 7:

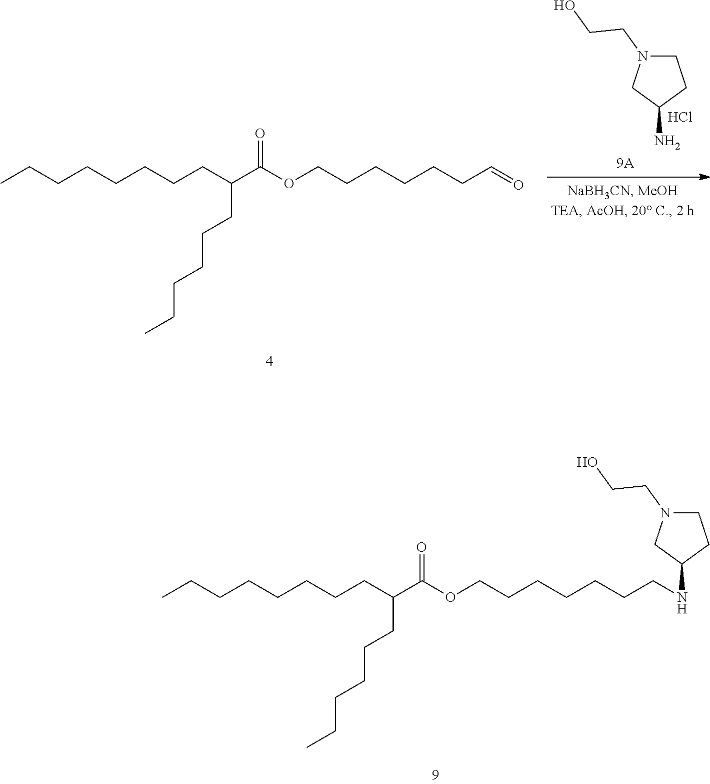 [SMILES: CCCCCCCCC(CCCCCC)C(=O)OCCCCCCC=O ; N[C@@H]1CCN(CCO)C1 ; Cl ; CCCCCCCCC(CCCCCC)C(=O)OCCCCCCCN[C@@H]1CCN(CCO)C1]

Add compound 9A (200 mg, 0.0015 mol) to a 100 mL single-neck flask. Add MeOH (15 mL) to dissolve, then add TEA (0.1 mL). Under stirring, add compound 4 (510.0 mg, 0.0014 mol). Add 50 mg glacial acetic acid. Add sodium cyanoborohydride (283.5 mg, 0.0045 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate to obtain compound 9 (550 mg, 0.0014 mol, crude product) as a colorless oil.

### Step 8:

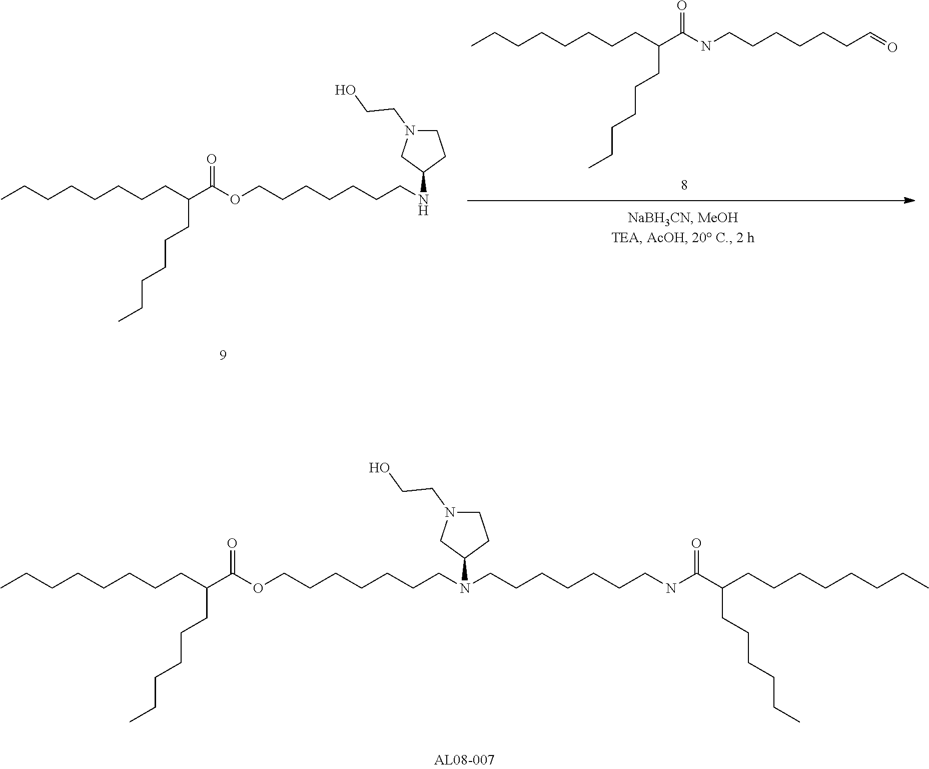 [SMILES: CCCCCCCCC(CCCCCC)C(=O)OCCCCCCCN[C@@H]1CCN(CCO)C1 ; CCCCCCCCC(CCCCCC)C(=O)NCCCCCCC=O ; CCCCCCCCC(CCCCCC)C(=O)NCCCCCCCN(CCCCCCCOC(=O)C(CCCCCC)CCCCCCCC)[C@@H]1CCN(CCO)C1]

Add compound 9 (550 mg, 0.0014 mol) to a 100 mL single-neck flask. Add MeOH (15 mL) to dissolve, then add TEA (0.1 mL). Under stirring, add compound 8 (512.88 mg, 0.0014 mol). Add 50 mg glacial acetic acid. Add sodium cyanoborohydride (283.5 mg, 0.0045 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, concentrate, dissolve, load sample. Column chromatography with mobile phase at MeOH (22%) yields the product. Compound AL08-027 (52.6 mg, 0.000063 mol) was obtained as a light yellow oil at room temperature.

### 1.3.6: Synthesis of AL08-025/026 Molecules

The synthesis steps for AL08-025/026 molecules are similar to those for AL08-024 and AL08-027 molecules, thus not repeated here.

### 1.3.7: Synthesis of AL08-009 Molecule

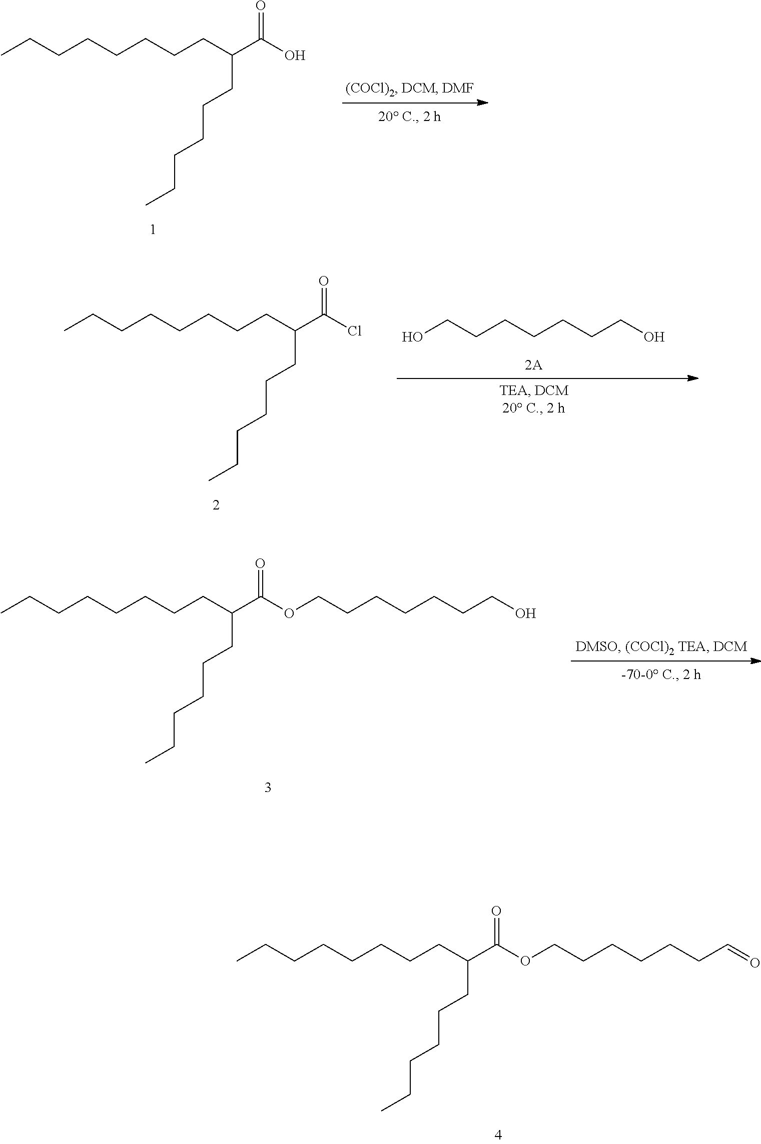 [SMILES: CCCCCCCCC(CCCCCC)C(=O)O ; CCCCCCCCC(CCCCCC)C(=O)Cl ; OCCCCCCCO ; CCCCCCCCC(CCCCCC)C(=O)OCCCCCCCO ; CCCCCCCCC(CCCCCC)C(=O)OCCCCCCC=O]

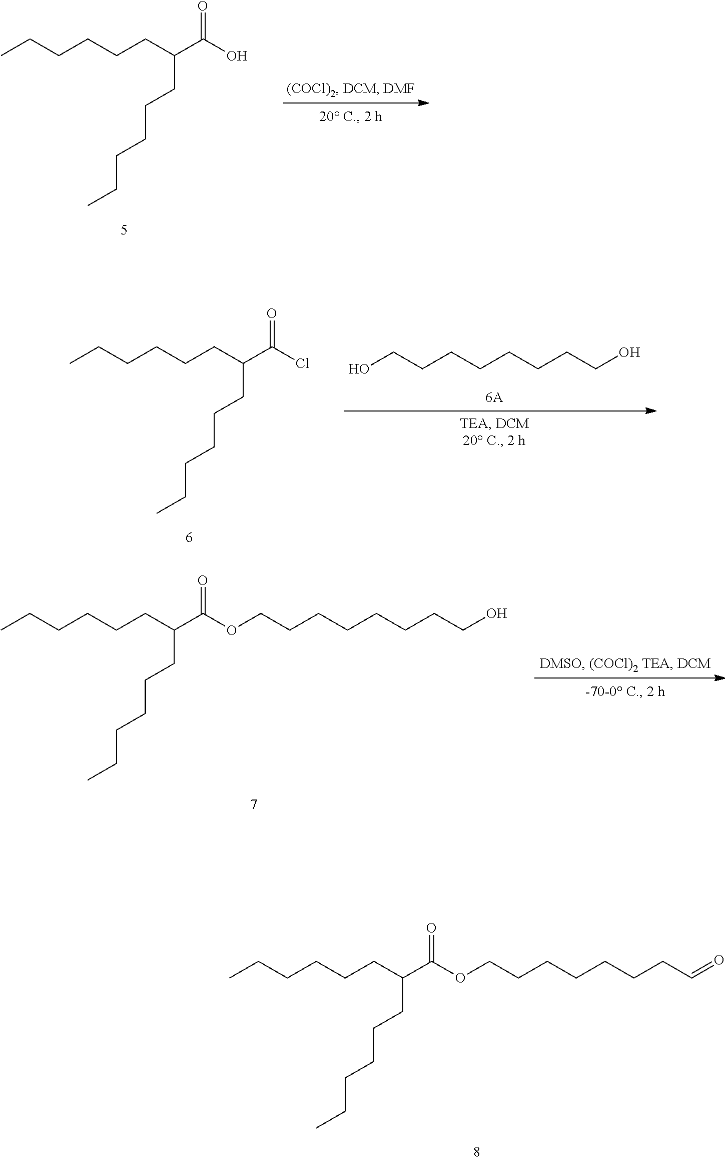 [SMILES: CCCCCCC(CCCCCC)C(=O)O ; CCCCCCC(CCCCCC)C(=O)Cl ; OCCCCCCCCO ; CCCCCCC(CCCCCC)C(=O)OCCCCCCCCO ; CCCCCCC(CCCCCC)C(=O)OCCCCCCCC=O]

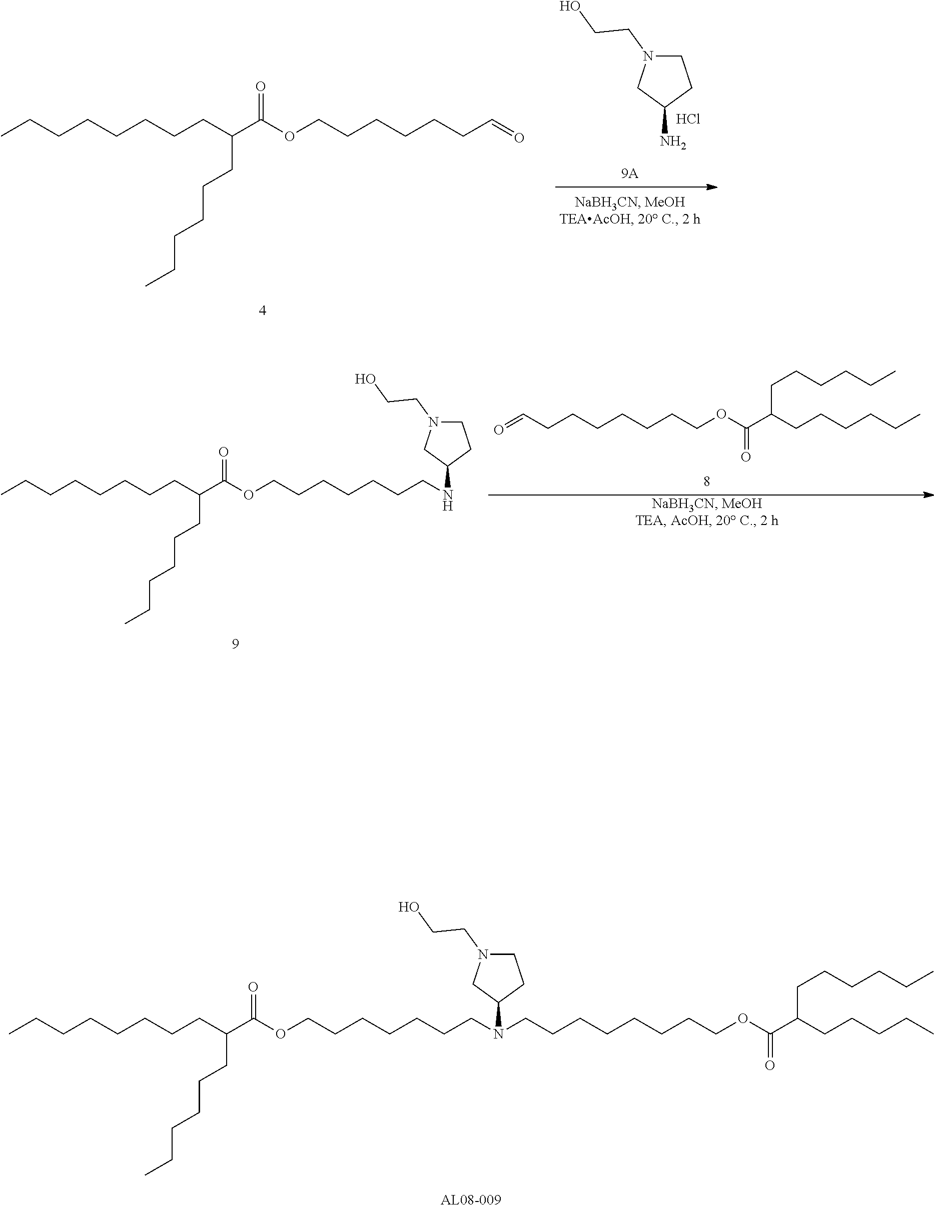

### Step 1:

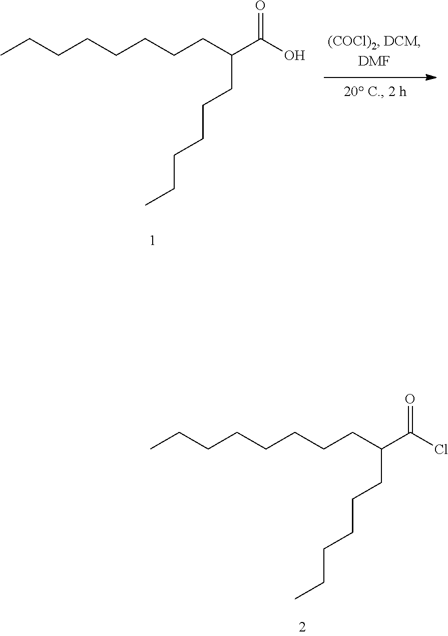 [SMILES: CCCCCCCCC(CCCCCC)C(=O)O ; CCCCCCCCC(CCCCCC)C(=O)Cl]

Dissolve compound 1 (5 g, 0.0195 mol) in DCM (150 mL) in a 250 mL three-neck flask. Replace with nitrogen three times. Cool the reaction to 0° C. with an ice-water bath. Under stirring, dropwise add COCl₂ (5.00 g, 0.0390 mol) and 0.5 mL DMF. After addition, warm to room temperature and stir for 2 hours. After reaction, take 2 drops of reaction solution and add to 0.5 mL methanol. Sample and run TLC (PE:EtOAc=5:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 2 (4 g, 0.0146 mol) was obtained as a yellow liquid.

### Step 2:

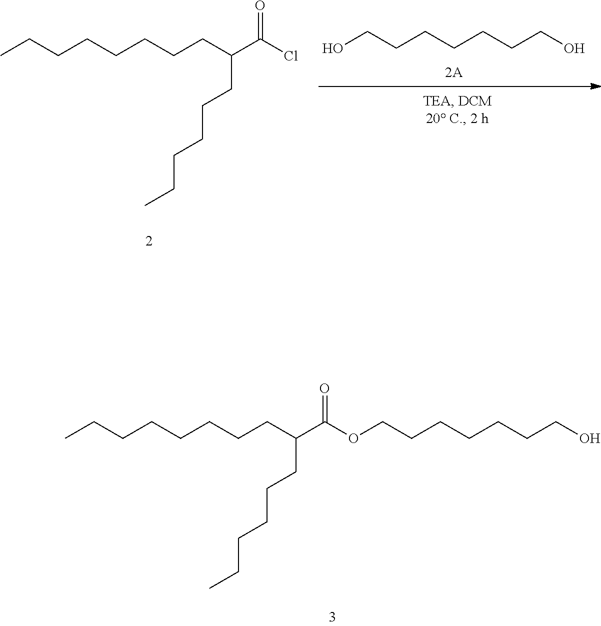 [SMILES: CCCCCCCCC(CCCCCC)C(=O)Cl ; OCCCCCCCO ; CCCCCCCCC(CCCCCC)C(=O)OCCCCCCCO]

Dissolve compound 2A (2.31 g, 0.0175 mol) in DCM (100 mL) in a 250 mL three-neck flask. Replace with nitrogen three times. Cool the reaction to 0° C. with an ice-water bath. Under stirring, dropwise add TEA (4.43 g, 0.0438 mol), then add compound 2 (4 g, 0.0146 mol). After addition, warm to room temperature and stir for 2 hours. After reaction, TLC (PE:EtOAc=3:1, Rf=0.6) shows complete consumption of starting material and formation of a new spot. Add 100 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (30%) yields the product. Compound 3 (4.5 g, 0.0122 mol) was obtained as a colorless oil.

### Step 3:

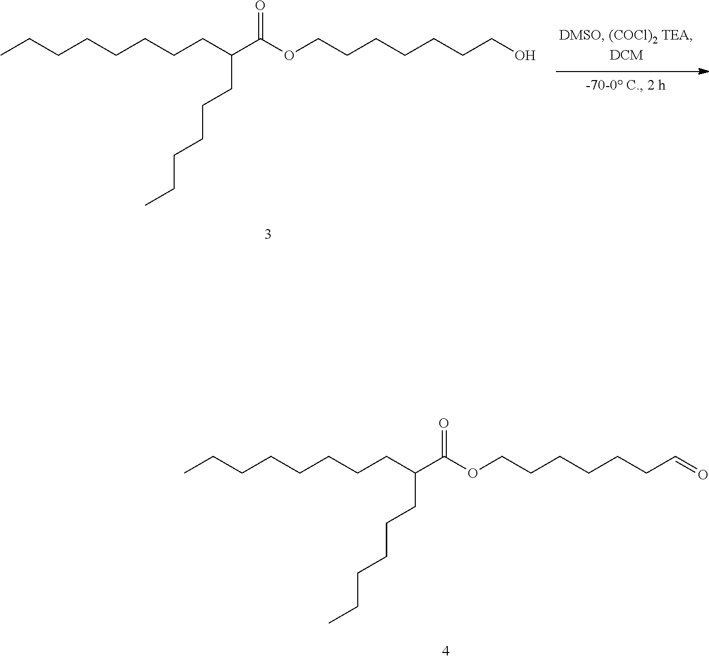 [SMILES: CCCCCCCCC(CCCCCC)C(=O)OCCCCCCCO ; CCCCCCCCC(CCCCCC)C(=O)OCCCCCCC=O]

Dissolve DMSO (2.149 g, 0.0366 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (4.68 g, 0.0366 mol). Stir at −60° C. for 30 min. Dissolve compound 3 (4.5 g, 0.0122 mol) in 10 mL DCM and slowly add dropwise to the above reaction solution. After addition, maintain temperature below −60° C. and stir for 1 h. Dropwise add TEA (5.26 g, 0.0520 mol) to the reaction solution below −60° C. Stir at 0° C. for 30 min after addition. Take 2 drops of reaction solution, add dichloromethane and water, extract and spot the organic phase. TLC (PE:EtOAc=5:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 4 (2.5 g, 0.0068 mol) was obtained as a light yellow oil at room temperature.

### Step 4:

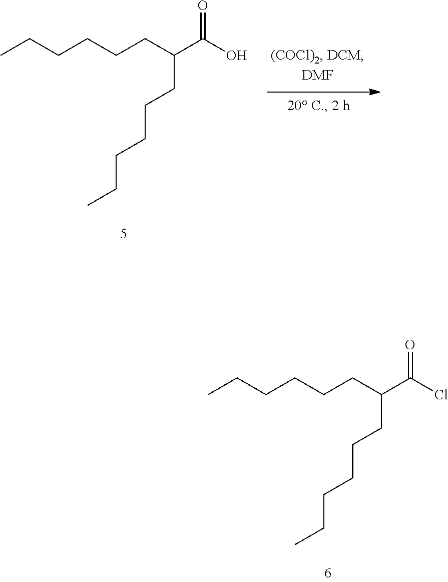 [SMILES: CCCCCCC(CCCCCC)C(=O)O ; CCCCCCC(CCCCCC)C(=O)Cl]

Dissolve compound 5 (5 g, 0.0219 mol) in DCM (150 mL) in a 250 mL three-neck flask. Replace with nitrogen three times. Cool the reaction to 0° C. with an ice-water bath. Under stirring, dropwise add COCl₂ (5.61 g, 0.0438 mol) and 0.5 mL DMF. After addition, warm to room temperature and stir for 2 hours. After reaction, take 2 drops of reaction solution and add to 0.5 mL methanol. Sample and run TLC (PE:EtOAc=5:1, Rf=0.6), which shows complete consumption of starting material and formation of a new spot. Pour the reaction solution into a 250 mL single-neck flask and concentrate under reduced pressure. Wash and concentrate with DCM (100 mL)×2 twice. Compound 6 (5 g, 0.0203 mol) was obtained as a yellow liquid.

### Step 5:

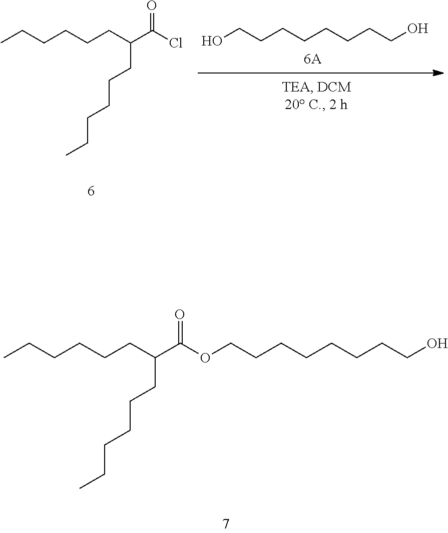 [SMILES: CCCCCCC(CCCCCC)C(=O)Cl ; OCCCCCCCCO ; CCCCCCC(CCCCCC)C(=O)OCCCCCCCCO]

Dissolve compound 6A (3.56 g, 0.0244 mol) in DCM (100 mL) in a 250 mL three-neck flask. Replace with nitrogen three times. Cool the reaction to 0° C. with an ice-water bath. Under stirring, dropwise add TEA (4.43 g, 0.0609 mol), then add 6 (5 g, 0.0203 mol). After addition, warm to room temperature and stir for 2 hours. After reaction, TLC (PE:EtOAc=3:1, Rf=0.5) shows complete consumption of starting material and formation of a new spot. Add 100 mL water to the reaction solution. Extract the aqueous phase with 100 mL DCM three times. Combine organic phases, dry over anhydrous sodium sulfate, and concentrate under reduced pressure to obtain crude product. Dissolve crude product with silica gel for sample loading. Column chromatography with mobile phase at EtOAc (30%) yields the product. Compound 7 (4.5 g, 0.0126 mol) was obtained as a colorless oil.

### Step 6:

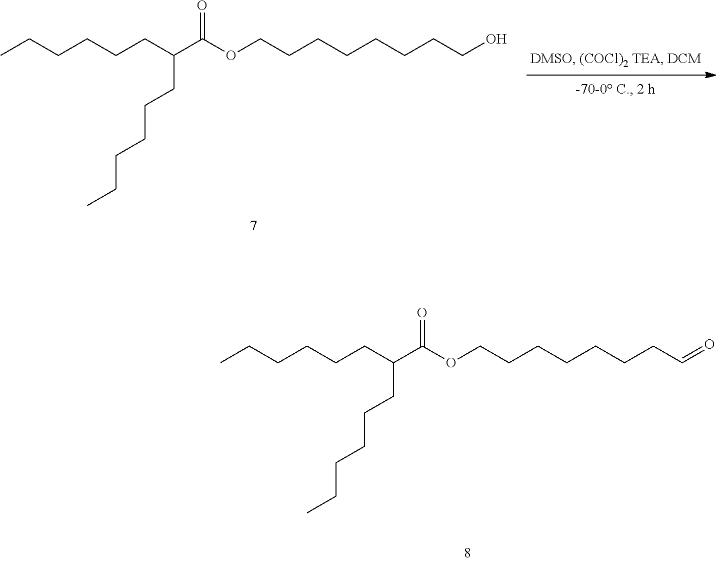 [SMILES: CCCCCCC(CCCCCC)C(=O)OCCCCCCCCO ; CCCCCCC(CCCCCC)C(=O)OCCCCCCCC=O]

Dissolve DMSO (2.570 g, 0.0378 mol) in DCM (100 mL) in a 250 mL three-neck flask. Replace with nitrogen. Cool the reaction to −70° C. with a dry ice-ethanol bath. Maintain temperature below −60° C. and dropwise add COCl₂ (4.84 g, 0.0378 mol). Stir at −60° C. for 30 min. Dissolve compound 7 (4.5 g, 0.0126 mol) in 10 mL DCM and slowly add dropwise to the above reaction solution. After addition, maintain temperature below −60° C. and stir for 1 h. Dropwise add TEA (5.26 g, 0.0520 mol) to the reaction solution below −60° C. Stir at 0° C. for 30 min after addition. Take 2 drops of reaction solution, add dichloromethane and water, extract and spot the organic phase. TLC (PE:EtOAc=5:1, Rf=0.6) shows complete consumption of starting material with new spot formation. Pour reaction solution into ice-cold saturated 100 mL ammonium chloride aqueous solution. Add DCM (60 mL×3) for extraction. Combine organic phases, dry over anhydrous sodium sulfate, concentrate, dissolve, load sample. Column chromatography with mobile phase at EtOAc (8%) yields the product. Compound 8 (2.7 g, 0.0076 mol) was obtained as a light yellow oil at room temperature.

### Step 7:

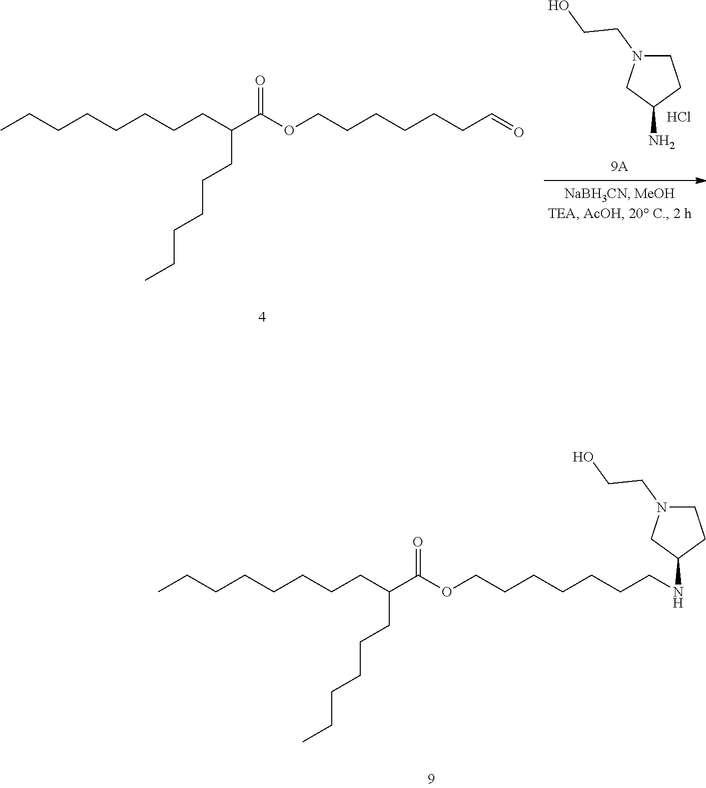 [SMILES: CCCCCCCCC(CCCCCC)C(=O)OCCCCCCC=O ; N[C@@H]1CCN(CCO)C1 ; Cl ; CCCCCCCCC(CCCCCC)C(=O)OCCCCCCCN[C@@H]1CCN(CCO)C1]

Add compound 9A (200 mg, 0.0015 mol) to a 100 mL single-neck flask. Add MeOH (15 mL) to dissolve, then add TEA (0.1 mL). Under stirring, add compound 4 (510 mg, 0.0014 mol). Add 50 mg glacial acetic acid. Add sodium cyanoborohydride (483.38 mg, 0.0077 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate to obtain compound 9 (225 mg, 0.0035 mol, crude product, not purified).

### Step 8:

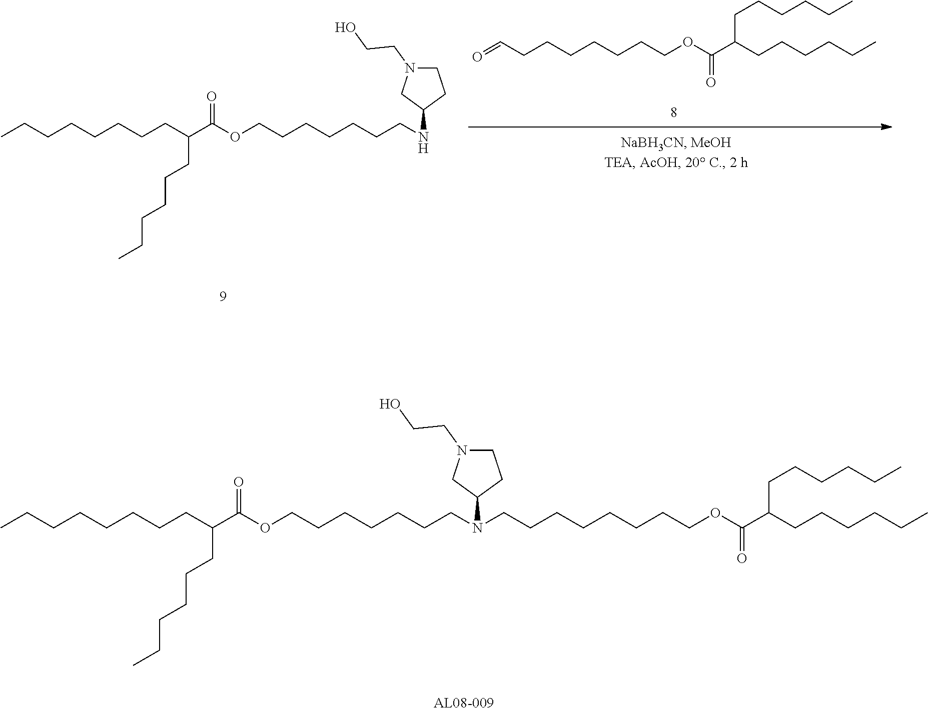 [SMILES: CCCCCCCCC(CCCCCC)C(=O)OCCCCCCCN[C@@H]1CCN(CCO)C1 ; CCCCCCC(CCCCCC)C(=O)OCCCCCCCC=O ; CCCCCCCCC(CCCCCC)C(=O)OCCCCCCCN(CCCCCCCCOC(=O)C(CCCCCC)CCCCCC)[C@@H]1CCN(CCO)C1]

Add compound 9 (225 mg, 0.0035 mol) to a 100 mL single-neck flask. Add MeOH (15 mL) to dissolve, then add TEA (0.1 mL). Under stirring, add 8 (531 mg, 0.0015 mol). Add 50 mg glacial acetic acid. Add sodium cyanoborohydride (483.38 mg, 0.0077 mol) to the above reaction solution. Stir at room temperature for one hour. LCMS shows no starting material remains, with main peak as product. Concentrate the reaction solution under reduced pressure, then add 30 mL water. Add saturated sodium bicarbonate solution to adjust pH to about 8. Extract with ethyl acetate (30 mL) three times. Combine organic phases, dry over anhydrous sodium sulfate, and concentrate under reduced pressure. Dissolve crude product for sample loading. Column chromatography with mobile phase of 12% MeOH:88% EtOAC. Concentrate to obtain compound AL08-009 (222 mg, 0.00027 mol) as a light yellow oil.

### 1.3.8: Synthesis of AL08-010/014/015 Molecules

The synthesis steps for AL08-010/014/015 molecules are similar to those for AL08-009 molecules, thus not repeated here.

### 1.4: Characterization Data

### 1.4.1: AL08-001

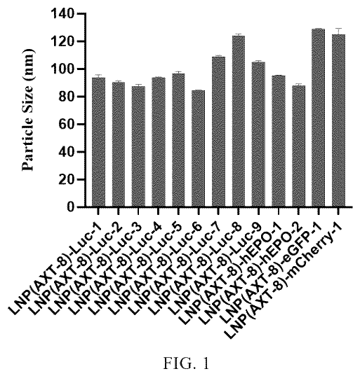

The ¹H NMR spectrum of AL08-001 is shown in FIG. 1. Mass spectrometry and NMR data are as follows:

LC-MS: (ES, m/z): 780.0 [M+H]⁺;

¹H NMR (600 MHz, Chloroform-d) δ 4.08 (t, J=6.7 Hz, 4H), 3.75 (t, J=5.2 Hz, 2H), 3.36-3.12 (m, 6H), 3.11-2.73 (m, 6H), 2.60-2.48 (m, 3H), 2.38-2.30 (m, 2H), 2.11-2.03 (m, 1H), 1.71-1.55 (m, 9H), 1.50-1.42 (m, 7H), 1.40-1.18 (m, 48H), 0.89 (t, J=7.0 Hz, 13H).

### 1.4.2: AL08-003

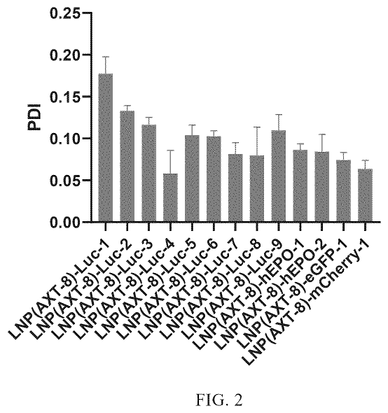

The ¹H NMR spectrum of AL08-003 is shown in FIG. 2. Mass spectrometry and NMR data are as follows:

LC-MS: (ES, m/z): 836.2 [M+H]⁺;

¹H NMR (600 MHz, Chloroform-d) δ 4.07 (t, J=6.7 Hz, 1H), 3.68-3.38 (m, 1H), 2.95-2.72 (m, 0H), 2.48 (q, J=9.2, 7.4 Hz, 1H), 2.32 (dt, J=8.9, 4.0 Hz, 0H), 2.12-1.71 (m, 1H), 1.76-1.51 (m, 3H), 1.53-1.33 (m, 3H), 1.29 (d, J=22.4 Hz, 12H), 0.89 (t, J=6.9 Hz, 3H).

### 1.4.3: AL08-004

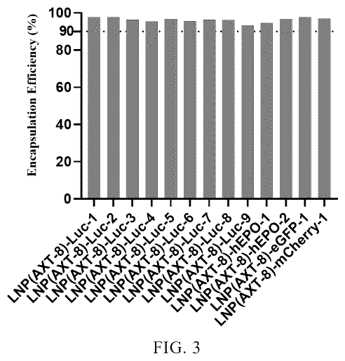

The ¹H NMR spectrum of AL08-004 is shown in FIG. 3. Mass spectrometry and NMR data are as follows:

LC-MS: (ES, m/z): 808.1 [M+H]⁺;

¹H NMR (600 MHz, Chloroform-d) δ 4.08 (t, J=6.6 Hz, 3H), 3.73 (t, J=5.2 Hz, 2H), 3.55-3.46 (m, 1H), 3.07-2.71 (m, 4H), 2.61-2.46 (m, 2H), 2.33 (tt, J=9.0, 5.3 Hz, 1H), 1.67-1.56 (m, 6H), 1.54-1.13 (m, 45H), 0.89 (t, J=7.0 Hz, 9H).

### 1.4.4: AL08-005

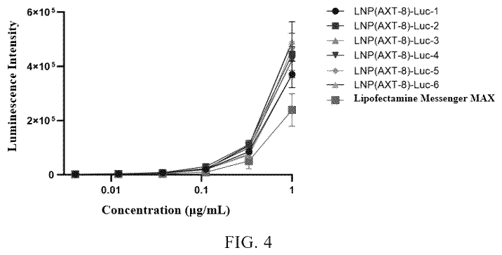

The ¹H NMR spectrum of AL08-005 is shown in FIG. 4. Mass spectrometry and NMR data are as follows:

LC-MS: (ES, m/z): 864.1 [M+H]⁺;

¹H NMR (400 MHz, Chloroform-d) δ 4.08 (t, J=6.7 Hz, 1H), 3.68 (t, J=5.3 Hz, OH), 3.50-3.38 (m, 0H), 3.02-2.38 (m, 3H), 2.32 (t, J=5.3 Hz, 0H), 2.01 (s, 0H), 1.82 (dd, J=13.0, 7.3 Hz, 0H), 1.63 (d, J=6.7 Hz, 1H), 1.52-1.15 (m, 15H), 0.89 (t, J=6.7 Hz, 3H).

### 1.4.5: AL08-006

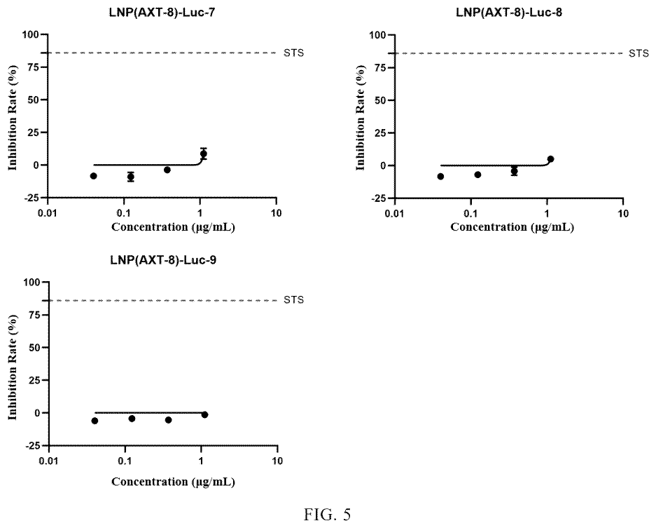

The ¹H NMR spectrum of AL08-006 is shown in FIG. 5. Mass spectrometry and NMR data are as follows:

LC-MS: (ES, m/z): 920.0 [M+H]⁺;

¹H NMR (600 MHz, Chloroform-d) δ 4.08 (t, J=6.7 Hz, 0H), 3.73 (t, J=5.2 Hz, OH), 3.07-2.44 (m, 0H), 2.35-2.26 (m, 0H), 2.04 (s, 0H), 1.69-1.53 (m, 0H), 1.50-1.21 (m, 1H), 0.90 (t, J=7.0 Hz, 0H).

### 1.4.6: AL08-007

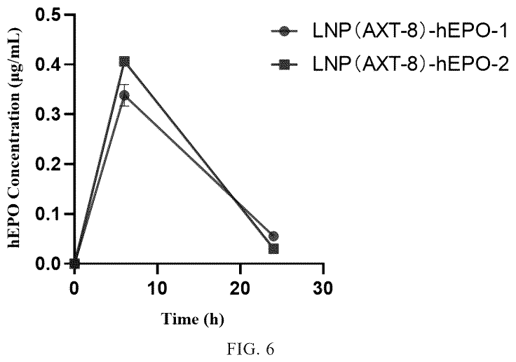

The ¹H NMR spectrum of AL08-007 is shown in FIG. 6. Mass spectrometry and NMR data are as follows:

LC-MS: (ES, m/z): 892.2 [M+H]⁺;

¹H NMR (600 MHz, Chloroform-d) δ 4.08 (t, J=6.6 Hz, 3H), 3.73 (t, J=5.2 Hz, 2H), 3.55-3.46 (m, 1H), 3.07-2.71 (m, 4H), 2.61-2.46 (m, 2H), 2.33 (tt, J=9.0, 5.3 Hz, 1H), 1.67-1.56 (m, 6H), 1.54-1.13 (m, 45H), 0.89 (t, J=7.0 Hz, 9H).

### 1.4.7: AL08-008

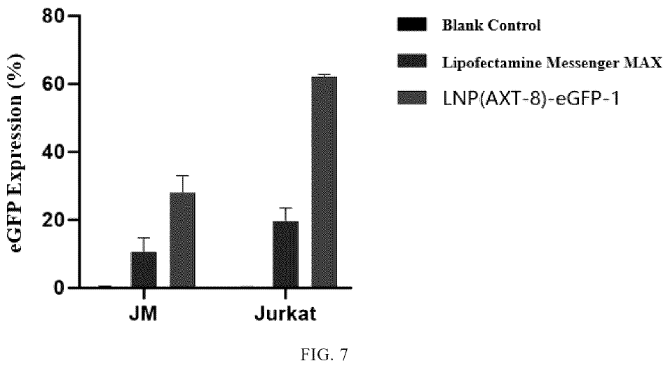

The ¹H NMR spectrum of AL08-008 is shown in FIG. 7. Mass spectrometry and NMR data are as follows:

LC-MS: (ES, m/z): 864.1 [M+H]⁺;

¹H NMR (600 MHz, Chloroform-d) δ 4.08 (t, J=6.7 Hz, 4H), 3.70 (t, J=5.3 Hz, 2H), 3.53 (p, J=7.3 Hz, 1H), 3.09 (s, 2H), 2.88-2.54 (m, 10H), 2.33 (tt, J=8.9, 5.3 Hz, 2H), 1.91-1.83 (m, 1H), 1.72-1.51 (m, 13H), 1.47-1.20 (m, 69H), 0.90 (t, J=7.0 Hz, 14H).

### 1.4.8: AL08-009

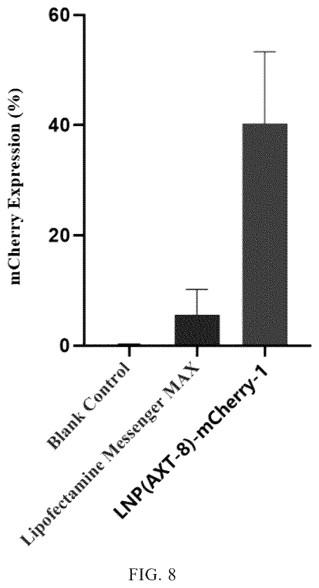

The ¹H NMR spectrum of AL08-009 is shown in FIG. 8. Mass spectrometry and NMR data are as follows:

LC-MS: (ES, m/z): 822.1 [M+H]⁺;

¹H NMR (600 MHz, Chloroform-d) δ 4.08 (t, J=6.6 Hz, 2H), 3.73 (t, J=5.2 Hz, 1H), 3.49 (p, J=7.5 Hz, 1H), 3.05-2.70 (m, 3H), 2.52 (p, J=7.2, 5.6 Hz, 2H), 2.38-2.29 (m, 1H), 2.10-2.02 (m, 1H), 1.92-1.85 (m, 0H), 1.68-1.20 (m, 35H), 0.94-0.82 (m, 6H).

### 1.4.9: AL08-010

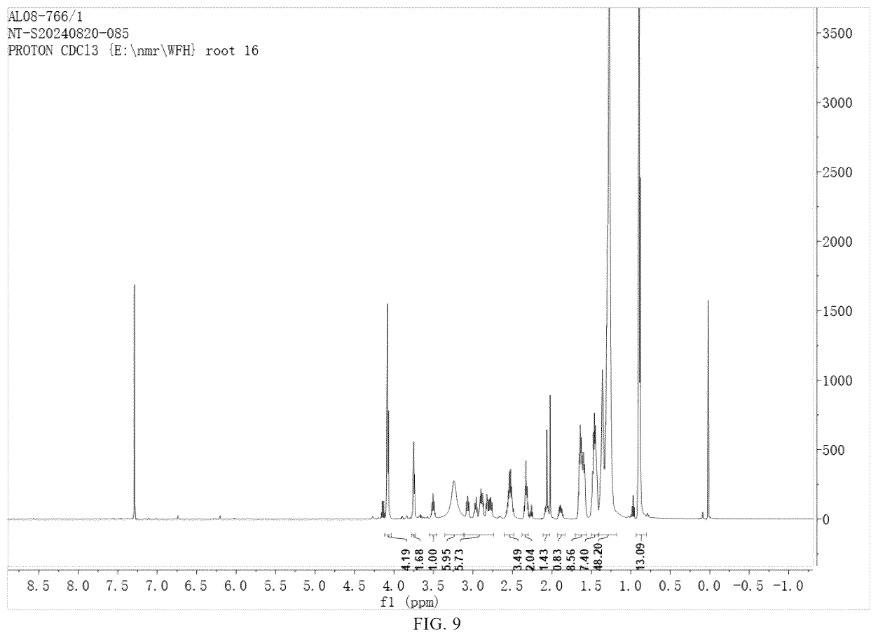

The ¹H NMR spectrum of AL08-010 is shown in FIG. 9. Mass spectrometry and NMR data are as follows:

LC-MS: (ES, m/z): 808.1 [M+H]⁺;

¹H NMR (600 MHz, Chloroform-d) δ 4.08 (t, J=6.7 Hz, 0H), 3.73 (t, J=5.2 Hz, OH), 3.47 (q, J=7.7 Hz, 0H), 3.04-2.97 (m, 0H), 2.97-2.65 (m, 0H), 2.56-2.39 (m, 0H), 2.32 (dq, J=9.3, 5.3, 4.6 Hz, 0H), 2.08-2.00 (m, 0H), 1.91-1.80 (m, 0H), 1.71-1.53 (m, 0H), 1.52-1.16 (m, 1H), 0.89 (t, J=7.0 Hz, 0H).

### 1.4.10: AL08-011

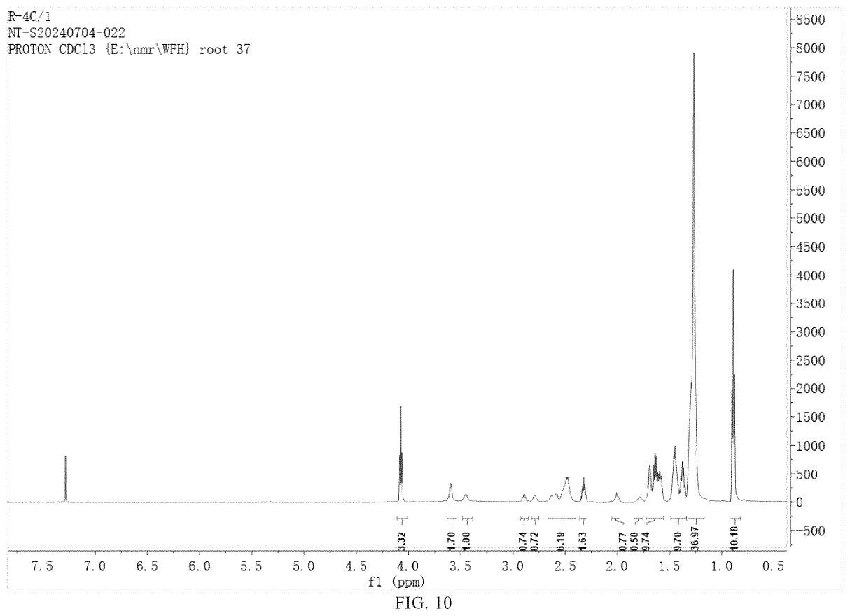

The ¹H NMR spectrum of AL08-011 is shown in FIG. 10. Mass spectrometry and NMR data are as follows:

LC-MS: (ES, m/z): 976.2 [M+H]+;

¹H NMR (600 MHz, Chloroform-d) δ 4.08 (t, J=6.7 Hz, 5H), 3.72 (t, J=5.2 Hz, 2H), 3.56-3.46 (m, 1H), 3.32-3.10 (m, 4H), 2.98-2.91 (m, 1H), 2.88-2.80 (m, 2H), 2.75-2.68 (m, 1H), 2.63-2.49 (m, 3H), 2.36-2.29 (m, 2H), 2.06 (q, J=6.7 Hz, 1H), 1.88-1.83 (m, 1H), 1.71-1.53 (m, 9H), 1.54-1.38 (m, 12H), 1.34-1.21 (m, 75H), 0.90 (t, J=7.0 Hz, 13H).

### 1.4.11: AL08-012

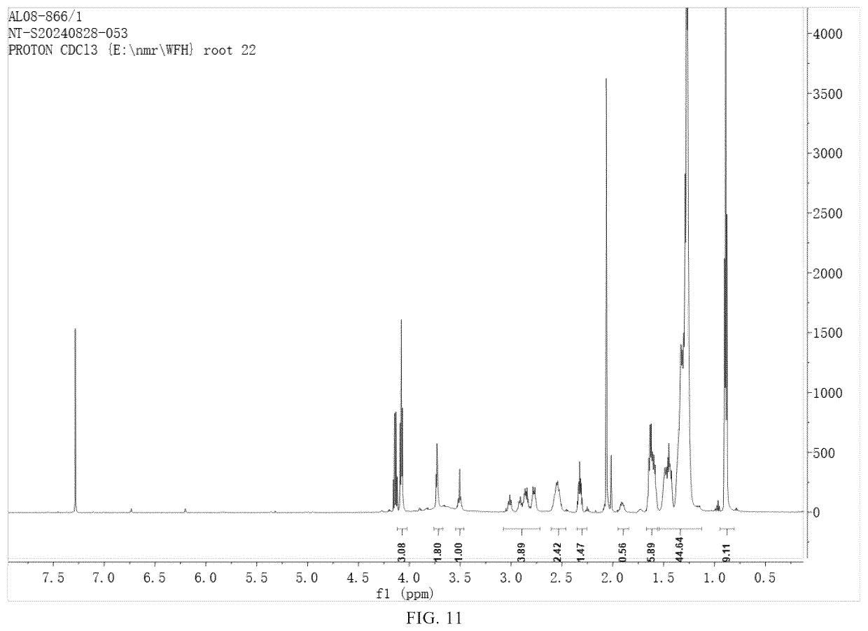

The ¹H NMR spectrum of AL08-012 is shown in FIG. 11. Mass spectrometry and NMR data are as follows:

LC-MS: (ES, m/z): 836.1 [M+H]⁺;

¹H NMR (600 MHz, Chloroform-d) δ 4.08 (t, J=6.7 Hz, 4H), 3.69 (t, J=5.3 Hz, 2H), 3.47-3.40 (m, 1H), 2.92-2.87 (m, 1H), 2.85-2.56 (m, 11H), 2.53-2.43 (m, 4H), 2.37-2.26 (m, 2H), 2.02 (s, 2H), 1.85-1.77 (m, 1H), 1.70-1.56 (m, 9H), 1.51-1.41 (m, 9H), 1.39-1.20 (m, 58H), 0.93-0.83 (m, 13H).

### 1.4.12: AL08-014

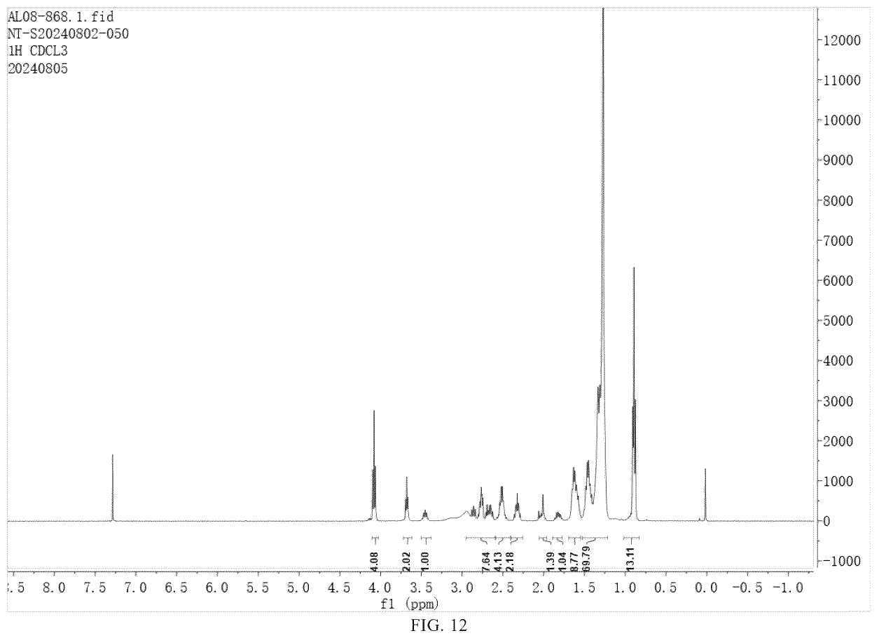

The ¹H NMR spectrum of AL08-014 is shown in FIG. 12. Mass spectrometry and NMR data are as follows:

LC-MS: (ES, m/z): 807.0 [M+H]⁺;

¹H NMR (600 MHz, Chloroform-d) δ 4.08 (td, J=6.7, 1.8 Hz, 1H), 3.70 (t, J=5.3 Hz, 0H), 3.50-3.35 (m, 0H), 3.01-2.57 (m, 1H), 2.57-2.43 (m, 1H), 2.31 (t, J=7.6 Hz, 1H), 2.08-1.81 (m, 0H), 1.83-1.75 (m, 0H), 1.70-1.58 (m, 1H), 1.47-1.40 (m, 1H), 1.40-1.20 (m, 12H), 0.90 (td, J=7.1, 2.1 Hz, 2H).

### 1.4.13: AL08-015

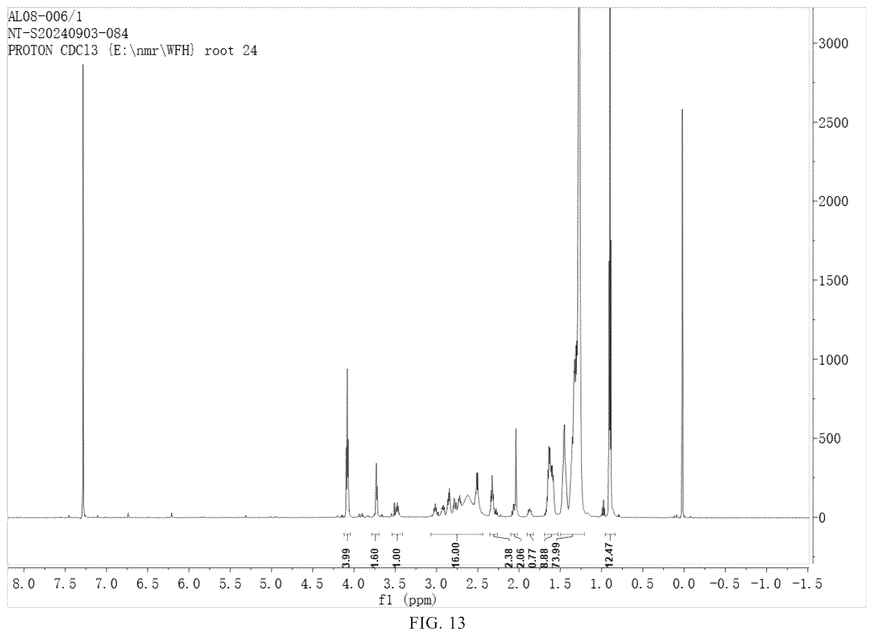

The ¹H NMR spectrum of AL08-015 is shown in FIG. 13. Mass spectrometry and NMR data are as follows:

LC-MS: (ES, m/z): 780.0 [M+H]⁺;

¹H NMR (600 MHz, Chloroform-d) δ 4.14-4.01 (m, 1H), 3.78 (t, J=5.1 Hz, 0H), 3.56 (t, J=7.6 Hz, 0H), 3.20-2.73 (m, 1H), 2.61-2.50 (m, 1H), 2.31 (t, J=7.6 Hz, 1H), 2.14-2.05 (m, 0H), 2.00-1.87 (m, 0H), 1.64 (d, J=7.2 Hz, 1H), 1.54-1.16 (m, 14H), 1.10-0.80 (m, 3H).

### 1.4.14: AL08-018

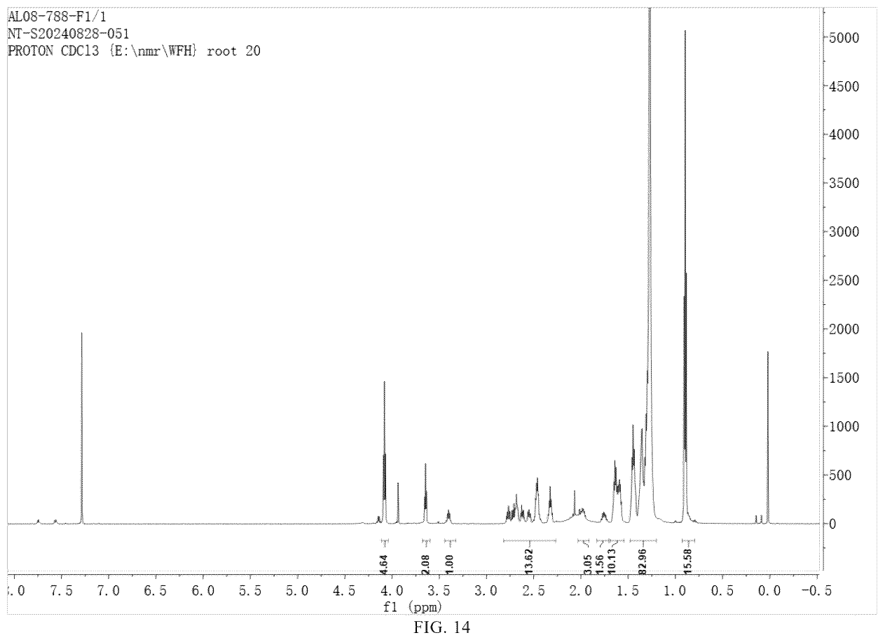

The ¹H NMR spectrum of AL08-018 is shown in FIG. 14. Mass spectrometry and NMR data are as follows:

LC-MS: (ES, m/z): 836.1 [M+H]⁺;

¹H NMR (600 MHz, Chloroform-d) δ 4.08 (t, J=6.7 Hz, 4H), 3.77 (t, J=5.2 Hz, 2H), 3.16-2.78 (m, 10H), 2.67-2.53 (m, 3H), 2.39-2.28 (m, 2H), 2.19-2.08 (m, 1H), 1.96 (s, 1H), 1.71-1.58 (m, 7H), 1.57-1.18 (m, 63H), 0.98-0.84 (m, 12H).

### 1.4.15: AL08-019

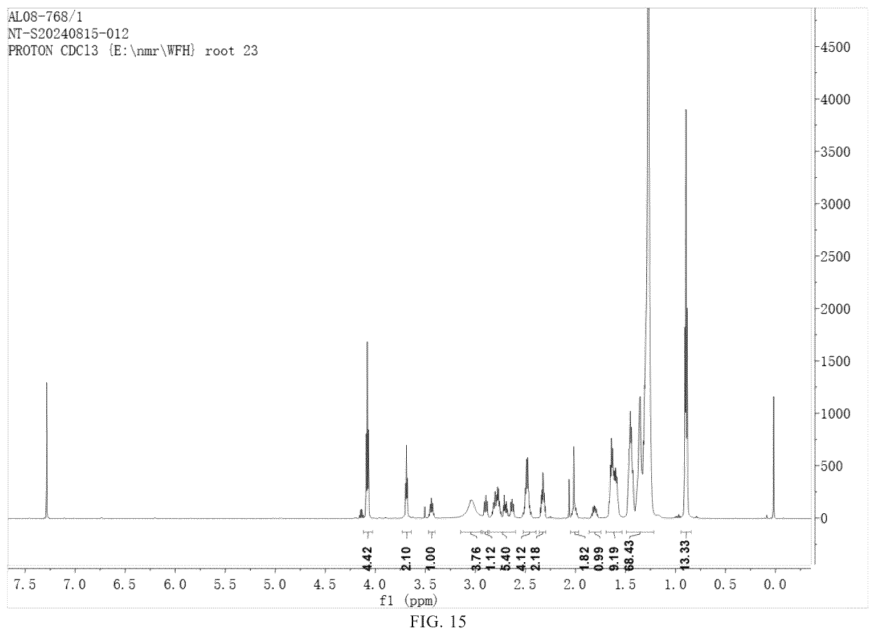

The ¹H NMR spectrum of AL08-019 is shown in FIG. 15. Mass spectrometry and NMR data are as follows:

LC-MS: (ES, m/z): 780.0 [M+H]⁺;

¹H NMR (600 MHz, Chloroform-d) δ 4.09 (t, J=6.7 Hz, 1H), 3.75 (q, J=6.7, 5.9 Hz, 1H), 3.08-2.68 (m, 2H), 1.73-1.50 (m, 3H), 1.50-1.20 (m, 16H), 0.90 (t, J=7.1 Hz, 3H).

### 1.4.16: AL08-020

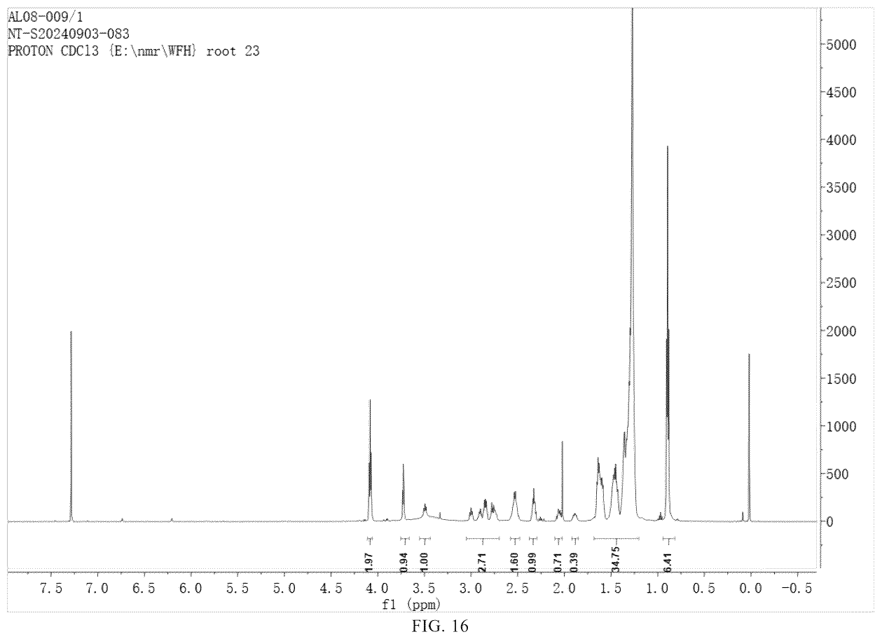

The ¹H NMR spectrum of AL08-020 is shown in FIG. 16. Mass spectrometry and NMR data are as follows:

LC-MS: (ES, m/z): 780.0 [M+H]⁺;

¹H NMR (600 MHz, Chloroform-d) δ 4.19-4.07 (m, 3H), 3.78 (t, J=5.2 Hz, 1H), 3.08-3.01 (m, 1H), 2.99-2.81 (m, 2H), 2.77 (q, J=7.0 Hz, 1H), 2.73-2.65 (m, 1H), 2.37-2.29 (m, 3H), 1.69 (p, J=6.9 Hz, 2H), 1.60 (ddd, J=21.4, 11.1, 7.1 Hz, 4H), 1.50-1.34 (m, 5H), 1.34-1.22 (m, 22H), 0.90 (t, J=7.0 Hz, 6H).

### 1.4.17: AL08-021

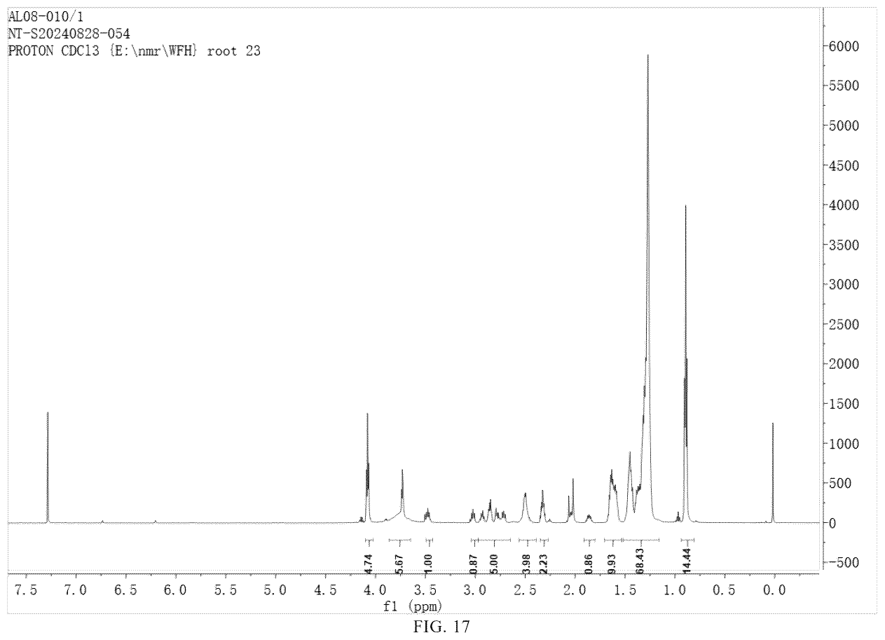

The ¹H NMR spectrum of AL08-021 is shown in FIG. 17. Mass spectrometry and NMR data are as follows:

LC-MS: (ES, m/z): 864.1 [M+H]⁺;

¹H NMR (600 MHz, Chloroform-d) δ 4.14 (q, J=7.1 Hz, 1H), 4.09 (t, J=6.7 Hz, 2H), 3.75 (q, J=6.7, 5.9 Hz, 1H), 3.08-3.01 (m, 1H), 2.98-2.91 (m, 1H), 2.89-2.77 (m, 2H), 2.37-2.29 (m, 1H), 2.07 (s, 2H), 1.65-1.56 (m, 4H), 1.49-1.40 (m, 2H), 1.36 (s, 7H), 1.34-1.23 (m, 23H), 0.90 (t, J=7.1 Hz, 6H).

### 1.4.18: AL08-022

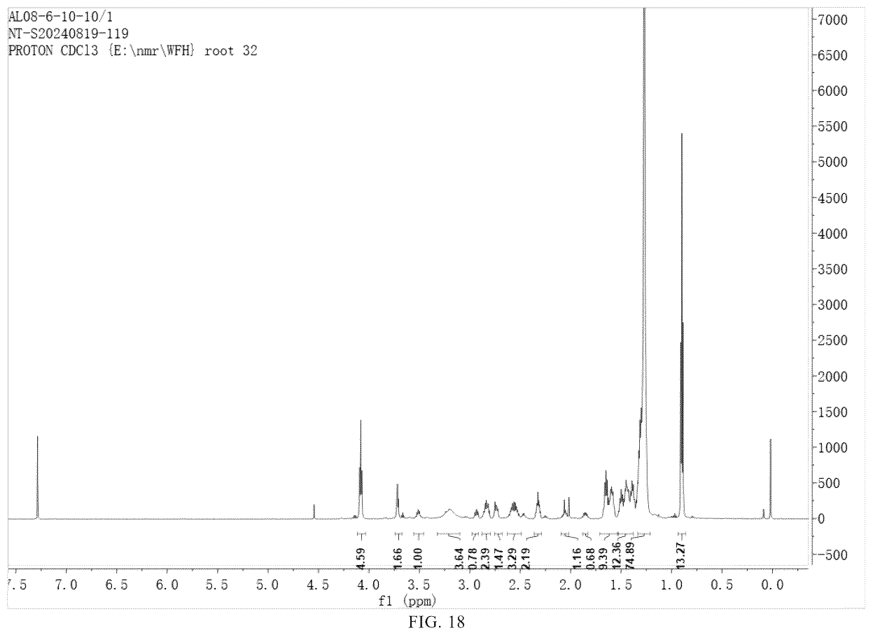

The ¹H NMR spectrum of AL08-022 is shown in FIG. 18. Mass spectrometry and NMR data are as follows:

LC-MS: (ES, m/z): 808.0 [M+H]⁺;

¹H NMR (600 MHz, Chloroform-d) δ 4.08 (t, J=6.7 Hz, 4H), 3.67 (t, J=5.3 Hz, 2H), 3.00 (s, 2H), 2.78-2.72 (m, 3H), 2.69-2.61 (m, 2H), 2.52 (dq, J=13.6, 6.8, 5.7 Hz, 4H), 2.33 (tt, J=9.0, 5.3 Hz, 2H), 1.68-1.56 (m, 8H), 1.52-1.19 (m, 61H), 0.89 (t, J=7.0 Hz, 13H).

### 1.4.19: AL08-023

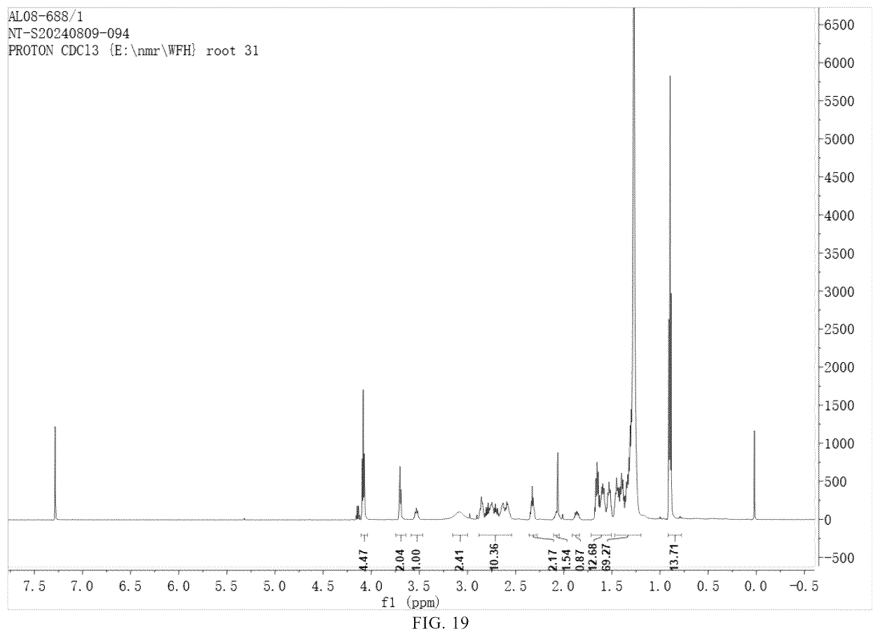

The ¹H NMR spectrum of AL08-023 is shown in FIG. 19. Mass spectrometry and NMR data are as follows:

LC-MS: (ES, m/z): 864.1 [M+H]⁺;

¹H NMR (600 MHz, Chloroform-d) δ 4.09 (t, J=6.7 Hz, 2H), 3.78 (t, J=5.2 Hz, 1H), 3.09-2.98 (m, 1H), 2.93-2.41 (m, 8H), 2.33 (tt, J=8.8, 5.3 Hz, 1H), 2.17 (s, 1H), 1.73-1.53 (m, 7H), 1.51-1.34 (m, 7H), 1.33-1.19 (m, 29H), 0.90 (t, J=7.0 Hz, 7H).

### 1.4.20: AL08-024

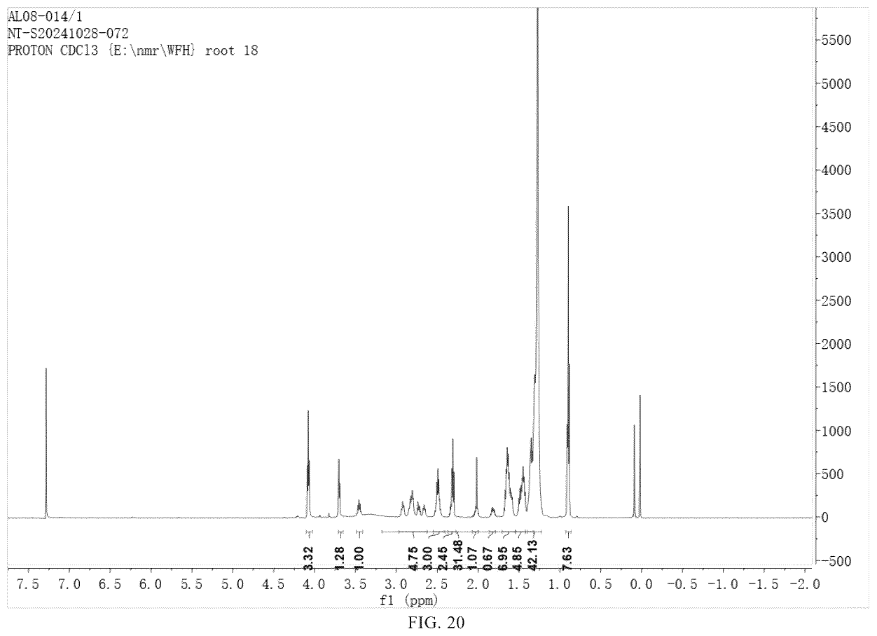

The ¹H NMR spectrum of AL08-024 is shown in FIG. 20. Mass spectrometry and NMR data are as follows:

LC-MS: (ES, m/z): 780.0 [M+H]⁺;

¹H NMR (600 MHz, Chloroform-d) δ 4.08 (t, J=6.7 Hz, 1H), 3.63 (t, J=5.4 Hz, 1H), 3.40 (t, J=7.6 Hz, 0H), 3.27 (q, J=6.7 Hz, 1H), 2.80-2.49 (m, 1H), 2.48-2.37 (m, 1H), 2.35-2.27 (m, 0H), 1.97 (d, J=4.8 Hz, 0H), 1.83-1.71 (m, 0H), 1.74-1.58 (m, 2H), 1.52 (t, J=7.4 Hz, 1H), 1.47-1.36 (m, 2H), 1.37-1.20 (m, 22H), 0.99-0.78 (m, 6H).

### 1.4.21: AL08-025

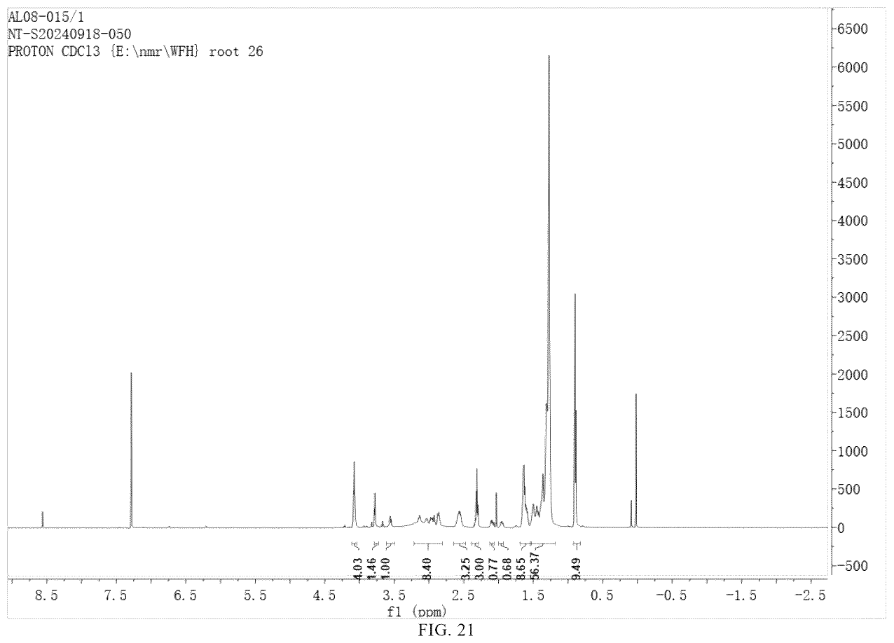

The ¹H NMR spectrum of AL08-025 is shown in FIG. 21. Mass spectrometry and NMR data are as follows:

LC-MS: (ES, m/z): 807.1 [M+H]⁺;

¹H NMR (600 MHz, Chloroform-d) δ 5.66 (t, J=6.0 Hz, 1H), 4.09 (t, J=6.7 Hz, 2H), 3.73 (t, J=5.2 Hz, 2H), 3.63 (q, J=7.7, 7.2 Hz, 1H), 3.28 (q, J=6.4 Hz, 2H), 3.01-2.60 (m, 13H), 2.33 (tt, J=8.9, 5.3 Hz, 1H), 2.17-2.07 (m, 1H), 2.01 (dd, J=9.3, 5.0 Hz, 1H), 1.93 (dt, J=13.9, 7.4 Hz, 1H), 1.71-1.51 (m, 12H), 1.50-1.19 (m, 56H), 0.96-0.84 (m, 12H).

### 1.4.22: AL08-026

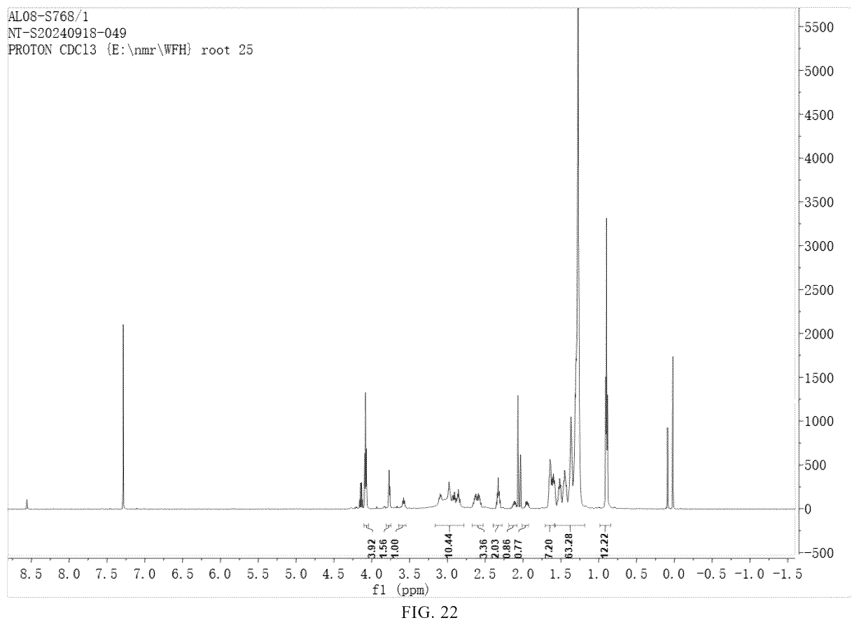

The ¹H NMR spectrum of AL08-026 is shown in FIG. 22. Mass spectrometry and NMR data are as follows:

LC-MS: (ES, m/z): 835.0 [M+H]⁺;

¹H NMR (600 MHz, Chloroform-d) δ 4.08 (t, J=6.7 Hz, 1H), 3.71 (t, J=5.3 Hz, 1H), 3.48 (t, J=7.6 Hz, 0H), 3.27 (q, J=6.7 Hz, 1H), 3.00-2.61 (m, 2H), 2.50 (q, J=8.0 Hz, 2H), 2.23-1.79 (m, 1H), 1.71-1.12 (m, 29H), 0.89 (td, J=7.1, 4.4 Hz, 6H).

### 1.4.23: AL08-027

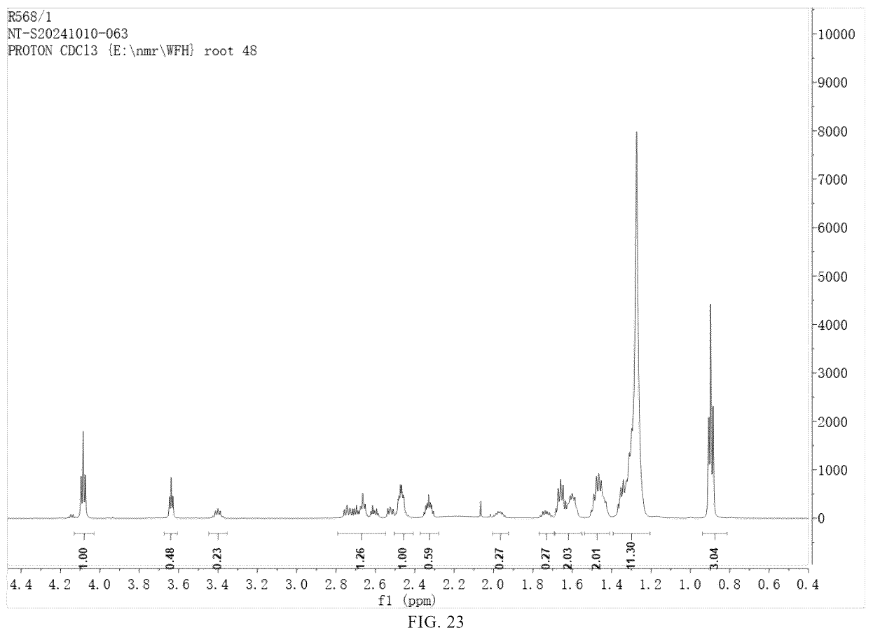

The ¹H NMR spectrum of AL08-027 is shown in FIG. 23. Mass spectrometry and NMR data are as follows:

LC-MS: (ES, m/z): 835.0 [M+H]⁺;

¹H NMR (600 MHz, Chloroform-d) δ 4.08 (t, J=6.7 Hz, 1H), 3.69 (t, J=5.3 Hz, 1H), 3.45 (d, J=7.6 Hz, 0H), 3.27 (q, J=6.7 Hz, 1H), 2.88 (t, J=8.7 Hz, 0H), 2.81-2.59 (m, 2H), 2.49 (d, J=8.8 Hz, 2H), 2.33 (dt, J=9.0, 4.0 Hz, 0H), 1.96 (dt, J=9.3, 4.5 Hz, OH), 1.82 (dd, J=13.2, 7.2 Hz, 0H), 1.73-0.98 (m, 30H), 0.99-0.71 (m, 6H).

### Example 2

LNP-mRNA was Assembled by Encapsulating mRNA with Ionizable Lipids

In the present disclosure, LNP-mRNA was prepared by encapsulating mRNA with ionizable lipids, and the steps included:
1) Ionizable lipid (AXT-8), DSPC (distearoylphosphatidylcholine, 1,2-distearoyl-sn-glycero-3-phosphocholine), cholesterol, and DMG-PEG2000 were mixed at different ratios in an ethanol phase and were fully homogenized to serve as the organic phase;
2) mRNA was dissolved in a sodium citrate solution (pH=4.0) and was fully homogenized to serve as the aqueous phase;
3) The aqueous phase and the organic phase were separately transferred into suitable BD syringes, with air bubbles exhausted as thoroughly as possible;
4) Using a PNI nanoparticle preparation instrument, mixing was performed under the conditions of an aqueous-to-organic phase volume ratio of 3:1 and a flow rate of 12 mL/min. Purification, concentration, and sterile filtration were sequentially carried out to obtain the mRNA-encapsulated lipid nanoparticle (LNP-mRNA) product; and
5) Physicochemical quality control was conducted on the final LNP-mRNA product.

Physicochemical quality control methods and results for LNP-mRNA:
1) Particle size and PDI (Polydispersity Index): The particle size and polydispersity of LNPs were determined using a nanoparticle size analyzer, with the results detailed in FIG. 1 and FIG. 2.
2) Encapsulation Efficiency (EE %): Total mRNA and free mRNA were separately stained with RiboGreen, followed by detection using a microplate reader. The encapsulation efficiency was calculated accordingly, with the results detailed in FIG. 3.
3) pH: The pH value of the final product was measured using a pH meter, and the result was 7.2-7.4.
4) Osmotic Pressure: The osmotic pressure of LNP-mRNA was determined using a freezing point osmometer. At a detection concentration of 100 g/mL, the osmotic pressure was 280-310 mOsmol/kg.
5) mRNA Integrity: The integrity (i.e., purity of mRNA) of the encapsulated mRNA was tested using Agilent Fragment Analyzers. The purity was determined to be greater than 90% with an RNA kit (15 nt).

Further, the AL08-001, AL08-003 to AL08-012, AL08-014, AL08-015, AL08-018 to AL08-027 prepared in Example 1 above, and AXT-8 encapsulation tool mRNA (mCherry) were assembled into LNP-mRNA. The steps included:
1) The ionizable lipids (AL08-001, AL08-003 to AL08-012, AL08-014, AL08-015, AL08-018 to AL08-027 prepared in Example 1, as well as AXT-8), DSPC (distearoylphosphatidylcholine, 1,2-distearoyl-sn-glycero-3-phosphocholine), cholesterol, and DMG-PEG2000 were mixed at different ratios in an ethanol phase and fully homogenized to serve as the organic phase;
2) mRNA was dissolved in a sodium acetate buffer (pH=4.0) solution and fully homogenized to serve as the aqueous phase;
3) The aqueous phase and the organic phase were separately transferred into appropriate syringes, with air bubbles exhausted as thoroughly as possible;
4) Using a PNI nanoparticle preparation instrument, mixing was performed under the conditions of an aqueous-to-organic phase volume ratio of 3:1 and a flow rate of 12 mL/min. Purification, concentration, and sterile filtration were sequentially carried out to obtain the mRNA-encapsulated lipid nanoparticle (LNP-mRNA) product;
5) Physicochemical quality control was conducted on the final LNP-mRNA product.

Physicochemical quality control methods and results for LNP-mRNA:
1) Particle size and Polydispersity Index (PDI): The particle size and polydispersity of LNPs were determined using a nanoparticle size analyzer. The results are detailed in Table 2, showing that the particle size was approximately 100 nm and PDI was mostly around 0.1.
2) Encapsulation Efficiency (EE %): Total mRNA and free mRNA were separately stained with RiboGreen, followed by detection using a microplate reader. The encapsulation efficiency was calculated accordingly, and the results are detailed in Table 2, showing that most encapsulation efficiencies were maintained at 95%.

The above results indicate that the ionizable lipids of the present invention possess favorable properties for LNP formation.

TABLE 2
Physicochemical properties and cell experimental data of different N/P formulations

| | | | Encapsulation | | | |
|---|---|---|---|---|---|---|
| | Particle Size | | Efficiency | | | Cell Viability |
| Formulation No. | (nm) | PDI | (%) | mCherry+^{a} | MFI^{a} | (%) |
| ALP08-001-1 | 100.52 | 0.072 | 85.59 | 0.0883 | 0.0998 | 81.48 |
| ALP08-001-2 | 103.94 | 0.094 | 96.31 | 0.0657 | 0.0828 | 89.94 |
| ALP08-001-3 | 117.61 | 0.139 | 97.52 | 0.0204 | 0.057 | 50.04 |
| ALP08-002-1 | 116.29 | 0.089 | 75.78 | 0.4293 | 0.4738 | 88.84 |
| ALP08-002-2 | 112.33 | 0.102 | 96.13 | 0.5518 | 0.4937 | 93.96 |
| ALP08-002-3 | 113.02 | 0.139 | 96 | 0.5103 | 0.4865 | 93.07 |
| ALP08-002-4 | 94.84 | 0.179 | 86.85 | 0.8281 | 0.5952 | 95.49 |
| ALP08-002-5 | 130.82 | 0.118 | 66.43 | 0.5551 | 0.5012 | 94.41 |
| ALP08-003-1 | 99.84 | 0.075 | 90.5 | 0.7191 | 1.0911 | 97.86 |
| ALP08-003-2 | 103.71 | 0.076 | 94.52 | 0.6179 | 1.1137 | 98.2 |
| ALP08-003-3 | 108.71 | 0.14 | 95.77 | 0.5606 | 1.1375 | 98.09 |
| ALP08-003-4 | 88.68 | 0.111 | 88.10 | 0.9049 | 0.9488 | 94.58 |
| ALP08-003-5 | 111.65 | 0.073 | 69.73 | 0.9176 | 1.0415 | 93.37 |
| ALP08-004-1 | 108.41 | 0.099 | 76.84 | 0.5763 | 0.2943 | 96.44 |
| ALP08-004-2 | 116.97 | 0.122 | 91.99 | 0.2989 | 0.1726 | 97.33 |
| ALP08-004-3 | 117 | 0.099 | 92.77 | 0.3188 | 0.1866 | 96.96 |
| ALP08-004-4 | 90.56 | 0.166 | 86.97 | 0.1880 | 0.3165 | 98.71 |
| ALP08-004-5 | 122.50 | 0.139 | 68.09 | 0.4510 | 0.3972 | 95.56 |
| ALP08-005-1 | 112.98 | 0.078 | 89.48 | 1.1166 | 1.9487 | 92.46 |
| ALP08-005-2 | 121.38 | 0.081 | 96.28 | 0.893 | 1.1145 | 93.27 |
| ALP08-005-3 | 138.44 | 0.136 | 96.16 | 0.9012 | 0.8981 | 93.74 |
| ALP08-005-4 | 88.08 | 0.118 | 86.13 | 0.8956 | 1.1201 | 94.67 |
| ALP08-005-5 | 132.78 | 0.125 | 66.53 | 0.9572 | 1.0156 | 92.26 |
| ALP08-006-1 | 111.37 | 0.127 | 88.46 | 0.4537 | 0.3458 | 92.84 |
| ALP08-006-2 | 122.66 | 0.147 | 94.23 | 0.0607 | 0.2224 | 94.59 |
| ALP08-006-3 | 129.33 | 0.063 | 96.77 | 0.0833 | 0.223 | 94.74 |
| ALP08-006-4 | 99.72 | 0.158 | 86.99 | 0.2354 | 0.3174 | 98.8 |
| ALP08-006-5 | 129.17 | 0.049 | 84.85 | 0.4984 | 0.5882 | 97.85 |
| ALP08-007-1 | 109.8 | 0.07 | 87.38 | 0.3058 | 0.6323 | 98.73 |
| ALP08-007-2 | 112.62 | 0.089 | 91.25 | 0.2445 | 0.5381 | 98.63 |
| ALP08-007-3 | 116.61 | 0.085 | 93.17 | 0.1677 | 0.4072 | 98.76 |
| ALP08-008-1 | 111.86 | 0.108 | 91.09 | 0.7563 | 0.894 | 98.45 |
| ALP08-008-2 | 127.27 | 0.132 | 96.2 | 0.565 | 0.8004 | 98.55 |
| ALP08-008-3 | 126.33 | 0.14 | 95.26 | 0.4904 | 0.6654 | 98.63 |
| ALP08-008-4 | 95.49 | 0.163 | 88.13 | 0.7764 | 0.792 | 95.72 |
| ALP08-008-5 | 120.44 | 0.081 | 70.44 | 0.8401 | 1.0415 | 93.88 |
| ALP08-009-1 | 109.97 | 0.134 | 93.17 | 1.0048 | 0.5478 | 94.18 |
| ALP08-009-2 | 110.06 | 0.157 | 96.01 | 0.8651 | 0.4554 | 95.36 |
| ALP08-009-3 | 131.43 | 0.155 | 97.49 | 0.6829 | 0.4269 | 94.94 |
| ALP08-009-4 | 88.24 | 0.156 | 87.10 | 0.7778 | 0.463 | 98.31 |
| ALP08-009-5 | 118.58 | 0.086 | 70.35 | 0.5166 | 0.459 | 98 |
| ALP08-010-1 | 104.37 | 0.096 | 93.04 | 0.8261 | 0.4362 | 93.19 |
| ALP08-010-2 | 116.81 | 0.173 | 97.08 | 0.5051 | 0.3462 | 93.45 |
| ALP08-010-3 | 142.81 | 0.149 | 97.06 | 0.3929 | 0.3151 | 91.69 |
| ALP08-010-4 | 106.20 | 0.137 | 88.19 | 0.5228 | 0.3632 | 97.43 |
| ALP08-010-5 | 119.61 | 0.104 | 68.43 | 0.2291 | 0.3531 | 94.9 |
| ALP08-011-1 | 118.32 | 0.042 | 94.99 | 0.0072 | 0.1449 | 98.77 |
| ALP08-011-2 | 111.15 | 0.131 | 96.29 | 0.0202 | 0.0975 | 98.86 |
| ALP08-011-3 | 108.34 | 0.119 | 96.6 | 0.0132 | 0.0921 | 98.9 |
| ALP08-012-1 | 100.17 | 0.063 | 81.55 | 1.0693 | 1.1286 | 98.31 |
| ALP08-012-2 | 105.18 | 0.075 | 92.8 | 0.8078 | 0.7981 | 98.37 |
| ALP08-012-3 | 142.26 | 0.165 | 92.87 | 0.6944 | 0.3926 | 98.6 |
| ALP08-012-4 | 97.54 | 0.182 | 87.70 | 0.8738 | 0.7384 | 93.9 |
| ALP08-012-5 | 131.27 | 0.085 | 68.04 | 0.8243 | 0.7025 | 92.83 |
| ALP08-015-1 | 126.94 | 0.1 | 80.67 | 0.7893 | 0.5632 | 91.09 |
| ALP08-015-2 | 118.33 | 0.126 | 95.69 | 0.9387 | 0.4496 | 92.24 |
| ALP08-015-3 | 125.83 | 0.162 | 96.95 | 0.8256 | 0.4015 | 84.91 |
| ALP08-015-4 | 96.81 | 0.159 | 91.16 | 0.6214 | 0.378 | 95.56 |
| ALP08-015-5 | 160.96 | 0.129 | 78.10 | 0.5875 | 0.5224 | 93.96 |
| ALP08-016-1 | 108.46 | 0.114 | 84.04 | 0.9757 | 1.4308 | 96.02 |
| ALP08-016-2 | 106.96 | 0.12 | 96.88 | 0.7646 | 1.0753 | 97.27 |
| ALP08-016-3 | 124.95 | 0.158 | 97.81 | 0.8307 | 1.0175 | 96.98 |
| ALP08-017-1 | 119.09 | 0.131 | 74.49 | 1.0721 | 0.8529 | 83.06 |
| ALP08-017-2 | 97.77 | 0.115 | 95.71 | 1.0815 | 1.0443 | 89.22 |
| ALP08-017-3 | 118.26 | 0.115 | 96.63 | 1.022 | 0.6234 | 89.42 |
| ALP08-018-1 | 102.61 | 0.104 | 81.37 | 0.5566 | 0.4325 | 91.06 |
| ALP08-018-2 | 120.61 | 0.164 | 93.68 | 0.287 | 0.3474 | 92.55 |
| ALP08-018-3 | 116.3 | 0.121 | 95.49 | 0.2057 | 0.3492 | 90.03 |
| ALP08-019-1 | 102.25 | 0.130 | 79.88 | 1.0187 | 1.0735 | 98.45 |
| ALP08-019-2 | 122.57 | 0.171 | 94.04 | 0.9602 | 0.8906 | 98.9 |
| ALP08-019-3 | 118.28 | 0.133 | 95.39 | 0.7762 | 0.8448 | 99.38 |
| ALP08-020-1 | 96.52 | 0.148 | 79.02 | 0.5765 | 0.3763 | 89.71 |
| ALP08-020-2 | 112.08 | 0.184 | 94.87 | 0.4717 | 0.3755 | 91.17 |
| ALP08-020-3 | 114.17 | 0.174 | 95.42 | 0.4408 | 0.3736 | 88.78 |
| ALP08-021-1 | 102.01 | 0.136 | 81.71 | 1.0280 | 0.6948 | 97.65 |
| ALP08-021-2 | 112.29 | 0.152 | 93.06 | 0.7347 | 0.5904 | 97.32 |
| ALP08-021-3 | 110.78 | 0.148 | 94.60 | 0.6421 | 0.5340 | 96.77 |
| ALP08-022-1 | 115.82 | 0.140 | 82.06 | 1.0236 | 1.1157 | 99.09 |
| ALP08-022-2 | 110.62 | 0.107 | 93.60 | 1.3376 | 1.2971 | 99.02 |
| ALP08-022-3 | 130.00 | 0.102 | 94.39 | 1.2684 | 1.0899 | 98.81 |
| ALP08-023-1 | 116.78 | 0.114 | 81.74 | 0.5052 | 0.6163 | 97.31 |
| ALP08-023-2 | 114.68 | 0.154 | 86.18 | 0.5174 | 0.5505 | 96.48 |
| ALP08-023-3 | 117.34 | 0.111 | 88.74 | 0.4651 | 0.4841 | 92.61 |
| ALP08-024-1 | 87.06 | 0.212 | 81.75 | 1.0908 | 0.6337 | 78.17 |
| ALP08-024-2 | 90.71 | 0.169 | 90.33 | 1.1707 | 0.9114 | 79.06 |
| ALP08-024-3 | 95.91 | 0.160 | 93.60 | 1.1664 | 0.9832 | 89.7 |
| ALP08-025-1 | 92.37 | 0.089 | 89.44 | 1.6189 | 0.8949 | 96.98 |
| ALP08-025-2 | 96.79 | 0.149 | 94.18 | 1.1885 | 0.6945 | 98.37 |
| ALP08-025-3 | 127.01 | 0.131 | 87.46 | / | / | / |
| ALP08-026-1 | 104.01 | 0.108 | 84.42 | 0.9484 | 0.6871 | 77.23 |
| ALP08-026-2 | 102.06 | 0.115 | 93.43 | 0.8358 | 0.5775 | 88.27 |
| ALP08-026-3 | 118.38 | 0.147 | 91.86 | 0.1246 | 0.3658 | 73.36 |
| ALP08-027-1 | 106.51 | 0.085 | 83.79 | 0.9349 | 0.6758 | 76.35 |
| ALP08-027-2 | 108.93 | 0.135 | 93.52 | 0.8977 | 0.5750 | 89.06 |
| ALP08-027-3 | 145.26 | 0.160 | 87.21 | / | / | / |

Note:
^{a}represents ALP08-X-Y/AXT-8, i.e., the ratio of the corresponding parameters of ALP08-X-Y formulation to those of AXT-8 formulation, wherein X represents a positive integer from 1 to 27, indicating the compound number, and Y represents a positive integer from 1 to 5, indicating the formulation number. The formulation of AXT-8 LNP is: AXT08:DSPC:Chol:DMG-PEG2000 = 50:10:38.5:1.5, with an N/P ratio of 3:1.
“/” indicates that the physicochemical properties did not meet the specifications, and cellular experiments were not performed.

Y corresponds to different formulations. Y being 1, 2, and 3 represents the corresponding formulation as 50:10:38.5:1.5 (ionizable lipid:DSPC:cholesterol:DMG-PEG2000, molar ratio), with N/P ratios of 3:1, 4:1, and 5:1, respectively (wherein the N/P ratio for AL08-024-3 and AL08-025-3 is 6:1). Y4 corresponds to the formulation ratio of 40:20:38.5:1.5, with an N/P ratio of 3:1. Y being 5 corresponds to the formulation ratio of 60:10:28.5:1.5, with an N/P ratio of 3:1

### Example 3

### In Vitro Expression Assay of LNP-mRNA

The present disclosure also conducted cell expression screening experiments for LNP-mRNA. The specific steps were as follows:
1) Cell Plating: 293T cells were digested, and the cell density was adjusted to 2×10⁴/well, then seeded into a 96-well plate at a volume of 100 L/well. The plate was then incubated overnight in a cell culture incubator.
2) Experimental Grouping: LNP (AXT-8)-Luc series products were used as the sample group; Lipofectamine Messenger MAX was set as the positive control group; and a mixture containing only 100 L of medium and cells was used as the negative control group.
3) Cell Transfection: LNP-Luc did not require transfection. Taking 100 ng of mRNA per well as the maximum concentration, three-fold serial dilutions were performed to obtain six gradients in total, with three replicate wells set up for each sample. 10 L of LNP-Luciferase at different dilution ratios was uniformly added to the 96-well cell plate, and the plate was incubated in the incubator after thorough mixing.
4) Kit Detection: After 48 h of incubation, the assay was performed in accordance with the protocol of the ONE-Glo™ EX Luciferase Assay System kit. The luciferase luminescence intensity was detected using a BioTek SYNERGY microplate reader, and the OD values were averaged for comparison.

The results were as shown in FIG. 4. LNP (AXT-8)-Luc samples with different formulations were all capable of normal expression in cells, and the expression level increased with the rise in mRNA concentration. It can thus be concluded that the LNP-mRNA prepared from AXT-8 achieved high-efficiency expression in vitro.

### Example 4

### In Vitro Toxicity Assay of LNP-mRNA

Cytotoxicity assay, the specific steps were as follows:
1) Cell Plating: 293T cells were digested, and the cell density was adjusted to 2×10⁴/well, then seeded into a 96-well plate at 100 μL per well. The plate was then incubated overnight in a cell culture incubator.
2) Experimental Grouping: LNP (AXT-8)-Luc was used as the sample group; 100 L of medium alone was used as the blank control group; a mixture of 100 L of medium and cells was used as the negative control group; and a mixture of 100 L of medium and cells supplemented with the apoptosis inducer Staurosporine (STS) was used as the positive control group.
3) Cell Transfection: LNP-Luc did not require transfection. Using 100 ng of mRNA per well as the maximum concentration, three-fold serial dilutions were performed with buffer to obtain four gradients in total. 10 L of LNP-Luc at each dilution was uniformly added to the 96-well cell plate, with three replicate wells set up for each sample, and the plate was incubated in the incubator after thorough mixing. 10 μL of 100 M STS (diluted in PBS) was added to the 96-well cell plate, with six replicate wells set up. The plate was incubated in the incubator after thorough mixing.
4) Kit Detection: After 48 h of incubation, the assay was performed in accordance with the protocol provided by the CellTiter-Glo® Luminescent Cell Viability Assay kit. The luminescence intensity was detected using a BioTek SYNERGY microplate reader, and the OD values were averaged for comparison. The cell inhibition rate was calculated using the formula: (Negative Control Group-Sample Group/Positive Control Group)+(Negative Control Group−Blank control group)×100%, and the dose-response-inhibition curve was plotted.

The results were as shown in FIG. 5. For the series of LNP-mRNA products formulated with AXT-8, no significant effect on cell proliferation was observed when the concentration was less than 1 μg/mL, indicating that there was no obvious cytotoxicity within the tested concentration range.

### Example 5

### In Vivo Expression Assay of LNP-mRNA

To illustrate the in vivo expression capacity and toxicity of LNP-mRNA formulated with AXT-8, hEPO ELISA expression assays and toxicity experiments were further designed. The expression level and toxicity were determined in mice with intact innate immunity. The specific steps were shown as follows:
1) Encapsulation of LNP-mRNA: hEPO mRNA was dissolved in an aqueous buffer and mixed thoroughly to serve as the aqueous phase. Ionizable lipid (AXT-8), DSPC, cholesterol, and DMG-PEG2000 were separately dissolved in anhydrous ethanol, and mixed thoroughly at a specific ratio to serve as the organic phase. The aqueous phase and the organic phase were separately transferred into syringes. hEPO-LNP was prepared using a PNI microfluidic nanoparticle preparation instrument (the volume ratio of aqueous phase to organic phase was 3:1, and the flow rate was 12 mL/min). After concentration, purification, sterile filtration, and passing quality control, the LNP-hEPO was administered to mice via injection.
2) Mouse Tail Vein Injection: Mice were immobilized, and the tail vein was selected for injection. The injection dose of LNP-hEPO was 5 μg. LNP (AXT-8)-hEPO was designated as the sample group, while mice injected with solvent alone served as the negative control group. Two mice were injected per LNP-hEPO sample.
3) Submandibular Blood Collection in Mice: At 6 h and 24 h post-injection, submandibular blood collection was performed. The collected blood was placed into EDTA anticoagulant tubes and mixed gently, then labeled for subsequent use.
4) Serum Extraction: After whole blood collection, centrifugation was carried out at 2000×g for 10 minutes. The supernatant was collected as plasma, aliquoted, and stored at −80° C.
5) Determination of hEPO Expression Level Using Human Erythropoietin ELISA Kit: The assay was performed in accordance with the kit instructions, with two replicate wells set up for each serum sample. The OD values were measured using a microplate reader, and the expression levels were calculated based on the standard curve and dilution ratio. The conversion factor between IU and g for the kit used was exemplified as follows: 1650IU=11 kg.

The physicochemical property values of the LNP-hEPO formulation prepared with AXT-8, which can achieve successful expression in mice, as well as the in vivo hEPO expression concentrations were shown in Table 3.

TABLE 3

| | | | | Expression |
|---|---|---|---|---|
| | Particle | | Encapsulation | Level |
| | Size | | Efficiency | at 6 h |
| Formulation | (nm) | PDI | (%) | (μg/mL) |
| LNP(AXT-8)-hEPO-1 | 95.19 | 0.091 | 94.81 | C |
| LNP(AXT-8)-hEPO-2 | 87.64 | 0.074 | 96.75 | C |

C: ≥0.1 and <1;
D: <0.1

The results were as shown in FIG. 6. The series of LNP (AXT-8)-hEPO products formulated with AXT-8 achieved successful expression in mice. This experiment demonstrated that the LNPs formed with AXT-8 could efficiently mediate the in vivo expression of mRNA.

### Example 6

### Expression Assay of LNP-mRNA in T Cell Lines

To illustrate the capacity of LNP-mRNA formulated with AXT-8 to transfect human T cell lines in vitro, experiments were designed to transfect JM cells and Jurkat cells with LNP-eGFP. Meanwhile, Lipofectamine™ MessengerMAX™ (Lipofectamine Messenger MAX, purchased from Thermo Fisher Scientific, product code: LMRNA001) was used as the positive control group to determine the eGFP expression level in the cells. The specific steps were as shown as follows:
1) Cell Plating: Cells were counted using the AO/PI method. The culture medium contained only FBS without antibiotics. JM cells or Jurkat cells were adjusted to a density of 1*10⁶/mL and seeded into a 24-well plate at 200 μL per well, corresponding to 2*10⁵/well.
2) Cell Transfection: mRNA encapsulated with AXT-8 was added at a dose of 6 ug/well, and the medium was supplemented to 1 mL. The medium contained only FBS without adding antibiotics.
3) Flow Cytometry Analysis: After incubation at 37° C. for 24 h, flow cytometry analysis was performed. The cells were harvested separately into 1.5 mL EP tubes, labeled with sample names, centrifuged at 400 g for 5 min, and the supernatant was discarded. The cells were washed once with 1 mL of 2% FBS (prepared in PBS), centrifuged at 400 g for 5 min, and the supernatant was discarded. The corresponding antibody was added to the cell suspension and incubated for 20 min for staining. The cells were washed again with 1 mL of 2% FBS (prepared in PBS), centrifuged at 400 g for 5 min, and the supernatant was discarded. The cells were resuspended in 50 μL of 2% FBS (prepared in PBS) and subjected to flow cytometry analysis.

The results were as shown in FIG. 7. The LNP-eGFP formulated with AXT-8 achieved successful expression in both JM cells and Jurkat cells.

### Example 7

### Expression Assay of LNP-mRNA in Human Primary T Cells

To illustrate the capacity of LNP-mRNA formulated with AXT-8 to transfect human T cell lines in vitro, experiments were designed to transfect human primary T cells with LNP-mCherry. Meanwhile, the transfection reagent Lipofectamine™ MessengerMAX™ (Lipofectamine Messenger MAX) was used as the positive control group to determine the mCherry expression level in the cells. The specific steps were shown below:
1) Isolation of Primary T Cells from Human PBMCs: Magnetic bead labeling was performed (the following procedure is exemplified with 10⁷ cells; the amount of corresponding reagents should be scaled up proportionally according to the total cell number). Every 10⁷ total cells were resuspended in 80 L of buffer, followed by the addition of 20 L of CD3 MicroBeads. The cell suspension was incubated for 15 minutes (2 to 8° C.) in a refrigerator, then washed with buffer. Up to 10⁸ cells were resuspended in 500 μL of buffer. The MS column was placed on a magnetic separator, and T cells were sorted using the MS column.
2) Transfection of Primary T Cells with AXT-8: The T cells were adjusted to a density of 1*10⁶/mL and seeded into a 24-well plate at 200 μL per well, corresponding to 2*10⁵/per well. AXT-8 was added at a concentration of 6 μg/mL, and the medium was supplemented with X-vivo medium to 1 mL. IL-2 was added to each well to a final concentration of 100 IU/mL. The cells were incubated at 37° C. for 24 hours prior to flow cytometry analysis.
3) Flow Cytometry Analysis: The cells were harvested separately into 1.5 mL EP tubes and labeled with sample names. The cell suspension was centrifuged at 400 g for 5 min, and the supernatant was discarded. The cells were washed once with 1 mL of 2% FBS (prepared in PBS), centrifuged again at 400 g for 5 min, and the supernatant was discarded. The corresponding antibody was added to the cell suspension and incubated for 20 min for staining. The cells were washed again with 1 mL of 2% FBS (prepared in PBS), centrifuged at 400 g for 5 min, and the supernatant was discarded. The cells were resuspended in 50 L of 2% FBS (prepared in PBS) and subjected to flow cytometry analysis.

The results were as shown in FIG. 8. The LNP-mCherry formulated with AXT-8 achieved successful expression in human primary T cells.

Further, the expression of LNPs prepared from more ionizable lipids of the present invention in human primary T cells was verified:
1) Isolation of primary T cells from human PBMCs: Magnetic bead labeling (the following procedure is exemplified with 10⁷ cells; the amount of corresponding reagents should be increased proportionally according to the total cell number). Every 10⁷ total cells were resuspended in 80 μL of buffer, followed by the addition of 20 μL of CD3 MicroBeads. The cell suspension was incubated for 15 minutes (2-8° C.) in a refrigerator, then washed with buffer. Up to 10⁸ cells were resuspended in 500 μL of buffer. The MS column was placed on a magnetic separator, and T cells were sorted using the MS column.
2) Transfection of primary T cells with LNP-mRNA: The T cells were adjusted to a density of 1×10⁶/mL and seeded into a 24-well plate at 200 μL per well, corresponding to 2×10⁵ cells per well. LNP-mRNA was added at a concentration of 6 μg/mL (quantified by mRNA), and the medium was supplemented with X-vivo medium to 1 mL. IL-2 was added to each well to a final concentration of 100 IU/mL. After incubation at 37° C. for 24 h, flow cytometry analysis was performed.
3) Flow cytometry analysis: The cells were harvested separately into 1.5 mL EP tubes and centrifuged at 400 g for 5 min, and the supernatant was aspirated and discarded. Cells were washed once with 1 mL of 2% FBS (prepared in PBS), centrifuged at 400 g for 5 min, and the supernatant was aspirated and discarded. The corresponding antibody was added to the cell suspension and incubated for 20 min for staining. Cells were washed once with 1 mL of 2% FBS (prepared in PBS), centrifuged at 400 g for 5 min, and the supernatant was aspirated and discarded. The cells were resuspended in 50 μL of 2% FBS (prepared in PBS) and subjected to flow cytometry analysis. Toxicity was assessed by cell viability (see Table 4 for details).

As shown by the data in Table 4, after transfection of primary T cells with different formulations (at a dose of 6 μg/mL), the cell viability of most formulations was higher than 90%, demonstrating their high safety.

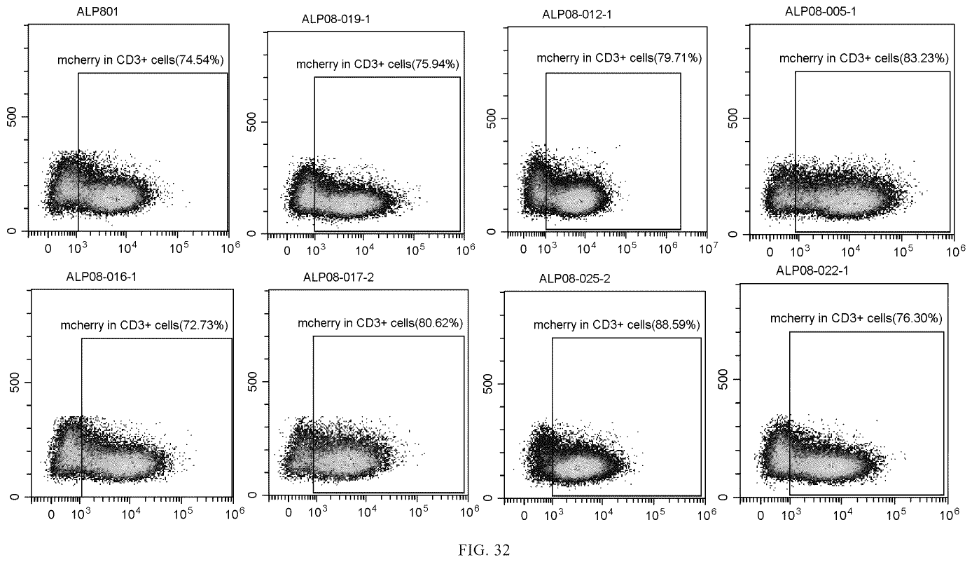

Among them, the flow cytometry data for some LNP-mRNA (AXT-8 LNP, ALP08-005-1, ALP08-019-1, ALP08-012-1, ALP08-022-1, and ALP08-025-2) are shown in FIG. 32 and Table 4. As shown in FIG. 32, all listed formulations achieved transfection efficiencies of over 70%.

TABLE 4
Transfection efficiency and expression levels of different
formulations in transfected primary T cells

| | Transfection | MFI (Expression |
|---|---|---|
| Formulation | Efficiency (%) | Level, RFU) |
| AXT-8 LNP | 74.54 | 6338.1 |
| ALP08-005-1 | 83.23 | 12351.1 |
| ALP08-019-1 | 75.94 | 6803.9 |
| ALP08-012-1 | 79.71 | 7153.3 |
| ALP08-022-1 | 76.3 | 7071.6 |
| ALP08-025-2 | 88.59 | 4402 |
| ALP08-016-1 | 72.73 | 9068.5 |
| ALP08-017-2 | 80.62 | 6618.8 |

In Table 4, the transfection efficiency of other LNP-mRNA was evaluated relative to the positive control AXT-8 LNP as the baseline. The formulation of AXT-8 LNP is: AXT-8:DSPC:Chol:DMG-PEG2000=50:10:38.5:1.5, with an N/P ratio of 4:1.

Table 4 shows the ratio of CD3+ positivity after transfection of human primary T cells by different formulations compared to the positive control AXT-8 LNP (mCherry). Based on the ratio results, the T cell transfection efficiency of formulations such as ALP08-019-1, ALP08-012-1, ALP08-005-1, ALP08-022-1, ALP08-025-2, and ALP08-017-2 is higher than or comparable to that of AXT-8 LNP.

Table 4 shows the ratio of MFI (mean fluorescence intensity) after transfection of human primary T cells by different formulations compared to the positive control AXT-8 LNP (mCherry). As shown in FIG. 32, the mean fluorescence intensity of T cells transfected by ALP08-005-1, ALP08-019-1, ALP08-012-1, ALP08-022-1, ALP08-016-1, and ALP08-017-2 is higher than or comparable to that of AXT-8 LNP.

### Example 8: Preparation of Ab-LNP

### Preparation of LNP-Mal:

Ionizable lipid:DSPC:Chol:DSPE-PEG2k:DSPE-PEG5k-Mal: (50:10:38:X:Y, molar ratio; the ionizable lipid is exemplified by AL08-005 here, X+Y=2, Y can range from 0.25 to 2). The lipids were dissolved in ethanol, mCherry was dissolved in a sodium acetate aqueous solution at pH 4.0, and LNP-mal was prepared by microfluidics.

### Antibody Activation:

A certain volume of LNP-mal solution was measured, and the molar amount of DSPE-PEG5k-mal therein was calculated. Using a molar ratio of Ab antibody to mal derivative of 1:2, the molar amount of Ab antibody was calculated. Based on a molar ratio of TCEP to Ab antibody (the antibody used here is hCD8) of 20:1 for activation, the corresponding volume of TCEP solution was measured and mixed uniformly with the aforementioned Ab antibody, incubated at room temperature for 0.5 hours, and excess TCEP was removed using a G25 desalting column.

A certain amount of LNP-mal solution was added to the above TCEP-activated antibody solution and incubated for 1 h to form Ab-LNP conjugated with antibody. The antibody concentration was detected using a BCA protein assay kit.

The physicochemical characterization results of Ab-LNP are shown in Table 5.

TABLE 5
Physicochemical properties results of Ab-LNP

| | Particle Size | | | Antibody Concentration |
|---|---|---|---|---|
| Sample Name | (nm) | PDI | EE % | (mg/mL) |
| LNP | 130.17 | 0.114 | 94.04 | / |
| Ab-LNP | 143.55 | 0.115 | 91.15 | 1.678 |

The physicochemical characterization results of Ab-LNP demonstrated that, compared to LNP without antibody conjugation, its particle size increased by approximately 15 nm, PDI was less than 0.2, encapsulation efficiency reached as high as 91.15%, and antibody concentration was 1.678 mg/mL, proving its favorable properties for forming nanoparticles.

### Example 9: Transfection of hPBMC with Ab-LNP

Prepared primary PBMCs (without activation treatment, e.g., not activated with anti-CD3/anti-CD28 antibodies) were rapidly thawed in a 37° C. water bath, added to medium pre-warmed to 37° C., centrifuged at 300 g for 10 min, and resuspended in X-vivo 15 medium for AO/PI cell counting. Cells were adjusted to 2.5×10⁶/mL and seeded into a 24-well plate at 200 L per well (i.e., 5×10⁵ cells/well). Ab-LNP (containing CD8 antibody) prepared in Example 4 was added at 6 g/mL, and X-vivo medium was supplemented to 1 mL. After culturing at 37° C. for 24 h, cells were collected into 1.5 mL EP tubes, centrifuged at 400 g for 5 min, and the supernatant was aspirated and discarded. Cells were washed once with 1 mL of 2% FBS (prepared in PBS), and repeatedly washed twice. Flow cytometry detection antibodies were added to the cell suspension and stained at room temperature for 20 min, followed by washing once with 1 mL of 2% FBS (prepared in PBS), centrifuging at 400 g for 5 min, aspirating and discarding the supernatant, and resuspending the cells in 100 L of 2% FBS (prepared in PBS) for flow cytometry analysis.

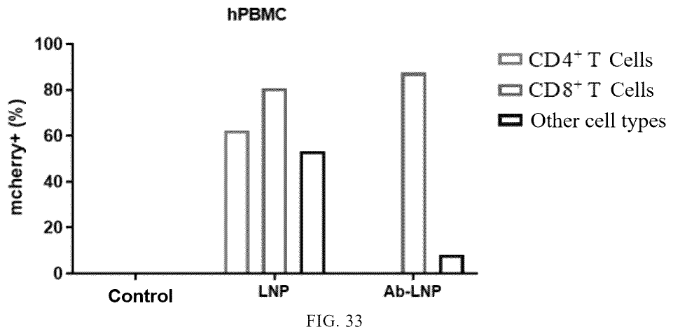

The results of Ab-LNP transfection of hPBMC are shown in FIG. 33. Based on the transfection results of LNP and Ab-LNP in hPBMC, the LNP of the present invention exhibits excellent transfection efficiency in immune cells. Compared to LNP without antibody modification, Ab-LNP demonstrates superior and more precise targeting capability toward CD8+ T cells, reaching up to 86%.

All documents mentioned in the present disclosure are incorporated herein by reference, as if each individual document were specifically and individually indicated to be incorporated by reference. In addition, it should be understood that, after reading the above teachings of the present disclosure, those skilled in the art can make various alterations or modifications to the present disclosure, and such equivalent forms shall also fall within the scope defined by the appended claims of the present disclosure.

What is claimed is:

1. An ionizable lipid, or a pharmaceutically acceptable salt, a tautomer or a stereoisomer thereof, wherein, the ionizable lipid has the structure of Formula I below: 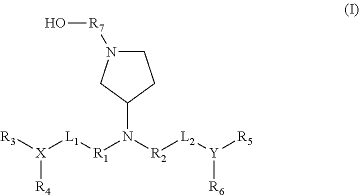
wherein,
R₁ and R₂ are each independently selected from —(CH₂)ₙ—, wherein n is a positive integer ranging from 1 to 14;
X and Y are each independently —CH— or N;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃, R₄, R₅ and R₆ are each independently H, CH₃, C₂-C₃₀ hydrocarbyl group, or —(CH₂)ₛ—R^{a}—(CH₂)_{g}—R^{b}—(CH₂)ₘ—CH₃, wherein, s, g are each independently selected from a positive integer ranging from 1 to 20, m is selected from a integer ranging from 0 to 20;
R^{a}, R^{b} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—;
and, R₃ and R₄ are not both H; and R₅ and R₆ are not both H;
R₇ is selected from C₁-C₅ hydrocarbyl group, or —(CH₂)ₘO(CH₂)ₙ—, wherein m, n are each independently selected from 1, 2, 3.

2. The ionizable lipid, or pharmaceutically acceptable salt, tautomer or stereoisomer thereof according to claim 1, wherein, the ionizable lipid has the structure represented by Formula (I-1) 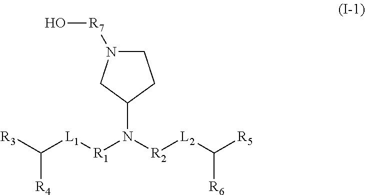
wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃, R₄, R₅ and R₆ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3;
preferably, L₁ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—;
L₂ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—;
R₃, R₄, R₅ and R₆ are each independently selected from: H, C₂-C₂₀ hydrocarbyl group;
R₇ is selected form C₁-C₅ hydrocarbyl group;
preferably, n is selected from 1, 2, 3, 4, 5, 6, 7, 8, 9, 10, 11, 12, 13, 14;
preferably, n is selected from an integer ranging from 5 to 8, such as 5, 6, 7, 8;
preferably, L₁ is selected from: —(C═O)O—, —O(C═O)—, —(C═O)NH—, —NH(C═O)—;
preferably, L₂ is selected from: —(C═O)O—, —O(C═O)—, —(C═O)NH—, —NH(C═O)—;
preferably, R₇ is C₂-C₄ hydrocarbyl group.

3. The ionizable lipid, or pharmaceutically acceptable salt, tautomer or stereoisomer thereof according to claim 1, wherein, the ionizable lipid has the structure represented by Formula (I-2) below: 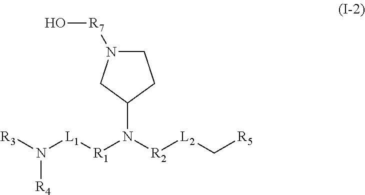
wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ, R₅ has the structure of —R₅ₐ—R_{5b}—R_{5c}—R_{5d}—R₅ₑ;
wherein, R₃ₐ, R_{3c}, R₄ₐ, R_{4c}, R₅ₐ, R_{5c} are each independently —(CH₂)ₙ—, wherein n is selected from a positive integer ranging from 1 to 14;
R_{3b}, R_{3d}, R_{4b}, R_{4d}, R_{5b}, R_{5d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—;
R₃ₑ, R₄ₑ, R₅ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

4. The ionizable lipid, or pharmaceutically acceptable salt, tautomer or stereoisomer thereof according to claim 1, wherein, the ionizable lipid has the structure represented by Formula (I-3) below: 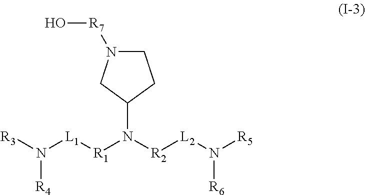
wherein,
R₁ and R₂ are each independently selected from —(CH₂)ₙ—, wherein n is a positive integer ranging from 1 to 14;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14;
R_{3b}, R_{3d}, R_{4b}, R_{4d}, R_{5b}, R_{5d}, R_{6b} and R_{6d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—;
R₃ₑ, R₄ₑ, R₅ₑ, R₆ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

5. The ionizable lipid, or pharmaceutically acceptable salt, tautomer or stereoisomer thereof according to claim 1, wherein, R₃, R₄, R₅ and R₆ are each independently C₅-C₁₅ alkyl.

6. The ionizable lipid, or pharmaceutically acceptable salt, tautomer or stereoisomer thereof according to claim 1, wherein, the ionizable lipid has the structure represented by Formula (I-1) below: 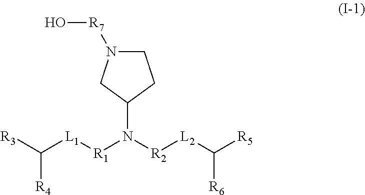
wherein,
R₁ and R₂ are each independently selected from —(CH₂)ₙ—, wherein n is a positive integer ranging from 4 to 8;
L₁ is selected from the following functional groups: —O—, —(C═O)O—, —O(C═O)—, —CH(OH)—;
L₂ is selected from the following functional groups: —O—, —(C═O)O—, —O(C═O)—, —CH(OH)—;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14; and R₃, R₄, R₅ and R₆ each independently has 4-20 CH₂ structural moieties;
R_{3b}, R_{3d}, R_{4b}, R_{4d}, R_{5b}, R_{5d}, R_{6b} and R_{6d} are each independently absent or selected from the following functional groups: —(C═O)O—, —O(C═O)—, —(S—S)—, —(C═C)—(CH₂)—(C═C)—, —CH(OH)—;
R₃ₑ, R₄ₑ, R₅ₑ, R₆ₑ are each independently C₂-C₂₀ hydrocarbyl group;
R₇ is C₁-C₃ hydrocarbyl group, or —(CH₂)₂—O—(CH₂)₂—.

7. The ionizable lipid, or pharmaceutically acceptable salt, tautomer or stereoisomer thereof according to claim 1, wherein, the ionizable lipid has the structure represented by Table 2a, Table 2b or the Formula below:

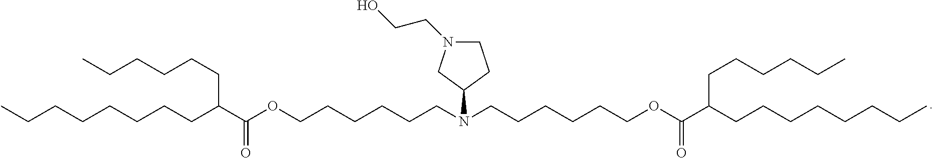 [SMILES: CCCCCCCCC(CCCCCC)C(=O)OCCCCCCN(CCCCCCOC(=O)C(CCCCCC)CCCCCCCC)[C@@H]1CCN(CCO)C1]

8. A method for preparing the ionizable lipid, or pharmaceutically acceptable salt, tautomer or stereoisomer thereof according to claim 1, wherein, the method comprises: method I, method II and method III;
wherein, method I comprises the following steps:
(S1) under the protection of an inert gas, compounds A1 and A2 are reacted to obtain compound A3;
(S2) under the protection of an inert gas, the tert-butoxycarbonyl (Boc) group of A3 is removed to obtain A4;
(S3) under the protection of an inert gas, A4 is reacted with A5 or A6 to obtain A7 (i.e., the compound represented by (I-1)); 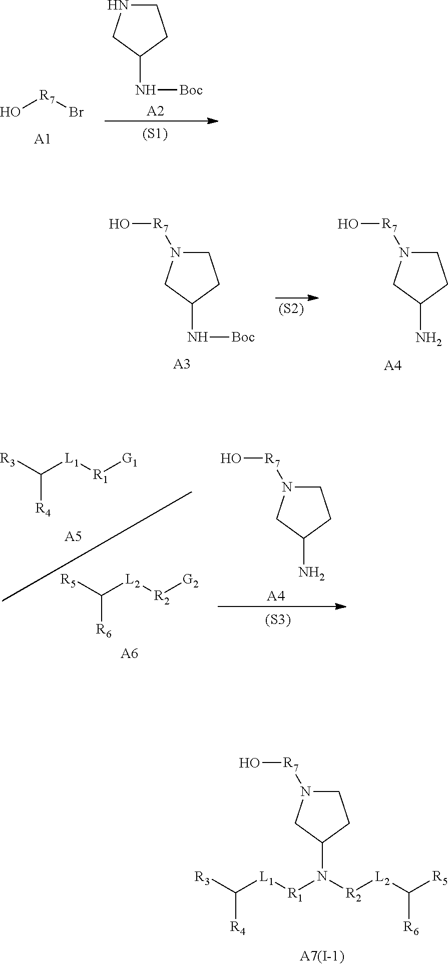
wherein, R₁ and R₂ are each independently selected from —(CH₂)ₙ—, wherein n is a positive integer ranging from 1 to 14;
G₁ and G₂ are each independently selected from an active functional group;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: O, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃, R₄, R₅ and R₆ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3;
preferably, R₁ and R₂ are each independently —(CH₂)ₙ—, wherein n is an integer ranging from 1 to 14, prefer an integer ranging from 5-8;
G₁ and G₂ are each independently selected from aldehyde group, halogen;
L₁ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, preferably selected from —(C═O)O—, —O(C═O)—, —(C═O)NH—, —NH(C═O)—;
L₂ is selected from: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; preferably selected from —(C═O)O—, —O(C═O)—, —(C═O)NH—, —NH(C═O)—;
R₃, R₄, R₅ and R₆ are each independently selected from: H, C₂-C₂₀ hydrocarbyl group;
R₇ is selected form C₁-C₅ hydrocarbyl group, preferably selected from C₂-C₄ hydrocarbyl group;
method II comprises the following steps:
(D1) under the protection of an inert gas, compound B1 is reacted with B2 to obtain compound B3;
(D2) under the protection of an inert gas, compound B3 is reacted with B4 to obtain compound B5;
(D3) under the protection of an inert gas, compound B5 is reacted with B6 to obtain compound B7, i.e., the compound represented by (I-2); 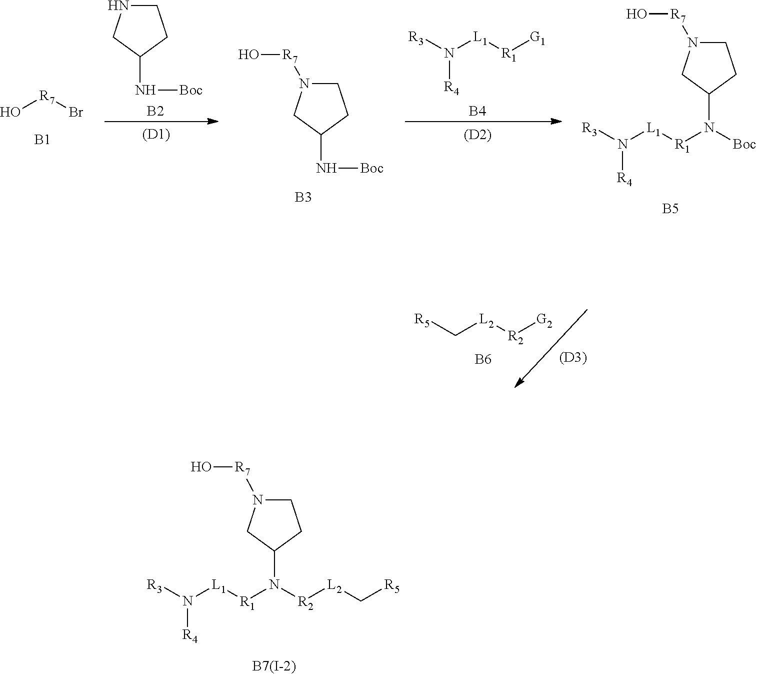
wherein, R₁ and R₂ are each independently selected from —(CH₂)ₙ—, wherein n is a positive integer ranging from 1 to 14;
G₁ and G₂ are each independently selected from an active functional group;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ;
wherein, R₃ₐ, R_{3c}, R₄ₐ, R_{4c}, R₅ₐ, R_{5c} are each independently —(CH₂)ₙ—, wherein n is selected from a positive integer ranging from 1 to 14;
R_{3b}, R_{3d}, R_{4b}, R_{4d}, R_{5b}, R_{5d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—;
R₃ₑ, R₄ₑ, R₅ₑ are each independently C₂-C₂₀ hydrocarbyl group;
R₆ is H;
R₇ is selected from C₁-C₅ hydrocarbyl group, or —(CH₂)ₘO(CH₂)ₙ—, wherein m, n are each independently selected from 1, 2, 3;
method III comprises the following steps:
(M1) under the protection of an inert gas, compounds C1 and C2 are reacted to obtain C3;
(M2) under the protection of an inert gas, compound C3 undergoes de-Boc deprotection to obtain compound C4;
(M3) under the protection of an inert gas, compound C4 is reacted with C5 or C6 to obtain compound C7, i.e., the compound represented by Formula (I-3); 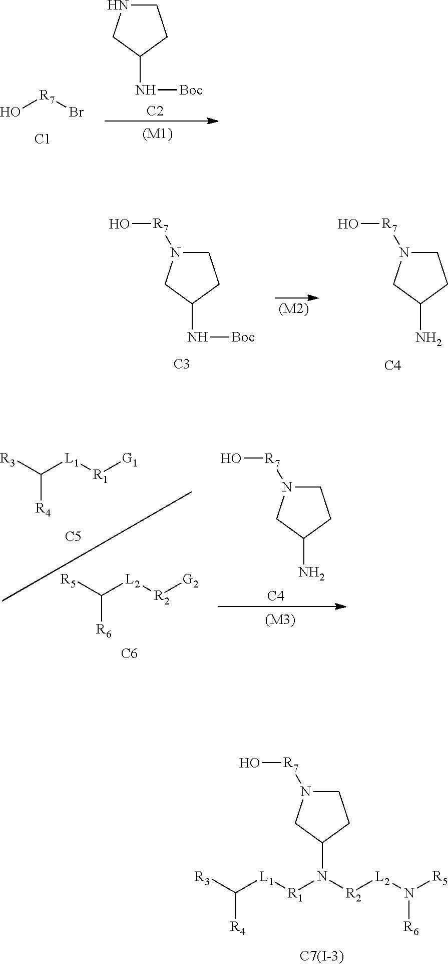
wherein, R₁ and R₂ are each independently selected from —(CH₂)ₙ—, wherein n is a positive integer ranging from 1 to 14;
G₁ and G₂ are each independently selected from an active functional group;
L₁ has the structure of -(L₁ₐ-L_{1b})- from right to left, or is absent, wherein, L₁ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{1b} is —(CH₂)ₙ—, wherein n is selected from 0, 1, 2, 3 or 4;
L₂ has the structure of -(L₂ₐ-L_{2b})- from left to right, or is absent; wherein, L₂ₐ is selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—; L_{2b} is —(CH₂)ₙ—, wherein n is selected from 0, 1, 2, 3 or 4;
R₃ has the structure of —R₃ₐ—R_{3b}—R_{3c}—R_{3d}—R₃ₑ, R₄ has the structure of —R₄ₐ—R_{4b}—R_{4c}—R_{4d}—R₄ₑ; R₅ has the structure of —R₅ₐ—R_{5b}—R_{5c}—R_{5d}—R₅ₑ, R₆ has the structure of —R₆ₐ—R_{6b}—R_{6c}—R_{6d}—R₆ₑ;
wherein, R₃ₐ, R_{3c}, R₄ₐ, R_{4c}, R₅ₐ, R_{5c}, R₆ₐ and R_{6c} are each independently —(CH₂)ₙ—, wherein n is selected from a positive integer ranging from 1 to 14;
R_{3b}, R_{3d}, R_{4b}, R_{4d}, R_{5b}, R_{5d}, R_{6b} and R_{6d} are each independently absent or selected from the following functional groups: —O—, —(C═O)O—, —O(C═O)—, —(S—S)—, —O(S═O)—, —(C═O)S—, —S(C═O)—, —(C═S)O—, —NH(C═O)—, —(C═S)NH—, —NH(C═S)—, —(C═O)NH—, —CH(OH)—, —(C═C)—(CH₂)—(C═C)—, —(C═C)—, —CH₂—, —(C≡C)—;
R₃ₑ, R₄ₑ, R₅ₑ, R₆ₑ are each independently C₂-C₂₀ hydrocarbyl group;
R₇ is selected from C₁-C₅ hydrocarbyl group, or —(CH₂)ₘO(CH₂)ₙ—, wherein m, n are each independently selected from 1, 2, 3.

9. A lipid nanoparticle (LNP), wherein, the lipid nanoparticle comprises the ionizable lipid, or pharmaceutically acceptable salt, tautomer or stereoisomer thereof according to claim 1;
preferably, the content of the ionizable lipid accounts for 30-65 mol % of the total lipid content in the lipid nanoparticle;
preferably, the lipid nanoparticle further comprises an auxiliary lipid;
preferably, the auxiliary lipid includes helper phospholipids, sterols, polymer-conjugated lipids, or a combination thereof;
preferably, the auxiliary lipid is a combination of helper phospholipids, sterols and polymer-conjugated lipids;
preferably, the helper phospholipid is preferably selected from: 1,2-distearoyl-sn-glycero-3-phosphocholine (DSPC), 1,2-dioleoyl-sn-glycero-3-phosphoethanolamine (DOPE), dioleoylphosphatidylcholine (DOPC), 1,2-dioleoyl-sn-glycero-3-phosphocholine, 1,2-dipalmitoyl-sn-glycero-3-phosphocholine, 1,2-dipalmitoyl-sn-glycero-3-phosphoethanolamine, 1,2-dimyristoyl-sn-glycero-3-phosphoethanolamine, sodium 1,2-dioleoyl-sn-glycero-3-phospho-rac-(1-glycerol), 1,2-dipalmitoylphosphatidylglycerol, 1-palmitoyl-2-oleoylphosphatidylcholine, 1-palmitoyl-2-oleoylphosphoethanolamine, distearoylphosphoethanolamine, 1-stearoyl-2-oleoylphosphatidylcholine, 1-stearoyl-2-oleoylphosphoethanolamine, or a combination thereof,
preferably, the sterol includes cholesterol or cholesterol derivatives;
preferably, the polymer-conjugated lipid is pegylated (PEG) lipids;
preferably, the pegylated lipid is preferably selected from the following group: DMG-PEG2000, DSPE-PEG2000, DSG-PEG2000, DSPE-PEG-Mannose, DMG-PEG2000-(polypeptides, proteins, amino acids, vitamins and the other active substances), or a combination thereof,
preferably, the lipid nanoparticle comprises an ionizable lipid, DSPC, cholesterol and DMG-PEG2000, wherein the molar ratio of the ionizable lipid:DSPC:cholesterol:DMG-PEG2000 is (30-65):(5-30):(30-55):(1-5), preferably (30-65):(10-30):(20-50):(1-5), (40-50):(10-15):(38-45):(1.5-2), (40-60):(10-20):(25-45):(1-2) or (40-60):(10-20):(28.5-38.5):1.5.

10. A lipid nanoparticle pharmaceutical formulation, wherein, the lipid nanoparticle pharmaceutical formulation comprises:
i) the lipid nanoparticle according to claim 9;
ii) a biologically active substance encapsulated in the lipid nanoparticle; and
iii) a pharmaceutically acceptable carrier.

11. A method for preparing the lipid nanoparticle pharmaceutical formulation according to claim 10, wherein, the method comprises:
(a) mixing the ionizable lipid, or pharmaceutically acceptable salt, tautomer or stereoisomer, and optional auxiliary lipids with an organic solvent to obtain a lipid organic phase;
(b) mixing a biologically active substance with an aqueous solvent to obtain an aqueous phase containing the biologically active substance;
(c) mixing the lipid organic phase in step (a) with the aqueous phase in step (b) to obtain the lipid nanoparticle pharmaceutical formulation;
wherein, the ionizable lipid has the structure of Formula I, Formula I-1, Formula I-2, or Formula I-3.

12. A drug delivery system, wherein the drug delivery system comprises the lipid nanoparticle according to claim 9, or the lipid nanoparticle pharmaceutical formulation comprises the lipid nanoparticle.

13. The lipid nanoparticle according to claim 9, the lipid nanoparticle pharmaceutical formulation comprises the lipid nanoparticle, or drug delivery system comprises the lipid nanoparticle or the lipid nanoparticle pharmaceutical formulation, wherein the lipid nanoparticle is modified with a targeting moiety;
preferably, the lipid nanoparticle modified with a targeting moiety comprises an ionizable lipid, DSPC, cholesterol, DSPE-PEG2000 or DMG-PEG2000, and DSPE-PEG5000-MAL conjugates (or targeting moieties), wherein the molar ratio is (30-65):(10-30):(20-50):(0.5-5):(0.1-2), preferably (40-60):(10-20):(25-45):(0.5-2):(0.1-2);
preferably, the lipid nanoparticle modified with a targeting moiety comprises an ionizable lipid, DSPC, cholesterol, DSPE-PEG2000 or DMG-PEG2000, and DSPE-PEG5000-MAL conjugates (or targeting moieties), wherein the molar ratio is (40-60):(10-20):(28.5-38.5):(0.5-1.5):(0.1-1);
preferably, the targeting moiety is selected from at least one from: ligands, receptors, antibodies, antigen-binding fragments of antibodies, aptamers, and polypeptides;
preferably, the targeting moiety comprises antibodies and antigen-binding fragments thereof that target the target protein;
preferably, the antibodies comprise monoclonal antibodies and polyclonal antibodies;
preferably, the antibodies and antigen-binding fragments thereof are selected from: an intact antibody, a nanobody (VHH), a Fab fragment, a Fab′ fragment, an F(ab′)2 fragment, an F(ab′)3 fragment, Fv, a single-chain variable fragment (“scFv”), a di-scFv, and a (scFv)2;
preferably, the modification comprises covalent conjugation, non-covalent mixing, and/or other chemical bonding interactions;
preferably, the target protein is an immune cell surface protein;
preferably, the immune cells include T cells, B cells, NK cells, or a combination thereof;
preferably, the T cells are selected from: primary human T cells, JM cells, Jurkat cells, or a combination thereof,
preferably, the primary human T cells comprise helper T cells, cytotoxic T cells, regulatory T cells, memory T cells;
preferably, the target protein is a cell surface protein selected from: CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR1, CCR2, CCR4, CCR6, CCR7, or a combination thereof,
preferably, the target protein is a cell surface protein selected from: CD3, CD4, CD5, CD8, CD7, CD28, CD45, CD2, or a combination thereof.

14. A method for treating and/or preventing a tumor, an infectious disease and a rare disease, comprising administering the lipid nanoparticle according to claim 9 to a subject in need thereof.

15. A method for treating and/or preventing a tumor, an infectious disease and a rare disease, comprising administering the lipid nanoparticle pharmaceutical formulation according to claim 10 to a subject in need thereof.
